(19) United States
(12) Patent Application Publication
(10) Pub. No.: US 2024/0092798 A1
(43) Pub. Date: Mar. 21, 2024

(54) PROCESS FOR THE PREPARATION OF ERIBULIN

(71) Applicant: FineTech Pharmaceutical Ltd., Nesher (IL)

(72) Inventors: Igor RUKHMAN, Yoqneam Illit (IL); Igor ZALTSMAN, Haifa (IL); Lev YUDOVICH, Haifa (IL); Olga GROSSMAN, Kibbutz Adamit (IL); Irina FEDOTEV, Nesher (IL); Arie GUTMAN, Nesher (IL)

(73) Assignee: FineTech Pharmaceutical Ltd., Nesher (IL)

(21) Appl. No.: 18/039,815

(22) PCT Filed: Dec. 2, 2021
(86) PCT No.: PCT/IL2021/051440

§ 371 Date: Jun. 1, 2023

(30) Foreign Application Priority Data
Dec. 2, 2020 (IL) 279168

(51) Int. Cl. C07D 493/22; C07D 405/14

(52) U.S. Cl. CPC C07D 493/22; C07D 405/14

(57) ABSTRACT

The present invention relates to a process for the preparation of Eribulin.

### FIELD OF THE INVENTION

This invention is directed to a process for preparation of Eribulin.

### BACKGROUND OF THE INVENTION

Eribulin is a synthetic macrocyclic ketone analog of halichondrin B with potent antiproliferative activity as an anticancer drug. Eribulin is marketed by Eisai Co, under the trade name Halaven and it is also known as E7389, B1939 and ER-086526.

A total synthesis of Halichondrin B was published in 1992 (Aicher, T. D. et al; J. Am. Chem. Soc. 114, 3162-3164). Eribulin was first reported in U.S. Pat. No. 6,214,865. Accordingly, new methods for the synthesis of halichondrin B analogs and particularly, Eribulin useful as an anti-cancer agent are desirable.

### SUMMARY OF THE INVENTION

The present invention is directed to a new process and intermediates for the preparation of Eribulin.

In one aspect, provided herein is a compound represented by the structure of Formula IV12:

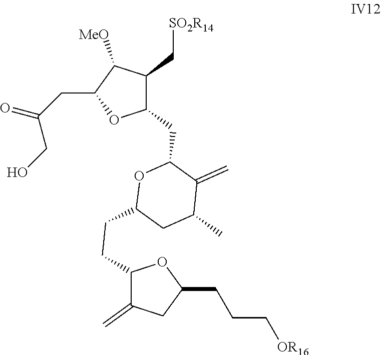

or isomer thereof,
wherein R₁₄ is an alkyl or an aryl; R₁₆ is an alcohol protecting group.

In some embodiments, provided herein the process for the preparation of preparation of a compound of Formula IV12; wherein the compound of Formula IV12 is prepared from a compound of Formula IV7:

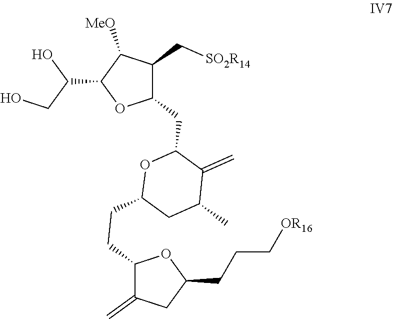

or isomer thereof,
wherein R₁₄ is an alkyl or an aryl; R₁₆ is an alcohol protecting group.

In some embodiments, provided herein is a process for the preparation of Eribulin, wherein the process comprises the following steps:
(i) preparing a compound of formula IV12:

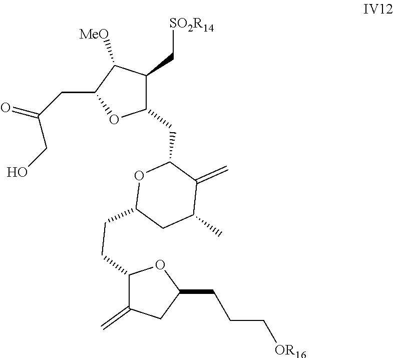

or isomer thereof,
wherein R₁₄ is an alkyl or an aryl; R₁₆ is an alcohol protecting group; from a compound of formula IV7:

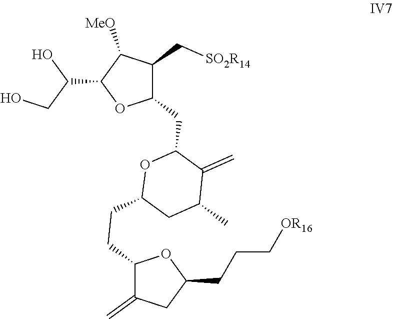

or isomer thereof,
wherein R₁₄ is an alkyl or an aryl; R₁₆ is an alcohol protecting group.
by elongating the chain of the diol; and
(ii) preparing Eribulin from a compound of formula IV12, wherein the process comprises reducing the ketone group of IV12 to form diol, protecting the diol group and coupling reaction to obtain Eribulin.

In some embodiments, provided herein is a compound represented by the structure of Formula IV7:

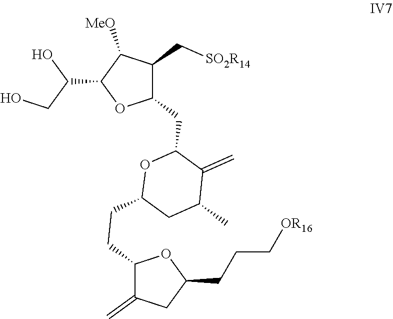

or isomer thereof,
wherein R₁₄ is an alkyl or an aryl; R₁₆ is an alcohol protecting group

In some embodiments, provided herein is a compound represented by the structure of Formula III12:

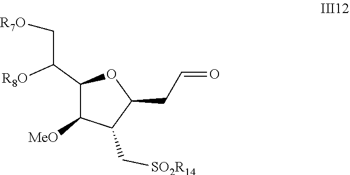

or isomer thereof,
wherein R₇ and R₈ are each independently an alcohol protecting group or R₇ and R₈ form together with the oxygen a 5-6 member ring optionally substituted; and R₁₄ is an alkyl or an aryl.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 presents a synthetic scheme for the preparation of Compound A12 via Compound A7(S) as an intermediate. The processes from A4(S) to A11 are processes of this invention. The processes from Diacetone-D-Glucose to A4, and from A11 to A12—were prepared according to a process described in references [1-6].

FIG. 2 presents a synthetic scheme for the preparation of Compound A12 via Compound A7(R). The processes from A14 to A11 are the processes of this invention. The processes from A2 to A14, and from A11 to A12—were prepared according to a process described in references [1-6].

FIG. 3 presents a synthetic scheme for alternative way for the preparation of Compound A7(R). These processes are processes of this invention.

FIG. 4 presents a synthetic scheme for the preparation of Compound A29 from Compound A12. Compound A29 was prepared according to a process described in references [7-10].

FIG. 5 presents a synthetic scheme for the preparation of Compound B14. Compound B14 was prepared according to a process described in references [11-14]. The purification of Compound B14, is a process of this invention, achieved by crystallization and obtained with more than 99% de.

FIG. 6 presents a synthetic scheme for the preparation of Compound B20 from Compound B15 and Compound B14. Compound B20 was prepared according to a process described in references [15-18].

FIG. 7 presents a synthetic scheme for the preparation of Compound B28(1). The process from Compound B25 to Compound B28(1) is a process of this invention. The process from Compound B21 to Compound B25 was prepared according to a process described in references [19-30].

FIG. 8 presents a synthetic scheme for the preparation of Compound B28(2). See references [31-35]. The process from Compound B30 to Compound B31 is a process of this invention.

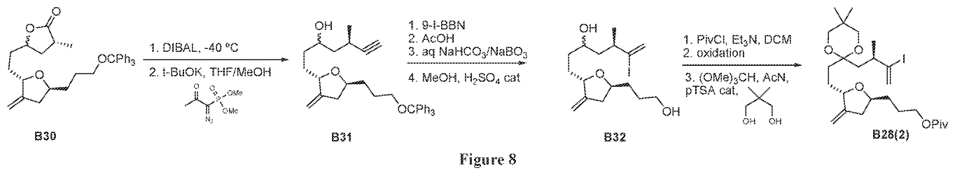

FIG. 9 presents a synthetic scheme for the preparation of Compound C12. The process from Compound C4 to Compound C12 is a process of this invention. The process from Diacetone-D-glucose to Compound C4 was prepared according to a process described in references [36-39].

FIG. 10 presents a synthetic scheme for the preparation of Compound D15. The process from Compounds C12 and B20 to obtain Compound D13 is a process of this invention. The process from Compound D13 to obtain Compound D15 was prepared according to a process described in references [36-39].

FIG. 11 presents a synthetic scheme for the preparation of Compound D6.

FIG. 12 presents a synthetic scheme of for the preparation of Compound D7.

FIG. 13 presents a synthetic scheme of for the preparation of Eribulinfrom the reaction of D15 and A29 was prepared according to a process described in references [36-39].

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### REFERENCES

[1] Boeckel C. A. A. et al; Journal of Carbohydrate Chemistry, 4, 293-321.
[2] Tatai, J. et al: Carbohydrate Research, 2008, 343(4), 596-606.
[3] Del Carmen Cruzado, M. & Martin-Lomas M., Tetrahedron Letters, 1986, 27(22), 2497-2500.
[4] Zou, W & Veembaiyan, K., Journal of Organic Chemistry, 2013, 78(6), 2703-2709.
[5] Hager, D. et al; Angewandte Chemie, International Ed., 2012, 51(26), 6525-6528.
[6] Fleming, I. et al; Organic Reactions (Hoboken NJ, US), 1989, 37, 53.
[7] Austad, B. C. et al; Synlett, 2013, 24(3), 333-337 and references therein.
[8] PCT Publication No. WO 2005118565.
[9] PCT Publication No. WO 2013142999.
[10] Choi, H-W. et al; Pure and Applied Chemistry, 203, 73(1), 1-17.
[11] Capobianco, M. et al; Tetrahedron Letters, 1986, 27(12), 1387-90.
[12] Sayyad, A. A. & Kaliappan, K. European Journal of Organic Chemistry, 2017, 34, 5055-5065.
[13] Mallareddy, K. et al; Tetrahedron, 1996, 52(25), 8535-8544.
[14] PCT Publication No. WO 2014183211.
[15] Jakson, K. L. et al; Angewandte Chemie, International Ed., 2009, 48(13), 2346.
[16] Kim, D.-S. et al; Journal of the American Chemical Society, 2009, 131(49), 15636-41.
[17] Choi, H-W. et al; Organic Letters 2002, 4(25), 4435-38.
[18] Guo, H. et al; Journal of the American Chemical Society, 2009, 131(42), 15387-93.
[19] Lang, J.-H. & Lindel, T., Beilstein Journal of Organic Chemistry, 2019, 15, 577-583.
[20] Hugelshofer, C. L et al; Chemistry A-European Journal, 2016, 22(42), 15125-36.
[21] Price, G. A et al; Catalysis Science & Technology, 2016, 6(13), 4733-42.
[22] Edeson, S. J. et al; European Journal of Organic Chemistry, 2016, 1, 83-86.
[23] Wang, J.-X. et al; Synthesis, 2003, 10, 1506-10.
[24] Charette, A. et al; Journal of the American Chemical Society, 1998, 120(20), 5114-15.
[25] Ochiai, H. et al; Journal of Organic Chemistry, 1988, 53(6), 1343-4.
[26] PCT Publication No. WO 2019009956.
[27] Yahata, K. et al; Journal of Organic Chemistry, 2017, 82(17), 8792-8807.
[28] PCT Publication No. WO 2016176560.
[29] Ueda, A. et al; Journal of American Chemical Society, 2014, 136(13), 5171-76.
[30] Valot, G. et al; Chemistry A-European Journal, 2015, 21(6), 2398-2408.
[31] PCT Publication No. WO20070244187.
[32] Trost, B. M. et al; Journal of American Chemical Society, 2004, 126, 13618-19.
[33] Tan, D.-X. et al; European Journal of Organic Chemistry, 2016, 5, 946-57.
[34] Bedel, O. et al; Synthetic Letters, 2005, 15, 2313-16.
[35] Ramana, C. V. et al; Journal of Organic Chemistry, 2008, 73(10), 3915.
[36] Austad, B. C. et al; Synlett, 2013, 24, 327-332.
[37] Austad, B. C. et al; Synlett, 2013, 24, 333-337.
[38] PCT Publication No. WO199965894.
[39] PCT Publication No. WO2005118565.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, provided herein is a process for the preparation of Eribulin, wherein the process comprises preparing Eribulin from a compound of Formula IV12:

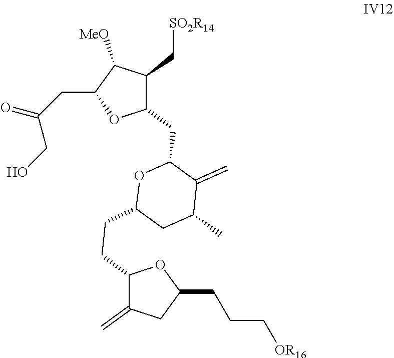

or isomer thereof,
wherein R₁₄ is an alkyl or an aryl; R₁₆ is an alcohol protecting group;
wherein the compound of Formula IV12 is prepared from a compound of Formula III12:

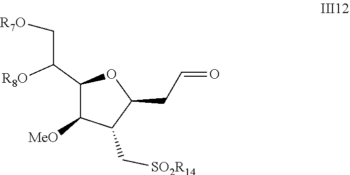

or isomer thereof,
wherein R₇ and R₈ are each independently an alcohol protecting group or R₇ and R₈ form together with the oxygen a 5-6 member ring optionally substituted; and R₁₄ is an alkyl or an aryl.

In some embodiments, a process for the preparation of Eribulin, wherein the process comprises preparing Eribulin from a compound of formula IV12:

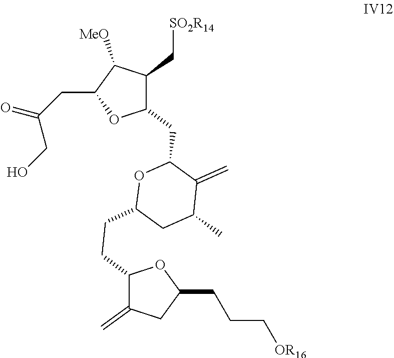

or isomer thereof,
wherein R₁₄ is an alkyl or an aryl; R₁₆ is an alcohol protecting group;
wherein the compound of Formula IV12 is prepared from a compound of formula IV7:

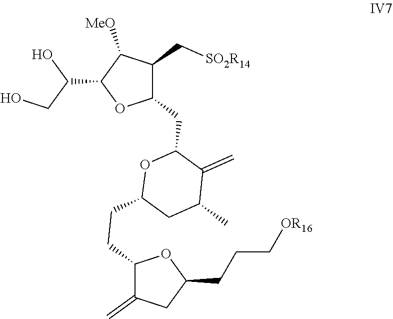

or isomer thereof,
wherein R₁₄ is an alkyl or an aryl; R₁₆ is an alcohol protecting group.

In some embodiments, a process for the preparation of Eribulin, wherein the process comprises preparing Eribulin from a compound of formula IV12:

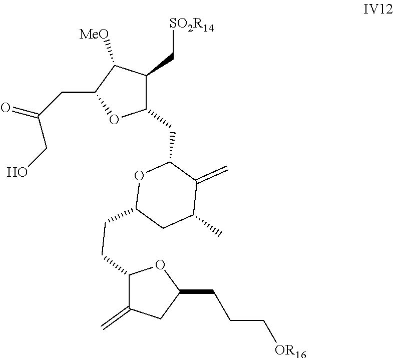

or isomer thereof,
wherein R₁₄ is an alkyl or an aryl; R₁₆ is an alcohol protecting group;
wherein the compound of Formula IV12 is prepared from a compound of formula IV6:

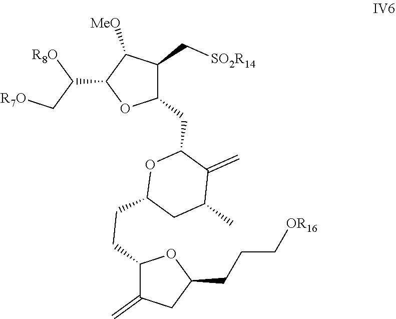

or isomer thereof,
wherein R₇ and R₈ are each independently an alcohol protecting group; or R₇ and R₈ form together with the oxygen a 5-6-member ring optionally substituted; and R₁₆ is an alcohol protecting group.

In some embodiments, provided herein is a process for the preparation of Eribulin, wherein the process comprises preparing Eribulin from a compound of Formula IV12.

In some embodiments, provided herein is a process for the preparation of a compound of Formula IV12 from a compound of Formula IV7.

In some embodiments, provided herein is a process for the preparation of a compound of Formula IV12 from a compound of Formula IV6.

In some embodiments, provided herein is a process for the preparation of Eribulin, wherein the process comprises preparing Eribulin from a compound of Formula III12.

In some embodiments, provided herein is a process for the preparation of a compound of Formula III12 from a compound of Formula III4.

In some embodiments, provided herein is a process for the preparation of a compound of Formula IV7 from a compound of Formula III12.

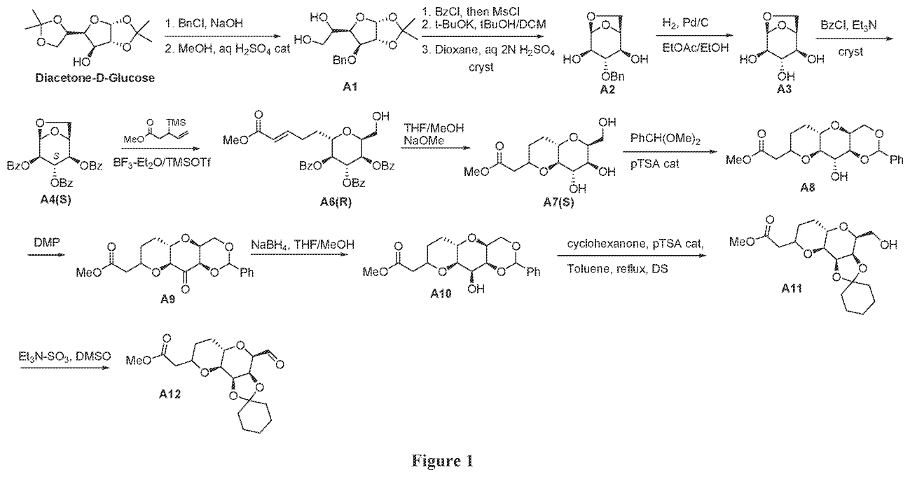

In some embodiments, provided herein is a process for the preparation of a compound of Formula I10(S) (see also FIG. 1):

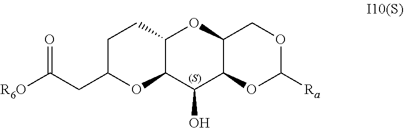

or isomer thereof,
wherein R₆ is an alkyl; and Rₐ is substituted or unsubstituted alkyl, substituted or unsubstituted aryl, or substituted or unsubstituted heteroaryl,
wherein the process (Process 1) comprises the following steps:
a) preparing a compound of Formula I6(R) or isomer thereof, from a compound of Formula 14 or isomer thereof,
according to scheme 1;

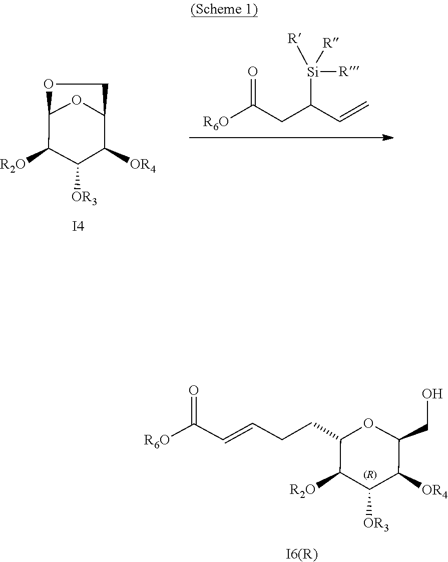

wherein R₂ and R₄ are each independently an acyl group; R₃ is an acyl group or benzyl; R₆ is an alkyl; R′, R″, and R′″ are each independently an alkyl or an aryl;
b) preparing a compound of Formula I7(S) or isomer thereof from a compound of Formula I6(R) or isomer thereof according to scheme 2;

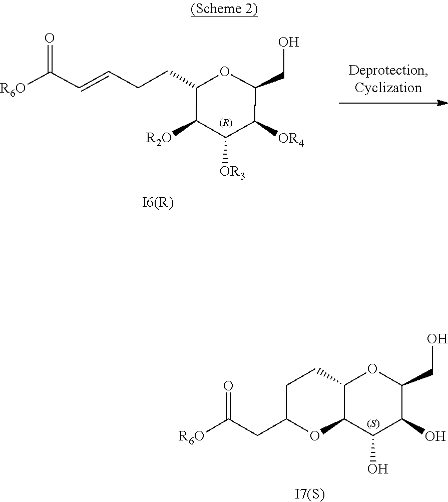

wherein R₂ and R₄ are each independently an acyl group; R₃ is an acyl group or benzyl; and R₆ is an alkyl;
c) preparing a compound of Formula I8(R) or isomer thereof from a compound of Formula I7(S) or isomer thereof according to scheme 3;

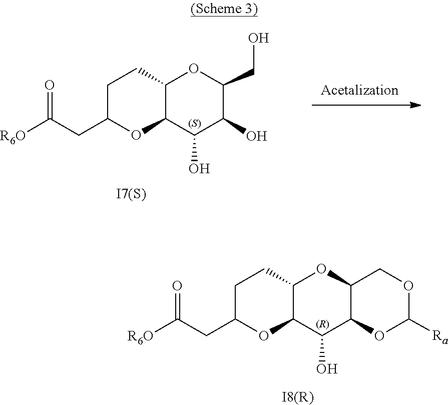

wherein Rₐ is substituted or unsubstituted alkyl, substituted or unsubstituted aryl, or substituted or unsubstituted heteroaryl; and R₆ is an alkyl;
d) preparing a compound of Formula I9 or isomer thereof from a compound of Formula I8(R) or isomer thereof according to scheme 4;

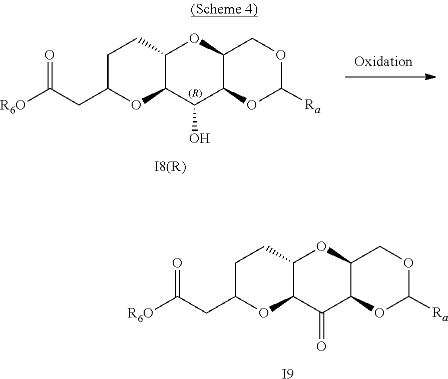

wherein Rₐ is substituted or unsubstituted alkyl, substituted or unsubstituted aryl, or substituted or unsubstituted heteroaryl; and R₆ is an alkyl; and
e) preparing a compound of Formula I10(S) or isomer thereof from a compound of Formula I9 or isomer thereof according to scheme 5:

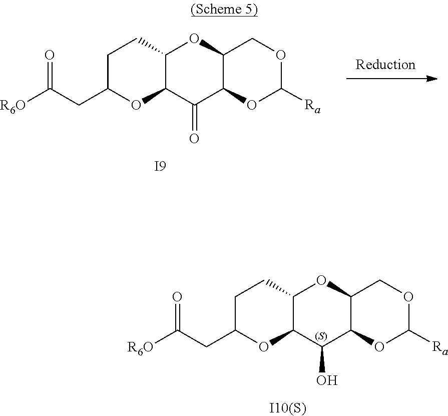

wherein Rₐ is substituted or unsubstituted alkyl, substituted or unsubstituted aryl, or substituted or unsubstituted heteroaryl; and R₆ is an alkyl.

In one embodiment, R′, R″ and R′″ of scheme 1 are each independently a methyl.

In one embodiment, R₂ an R₄ of a compound of Formula I4 are each independently benzoyl group. In one embodiment, R₂ an R₄ of a compound of Formula I6(R) are each independently benzoyl group. In one embodiment, R₃ of a compound of Formula I4 is benzoyl. In another embodiment, R₃ is benzyl.

In one embodiment, R₃ of a compound of Formula I6(R) is benzoyl. In another embodiment, R₃ is benzyl.

In one embodiment, R₆ of a compound of Formula I6(R), I7(S), I8(R), I9 and I10 is an C1-C5 alkyl. Each represent a separate embodiment of this invention.

In another embodiment, R₆ of a compound of Formula I6(R), I7(S), I8(R), I9 and I10 is methyl. Each represent a separate embodiment of this invention.

In one embodiment, Rₐ of a compound of Formula I8(R), I9 and I10 is phenyl. Each represent a separate embodiment of this invention.

In some embodiments, this invention provides scheme 1 for the preparation of I6(R). In some embodiments, this invention provides scheme 2 for the preparation of I7(S). In some embodiments, this invention provides scheme 3 for the preparation of I8(R). In some embodiments, this invention provides scheme 4 for the preparation of I9. In some embodiments, this invention provides scheme 5 for the preparation of I10(S).

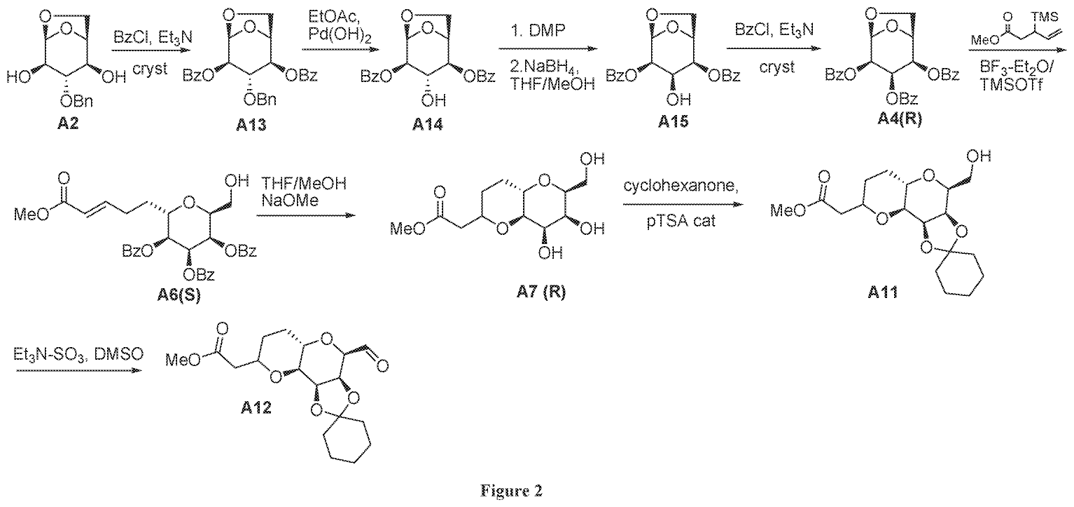

In some embodiments, provided herein is a process for the preparation of a compound of Formula I7(R) or isomer thereof (see also FIG. 2):

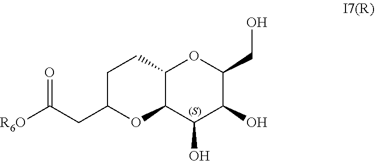

wherein R₆ is an alkyl group;
wherein the process (Process 2) comprises the following steps:
a) preparing a compound of Formula I15 or isomer thereof from a compound of Formula I14 or isomer thereof according to scheme 6;

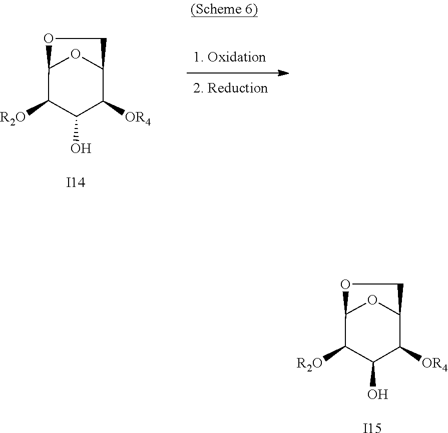

wherein R₂ and R₄ are each independently an acyl group;
b) preparing a compound of Formula I4(R) or isomer thereof from a compound of Formula I15 or isomer thereof according to scheme 7;

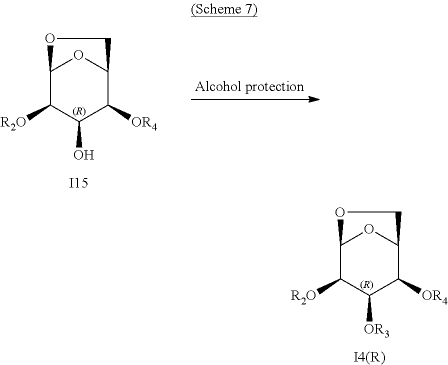

wherein R₂ and R₄ are each independently an acyl group; and R₃ is an acyl or a benzyl group;
c) preparing a compound of Formula I6(S) or isomer thereof from a compound of Formula I4 or isomer thereof according to scheme 8;

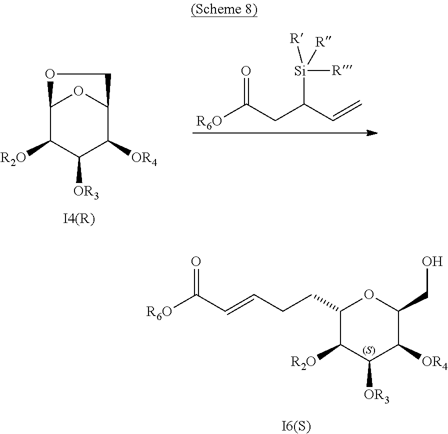

wherein R₂ and R₄ are each independently an acyl group; R₃ is an acyl or a benzyl group; R′, R″, and R′″ are each independently an alkyl or an aryl; and R₆ is an alkyl; and
d) preparing a compound of Formula I7(R) or isomer thereof from a compound of Formula I6(S) or isomer thereof according to scheme 9:

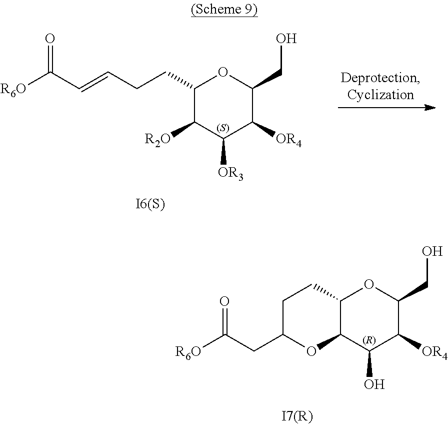

wherein R₂ and R₄ are each independently an acyl group; R₃ is an acyl or a benzyl group; and R₆ is an alkyl.

In another embodiment, R′, R″ and R′″ of scheme 8, are each independently a methyl.

In one embodiment, R₆ of a compound of Formula I7(R) is C1-C5 alkyl. In another embodiment, R₆ is methyl.

In one embodiment, R₂ an R₄ of a compound of Formula I14, I15, I4(R), I6(S) are each independently an acyl group. In another embodiment, R₂ an R₄ of a compound of Formula I14, I15, I4(R), I6(S) are each independently a benzoyl group. Each represent a separate embodiment of this invention.

In one embodiment, R₃ of a compound of Formula I4(R) is benzoyl. In another embodiment, R₃ is benzyl.

In one embodiment, R₃ of a compound of Formula I6(R) or I6(S) is benzoyl. In another embodiment, R₃ is benzyl. Each represent a separate embodiment of this invention.

In one embodiment, R₆ of a compound of scheme 8, and a compound of Formula I6(S), I7(R) is an C1-C5 alkyl. Each independently represent a separate embodiment of this invention. In another embodiment, R₆ is methyl. Each represent a separate embodiment of this invention. In one embodiment, FIG. 2 presents a process of this invention from a compound of formula I14 to I11.

In some embodiments, this invention provides scheme 6 for the preparation of I15 or isomer thereof. In some embodiments, this invention provides scheme 7 for the preparation of I4(R) or isomer thereof. In some embodiments, this invention provides scheme 8 for the preparation of I6(S) or isomer thereof. In some embodiments, this invention provides scheme 9 for the preparation of I7(R) or isomer thereof.

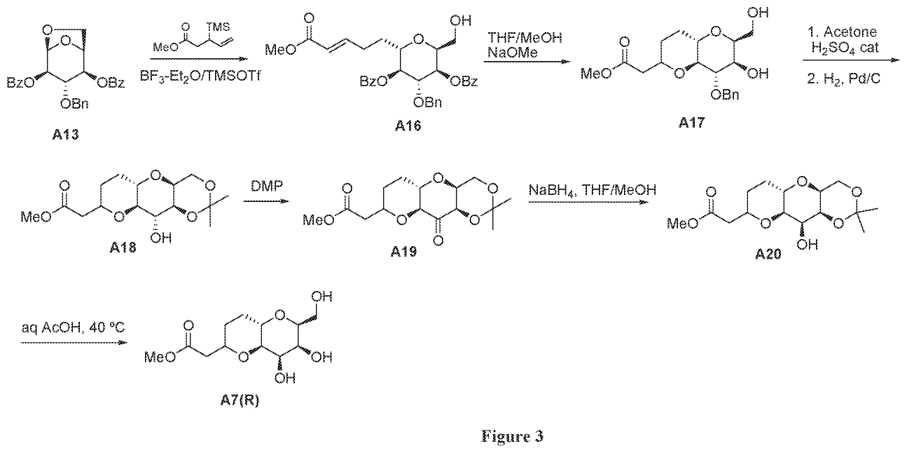

In some embodiments, provided herein is a process for the preparation of a compound of Formula I7(R) or isomer thereof (see also FIG. 3):

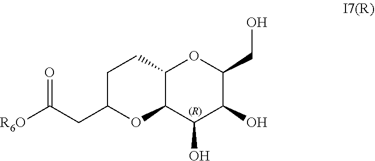

wherein R₆ is an alkyl group;
wherein the process (Process 3) comprises the following steps:
a) preparing a compound of Formula I16(R)(1) or isomer thereof from a compound of Formula I4(S)(1) or isomer thereof according to scheme 10;

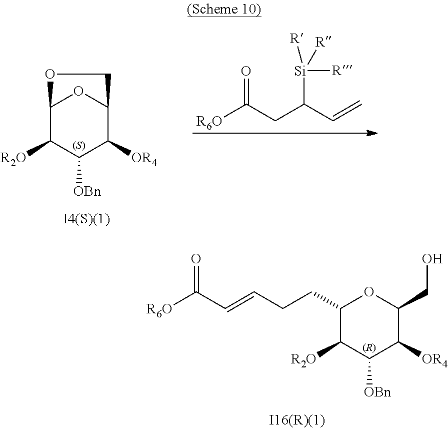

wherein R₂ and R₄ are each independently an acyl group; R′, R″, and R′″ are each independently an alkyl or an aryl; and R₆ is an alkyl;
b) preparing a compound of Formula I17(1) or isomer thereof from a compound of Formula I16(R)(1) or isomer thereof according to scheme 11;

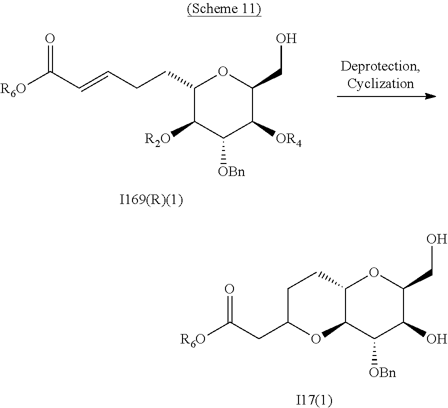

wherein R₂ and R₄ are each independently an acyl group, and R₆ is an alkyl;
c) preparing a compound of Formula I18 or isomer thereof from a compound of Formula I17(1) or isomer thereof according to scheme 12:

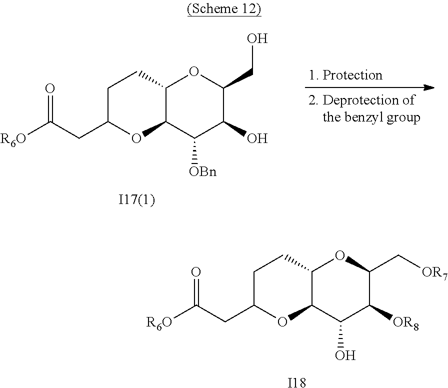

wherein R₇ and R₈ are each independently an alcohol protecting group or R₇ and R₈ form together with the oxygen a 5-6 member ring optionally substituted; R₆ is an alkyl;
d) preparing a compound of Formula I20 from a compound of Formula I18 according to scheme 13:

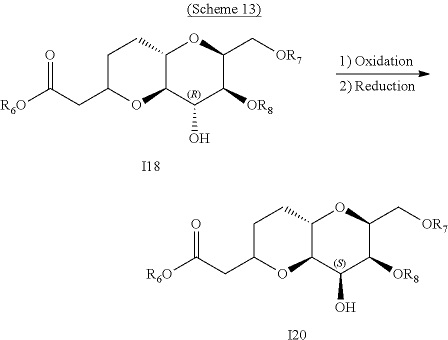

wherein R₇ and R₈ are each independently an alcohol protecting group or R₇ and R₈ form together with the oxygen a 5-6 member ring optionally substituted; R₆ is an alkyl;
wherein R₆ is as defined in step (a); R₇ and R₈ are as defined in step (c); and
e) preparing a compound of Formula I7(R) or isomer thereof from a compound of Formula I20 or isomer thereof according to scheme 14:

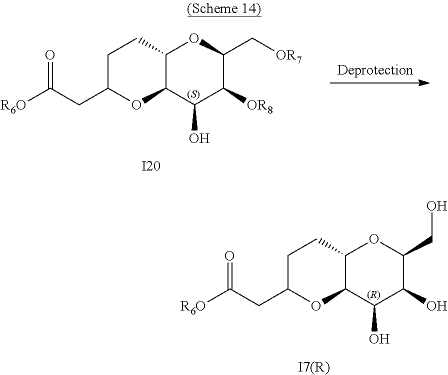

wherein R₆ is as defined in step (a); and R₇ and R₈ are as defined in step (c).

In another embodiment, R₇ and R₈ of Formula I18 or isomer thereof and I20 or isomer thereof are each independently stable to hydrogenation. In another embodiment, R₇ and R₈ form together 5-6-member ring. In another embodiment, R₇ and R₈ form together a 6 member ring as O—R_{b}—Si—R_{c}—O, wherein R_{b} and R_{c} are each independently an alkyl. In another embodiment, R₇ and R₈ of Formula I18 and I20 form together a 6-member ring as shown below:

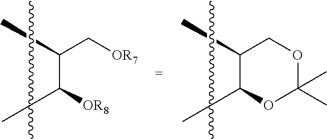

In another embodiment, R′, R″ and R′″ of scheme 10, are each independently a methyl. In one embodiment, R₆ of a compound of Formula I7(R), I16(R)(1) or I17(1) is C1-C5 alkyl. In another embodiment, R₆ is methyl.

In one embodiment, R₂ an R₄ of a compound of Formula I4(S)(1) or I16(R)(1) are each independently an acyl group. In another embodiment, R₂ an R₄ of a compound of Formula I4(S)(1) or I16(R)(1) are each independently a benzoyl group. Each represent a separate embodiment of this invention.

In one embodiment, R₃ of a compound of Formula I6(R) or I6(S) is benzoyl. In another embodiment, R₃ is benzyl. Each represent a separate embodiment of this invention.

In some embodiments, this invention provides scheme 10 for the preparation of I16(R)(1). In some embodiments, this invention provides scheme 11 for the preparation of I17(1). In some embodiments, this invention provides scheme 12 for the preparation of I18. In some embodiments, this invention provides scheme 13 for the preparation of I20. In some embodiments, this invention provides scheme 14 for the preparation of I7(R).

In some embodiments, provided herein is a process for the preparation of a compound of Formula II2 according to scheme 15 (see also in FIG. 1), scheme 16 (see also in FIG. 1) or scheme 17 (see also in FIG. 2):

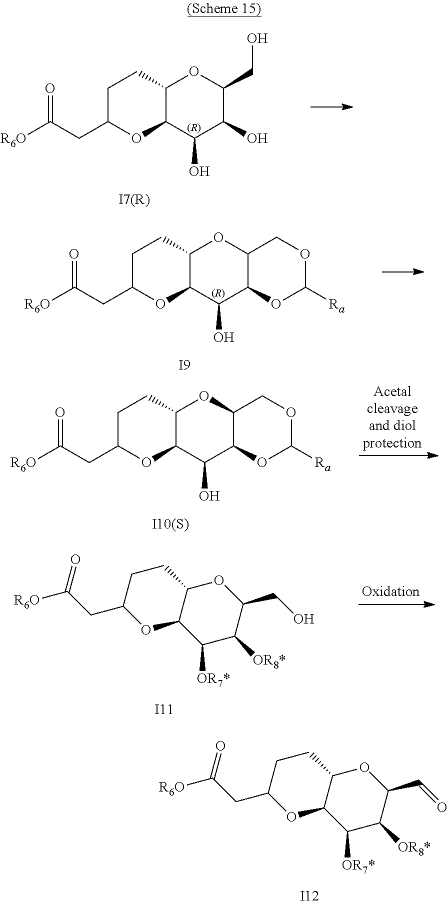

wherein R₇* and R₈* are each independently an alcohol protecting group or R₇* and R₈* form together with the oxygen a 5-6 member ring optionally substituted; R₆ is an alkyl; and Rₐ is substituted or unsubstituted alkyl, substituted or unsubstituted aryl, or substituted or unsubstituted heteroaryl;

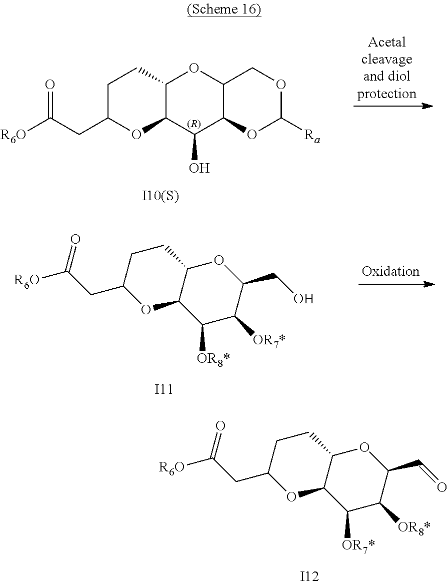

wherein R₇* and R₈* are each independently an alcohol protecting group; or R₇* and R₈* form together with the oxygen a 5-6 member ring optionally substituted; R₆ is an alkyl; and Rₐ is substituted or unsubstituted alkyl, substituted or unsubstituted aryl, or substituted or unsubstituted heteroaryl;

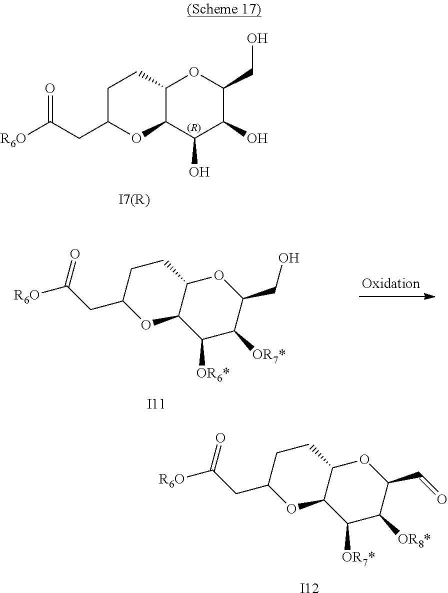

wherein R₇* and R₈* are each independently an alcohol protecting group; or R₇* and R₈* form together with the oxygen a 5-6 member ring optionally substituted; R₆ is an alkyl; and Rₐ is substituted or unsubstituted alkyl, substituted or unsubstituted aryl, or substituted or unsubstituted heteroaryl.

In another embodiment, R₇* and R₈* of Formula I11 and I12, form together with the oxygen a 5-6 member ring optionally substituted. In another embodiment, R₇* and R₈* form together a 5-member ring substituted with additional ring in a form of a spiro. In another embodiment, R₇* and R₈* of Formula I11 and I12, are as shown below:

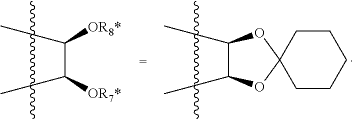

In another embodiment, a compound of Formula I7(R) or isomer thereof is prepared according to processes 2 and 3. In another embodiment, a compound of Formula I10(S) is prepared according to process 1. In another embodiment, a compound of Formula I10(S) or isomer thereof is prepared from a compound of I7(R) or isomer thereof. In one embodiment, the reaction conditions to obtain I11 or isomer thereof from I10 or isomer thereof as described in schemes 15 and 16 comprises acetal cleavage and diol protection. In another embodiment, the reaction condition comprises cyclohexanone and catalytic amount of acid. In another embodiment, the reaction condition comprises cyclohexanone and catalytic amount of p-TSA.

In some embodiments, provided herein is a process for the preparation of a compound of Formula I11 or isomer thereof according to scheme 15, scheme 16 or scheme 17.

In some embodiments, the process of the preparation of compound B14 or isomer thereof was prepared according to a process described in references [11-14].

In some embodiments, provided herein is a process for the purification of compound B14:

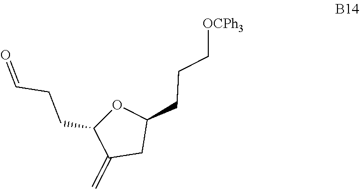

wherein the process comprises: crystallization in Heptane MTBE. In another embodiment, the purification of Compound B14 comprises dissolving the crude mixture of isomers in the mixture Heptane/MTBE at about 25-60° C., and cooled to about 0-30° C. In another embodiment, the purification of compound B14 comprises dissolving the crude mixture of isomers in the mixture Heptane/MTBE at about 30-50° C. and cooled to about 0-25° C. In another embodiment, the purification of compound B14 comprises dissolving the crude mixture of isomers in the mixture Heptane/MTBE at 40° C. and cooled to 25° C.

In another embodiment, the purification process comprises second crystallization in the same conditions.

In some embodiments, B14 was prepared according to known in the art as described in references [11-14].

In some embodiments, provided herein is a process for the preparation of a compound of Formula II28(1) or isomer thereof:

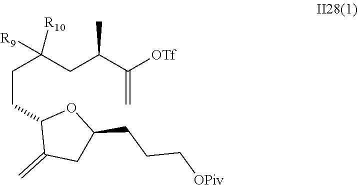

wherein R₉ and R₁₀ are each independently O-alkyl or S-alkyl; or R₉ and R₁₀ form together a 5-6-member acetal ring; and Piv refers to pivaloyl;
wherein the process (Process 4) comprises the following steps:
a) preparing Compound B26 or isomer thereof from Compound B25 or isomer thereof according to scheme 18;

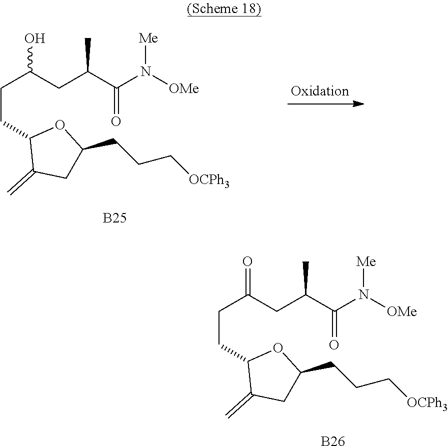

b) preparing a compound of Formula II27 or isomer thereof from Compound B26 or isomer thereof according to scheme 19;

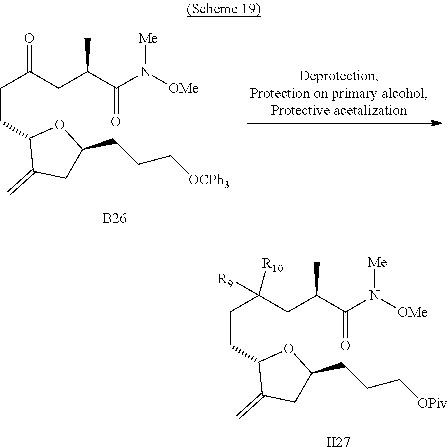

wherein R₉ and R₁₀ are each independently O-alkyl or S-alkyl; or R₉ and R₁₀ form together a 5-6-member acetal ring optionally substituted; and Piv refers to pivaloyl; and
c) preparing a compound of Formula II28 or isomer thereof from a compound of Formula I27 or isomer thereof according to scheme 20;

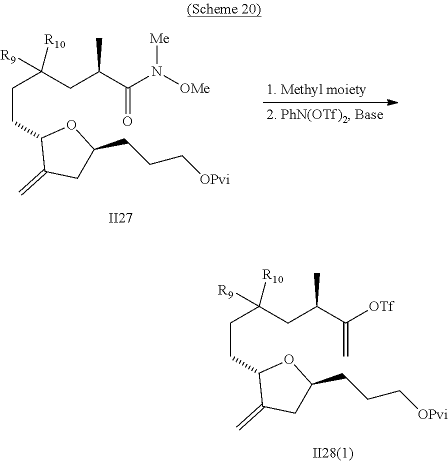

wherein R₉ and R₁₀ are each independently O-alkyl or S-alkyl; or R₉ and R₁₀ form together a 5-6-member acetal ring optionally substituted; and Piv refers to pivaloyl.

In another embodiment, R₉ and R₁₀ of Formula II27 or II28(1) are each independently O-alkyl or S-alkyl. In another embodiment, R₉ and R₁₀ form together a 5-6-member acetal ring optionally substituted. In another embodiment, R₉ and R₁₀ form together a 6-member acetal ring as shown.

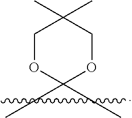

In one embodiment, methyl moiety of Process 4 step (c) refers to MeMgCl or MeLi.

In one embodiment, Process 4 step (c), scheme 20, comprises a base. In another embodiment, the base is LiHDMS or 2,6-Lutidine.

In some embodiments, this invention provides scheme 18 for the preparation of B26. In some embodiments, this invention provides scheme 19 for the preparation of II27. In some embodiments, this invention provides scheme 20 for the preparation of II28(1).

In some embodiments, provided herein is a process for the preparation of a compound of Formula II28(2) or isomer thereof:

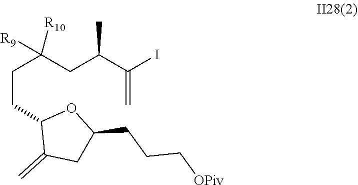

wherein R₉ and R₁₀ are each independently O-alkyl or S-alkyl; or R₉ and R₁₀ form together a 5-6-member acetal ring optionally substituted; and Piv refers to pivaloyl;
wherein the process (Process 5) comprises the following steps:
a) preparing Compound B31 or isomer thereof from Compound B30 or isomer thereof according to scheme 21;

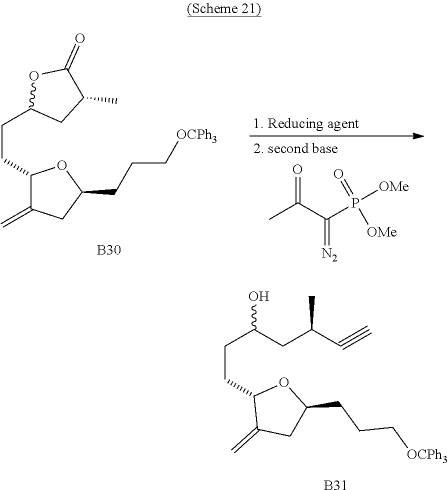

b) preparing Compound B32 or isomer thereof from Compound B31 or isomer thereof according to scheme 22;

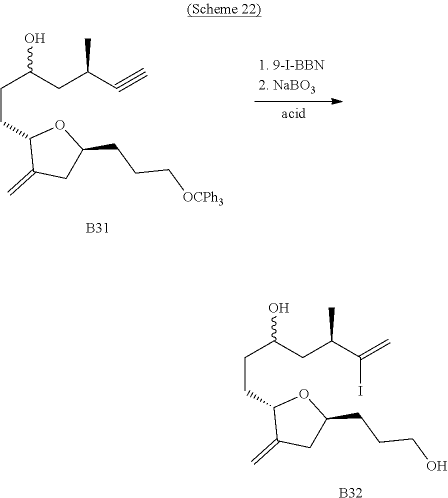

c) preparing a compound of Formula II28(2) or isomer thereof from Compound B32 or isomer thereof according to scheme 23;

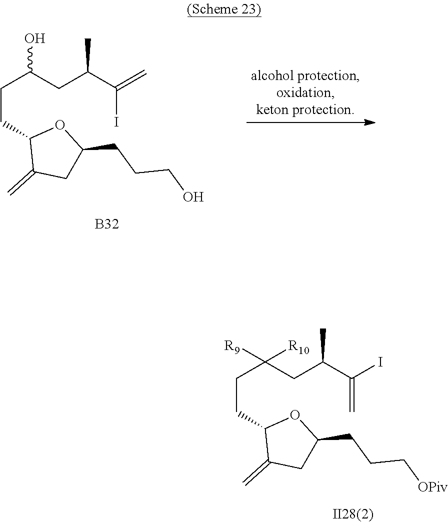

wherein R₉ and R₁₀ are each independently O-alkyl or S-alkyl; or R₉ and R₁₀ form together a 5-6-member acetal ring optionally substituted; and Piv refers to pivaloyl.

In another embodiment, R₉ and R₁₀ of Formula II28(2) are each independently O-alkyl or S-alkyl. In another embodiment, R₉ and R₁₀ form together a 5-6-member acetal ring optionally substituted. In another embodiment, R₉ and R₁₀ form together a 6-member acetal ring as shown:

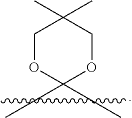

In some embodiments, this invention provides scheme 21 for the preparation of B31 or isomer thereof. In some embodiments, this invention provides scheme 22 for the preparation of B32 or isomer thereof.

In some embodiments, this invention provides scheme 23 for the preparation of II28(2) or isomer thereof.

In some embodiments, provided herein is a process preparing Compound B31 or isomer thereof from Compound B30 or isomer thereof according to scheme 21;

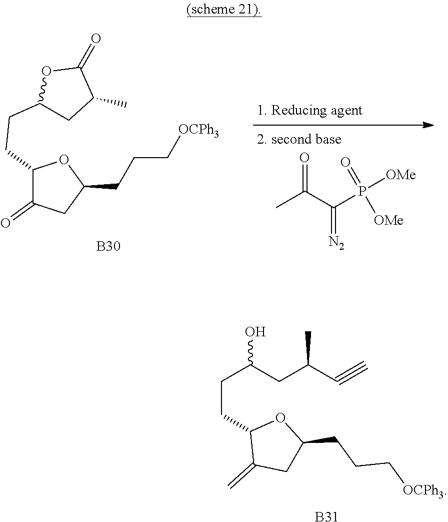

In some embodiments, provided herein is a process for the preparation of a compound of Formula III12 or isomer thereof:

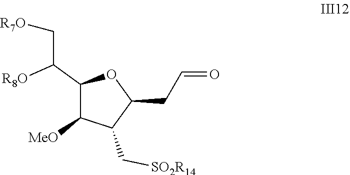

wherein R₇ and R₈ are each independently an alcohol protecting group or R₇ and R₈ form together with the oxygen a 5-6 member ring optionally substituted; and R₁₄ is an alkyl or an aryl;
wherein the process (Process 6) comprises the following steps:
a) preparing a compound of Formula III5 or isomer thereof from a compound of Formula III4 or isomer thereof according to scheme 24:

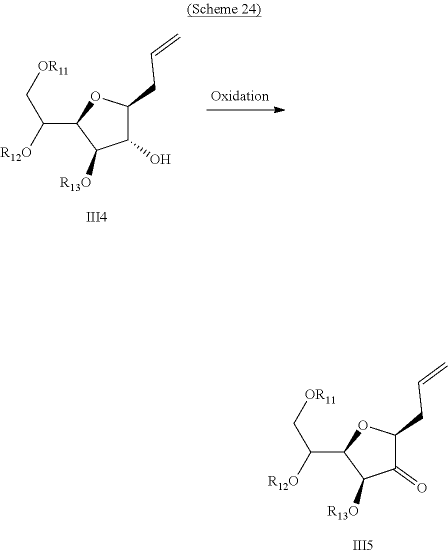

wherein OR₁₁ and OR₁₂ are each independently an ester group; R₁₃ is —CH₂-aryl or

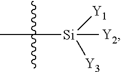

wherein Y₁, Y₂, and Y₃ are each independently an alkyl or an aryl;
b) preparing a compound of Formula III6 or isomer thereof from a compound of Formula III5 or isomer thereof according to scheme 25:

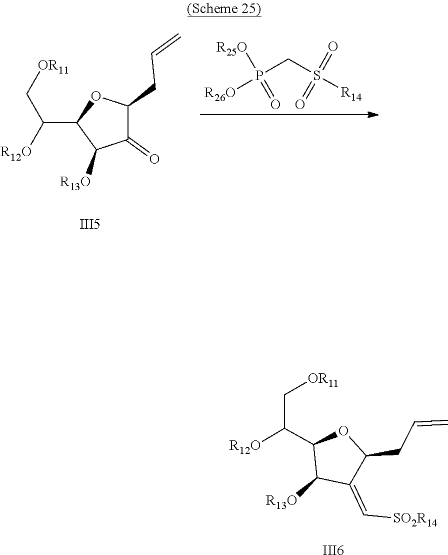

wherein R₁₄, R₂₅, and R₂₆ are each independently an alkyl or an aryl group; OR₁₁ and OR₁₂ are each independently an ester group; and R₁₃ is —CH₂-aryl or

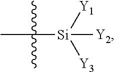

wherein Y₁, Y₂, and Y₃ are each independently an alkyl or an aryl;
(c) preparing a compound of Formula III9 or isomer thereof from a compound of Formula III6 or isomer thereof according to scheme 26:

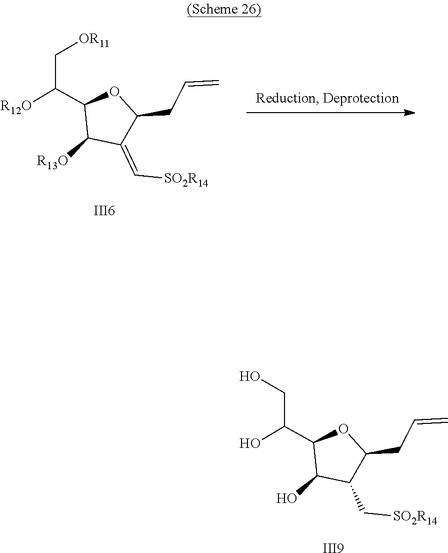

wherein R₁₄, is an alkyl or an aryl group; OR_{II} and OR₁₂ are each independently an ester group; and R₁₃ is —CH₂-aryl or

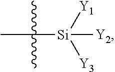

wherein Y₁, Y₂, and Y₃ are each independently an alkyl or an aryl;
d) preparing a compound of Formula III11 or isomer thereof from a compound of Formula III9 or isomer thereof according to scheme 27:

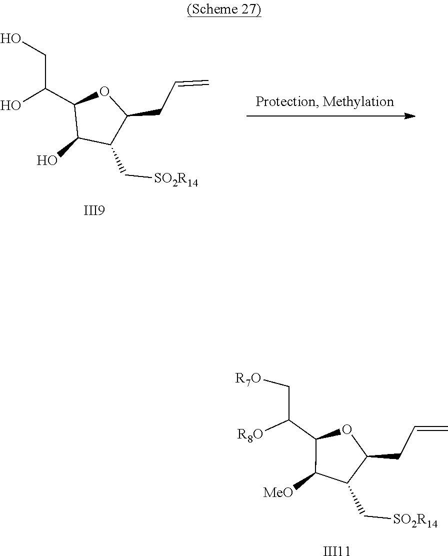

wherein R₇ and R₈ are each independently an alcohol protecting group or R₇ and R₈ form together with the oxygen a 5-6 member ring optionally substituted; and R₁₄ is an alkyl or an aryl; and
e) preparing a compound of Formula III12 or isomer thereof from a compound of Formula III11 or isomer thereof according to scheme 28:

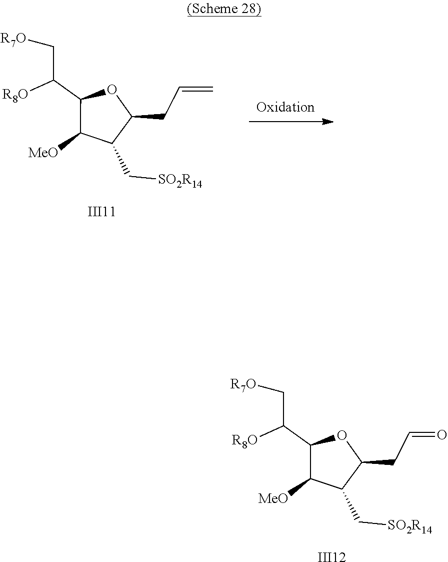

wherein R₇ and R₈ are each independently an alcohol protecting group or R₇ and R₈ form together with the oxygen a 5-6 member ring optionally substituted; and R₁₄ is an alkyl or an aryl.

In some embodiments, this invention provides scheme 24 for the preparation of III5 or isomer thereof. In some embodiments, this invention provides scheme 25 for the preparation of III6 or isomer thereof.

In some embodiments, this invention provides scheme 26 for the preparation of III9 or isomer thereof. In some embodiments, this invention provides scheme 27 for the preparation of III11 or isomer thereof.

In some embodiments, this invention provides scheme 28 for the preparation of III12 or isomer thereof.

In another embodiment, process (Process 6) for the preparation of a compound of a compound of Formula III12 or isomer thereof comprises the following steps:
(a) preparing a compound of Formula III9 or isomer thereof from a compound of Formula III6 or isomer thereof according to scheme 26:

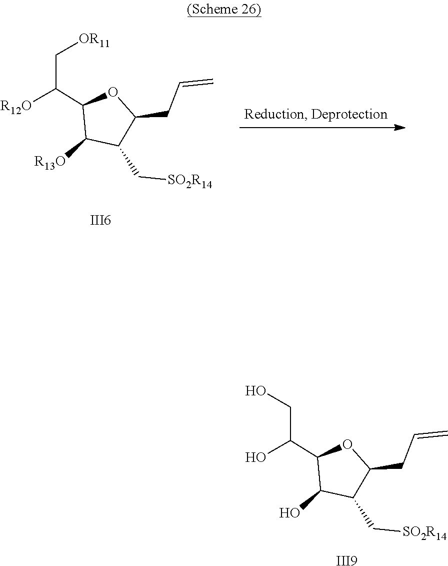

wherein OR₁₁ and OR₁₂ are each independently an ester group; R₁₃ is —CH₂-aryl or

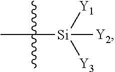

wherein Y₁, Y₂, and Y₃ are each independently an alkyl or an aryl; and R₁₄ is an alkyl or an aryl group;
wherein OR₁₁, OR₁₂ and OR₁₃ are deprotected and the non-terminal double bond is reduced in any sequence;
(b) preparing a compound of Formula III11 or isomer thereof from a compound of Formula III9 or isomer thereof according to scheme 27:

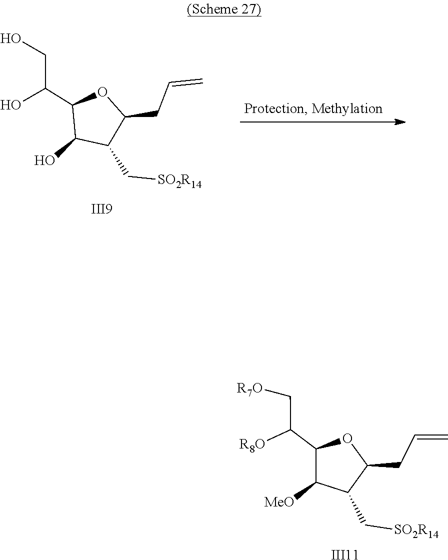

wherein R₇ and R₈ are each independently an alcohol protecting group or R₇ and R₈ form together with the oxygen a 5-6 member ring optionally substituted; and R₁₄ is an alkyl or an aryl; and
(c) preparing a compound of Formula III12 or isomer thereof from a compound of Formula III11 or isomer thereof according to scheme 28:

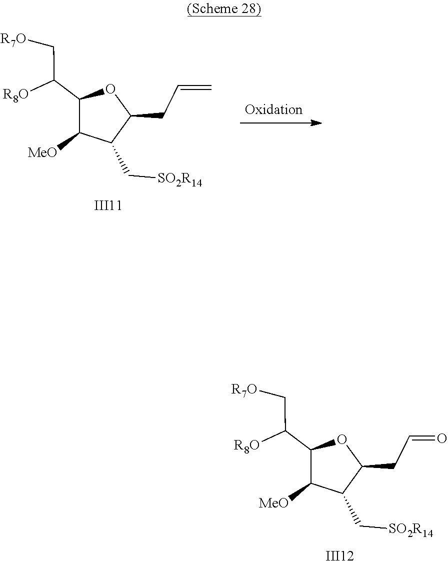

wherein R₇ and R₈ are each independently an alcohol protecting group or R₇ and R₈ form together with the oxygen a 5-6 member ring optionally substituted; and R₁₄ is an alkyl or an aryl.

In another embodiment, R₁₄ of Formula III6, III9, III11, and III12 is a phenyl group. In another embodiment, R₁₁ and Ru of Formula III4, III5, and III6 are each independently a benzoyl group. In another embodiment, R₁₃ of Formula III4, III5, and III6 is a benzyl group. In another embodiment, R₇ and R₈ form together with the oxygen a 5-member ring optionally substituted. In another embodiment, R₇ and R₈ of Formula III11, and III12, form together with the oxygen a 5-member ring as shown:

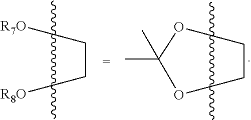

In some embodiments, provided herein is a process for the preparation of a compound of Formula IV12 or isomer thereof from a compound of Formula IV7 or isomer thereof:

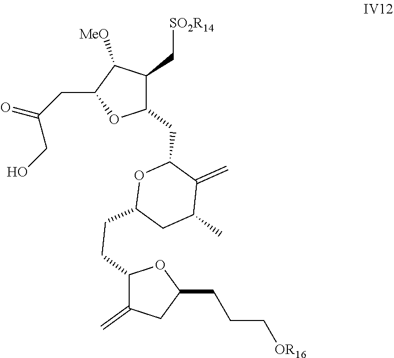

wherein R₁₄ is alkyl or aryl; and R₁₆ is an alcohol protecting group;
wherein the process (Process 7) comprises the following steps:
a) preparing a compound of Formula IV8 or isomer thereof from a compound of Formula IV7 or isomer thereof, according to scheme 29:

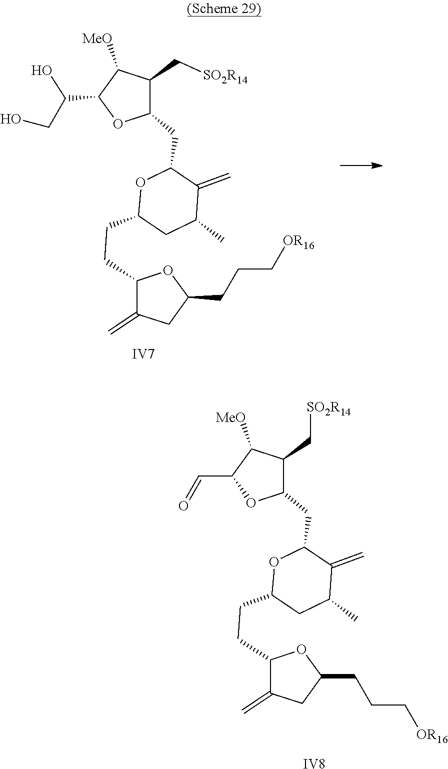

wherein R₁₄ is an alkyl or aryl; and R₁₆ is an alcohol protecting group;
b) preparing a compound of Formula IV9 or isomer thereof from a compound of Formula IV8 or isomer thereof, according to scheme 30:

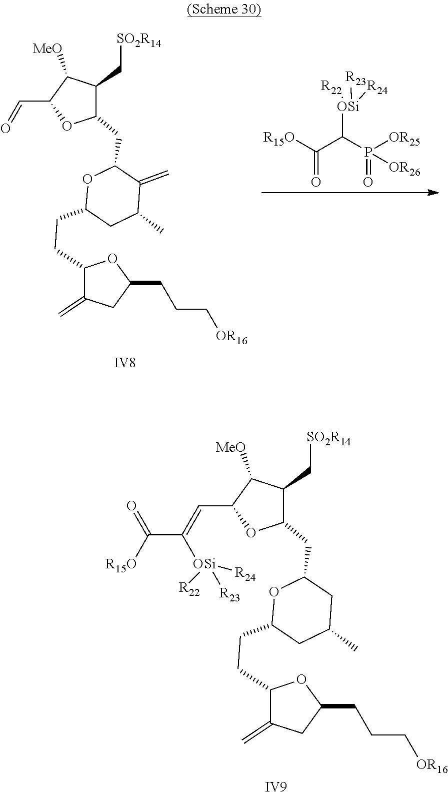

wherein R₁₄ is an alkyl or aryl; R₁₆ is an alcohol protecting group; and R₁₅, R₂₂, R₂₃, R₂₄, R₂₅ and R₂₆ are each independently an alkyl or an aryl group;
c) preparing a compound of Formula IV11 or isomer thereof from a compound of Formula IV9 or isomer thereof, according to scheme 31:

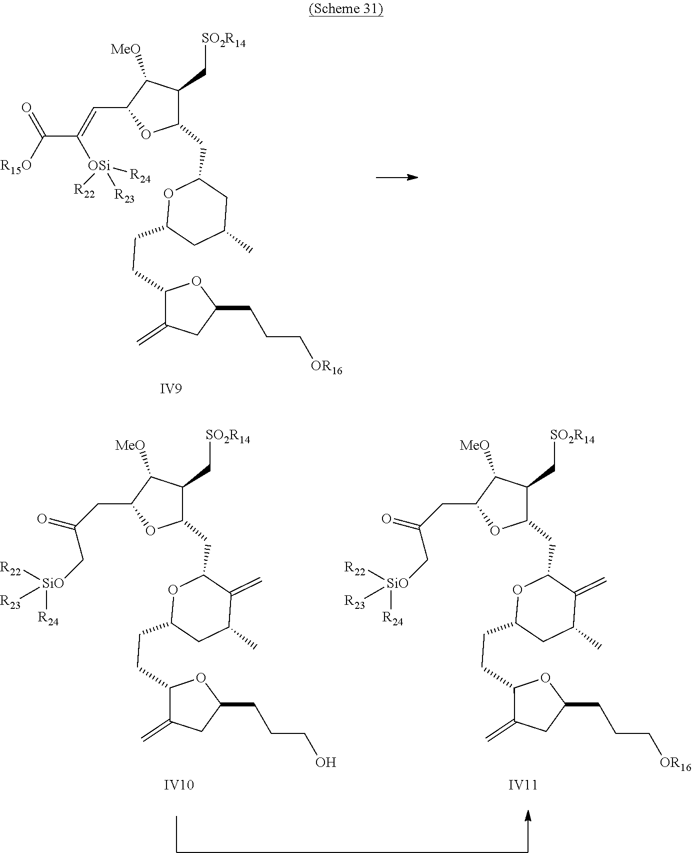

wherein R₁₄ is an alkyl or aryl; R₁₆ is an alcohol protecting group; R₁₅, R₂₂, R₂₃, and R₂₄, are each independently an alkyl or an aryl group; and
wherein,
the compound of Formula IV10 or isomer thereof is reacted with an alcohol protecting group moiety to obtain a compound of Formula IV11 or isomer thereof; and
d) preparing a compound of Formula IV12 or isomer thereof from a compound of Formula IV11 or isomer thereof, according to scheme 32:

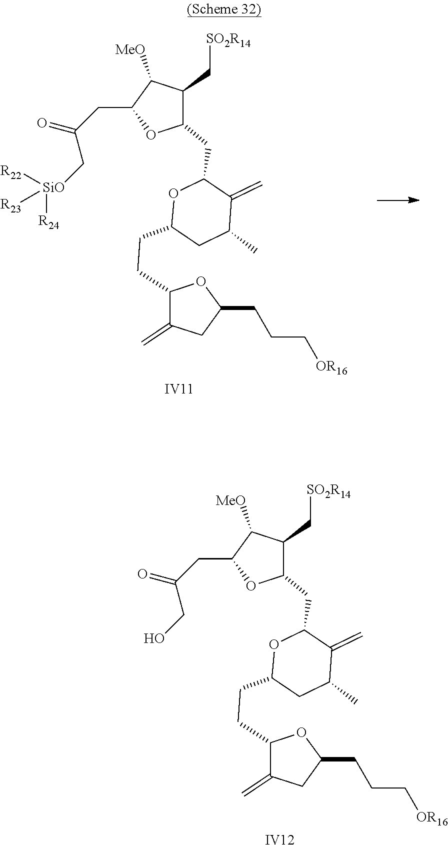

wherein R₁₄ is an alkyl or aryl; R₁₆ is an alcohol protecting group; R₂₂, R₂₃, and R₂₄ are each independently an alkyl or an aryl group;

In another embodiment, R₁₆ of Formula IV7, IV8, IV9, IV11, and IV12 are each independently an alcohol protective groups In another embodiment, R₁₆ of Formula IV7, IV8, IV9, IV11, and IV12 are each independently a protective groups which are stable to (+)-B-chlorodiisopinocampheylborane (DIP-Cl) and acidic hydrolysis. In another embodiment, R₁₆ is pivaloyl.

In another embodiment, R₁₄ of Formula IV7, IV8, IV9, IV10, IV11, and IV12 is phenyl group. In another embodiment, R₁₆ of Formula IV7, IV8, IV9, IV11, and IV12 is pivaloyl group.

In another embodiment, R₂₂, R₂₃ and R₂₄ of Formula IV9, IV10 and IV1 are each independently methyl group. In another embodiment, R₂₂, R₂₃ and R₂₄ are each independently t-butyl group. In another embodiment, at least two of R₂₂, R₂₃ and R₂₄ are methyl group. In another embodiment, two of R₂₂, R₂₃ and R₂₄ are methyl group, and one is t-butyl group. In another embodiment, R₁₅ of Formula IV9 is methyl.

In some embodiments, this invention provides scheme 29 for the preparation of IV8. In some embodiments, this invention provides scheme 30 for the preparation of IV9.

In some embodiments, this invention provides scheme 31 for the preparation of IV11. In some embodiments, this invention provides scheme 32 for the preparation of IV12.

In some embodiment, provided herein is a process for the preparation of a compound of Formula IV13 wherein the process comprises
(i) preparing a compound of Formula IV12 or isomer thereof according to the process disclosed in this invention; and
(ii) reducing the ketone of a compound of Formula IV12 or isomer thereof to obtain a compound of Formula IV13 or isomer thereof.

In another embodiment, the process for preparing a compound of Formula IV12 or isomer thereof is process 7 of this invention.

In another embodiment, the reduction of the ketone group of IV12 comprises a reducing agent. In another embodiment, the reducing agent is any reducing agent known in the art for reducing ketone. In another embodiment, the reducing agent is enantioselective ketone reductions convert prochiral ketones into chiral, non-racemic alcohols.

In another embodiment, the reducing agent is selected from (+) DIP-Cl Oxazaborolidine-borane reduction (CBS reduction), alpine-boranes, transition metal catalyzed reductions (for example, Najori (Ru-BINAP catalyst) and others, chiral aluminum and borohydrides.

In another embodiment, the reducing agent is any reducing agent known in the art for chiral reduction of ketone. In another embodiment, the reducing agent is any reducing agent known in the art for chiral reduction of ketone, non-limiting examples is found in Corey E. J., Helal C. J. Angew. Chem. Int. Ed. 1998, 37, 1986-2012 (CBS reduction); Singh V. K. Synthesis, 1992, 605-620 (boranes and BINAP-Ru); Deloux L., Srebnik M, Chem. Rev., 1993, 93, 763-777 (assymmetric boron-catalyzed reactions) of which are incorporated entirety herein by reference.

In another embodiment, the reducing agent is (+)-B-chlorodiisopinocampheylborane (DIP-Cl), (+) DIP-Cl.

In some embodiments, the preparation of a compound of Formula IV7 or isomer thereof is prepared from a compound of Formula III12 or isomer thereof.

In some embodiments, provide d herein is a process for the preparation of a compound of Formula IV7 or isomer thereof:

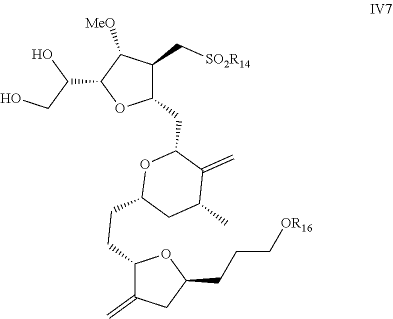

wherein R₁₄ is alkyl or aryl; and R₁₆ is an alcohol protecting group;
wherein the process (Process 8) comprises the following steps:
a) preparing a compound of Formula IV1 or isomer thereof by reacting a compound of Formula III12 or isomer thereof with a compound of Formula II20 or isomer thereof, according to scheme 33:

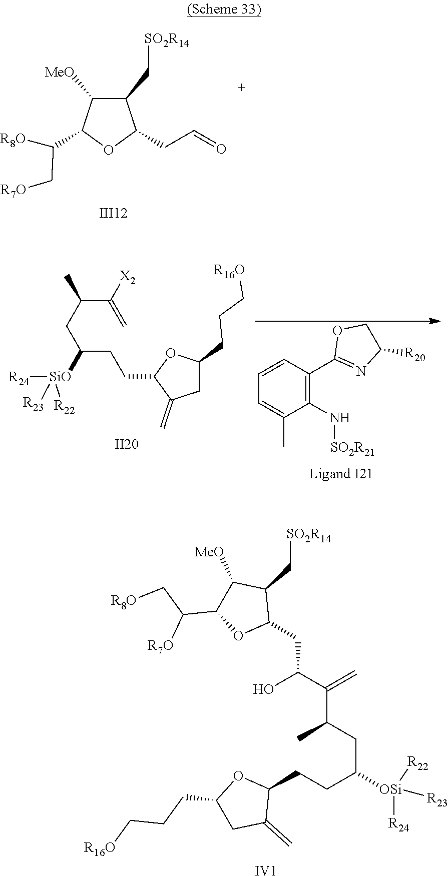

wherein R₇, R₈ and R₁₆ are each independently an alcohol protecting group; or R₇ and R₈ form together with the oxygen a 5-6-member ring optionally substituted; R₁₄, R₂₀, R₂₁ R₂₂, R₂₃ and R₂₄ are each independently an alkyl or aryl group; and X₂ is a halogen;
b) preparing a compound of Formula IV2 or isomer thereof from a compound of Formula IV1 or isomer thereof according to scheme 34:

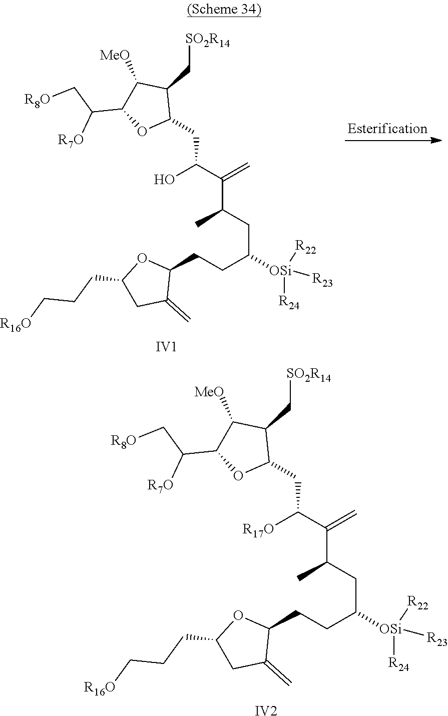

wherein OR₁₇ is an ester; R₇, R₈ and R₁₆ are each independently an alcohol protecting group; or R₇ and R₈ form together with the oxygen a 5-6-member ring optionally substituted; and R₁₄, R₂₂, R₂₃ and R₂₄ are each independently an alkyl or aryl group;
c) preparing a compound of Formula IV3 or isomer thereof from a compound of Formula IV2 or isomer thereof according to scheme 35:

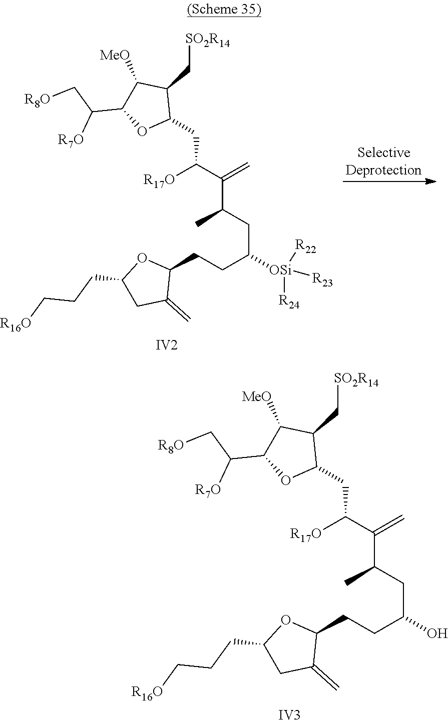

wherein OR₁₇ is an ester; R₇, R₈ and R₁₆ are each independently an alcohol protecting group; or R₇ and R₈ form together with the oxygen a 5-6-member ring optionally substituted; and R₁₄, R₂₂, R₂₃ and R₂₄ are each independently an alkyl or aryl group;
d) preparing a compound of Formula IV4 or isomer thereof from a compound of Formula IV3 or isomer thereof according to scheme 36:

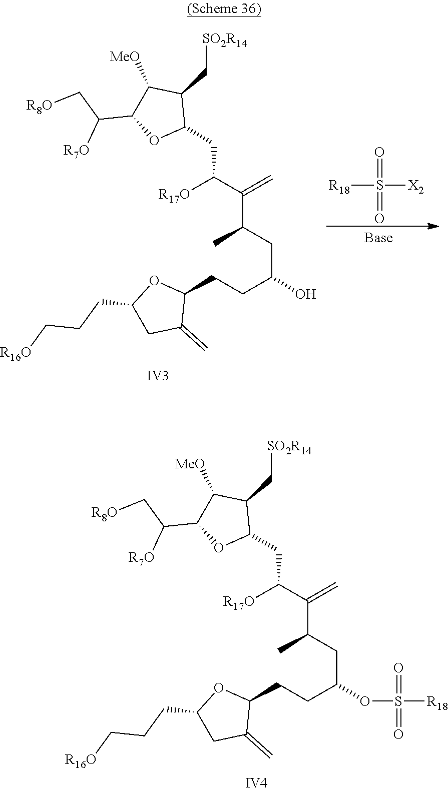

wherein X₂ is a halogen; R₁₈ is an alkyl or an aryl; OR₁₇ is an ester; R₇, R₈ and R₁₆ are each independently an alcohol protecting group; or R₇ and R₈ form together with the oxygen a 5-6-member ring optionally substituted; and R₁₄, is an alkyl or aryl group;
e) preparing a compound of Formula IV6 or isomer thereof from a compound of Formula IV4 or isomer thereof according to scheme 37:

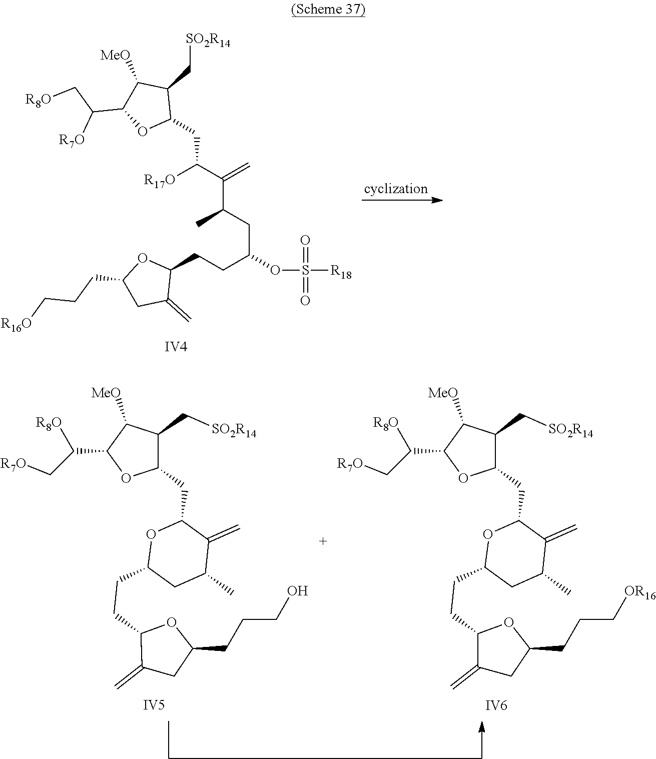

wherein R₁₈ is an alkyl or an aryl; OR₁₇ is an ester; R₇, R₈ and R₁₆ are each independently an alcohol protecting group; or R₇ and R₈ form together with the oxygen a 5-6-member ring optionally substituted; and wherein, the compound of Formula IV5 or isomer thereof is reacted with an alcohol protecting group moiety to obtain a compound of Formula IV6 or isomer thereof; and
f) preparing a compound of Formula IV7 or isomer thereof from a compound of Formula IV6 or isomer thereof according to scheme 38:

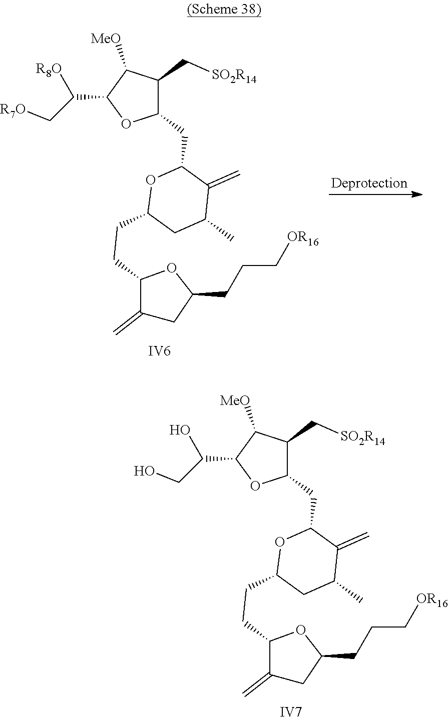

wherein R₇, R₈ and R₁₆ are each independently an alcohol protecting group; or R₇ and R₈ form together with the oxygen a 5-6-member ring optionally substituted;

In another embodiment, R₁₆ of a compound of Formula II20, IV1, IV2, IV3, IV4, IV6 or IV7 is independently an alcohol protecting group. In another embodiment, the alcohol protecting group of R₁₆ is a group stable to acidic conditions. In another embodiment, R₁₆ is acyl. In another embodiment, R₁₆ is pivaloyl. In another embodiment, R₁₆ is a protective groups which are stable to (+)-B-chlorodiisopinocampheylborane (DIP-Cl) and acidic hydrolysis. In another embodiment, R₁₆ is a pivaloyl group.

In another embodiment, R₇ and R₈ of a compound of Formula III12, IV1, IV2, IV3, IV4, IV5 or IV6 form together with the oxygen a 5-member ring as shown:

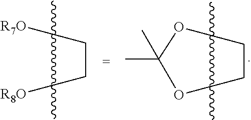

In another embodiment, R₁₇ of a compound of Formula IV2, IV3, or IV4 is —C(═O)CH₃ group. In another embodiment, R₁₈ of a compound of Formula IV4 is methyl. In another embodiment, R₁₄ of a compound of Formula IV1, IV2, IV3, IV4, IV5, IV6 or IV7 is phenyl. In another embodiment, R₂₂, R₂₃ and R₂₄ of Formula II20, IV1 and IV2 are each independently methyl group. In another embodiment, R₂₂, R₂₃ and R₂₄ are each independently t-butyl group. In another embodiment, at least two of R₂₂, R₂₃ and R₂₄ are methyl group. In another embodiment, two of R₂₂, R₂₃ and R₂₄ are methyl group, and one is t-butyl group. In some embodiments, this invention provides scheme 33 for the preparation of IV1 or isomer thereof. In some embodiments, this invention provides scheme 34 for the preparation of IV2 or isomer thereof.

In some embodiments, this invention provides scheme 35 for the preparation of IV3 or isomer thereof. In some embodiments, this invention provides scheme 36 for the preparation of IV4 or isomer thereof. In some embodiments, this invention provides scheme 37 for the preparation of IV6 or isomer thereof. In some embodiments, this invention provides scheme 38 for the preparation of IV7 or isomer thereof.

In some embodiments, provided herein is a process for the preparation of a compound of Formula IV7 or isomer thereof from a compound of Formula III12 or isomer thereof by process 8 provided herein. In some embodiments, provided herein is a process for the preparation of a compound of Formula IV7 or isomer thereof from a compound of Formula III12 or isomer thereof by process 9 provided herein. In some embodiments, provided herein is a process for the preparation of a compound of Formula IV7 or isomer thereof from a compound of Formula III12 or isomer thereof by process 10 provided herein.

In some embodiments, provided herein is a process for the preparation of a compound of Formula IV7 or isomer thereof:

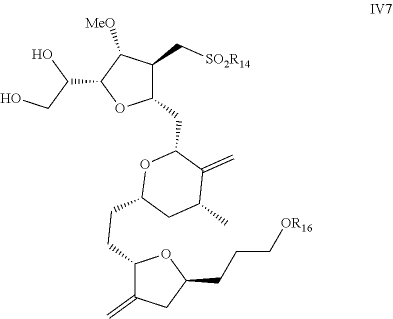

wherein R₁₄ is an alkyl or an aryl; and R₁₆ is an alcohol protecting group;
wherein the process (Process 9) comprises the following steps:
a) preparing a compound of Formula IV17 or isomer thereof by reacting a compound of Formula III12 or isomer thereof with a compound of Formula II28 or isomer thereof, according to scheme 39:

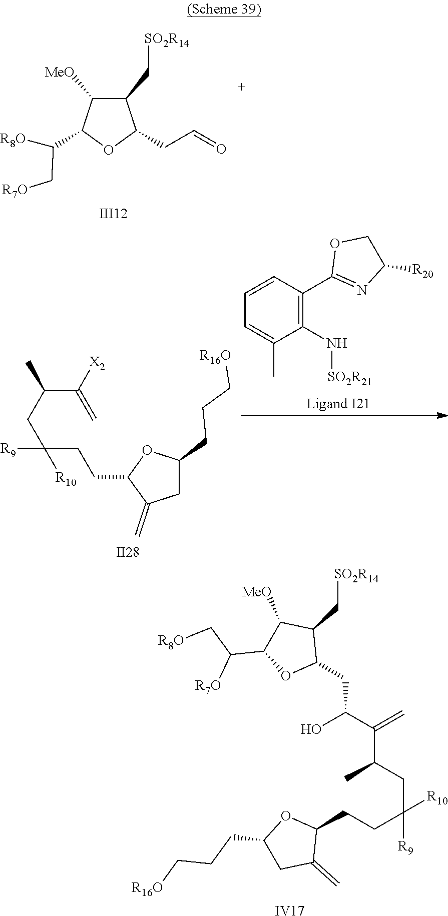

wherein R₇ and R₈ are each independently an alcohol protecting group; or R₇ and R₈ form together with the oxygen a 5-6-member ring optionally substituted; R₉ and R₁₀ are each independently O-alkyl or S-alkyl; or R₉ and R₁₀ form together a 5-6-member acetal ring optionally substituted; X₂ is a halogen; R₁₄ is an alkyl or an aryl; R₁₆ is an alcohol protecting group; and R₂₀ and R₂₁ are each individually an alkyl or an aryl group; and
b) preparing a compound of Formula IV7 or isomer thereof from a compound of Formula IV17 or isomer thereof, according to scheme 40:

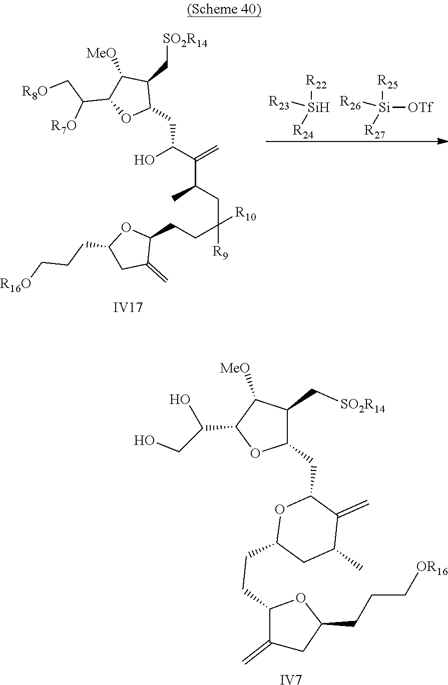

wherein R₇ and R₈ are each independently an alcohol protecting group; or R₇ and R₈ form together with the oxygen a 5-6-member ring optionally substituted; R₉ and R₁₀ are each independently O-alkyl or S-alkyl; or R₉ and R₁₀ form together a 5-6-member acetal ring optionally substituted; R₁₄ is an alkyl or an aryl; R₁₆ is an alcohol protecting group; and R₂₀ and R₂₁ are each individually an alkyl or an aryl group; and, R₂₂, R₂₃, R₂₄, R₂₅, R₂₆, and R₂₇ are each independently an alkyl or an aryl group.

In another embodiment, R₁₆ of a compound of Formula IV7, IV17, or II28, are each independently an alcohol protecting group. In another embodiment, R₁₆ is acyl. In another embodiment, R₁₆ is pivaloyl.

In another embodiment, R₇ and R₈ of a compound of Formula IV17 are each independently TBS group.

In another embodiment, R₉ and R₁₀ of Formula II28 or IV17 are each independently O-alkyl or S-alkyl. In another embodiment, R₉ and R₁₀ form together a 5-6-member acetal ring optionally substituted. In another embodiment, R₉ and R₁₀ form together a 6-member acetal ring as shown:

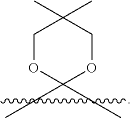

In another embodiment, R₁₄ of a compound of Formula IV17, or IV7 is phenyl.

In some embodiments, this invention provides scheme 39 for the preparation of IV17. In some embodiments, this invention provides scheme 40 for the preparation of IV7.

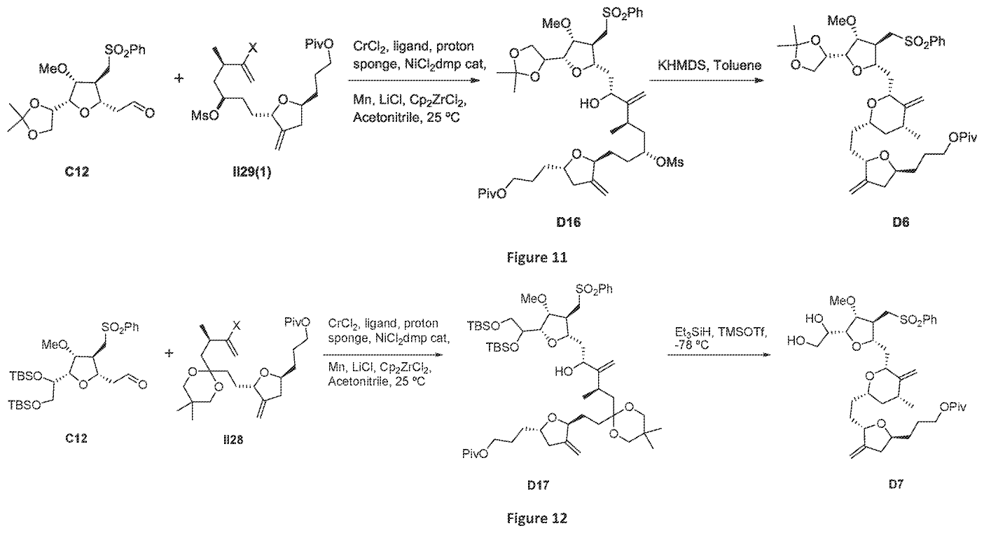

In some embodiments, provided herein is a process for the preparation of a compound of Formula IV7 or isomer thereof:

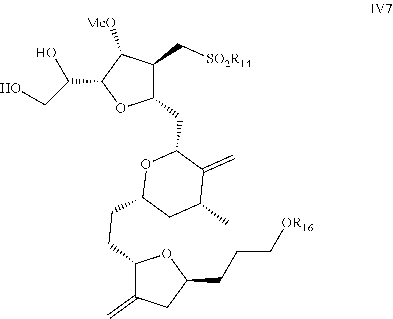

wherein R₁₄ is an alkyl or an aryl; and R₁₆ is an alcohol protecting group;
wherein the process (Process 10) comprises the following steps (see also FIG. 11):
a) preparing a compound of Formula IV16 or isomer thereof by reacting a compound of Formula III12 or isomer thereof with a compound of Formula II29 or isomer thereof, according to scheme 41:

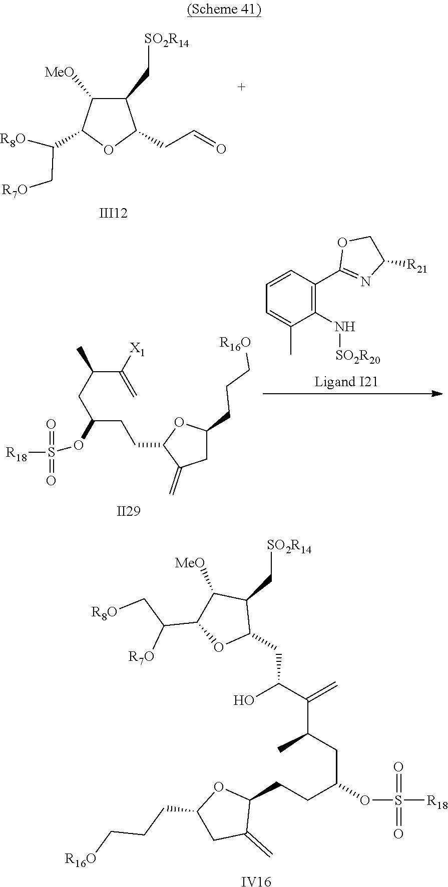

wherein R₇ and R₈ are each independently an alcohol protecting group; or R₇ and R₈ form together with the oxygen a 5-6-member ring optionally substituted; X₁ is a leaving group; R₁₄ and R₁₈ are each independently an alkyl or an aryl; R₁₆ is an alcohol protecting group; and R₂₀ and R₂₁ are each independently an alkyl or an aryl group;
b) preparing a compound of Formula IV6 from a compound of Formula IV16, according to scheme 42:

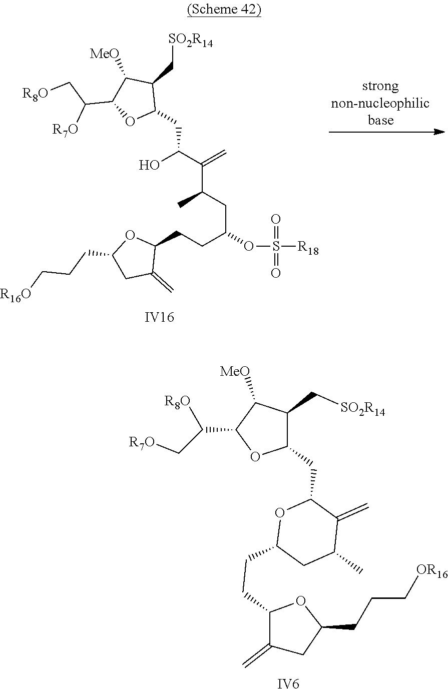

wherein R₇ and R₈ are each independently an alcohol protecting group; or R₇ and R₈ form together with the oxygen a 5-6-member ring optionally substituted; R₁₄ and R₁₈ are each independently an alkyl or an aryl; R₁₆ is an alcohol protecting group; and
c) preparing a compound of Formula IV7 or isomer thereof from a compound of Formula IV6 or isomer thereof, according to scheme 43:

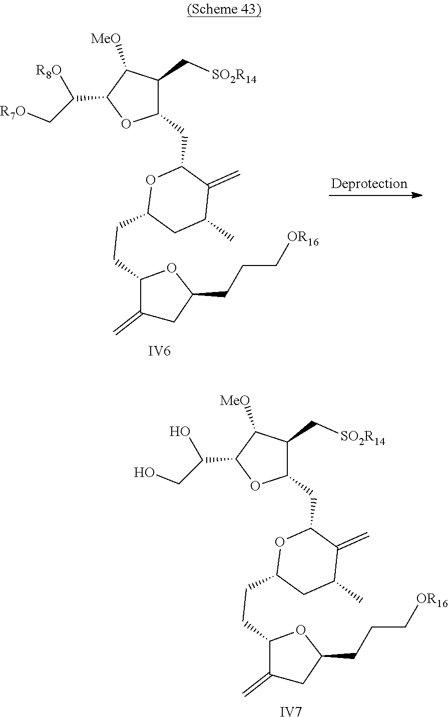

wherein R₇ and R₈ are each independently an alcohol protecting group; or R₇ and R₈ form together with the oxygen a 5-6-member ring optionally substituted; R₁₄ is an alkyl or an aryl; and R₁₆ is an alcohol protecting group.

In another embodiment, R₇ and R₈ of a compound of Formula III12, IV16, or IV6 form together with the oxygen a 5-member ring as shown:

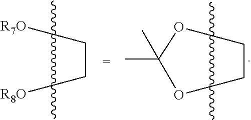

In another embodiment, R₁₄ of a compound of Formula III12, IV16, IV6, or IV7 is phenyl. In another embodiment, R₁₄ of a compound of Formula II29, IV16, IV6, or IV7 is pivaloyl group. In another embodiment, X₁ of a compound of Formula II29 is halogen.

In some embodiments, this invention provides scheme 41 for the preparation of IV16. In some embodiments, this invention provides scheme 42 for the preparation of IV6. In some embodiments, this invention provides scheme 43 for the preparation of IV7.

In some embodiments, provided herein, a process for the preparation of I11 or isomer thereof

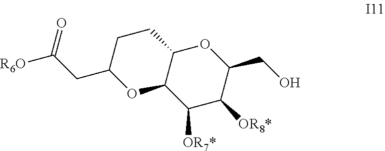

wherein R₇* and R₈* are each independently an alcohol protecting group or R₇* and R₈* form together with the oxygen a 5-6 member ring optionally substituted; and R₆ is an alkyl; wherein the process (process 11) comprises preparing a compound of Formula I11 or isomer thereof from I10(S) or isomer thereof

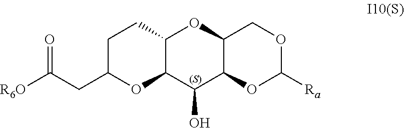

wherein R₆ is an alkyl; and Rₐ is substituted or unsubstituted alkyl, substituted or unsubstituted aryl, or substituted or unsubstituted heteroaryl;
by acetal cleavage and diol protection.

In some embodiments, provided herein, a process for the preparation of Ill or isomer thereof

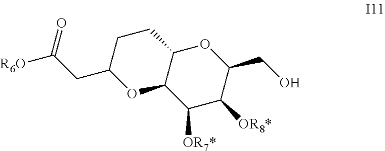

wherein R₇* and R₈* are each independently an alcohol protecting group or R₇* and R₈* form together with the oxygen a 5-6 member ring optionally substituted; and R₆ is an alkyl;
wherein the process (process 12) comprises preparing a compound of Formula I11 or isomer thereof from I7(R) or isomer thereof

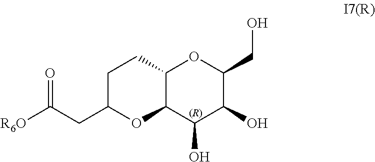

wherein R₆ is an alkyl;
by diol protection.

In some embodiments, the diol protection (protection of two alcohol groups) of processes 11 and 12 comprises any diol protecting group known in the art. In another embodiment, the diol protection of processes 11 and 12 comprises cyclohexanone. In another embodiment, the diol protection comprises acid. In another embodiment, the diol protection comprises cyclohexanone and catalytic amount of an acid. In another embodiment, the acid is para-toluene sulfonic acid.

In some embodiments, provided herein is a process for the preparation of Eribulin, wherein the process comprises the following steps:
(i) preparing a compound of formula IV12 or isomer thereof:

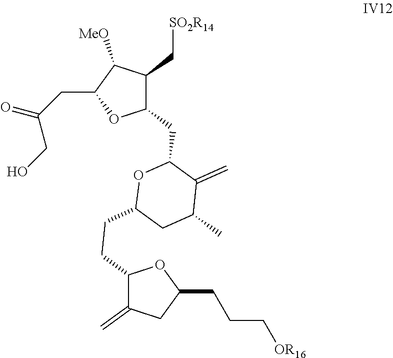

wherein R₁₄ is an alkyl or an aryl; R₁₆ is an alcohol protecting group; from a compound of formula IV7 or isomer thereof:

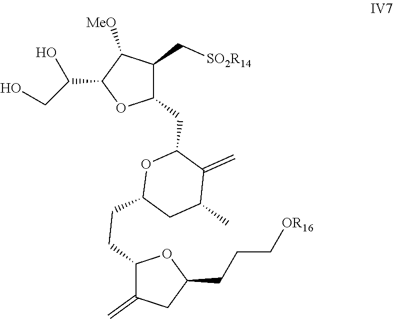

wherein R₁₄ is an alkyl or an aryl; R₁₆ is an alcohol protecting group.
by elongating the chain of the diol; and
(ii) preparing Eribulin from a compound of formula IV12 or isomer thereof, wherein the process comprises reducing the ketone group of IV12 to form diol, protecting the diol group and coupling reaction to obtain Eribulin.

In some embodiment, each scheme from 1-43 represent a different embodiment of this invention.

In some embodiments, Processes 1, 2, 3, 4, 5 and 6 include an oxidation step to oxidize alcohol to ketone. In one embodiment, the oxidation step comprises Dess Martin periodinane (DMP), DMSO-based oxidation, 2-Iodoxybenzoic acid (IBX), Swern oxidation, radical oxidation, Pyridinium Dichromate (PDC), Pyridinium chlorochromate (PCC) or bis(acetoxy)iodo]benzene (BAIB) and (2,2,6,6-Tetramethylpiperidin-1-yl)oxyl (TEMPO). Each represent a separate embodiment of this invention. In another embodiment, the oxidation step of Processes 1, 2, 3, 4, 5 and 6 comprises Dess Martin periodinane (DMP). Each represent a separate embodiment of this invention. In another embodiment, the oxidation step of Processes 1, 2, 3, 4, 5 and 6 comprises DMSO-based oxidation. Each represent a separate embodiment of this invention. In another embodiment, the oxidation step of Processes 1, 2, 3, 4, 5 and 6 comprises IBX. Each represent a separate embodiment of this invention. In another embodiment, the oxidation step of Processes 1, 2, 3, 4, 5 and 6 comprises Swern oxidation. Each represent a separate embodiment of this invention. In another embodiment, the oxidation step of Processes 1, 2, 3, 4, 5 and 6 comprises radical oxidation. Each represent a separate embodiment of this invention. In another embodiment, the oxidation step of Processes 1, 2, 3, 4, 5 and 6 comprises PDC. Each represent a separate embodiment of this invention. In another embodiment, the oxidation step of Processes 1, 2, 3, 4, 5 and 6 comprises PCC. Each represent a separate embodiment of this invention. In another embodiment, the oxidation step of Processes 1, 2, 3, 4, 5 and 6 comprises BAIB and TEMPO. Each represent a separate embodiment of this invention.

In one embodiment, Process 6 in step (e) (as described in scheme 28) comprises an oxidation step, that oxidize a terminal double bond to an aldehyde. In another embodiment, the oxidation comprises OsO₄ and NaIO₄. In another embodiment, the oxidation comprises a combination of O₃ with triphenylphosphine or dimethylsulfide or poisoned Pd. In another embodiment, the oxidation comprises OsO₄ and NaIO₄ and a base. In another embodiment, the base is pyridine or substituted pyridine. In another embodiment, the base is methyl pyridine. In another embodiment, the oxidation comprises OsO₄, NaIO₄ and a 2,6-lutidine.

In some embodiments, Process 1 (step (a)—as described in scheme 1), Process 2 (step (c)—as described in scheme 8), Process 3 (step (a)—as described in scheme 10) comprises a substitution reaction. In another embodiment, the reaction of Process 1, step (a) comprises (i) BF₃-Et₂O and (ii) trimethylsilyl trifluoromethanesulfonate (TMSOTf), TiCl₄ or TiCl₃(O-iPr).

In some embodiments, Process 1 (step (b) as described in scheme 2), Process 2 (step (d)—scheme 9), Process 3 (step (b) as described in scheme 11), comprises deprotection and cyclization reactions are done simultaneously. In another embodiment, the deprotection is done prior to the cyclization. In another embodiment, the deprotection and cyclization reactions comprises basic conditions. In another embodiment, the basic conditions of scheme 2 comprise alkali metal alkoxide, ammonium alkoxide, alkali metal hydroxide or ammonium hydroxide. In another embodiment, the basic conditions comprise alkali metal alkoxide. In another embodiment, the basic conditions comprise ammonium alkoxide. In another embodiment, the basic conditions comprise alkali metal hydroxide. In another embodiment, the basic conditions comprise ammonium hydroxide. In another embodiment, the basic conditions comprise NaOMe.

In one embodiment, the reaction of Process 1 step (c) (as described in scheme 3) comprises trans acetalization reaction. In one embodiment, Process 1 step (c) (as described in scheme 3) comprises RₐCH(OMe)₂, wherein Rₐ is substituted or unsubstituted alkyl, substituted or unsubstituted aryl, or substituted or unsubstituted heteroaryl, and acid catalyst. In one embodiment, Process 1 step (c) (as described in scheme 3) comprises PhCH(OMe)₂, and acid catalyst. In another embodiment, the acid catalyst is sulfonic acid. In another embodiment, the acid catalyst is p-TSA.

In some embodiments, Processes 1, 2 or 3, include a reduction step for reducing a ketone to an alcohol. In one embodiment, the reduction step comprises reaction with a reducing agent. In another embodiment, the reducing agent is NaBH₄, NaCNBH₃, Zn(BH₄)₂ or LiBH₄. In another embodiment, the reducing agent is NaBH₄. In another embodiment, the reducing agent is NaCNBH₃. In another embodiment, the reducing agent is Zn(BH₄)₂. In another embodiment, the reducing agent is LiBH₄.

In some embodiments, the reaction of Process 2 step (b) (as described in scheme 7) or Process 8 step (b) (as described in scheme 34) comprises an alcohol protection. In another embodiment, the reaction for protecting the alcohol group comprises acyl halide or acyl anhydride, each is a separate embodiment according to this invention. In another embodiment, the reaction comprises reacting a compound with acetyl halide, pivaloyl halide, butyryl halidepivaloyl anhydride, butyryl anhydride, or benzoic anhydride, each is a separate embodiment according to this invention. In another embodiment, the reaction comprises reacting a compound with benzyl halide or pivaloyl halide. In another embodiment, the reaction conditions comprise (i) acyl halid or acyl anhydride and (ii) a base. In another embodiment, the base comprise pyridine, alkyl substituted pyridine, tertiary amines, alkylmorpholines, DMAP, DBU or a combination thereof, each is a separate embodiment according to this invention. In another embodiment, the reaction conditions are benzoyl chloride and Et₃N.

In some embodiments, the reaction of Process 3 step c (as described in scheme 12), Process 6 step (d) (as described in scheme 27), Process 11, Process 12 comprises protection on two alcohol groups or diol group. In another embodiment, the reaction for protecting the alcohol group comprises reacting the compound of Formula I17(1), III9 methylated III9, I7(R), or I10 with (i) acetone and (ii) acid catalyst. In another embodiment, the reaction for protecting the alcohol group comprises reacting the compound of Formula I17(1), III9, methylated III9, I7(R), or I10 with (i) 2,2-Dimethoxypropane/acetone and (ii) acid catalyst. In another embodiment, the reaction for protecting the alcohol group comprises reacting the compound of Formula I17(1), III9 I7(R), or I10 methylated III9 with (i) 2-methoxypropene and (ii) acid catalyst. In another embodiment, the reaction for protecting the alcohol group comprises (i) 2-methoxypropene, and (ii) acid catalyst. In another embodiment, the reaction for protecting the alcohol group comprises reacting the compound of Formula I17(1), III9, methylated III9, I7(R), or I10 with (i) cyclohexanone and (ii) acid catalyst. In another embodiment, the acid catalyst comprises a catalytic amount of sulfuric acid, alkyl or aryl sulfonic acid. In another embodiment, the alkyl or aryl sulfonic acid comprises MsOH, camphorsulfuric acid or p-TsOH.

In one embodiment, the reaction of Process 3 step (c) (as described in scheme 12), comprises a protection on two alcohol groups of a compound of Formula I17 followed by deprotection of the benzyl group to obtain a compound of Formula I18.

In one embodiment, Process 3 step (c) (as described in scheme 12) includes deprotection of the benzyl group. In another embodiment, the deprotection comprises hydrogenation. In another embodiment, the hydrogenation conditions comprise hydrogen gas with palladium on carbon catalyst. In another embodiment, the hydrogenation conditions comprise hydrogen gas with platinum catalyst or ruthenium catalyst.

In one embodiment, Process 6 step (d) comprises protection on two alcohol groups with an alcohol protective group. In another embodiment, the reaction condition of the protection step comprises acyl halide or acyl anhydride. In another embodiment the reaction comprises benzoyl halide, acetic anhydride, acetyl halide, pivaloyl halide, benzoic anhydride, each is a separate embodiment according to this invention. In one embodiment, Process 6 step (d) comprises protection on two alcohol groups wherein R₇ and R₈ form together with the oxygen a 5-6-member ring optionally substituted.

In another embodiment, the protection step comprises any known procedures of protecting groups and include those described in detail in Protecting Groups in Organic Synthesis, T. W. Greene and P. G. M. Wuts, 3rd edition, John Wiley & Sons, 1999; Bruce, A., et al., WO199965894; Bruce, A., et al., WO2004034990; Bruce, A., et al., WO2007061874; and Austad, B., rt al., WO2005118565 of which are incorporated entirety herein by reference.

In one embodiment, Process 3 step (c) (as described in scheme 12) includes deprotection of the benzyl group. In another embodiment, the deprotection comprises hydrogenation. In another embodiment, the hydrogenation conditions comprise hydrogen gas with palladium on carbon catalyst. In another embodiment, the hydrogenation conditions comprise hydrogen gas with platinum catalyst or ruthenium catalyst.

In some embodiment, Process 3 step (e) as described in scheme 14 includes deprotection of the protecting group. In another embodiment, the reaction of the deprotection comprises acetal hydrolysis. In another embodiment, the acetal hydrolysis comprises aq AcOH. In another embodiment, the acetal hydrolysis reaction it conducted at 30-60° C. In another embodiment, the acetal hydrolysis reaction it conducted at 40° C. In another embodiment, the reaction of the deprotection comprises removal of acyl protecting groups. In another embodiment, the deprotection of the acyl group comprises basic conditions. In another embodiment, the basic condition comprises catalytic amount of alkali metal alkoxide. In another embodiment, the deprotection of the acyl group comprises reacting the compound with catalytic amount of NaOMe in MeOH.

In some embodiments, Process 4 (step (b) as described in scheme 19) comprises three steps: (i) deprotection of the trityl group, (ii) protection on the primary alcohol, and (iii) protection on the keton group. In another embodiment, the order of the steps is: (i) deprotection of the trityl group, (ii) protection on the primary alcohol, and (iii) protection on the ketone group. In another embodiment, the order of the steps is: (i) protection on the primary alcohol, (ii) deprotection of the trityl group, and (iii) protection on the primary alcohol. In another embodiment, the order of the steps is: (i) deprotection of the trityl group, (ii) protection on the ketone group, and (iii) protection on the primary alcohol. In another embodiment, the protection on the ketone group and the deprotection of the trityl group are conducted in simultaneously.

In some embodiments, the deprotection of the trityl group in Process 4 (step (b) as described in scheme 19) comprises aqueous strong acid. In another embodiment, the strong acid comprises sulfuric acid, TFA or AcOH.

In some embodiment, the protective acetalyzation (protection on the ketone group) of Process 4 step (b) (as described in scheme 19) and Process 5 step (c) (as described in scheme 23) comprises a reaction with (OR)₃CH (wherein R is an alkyl or an aryl), propane diol and catalytic amount of sulfonic acid. In another embodiment, the protective acetalization comprises reacting the compound with OR)₃CH (wherein R is an alkyl or an aryl), substituted ethane, and catalytic amount of sulfonic acid. In another embodiment, the protective acetalization comprises reacting the compound with (OMe)₃CH— and 2,2-dimethylpropane-1,3-diol. In another embodiment, the protective acetalization comprises reacting the compound with a propane-diol, catalytic amount of sulfonic acid and non polar solvent at Dean-stark conditions. In another embodiment, the protective acetalization comprises reacting the compound with a substituted ethane, catalytic amount of sulfonic acid and non-polar solvent at Dean-stark conditions. In another embodiment, the non-polar solvent is toluene, benzene, cyclohexane or combination thereof. Each represent a separate embodiment of this invention.

In some embodiments, Process 4 step (b) as described in scheme 19—the protection on the ketone group and the deprotection of the trityl group are conducted in “one pot”. In some embodiments, Process 4 step (b) as described in scheme 19—the protection on the ketone group and the deprotection of the trityl group are conducted in a two step reaction.

In some embodiments, the reaction for protecting the primary alcohol in Process 4 step (b) (as described in scheme 19), and Process 5 step c (as described in scheme 23) comprises pivaloyl halide or pivaloyl anhydride. Each represent a separate embodiment of this invention.

In another embodiment, the reaction comprises pivaloyl halide. In another embodiment the reaction comprises pivaloyl anhydride.

In one embodiment, the term “methyl moiety” of Process 4 step (c) (as described in scheme 20 comprises MeMgCl or MeLi.

In one embodiment, the base of Process 4 step (c) comprises 2,6-lutidine, potassium bis(trimethylsilyl)amide (KHMDS) or lithium bis(trimethylsilyl)amide (LiH/IDS).

In one embodiment, the first reaction of Process 5 (a) (as described in scheme 21) comprises reacting Compound B30 with reducing agent. In another embodiment, the reducing agent comprises diisobutylaluminium hydride (DIBAL). In another embodiment, the reducing reaction is conducted at −60° C. to 0° C. In another embodiment, the reducing reaction is conducted at about −40° C.

In one embodiment, the second reaction of Process 5 (a) (as described in scheme 21) comprises reaction with a second base. In another embodiment, the second base comprises potassium tert-butoxide (t-BuOK) or sodium tert-butoxide (t-BuONa). In another embodiment, the second base comprises a strong non-nucleophilic base.

In one embodiment, the reaction of Process 5 (b) (as described in scheme 22) comprises 9-Iodo-9-borabicyclo[3.3.1]nonane (9-I-BBN). In one embodiment, the reaction of Process 5 (b) (as described in scheme 22) comprises acid. In another embodiment, the acid is acetic acid. In one embodiment, the reaction of Process 5 (b) (as described in scheme 22) comprises NaBO₃. In one embodiment, the reaction of Process 5 (b) (as described in scheme 22) comprises alcohol and catalytic amount of acid. In another embodiment, the acid is sulfonic acid. In another embodiment, the acid is H₂SO₄.

In one embodiment, the reaction conditions of Process 5 (b) as described in scheme 22, to obtain Compound B32 from Compound B31 (as described in scheme 22) comprises: (i) 9-I-BBN, (ii) AcOH, (iii) aq NaHCO₃/NaBO3 and (iv) alcohol and catalytic amount of acid. In another embodiment, the acid of Process 5, scheme 22, is a strong acid. In another embodiment, the strong acid is catalytic amount of H₂SO₄.

In one embodiment, the reaction conditions of Process 6 step (b) (as described in scheme 25) comprises a non nucleophilic base. In another embodiment, the non nucleophilic base comprises LiHMDS or KHMDS.

In one embodiment, Process 6 step (c) (as described in scheme 26) comprises 3 steps: (i) selective deprotection (removal of R₁₃), (ii) reduction of the non-terminal double bond, and (iii) deprotection (removal of R₁₁ and R₁₂).

In one embodiment, Process 6 step (c) (as described in scheme 26) comprises the following steps: (i) reduction of the non-terminal double bond, and (ii) deprotection. In another embodiment, the reduction, and the removal of R₁₃ is done simultaneously.

In one embodiment, Process 6 step (c) (as described in scheme 26) comprises selective deprotection (removal of R₁₃). In another embodiment, the selective deprotection comprises reaction with TiCl₄ or combination of TiCl₄ with catalytic amount of 1,1,3,3-Tetramethylguanidine (TMG). In another embodiment, the selective deprotection comprises reaction a combination of TiCl₄ with strong non-nucleophilic base.

In another embodiment, the removal of R₁₃, wherein if R₁₃ is a silyl group, comprises any known standard methods in the art and include those described in detail in Protecting Groups in Organic Synthesis, T. W. Greene and P. G. M. Wuts, 3rd edition, John Wiley & Sons, 1999; Bruce, A., et al., WO199965894; Bruce, A., et al., WO2004034990; Bruce, A., et al., WO2007061874; and Austad, B., rt al., WO2005118565 of which are incorporated entirety herein by reference. In another embodiment, the removal of the silyl group comprises fluoride anion. In another embodiment, the removal of the silyl group comprises acidic conditions. In another embodiment, the removal of the silyl group comprises basic conditions.

In one embodiment, Process 6 step (c) (as described in scheme 26) comprises reduction of the non-terminal double bond. In another embodiment, the reduction comprises reaction with a reducing agent. In another embodiment, the reducing agent comprises NaBH(OAc)₃, BnMe₃NBH(OAc)₃ or combination of BnMe₃N-halide and NaBH(OAc)₃.

In one embodiment, Process 6 step (c) (as described in scheme 26) comprises deprotection of the protecting groups (removal of R₁₁, R₁₂ and/or R₁₃). In one embodiment, Process 6 step (C) (as described in scheme 26) comprises deprotection of the acyl protecting groups. In another embodiment, the deprotection comprises basic conditions. In another embodiment, the basic conditions comprise (i) metal and/or ammonium hydroxides, (ii) metal and/or ammonium alkoxides, (iii) Na₂CO₃, (iv) K₂CO₃, or (v) Cs₂CO₃. In another embodiment, the deprotection of the acyl protecting groups comprises any known standard methods in the art and include those described in detail in Protecting Groups in Organic Synthesis, T. W. Greene and P. G. M. Wuts, 3rd edition, John Wiley & Sons, 1999 of which is incorporated entirety herein by reference.

In another embodiment, the deprotection of the acyl protecting groups comprises reaction with reducing agent. In another embodiment, the reducing agent is DIBAL, LiBH₄ or LiAlH₄.

In one embodiment, the reaction of Process 6 step (d) (as described in scheme 27) comprises methylation. In another embodiment, the methylation conditions comprises: (i) halomethyl; and (ii) base. In another embodiment, the methylation conditions of Process 6 step (d) (as described in scheme 27) comprises: (i) MeOSO₂R, wherein R is an alkyl, an aryl, a substituted alkyl or a substituted aryl; and (ii) base. In another embodiment, the base comprises t-BuONa.

In one embodiment, the reaction conditions of Process 7 step (a) (as described in scheme 29) comprises an oxidizing agent. In another embodiment, the oxidizing agent comprises NaIO₄, H₅IO₆, KIO₄, LUI₄, HO₄ or combination of NMM and RuCl₃. In another embodiment, the oxidizing agent is NaIO₄. In another embodiment, the oxidizing agent is H₅IO₆. In another embodiment, the oxidizing agent is KIO₄. In another embodiment, the oxidizing agent is LUI₄. In another embodiment, the oxidizing agent is HIO₄.

In one embodiment, the reaction conditions of Process 7 step (b) (as described in scheme 30) comprises: (i) a strong base that is not a nucleophile and (ii) LiCl. In another embodiment, the strong base that is not a nucleophile comprises 1,1,3,3-Tetramethylguanidine (TMG), DBU, LiH, KH, diisopropylethylamine, or a combination thereof, or any base that is not a nucleophile, each is a separate embodiment according to this invention. In another embodiment, the reaction conditions of Process 7 step (b) comprises TMG and LiCl.

In one embodiment, the reaction conditions of Process 7 step (c) (as described in scheme 31) comprises a reducing agent. In another embodiment, the reducing agent comprises DIBAL, LiBH₄ or NaAlH₄. In another embodiment, the reducing agent is DIBAL. In another embodiment, the reducing agent is LiBH₄. In another embodiment, the reducing agent is NaAlH₄.

In one embodiment, the reaction conditions of Process 7 step (d) (as described in scheme 32) comprises a fluoride anion source. In another embodiment, the fluoride ion source comprises CsF, tetra-n-butylammonium fluoride, or Et₃N-3HF.

In some embodiments, the reaction conditions of Process 8 step (a) (as described in scheme 33), Process 9 step (a) (as described in scheme 39) and Process 10 step (a) (as described in scheme 41) comprises Ligand I21, CrCl₂, 1,8-Bis(dimethylamino)naphthalene (proton sponge), NiCl₂-dmp cat., Mn, LiCi and Zirconocene dichloride (Cp₂ZrCl₂). In another embodiment, this reaction conditions comprises Ligand I21, CrCl₂, proton sponge, NiCl₂-dmp cat, Mn, LiCi or Cp₂ZrCl₂ or any combination thereof. In another embodiment, this reaction conditions comprises I21. In another embodiment, this reaction conditions comprises CrCl₂. In another embodiment, this reaction conditions comprises 1,8-Bis(dimethylamino)naphthalene (proton sponge). In another embodiment, this reaction conditions comprises NiCl₂-dmp (2,9-dimethyl-1,10-phenanthroline (neocuproine))cat. In another embodiment, this reaction conditions comprises Mn. In another embodiment, this reaction conditions comprises LiCl. In another embodiment, this reaction conditions comprises Cp₂ZrCl₂.

In some embodiments, the reaction of Process 8 step (b) (as described in scheme 34) comprises esterification reaction. In one embodiment, the esterification reaction conditions comprises: (i) acyl anhydride or acyl halide; and (ii) base. In another embodiment, the esterification reaction conditions comprises: (i) acyl anhydride; and (ii) base. In another embodiment, the esterification reaction conditions comprises: (i) acyl halide; and (ii) base. In another embodiment, the esterification reaction conditions comprises (i) acetic anhydride, acetyl halide, benzoyl halide, pivaloyl halide, benzoic anhydride or butyryl halide and (ii) base. In another embodiment, the esterification reaction conditions comprises (i) acetic anhydride and (ii) base. In another embodiment, the esterification reaction comprises (i) acetyl halide and (ii) base. In another embodiment, the esterification reaction conditions comprises (i) benzoyl halide and (ii) base. In another embodiment, the esterification reaction comprises (i) acetic anhydride and (ii) base. In another embodiment, the esterification reaction conditions comprises (i) pivaloyl halide and (ii) base. In another embodiment, the esterification reaction conditions comprises (i) benzoic anhydride and (ii) base. In another embodiment, the esterification reaction conditions comprises (i) butyryl halide and (ii) base. In another embodiment, the base of the esterification reaction conditions of Process 8 step (b) comprises pyridine, alkyl substituted pyridine, tertiary amines, alkylmorpholines, DMAP, DBU or a combination thereof.

In some embodiments, the reaction of Process 8 step (c) (as describe in scheme 35) comprises selective deprotection of the Si group. In one embodiment, the selective deprotection comprises acidic conditions. In one embodiment, the acidic conditions comprise a catalytic amount of acid or a fluoride anion source. In one embodiment, the selective deprotection conditions comprises a catalytic amount of acid or fluoride anion source. In one embodiment, the selective deprotection conditions comprises a catalytic amount of acid. In another embodiment, the acid is sulfonic acid. In another embodiment, the acid is H₂SO₄. In another embodiment, the acid is HCl. In another embodiment, the acid is HBr. In another embodiment, the fluoride anion comprises, CsF, TBAF, Et₃N-3HF.

In some embodiments, the reaction conditions of Process 8 step (d) (as describe in scheme 36) comprises abase. In one embodiment, the base comprises tertiary alkyl amine, pyridine, alkyl substituted pyridine, alkylmorpholine, DBU, 4-DMAP, or combination thereof. In another embodiment, the base comprises any non-nucleophilic organic base. In another embodiment, the base is tertiary alkyl amines. In another embodiment, the base is diisopropylethylamine. In another embodiment, the base is triethylamine. In another embodiment, the base is alkyl substituted pyridine. In another embodiment, the base is 2,6-lutidine. In another embodiment, the base is collidine. In another embodiment, the base is pyridine. In another embodiment, the base is DBU. In another embodiment, the base is 4-DMAP. In another embodiment, the base is alkylmorpholines.

In some embodiments, the cyclization reaction conditions of Process 8 step (e) (as describe in scheme 37) comprises a base. In another embodiment, the base is a strong base. In another embodiment, the strong base is Na—(OR), K—(OR) or Li—(OR) wherein R is an alkyl. Each represent a separate embodiment of this invention. In another embodiment, the base is NaOMe.

In some embodiments, the deprotection conditions of —OR₇ to OH, and OR₈ to OH of process 8 step (f) (as describe in scheme 38) and Process 10 step (c) (as described in scheme 43) comprises an acid. In one embodiment, the acid comprises an alkyl sulfonic acid, an aryl sulfonic acid, an aqueous sulfuric acid or combination thereof. In another embodiment, the acid is an alkyl sulfonic acid. In another embodiment, the acid is an aryl sulfonic acid. In another embodiment, the acid comprises an aqueous sulfuric acid. In another embodiment, the alkyl sulfonic acid is methanesulfonic acid. In another embodiment, the aryl sulfonic acid is p-Toluenesulfonic acid.

In some embodiments, the cyclization and deprotection of the diol protecting group reaction conditions of Process 9 step (b) (as described in scheme 40) comprise a OTf moiety. In another embodiment, OTf moiety comprises TMSOTf, TBSOTf or a combination thereof. In another embodiment, OTf moiety comprises TBSOTf.

In some embodiment, R₇ and R₈ of a compound of Formula III12, IV17, IV16, IV6 are each independently an alcohol protecting group or R₇ and R₈ form together with the oxygen a 5-6-member ring optionally substituted. In another embodiment, the alcohol protecting group is acid-sensitive protective group.

In some embodiments, the reaction conditions of Process 10 step (b) (as described in scheme 42) comprise a strong non-nucleophilic base. In another embodiment, the strong non-nucleophilic base comprises potassium bis(trimethylsilyl)amide (KHMDS), KH in combination with 6-crown ether, lithium bis(trimethylsilyl)amide (LiHMDS), or combination thereof. In another embodiment, the strong non-nucleophilic base comprises KHMDS. In another embodiment, the strong non-nucleophilic base comprises KH in combination with 6-crown ether. In another embodiment, the strong non-nucleophilic base comprises LiHMDS. In another embodiment, the strong non-nucleophilic base comprises KH in combination with 6-crown ether and LiHMDS.

In some embodiments, the protection process on compounds provided herein and the deprotection steps on the compounds provided herein are described in detail in Protecting Groups in Organic Synthesis, T. W. Greene and P. G. M. Wuts, 3rd edition, John Wiley & Sons, 1999; Bruce, A., et al., WO199965894; Bruce, A., et al., WO2004034990; Bruce, A., et al., WO2007061874; and Austad, B., rt al., WO2005118565 of which are incorporated entirety herein by reference.

As used herein, the term alkyl, used alone or as part of another group, refers, in one embodiment, to a “C₁ to C₁₂ alkyl” and denotes linear and branched, saturated or unsaturated (e.g., alkenyl, alkynyl) groups, the latter only when the number of carbon atoms in the alkyl chain is greater than or equal to two, and can contain mixed structures. Non-limiting examples are alkyl groups containing from 1 to 6 carbon atoms (C₁ to C₆ alkyls), or alkyl groups containing from 1 to 4 carbon atoms (C₁ to C₄ alkyls). Examples of saturated alkyl groups include, but are not limited to, methyl, ethyl, n-propyl, isopropyl, n-butyl, iso-butyl, sec-butyl, tert-butyl, amyl, tert-amyl and hexyl. Examples of alkenyl groups include, but are not limited to, vinyl, allyl, butenyl and the like. Examples of alkynyl groups include, but are not limited to, ethynyl, propynyl and the like. Similarly, the term “C₁ to C₁₂ alkylene” denotes a bivalent radical of 1 to 12 carbons.

The alkyl group can be unsubstituted, or substituted with one or more substituents selected from the group consisting of halogen, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, or alkylsulfonyl groups. Any substituents can be unsubstituted or further substituted with any one of these aforementioned substituents.

The term “haloalkyl” used herein alone or as part of another group, refers to, in some embodiments, to an alkyl group as defined above, which is substituted by one or more halogen atoms, e.g. by F, Cl, Br or I. For Example Halo-methyl comprises MeF, Mel, MeCl or MeBr.

The term “aryl” used herein alone or as part of another group denotes an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. The aryl group can be unsubstituted or substituted through available carbon atoms with one or more groups such as halogen, hydroxy, alkoxy, aryloxy, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, or alkylsulfonyl groups. Any substituents can be unsubstituted or further substituted with any one of these aforementioned substituents.

The term “heteroaryl” refers to an aromatic ring system containing from 5-14 member ring having at least one heteroatom in the ring. Non-limiting examples of suitable heteroatoms which can be included in the aromatic ring include oxygen, sulfur, phospate and nitrogen. Non-limiting examples of heteroaryl rings include pyridinyl, pyrrolyl, oxazolyl, indolyl, isoindolyl, purinyl, furanyl, thienyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, etc. The heteroaryl group can be unsubstituted or substituted through available carbon atoms with one or more groups such as. halogen, alkyl, aryl, hydroxy, alkoxy, aryloxy, alkylaryloxy, heteroaryloxy, oxo, cycloalkyl, phenyl, heteroaryls, heterocyclyl, naphthyl, amino, amido, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, alkylthio, arylthio, alkylsulfonyl, —OCN, —SCN, —N═C═O, —NCS, —NO, —N3, —OP(═O)(OR*)2, —P(═O)(OR*)2, —P(═O)(O—)2, —P(═O)(OH)2, —P(O)(OR*)(O—), —C(═O)R*, —C(═O)X, —C(S)R*, —C(S)OR*, —C(O)SR*, C(S)SR*, —C(S)NR* 2 or —C(═NR*)NR* 2 groups, where each R* is independently H, alkyl, aryl, arylalkyl, a heterocycle, or a protecting group or prodrug moiety. Any substituents can be unsubstituted or further substituted with any one of these aforementioned substituents.

As used herein, the term “amino”, used alone or as part of another group, refers to any primary, secondary, tertiary or quaternary amine each independently substituted with H, substituted or unsubstituted straight or branched C1-C10 alkyl, straight or branched C2-C10 alkenyl, straight or branched C2-C10 alkynyl, substituted or unsubstituted carbocyclyl, substituted or unsubstituted aryl, substituted or unsubstituted heterocyclyl, etc. In some embodiments, the primary, secondary and tertiary amines where the point of attachment is through the nitrogen-atom. In case of the secondary or tertiary amines, the substituting groups on the nitrogen may be the same or different. Nonlimiting types of amino include —NH2, —N(alkyl)2, —NH(alkyl), —N(carbocyclyl)2, —NH(carbocyclyl), —N(heterocyclyl)2, —NH(heterocyclyl), —N(aryl)2, —NH(aryl), —N(alkyl)(aryl), —N(alkyl)(heterocyclyl), —N(carbocyclyl)(heterocyclyl), —N(aryl)(heteroaryl), N(alkyl)(heteroaryl), etc. The term “alkylamino” refers to an amino group substituted with at least one alkyl group. Nonlimiting examples of amino groups include —NH2, —NH(CH₃), —N(CH₃)2, —NH(CH₂CH₃), —N(CH₂CH₃)2, —NH(phenyl), —N(phenyl)2, —NH(benzyl), —N(benzyl)2, etc. Substituted alkylamino refers generally to alkylamino groups, as defined above, in which at least one substituted alkyl, as defined herein, is attached to the amino nitrogen atom. Non-limiting examples of substituted alkylamino includes —NH(alkylene-C(O)—OH), —NH(alkylene-C(O)—O-alkyl), —N(alkylene-C(O)—OH)2, —N(alkylene-C(O)—O— alkyl)2, etc.

The term “halogen”, “halo” or “halide” as used herein refers to —Cl, —Br, —F, or —I groups.

The term “acyl” as used herein refer to —(C═O)—R wherein R is substituted or unsubstituted straight or branched C1-C12 alkyl, straight or branched C2-C12 alkenyl, straight or branched C2-C12 alkynyl, substituted or unsubstituted carbocyclyl, substituted or unsubstituted aryl, substituted or unsubstituted heterocyclyl, etc. Examples of acyl group include, but are not limited to pivaloyl, acetyl, benzoyl or trityl.

The term “ester” as used herein refer to —O—(C═O)—R or —(C═O)—O—R wherein R is substituted or unsubstituted straight or branched C1-C12 alkyl, straight or branched C2-C12 alkenyl, straight or branched C2-C12 alkynyl, substituted or unsubstituted carbocyclyl, substituted or unsubstituted aryl, substituted or unsubstituted heterocyclyl, etc.

The term “bn” or “benzyl” as used herein refer phenyl substituted with a methylene group —CH-Ph. In another embodiment, phenyl group of the benzyl group may be substituted by a alkyl, aryl, halogen, haloalkyl, hydroxyl, alkoxy, carbonyl, amido, alkylamido, dialkylamido, cyano, nitro, CO₂H, amino, alkylamino, dialkylamino, carboxyl, sulfonyl, thio

In some embodiment, R₁₆, R₇, R₈, R₇*, R₈* and, R₉ of the compounds of this invention are each independently an alcohol protecting group. In another embodiment, the alcohol protecting group is stable to hydrogenation. In another embodiments, the alcohol protecting group is stable in oxidation conditions. In another embodiments, the alcohol protecting group is stable to NaIO₄ (oxidative cleavage conditions). In another embodiments, the alcohol protecting group is stable in reduction conditions. In another embodiments, the alcohol protecting group is stable in acidic condition. In another embodiments, the alcohol protecting group is stable in basic condition. In another embodiments, the alcohol protecting group is stable to strong non-nucleophilic base. In another embodiments, the alcohol protecting group is acyl. In another embodiments, the alcohol protecting group is acyl. Examples of alcohol protecting group include, but are not limited to, acetyl, benzoyl, benzyl, pivaloyl, silyl ether, p-Methoxybenzy, and trityl, each is a separate embodiment according to this invention. In another embodiments, the alcohol protecting group is benzyl. Examples of alcohol protecting group include, but are not limited to, acetyl, benzoyl, benzyl, pivaloyl, silyl ether and trityl, each is a separate embodiment according to this invention. In another embodiments, the alcohol protecting group is benzyl. In another embodiments, the alcohol protecting group is silyl ether, tert-butyldimethylsilyl (TBS), or trimethylsilyl. Examples of alcohol protecting group include, but are not limited to trimethylsilyl (TMS), triethylsilyl (TES), t-Butyldiphenylsilyl (TBDPS), Diethylisopropylsilyl (DEIPS), di-t-butyldimethylsilylene (DTBS), or Triisopropylsilyl (TIPS). In another embodiments, the alcohol protecting group is an acid-sensitive protective group. In another embodiments, the alcohol protecting group is a base-sensitive protective group. In another embodiment, the alcohol protecting group is ether. In another embodiment, the alcohol protecting group include those described in detail in Protecting Groups in Organic Synthesis, T. W. Greene and P. G. M. Wuts, 3rd edition, John Wiley & Sons, 1999 of which is incorporated entirety herein by reference, each is a separate embodiment according to this invention.

In some embodiments, R₁₆ is pivaloyl.

The term “alcohol protecting group moiety” used herein, at process 7 step (c) to obtain a compound of Formula IV11 from a compound of Formula IV10, and at process 8 step (e) to obtain a compound of Formula IV6 from a compound of Formula IV5, refer to any known alcohol protecting group reagent which is known in the art and include those described in detail in Protecting Groups in Organic Synthesis, T. W. Greene and P. G. M. Wuts, 3rd edition, John Wiley & Sons, 1999 of which is incorporated entirety herein by reference, each is a separate embodiment according to this invention. In one embodiment, the “alcohol protecting group moiety” refers to acyl halide or acyl anhydride. In one embodiment, the “alcohol protecting group moiety” refers to benzyl halide. In one embodiment, the “alcohol protecting group moiety” refers to Y₁,Y₂,Y₃Si— halide, wherein Y₁, Y₂ and Y₃ are each independently are an alkyl or an aryl.

The term “acyl halide” refer to —C═O which is connected to alky/aryl and also connected to Cl or Br or I or F. For Example, but not limiting, acetyl chloride—CH₃COCl or benzoyl chloride—C₆H₅COCl

The term “a leaving group” is well known in the art, e.g., see “Advanced Organic Chemistry,” Jerry March, 4th Ed., pp. 351-357, John Wiley and Sons, N.Y. (1992). Such leaving groups include, but are not limited to, halogen, alkoxy, sulphonyloxy, optionally substituted alkylsulphonyloxy, optionally substituted alkenylsulfonyloxy, optionally substituted arylsulfonyloxy, silyl, and diazonium moieties, each is a separate embodiment according to this invention. Examples of a leaving group includes chloro, iodo, bromo, fluoro, methanesulfonyl (mesyl), tosyl, triflate or nitro-phenylsulfonyl (nosyl); each is a separate embodiment according to this invention.

As used herein, the term “isomer thereof” refers to a compound made up of the same atoms bonded by the same bonds but having different three-dimensional structures, which are not interchangeable. The three-dimensional structures are called configurations. Therefore, any one of the structures of this invention or isomers thereof include a single enantiomer, a diastereomer, a racemic mixture, cis configuration or a trans configuration. In another embodiment, the term “isomer thereof” refer to configurational stereoisomer. In another embodiment, the term “isomer thereof” refer to optical stereoisomer. In another embodiment, the term “isomer thereof” refer to each chiral carbon of the compounds of this invention is in S-configuration or R-configuration or racemate mixture.

The term “about” in reference to a numerical value stated herein is to be understood as the stated value +/−10%.

In some embodiment X₁ of a compound of Formula II29 is —OSO₂CF₃. In another embodiment X₁ is Cl. In another embodiment X₁ is Br. In another embodiment X₁ is I.

The term “sulfonic acid” refers to HO(SO₂)R, wherein R is substituted or unsubstituted (C₁ to C₁₈)alkyl, substituted or unsubstituted (C₅-C₁₈)aryl, or substituted or unsubstituted heteroaryl.

The term “non nucleophilic base” is a sterically hindered organic base that is a poor nucleophile (the proton-removing ability of a base without any other functions). Examples of non nucleophilic base include, but are not limited are: N,N-Diisopropylethylamine (DIPEA), 1,8-Diazabicyclo[5.4.0]undec-7-ene (DBU), Lithium diisopropylamide (LDA) and (Li, Na, K) hexamethyldisilazide (HMDS).

In some embodiments, provided herein is a process for the preparation of Eribulin, wherein the process comprises preparing Eribulin from a compound of Formula I6(R) or isomer thereof. In some embodiments, provided herein is a process for the preparation of Eribulin, wherein the process comprises preparing Eribulin from a compound of Formula I7(S) or isomer thereof. In some embodiments, provided herein is a process for the preparation of Eribulin, wherein the process comprises preparing Eribulin from a compound of Formula I8(R) or isomer thereof. In some embodiments, provided herein is a process for the preparation of Eribulin, wherein the process comprises preparing Eribulin from a compound of Formula I9 or isomer thereof. In some embodiments, provided herein is a process for the preparation of Eribulin, wherein the process comprises preparing Eribulin from a compound of Formula I10(S) or isomer thereof. In some embodiments, provided herein is a process for the preparation of Eribulin, wherein the process comprises preparing Eribulin from a compound of Formula I15 or isomer thereof. In some embodiments, provided herein is a process for the preparation of Eribulin, wherein the process comprises preparing Eribulin from a compound of Formula I4(R) or isomer thereof. In some embodiments, provided herein is a process for the preparation of Eribulin, wherein the process comprises preparing Eribulin from a compound of Formula I6(S) or isomer thereof. In some embodiments, provided herein is a process for the preparation of Eribulin, wherein the process comprises preparing Eribulin from a compound of Formula I7(R) or isomer thereof. In some embodiments, provided herein is a process for the preparation of Eribulin, wherein the process comprises preparing Eribulin from a compound of Formula I16(R)(1) or isomer thereof. In some embodiments, provided herein is a process for the preparation of Eribulin, wherein the process comprises preparing Eribulin from a compound of Formula I17(1) or isomer thereof. In some embodiments, provided herein is a process for the preparation of Eribulin, wherein the process comprises preparing Eribulin from a compound of Formula I18 or isomer thereof. In some embodiments, provided herein is a process for the preparation of Eribulin, wherein the process comprises preparing Eribulin from a compound of Formula I20 or isomer thereof. In some embodiments, provided herein is a process for the preparation of Eribulin, wherein the process comprises preparing Eribulin from a compound of Formula I12 or isomer thereof. In some embodiments, provided herein is a process for the preparation of Eribulin, wherein the process comprises preparing Eribulin from compound B26 or isomer thereof. In some embodiments, provided herein is a process for the preparation of Eribulin, wherein the process comprises preparing Eribulin from a compound of Formula II27 or isomer thereof.

In some embodiments, provided herein is a process for the preparation of Eribulin, wherein the process comprises preparing Eribulin from a compound of Formula II28(1). In some embodiments, provided herein is a process for the preparation of Eribulin, wherein the process comprises preparing Eribulin from compound B32 or isomer thereof. In some embodiments, provided herein is a process for the preparation of Eribulin, wherein the process comprises preparing Eribulin from a compound of Formula II28(2) or isomer thereof. In some embodiments, provided herein is a process for the preparation of Eribulin, wherein the process comprises preparing Eribulin from compound B31 or isomer thereof. In some embodiments, provided herein is a process for the preparation of Eribulin, wherein the process comprises preparing Eribulin from a compound of Formula III5 or isomer thereof.

In some embodiments, provided herein is a process for the preparation of Eribulin, wherein the process comprises preparing Eribulin from a compound of Formula III6 or isomer thereof. In some embodiments, provided herein is a process for the preparation of Eribulin, wherein the process comprises preparing Eribulin from a compound of Formula III9 or isomer thereof. In some embodiments, provided herein is a process for the preparation of Eribulin, wherein the process comprises preparing Eribulin from a compound of Formula III11 or isomer thereof. In some embodiments, provided herein is a process for the preparation of Eribulin, wherein the process comprises preparing Eribulin from a compound of Formula III12 or isomer thereof. In some embodiments, provided herein is a process for the preparation of Eribulin, wherein the process comprises preparing Eribulin from a compound of Formula IV8 or isomer thereof. In some embodiments, provided herein is a process for the preparation of Eribulin, wherein the process comprises preparing Eribulin from a compound of Formula IV9 or isomer thereof. In some embodiments, provided herein is a process for the preparation of Eribulin, wherein the process comprises preparing Eribulin from a compound of Formula IV11 or isomer thereof.

In some embodiments, provided herein is a process for the preparation of Eribulin, wherein the process comprises preparing Eribulin from a compound of Formula IVI or isomer thereof. In some embodiments, provided herein is a process for the preparation of Eribulin, wherein the process comprises preparing Eribulin from a compound of Formula IV2 or isomer thereof. In some embodiments, provided herein is a process for the preparation of Eribulin, wherein the process comprises preparing Eribulin from a compound of Formula IV3 or isomer thereof. In some embodiments, provided herein is a process for the preparation of Eribulin, wherein the process comprises preparing Eribulin from a compound of Formula IV4 or isomer thereof. In some embodiments, provided herein is a process for the preparation of Eribulin, wherein the process comprises preparing Eribulin from a compound of Formula IV6 or isomer thereof. In some embodiments, provided herein is a process for the preparation of Eribulin, wherein the process comprises preparing Eribulin from a compound of Formula IV7 or isomer thereof.

In some embodiments, provided herein is a process for the preparation of Eribulin, wherein the process comprises preparing Eribulin from a compound of Formula IV17 or isomer thereof. In some embodiments, provided herein is a process for the preparation of Eribulin, wherein the process comprises preparing Eribulin from a compound of Formula II29 or isomer thereof. In some embodiments, provided herein is a process for the preparation of Eribulin, wherein the process comprises preparing Eribulin from a compound of Formula IV16 or isomer thereof. In some embodiments, provided herein is a process for the preparation of Eribulin, wherein the process comprises preparing Eribulin from a compound of Formula IV1 or isomer thereof.

In some embodiments, provided herein is a process for the preparation of Eribulin, wherein the process comprises
(i) reducing the ketone group of IV12 to form diol IV13 or isomer thereof,
(ii) preparing Eribulin from IV13 according as described in references [36-39].

In some embodiments, provided herein is a process for the preparation of Eribulin, wherein the process comprises the following steps:
(i) preparing a compound of formula IV12 or isomer thereof:

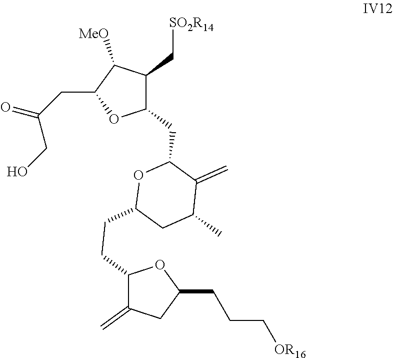

wherein R₁₄ is an alkyl or an aryl; R₁₆ is an alcohol protecting group; from a compound of formula IV7 or isomer thereof:

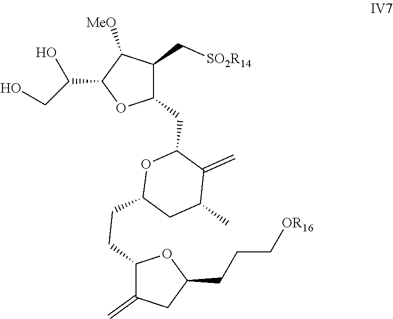

wherein R₁₄ is an alkyl or an aryl; R₁₆ is an alcohol protecting group.
by elongating the chain of the diol; and
(ii) preparing Eribulin from a compound of formula IV12 or isomer thereof, wherein the process comprises reducing the ketone group of IV12 or isomer thereof to form diol, protecting the diol group and coupling reaction to obtain Eribulin.

In some embodiments, provided herein is a process for the preparation of Eribulin, wherein the process comprises the following steps:
(i) preparing a compound of formula IV12 or isomer thereof:

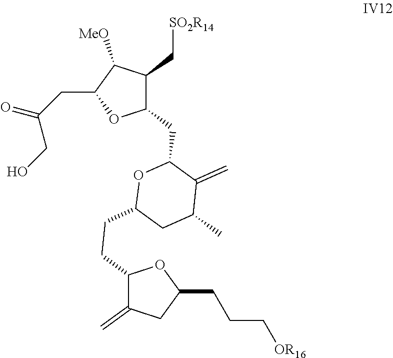

wherein R₁₄ is an alkyl or an aryl; R₁₆ is an alcohol protecting group; from a compound of formula IV7 or isomer thereof:

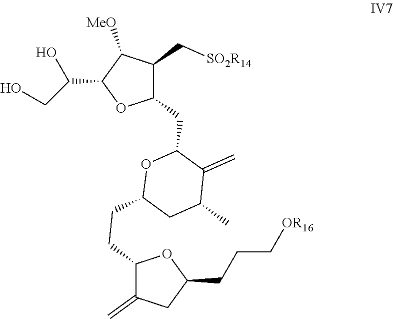

wherein R₁₄ is an alkyl or an aryl; R₁₆ is an alcohol protecting group.
by elongating the chain of the diol as described in process 7 of this invention; and
(iii) preparing Eribulin from a compound of formula IV12 or isomer thereof, wherein the process comprises reducing the ketone group of IV12 or isomer thereof to form diol, protecting the diol group and coupling reaction to obtain Eribulin.

In some embodiments, provided herein is a preparation of Eribulin, wherein the process comprises:
(i) reducing the ketone of IV12 to obtain IV13 or isomer thereof, followed by protecting the diol group and selective deprotection to remove R₁₆ to obtain IV15 or isomer thereof; and
(ii) preparing Eribulin from IV15 or isomer thereof as known in the art.

In one embodiment, the compound of Formula I6(R) of Process 1 is Compound A6(R) or isomer thereof:

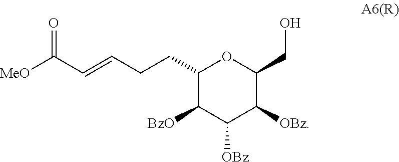

In one embodiment, Compound A6(R) is used for the preparation of Eribulin.

In one embodiment, the compound of Formula I7(S) of Process 1 is Compound A7(S) or isomer thereof:

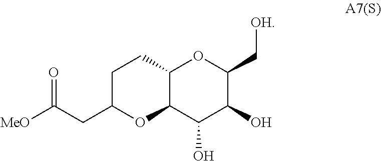

In one embodiment, Compound A7(S) is used for the preparation of Eribulin.

In one embodiment, the compound of Formula I8(R) of Process 1 is Compound A8(R) or isomer thereof:

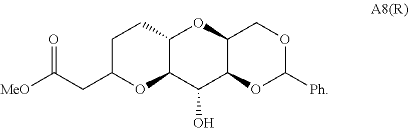

In one embodiment, Compound A8(R) is used for the preparation of Eribulin.

In one embodiment, the compound of Formula I9 of Process 1 is Compound A9 or isomer thereof:

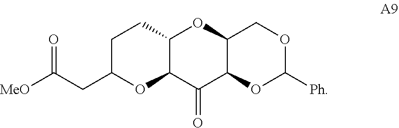

In one embodiment, Compound A9 is used for the preparation of Eribulin.

In one embodiment, the compound of Formula I10(S) of Process 1 is Compound A10(S) or isomer thereof:

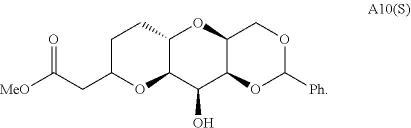

In one embodiment, Compound A10(S) is used for the preparation of Eribulin.

In one embodiment, the compound of Formula I15 of Process 2 is Compound A15 or isomer thereof:

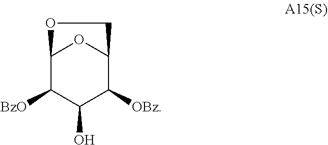

In one embodiment, Compound A15(S) is used for the preparation of Eribulin.

In one embodiment, the compound of Formula I4(R) of Process 2 is Compound A4(R) or isomer thereof:

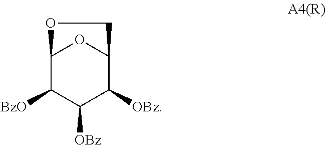

In one embodiment, Compound A4(R) is used for the preparation of Eribulin.

In one embodiment, the compound of Formula I6(S) of Process 2 is Compound A6(S) or isomer thereof:

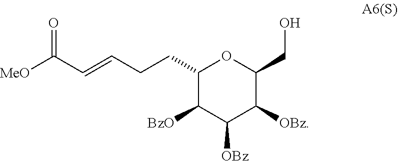

In one embodiment, Compound A6(S) is used for the preparation of Eribulin.

In one embodiment, the compound of Formula I7(R) of Process 2 and Process 3 is Compound A7(R) or isomer thereof:

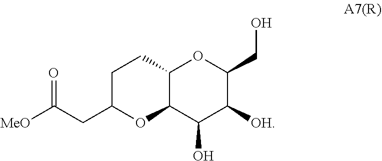

In one embodiment, Compound A7(R) is used for the preparation of Eribulin.

In one embodiment, the compound of Formula I16(R)(1) of Process 3 is Compound A16 or isomer thereof:

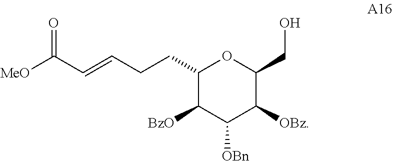

In one embodiment, Compound A16 is used for the preparation of Eribulin.

In one embodiment, the compound of Formula I17(1) of Process 3 is Compound A17 or isomer thereof:

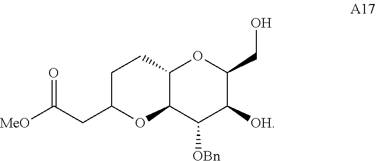

In one embodiment, Compound A17 is used for the preparation of Eribulin.

In one embodiment, the compound of Formula I18 of Process 3 is Compound A18 or isomer thereof:

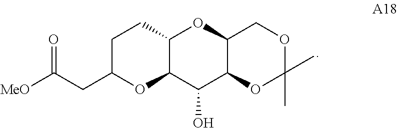

In one embodiment, Compound A18 is used for the preparation of Eribulin.

In one embodiment, the compound of Formula I20 of Process 3 is Compound A20 or isomer thereof

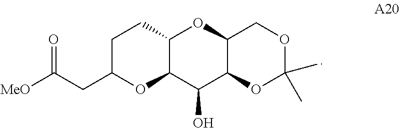

In one embodiment, Compound A20 is used for the preparation of Eribulin.

In one embodiment, the compound of Formula II27 of Process 4 is Compound B27 or isomer thereof:

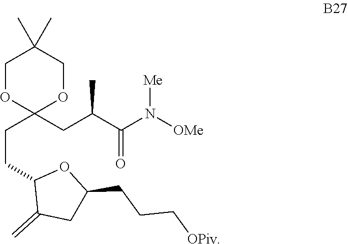

In one embodiment, Compound B27 is used for the preparation of Eribulin.

In one embodiment, a compound of Formula II28(1) of Process 4 is Compound B28(1) or isomer thereof:

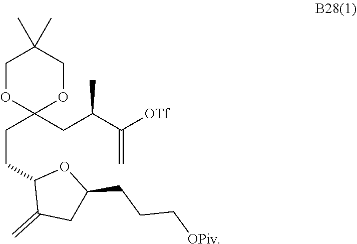

In one embodiment, Compound B28(1) is used for the preparation of Eribulin.

In one embodiment, the compound of Formula II28(2) of Process 5 is Compound B28(2) or isomer thereof:

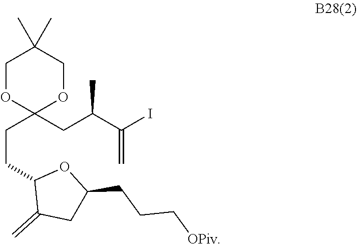

In one embodiment, Compound B28(2) is used for the preparation of Eribulin.

In one embodiment, the compound of Formula III5 of Process 6 is Compound C5 or isomer thereof:

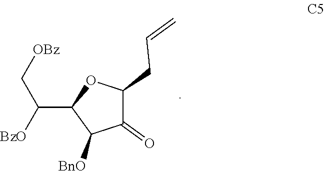

In one embodiment, Compound C5 is used for the preparation of Eribulin.

In one embodiment, the compound of Formula III6 of Process 6 is Compound C6 or isomer thereof:

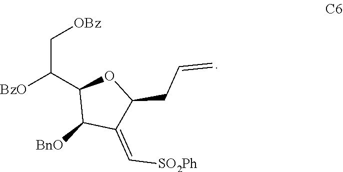

In one embodiment, Compound C6 is used for the preparation of Eribulin.

In one embodiment, the compound of Formula III9 of Process 6 is Compound C9 or isomer thereof:

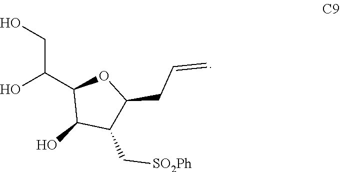

In one embodiment, Compound C9 is used for the preparation of Eribulin.

In one embodiment, the compound of Formula III11 of Process 6 is Compound C11 or isomer thereof:

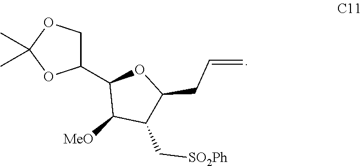

In one embodiment, Compound C11 is used for the preparation of Eribulin.

In one embodiment, the compound of Formula III12 of Process 6 is Compound C12 or isomer thereof:

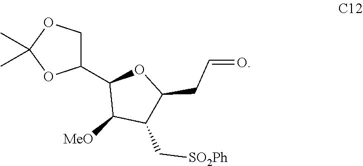

In one embodiment, Compound C12 is used for the preparation of Eribulin.

In one embodiment, the compound of Formula IV7 of any one of processes 7-10 is Compound D7 or isomer thereof:

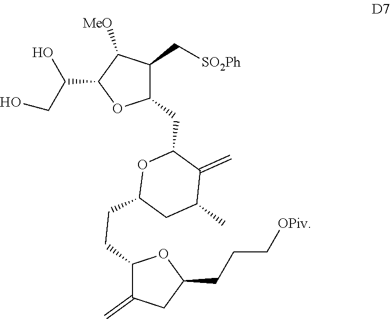

In one embodiment, Compound D7 is used for the preparation of Eribulin.

In one embodiment, the compound of Formula IV8 of Process 7 is Compound D8 or isomer thereof:

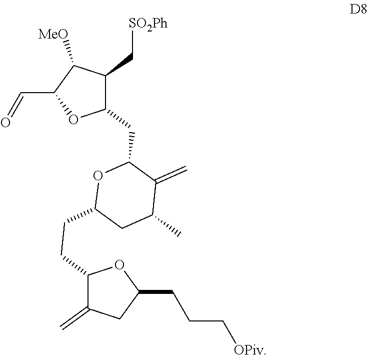

In one embodiment, Compound D8 is used for the preparation of Eribulin.

In one embodiment, the compound of Formula IV9 of Process 7 is Compound D9 or isomer thereof:

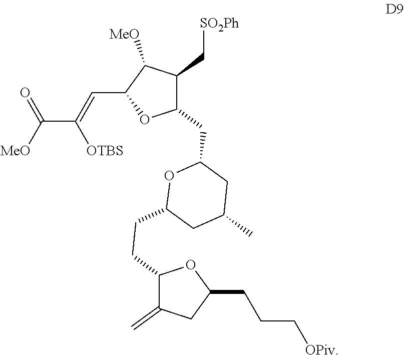

In one embodiment, Compound D9 is used for the preparation of Eribulin.

In one embodiment, the compound of Formula IV11 of Process 7 is Compound D11 or isomer thereof:

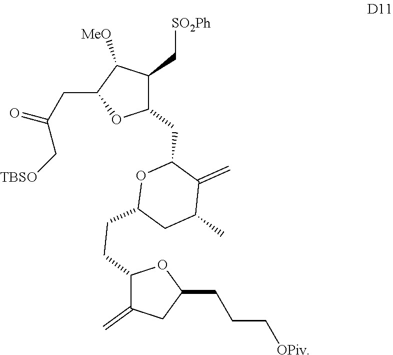

In one embodiment, Compound D11 is used for the preparation of Eribulin.

In one embodiment, the compound of Formula IV12 of Process 7 is Compound D12 or isomer thereof:

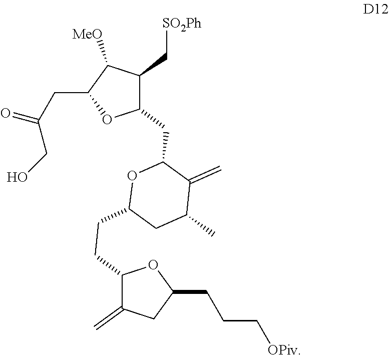

In one embodiment, Compound D12 is used for the preparation of Eribulin.

In one embodiment, the compound of Formula IV1 of Process 8 is Compound D1 or isomer thereof:

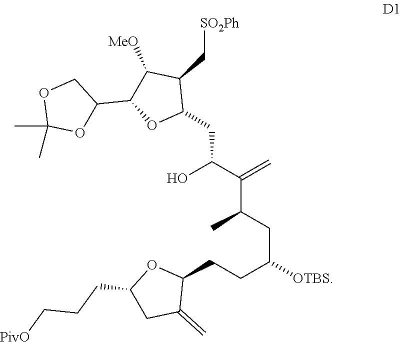

In one embodiment, Compound D1 is used for the preparation of Eribulin.

In one embodiment, the compound of Formula IV2 of Process 8 is Compound D2 or isomer thereof:

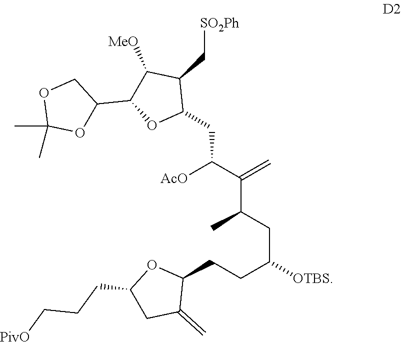

In one embodiment, Compound D2 is used for the preparation of Eribulin.

In one embodiment, the compound of Formula IV3 of Process 8 is Compound D3 or isomer thereof:

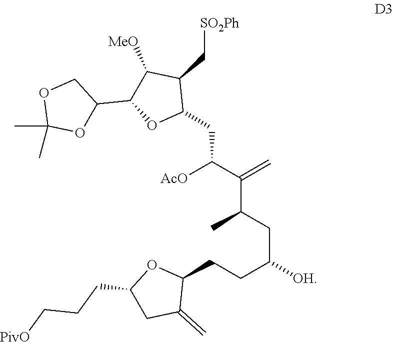

In one embodiment, Compound D3 is used for the preparation of Eribulin.

In one embodiment, the compound of Formula IV4 of Process 8 is Compound D4 or isomer thereof:

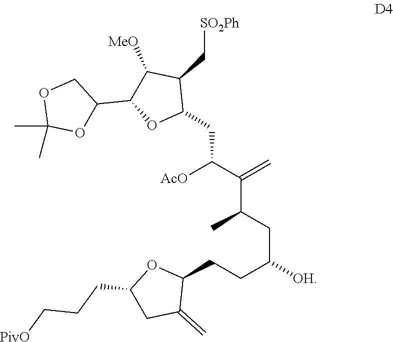

In one embodiment, Compound D4 is used for the preparation of Eribulin.

In one embodiment, the compound of Formula IV5 of Process 8 is Compound D5 or isomer thereof:

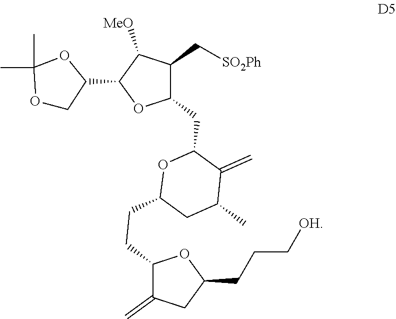

In one embodiment, Compound D5 is used for the preparation of Eribulin.

In one embodiment, the compound of Formula IV6 of Process 8 and Process 10 is Compound D6 or isomer thereof:

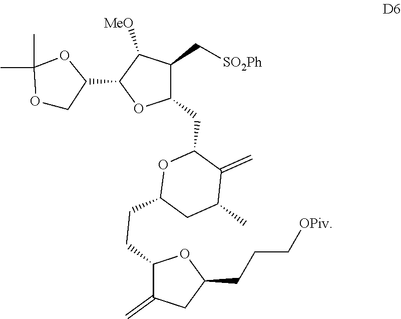

In one embodiment, Compound D6 is used for the preparation of Eribulin.

In one embodiment, the compound of Formula IV17 of Process 9 is Compound D17 or isomer thereof:

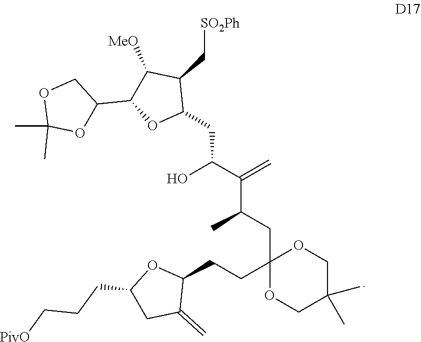

In one embodiment, Compound D17 is used for the preparation of Eribulin.

In one embodiment, the compound of Formula IV16 of Process 10 is Compound D16 or isomer thereof:

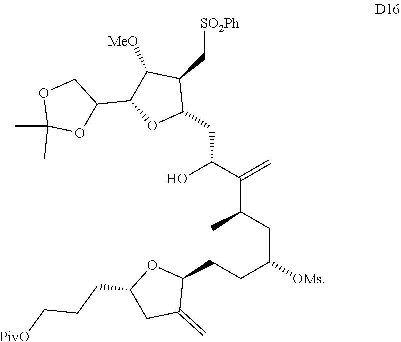

In one embodiment, Compound D16 is used for the preparation of Eribulin.

In one embodiment, provided herein is a compound represented by the structure of Formula I4(S) or isomer thereof:

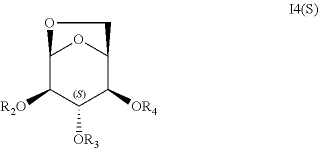

wherein R₂ and R₄ are each independently an acyl group, and R₃ is an acyl or benzyl group. In another embodiment, R₂, R₃ and R₄ are each independently benzoyl group. In another embodiment, R₂, and R₄ are each independently benzoyl group. In another embodiment, R₃ is benzoyl group. In another embodiment, R₃ is benzyl group. In another embodiment, the compound of Formula I4 is represented by the structure of Compound A4(S) or isomer thereof

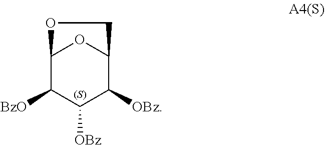

In one embodiment, provided herein is a compound represented by the structure of Formula I6(R) or isomer thereof:

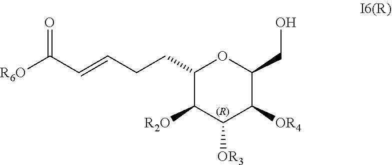

wherein R₂ and R₄ are each independently an acyl group, R₃ is an acyl or benzyl group; and R₆ is an alkyl. In another embodiment, R₂, R₃ and R₄ are each independently benzoyl group. In another embodiment, R₂, and R₄ are each independently benzoyl group. In another embodiment, R₃ is benzoyl group. In another embodiment, R₃ is benzyl group. In another embodiment, R₆ is methyl. In another embodiment, the compound of Formula I6(R) is represented by the structure of Compound A6(R) or isomer thereof:

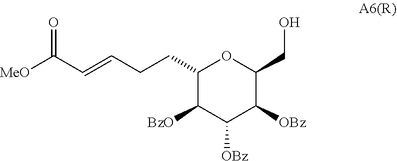

In one embodiment, provided herein is a compound represented by the structure of a Formula I7(S) or isomer thereof:

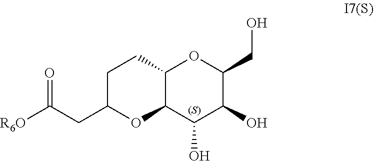

wherein R₆ is an alkyl group. In another embodiment, R₆ is methyl. In another embodiment, the compound of Formula I7(S) is represented by the structure of Compound A7(S):

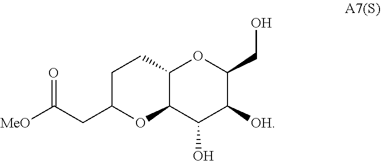

In one embodiment, provided herein is a compound represented by the structure of a Formula I7(R) or isomer thereof:

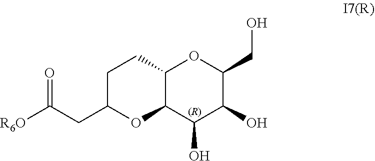

wherein R₆ is an alkyl group. In another embodiment, R₆ is methyl. In another embodiment, the compound of Formula I7(R) is represented by the structure of Compound A7(R):

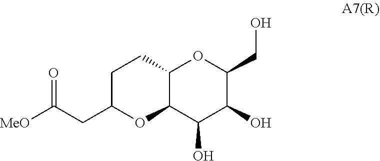

In one embodiment, provided herein is a compound represented by the structure of a Formula I8(R) or isomer thereof:

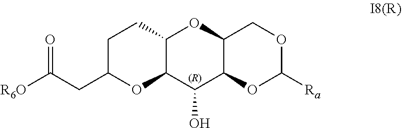

wherein Rₐ is substituted or unsubstituted alkyl, substituted or unsubstituted aryl, or substituted or unsubstituted heteroaryl; and R₆ is an alkyl.

In another embodiment, R₆ is methyl. In another embodiment, Rₐ is phenyl.

In another embodiment, the compound of Formula I8(R) is represented by the structure of Compound A8(R) or isomer thereof:

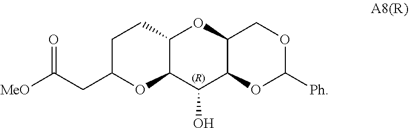

In one embodiment, provided herein is a compound represented by the structure of Formula I9 or isomer thereof:

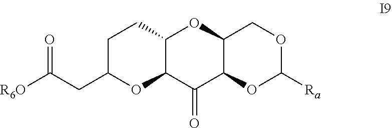

wherein Rₐ is substituted or unsubstituted alkyl, substituted or unsubstituted aryl, or substituted or unsubstituted heteroaryl; and R₆ is an alkyl group. In another embodiment, R₆ is methyl. In another embodiment, Rₐ is phenyl. In another embodiment, the compound of Formula I9 is represented by the structure of Compound A9:

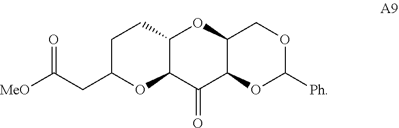

In one embodiment, provided herein is a compound represented by the structure of a Formula I10 or isomer thereof:

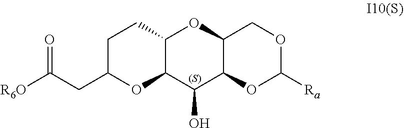

wherein Rₐ is substituted or unsubstituted alkyl, substituted or unsubstituted aryl, or substituted or unsubstituted heteroaryl; and R₆ is an alkyl group. In another embodiment, the compound of Formula I10(S) is represented by the structure of Compound A10(S) or isomer thereof:

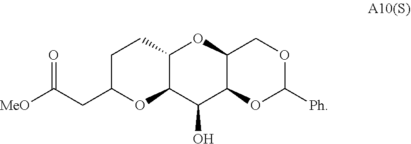

In one embodiment, provided herein is a compound represented by the structure of Formula I15(R) or isomer thereof:

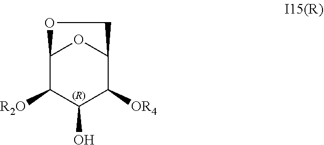

wherein R₂ and R₄ are each independently an acyl group. In another embodiment, R₂ and R₄ are each independently a benzoyl group.

In another embodiment, the compound of Formula I15(R) is a represented by the structure of Compound A15(R) or isomer thereof:

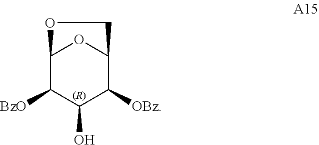

In one embodiment, provided herein is a compound represented by the structure of Formula I4(R) or isomer thereof:

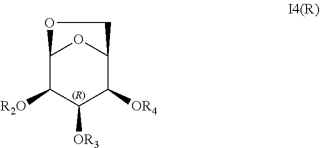

wherein R₂ and R₄ are each independently an acyl group; and R₃ is an acyl or benzyl group. In another embodiment, R₂, R₃ and R₄ are each independently benzoyl group. In another embodiment, R₂, and R₄ are each independently benzoyl group. In another embodiment, R₃ is benzoyl group. In another embodiment, R₃ is benzyl group. In another embodiment, the compound of Formula I4(R) is a represented by the structure of Compound A4(R) or isomer thereof

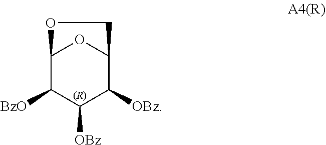

In one embodiment, provided herein is a compound represented by the structure of Formula I6(S) or isomer thereof

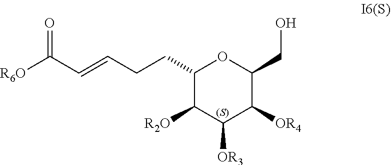

wherein R₂ and R₄ are each independently an acyl group; R₃ is an acyl or benzyl group; and R₆ is an alkyl. In another embodiment, R₂, R₃ and R₄ are each independently benzoyl group. In another embodiment, R₂, and R₄ are each independently benzoyl group. In another embodiment, R₃ is benzoyl group. In another embodiment, R₃ is benzyl group. In another embodiment, R₆ is methyl. In another embodiment, the compound of Formula I6(S) is a represented by the structure of Compound A6(S.) or isomer thereof

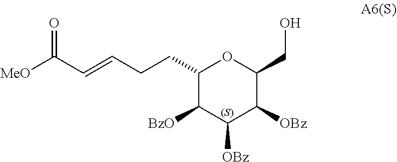

In one embodiment, provided herein is a compound represented by the structure of a Formula I7(R) or isomer thereof

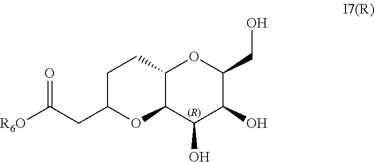

wherein R₆ is an alkyl group. In another embodiment, R₆ is a methyl group In another embodiment, the compound of Formula I7(R) is represented by the structure of Compound A7(R) or isomer thereof

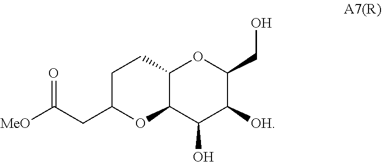

In one embodiment, provided herein is a compound represented by the structure of a Formula II1 or isomer thereof

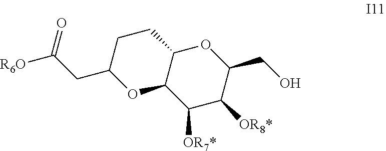

wherein R₇* and R₈* are each independently an alcohol protecting group or R₇* and R₈* form together with the oxygen a 5-6 member ring optionally substituted; and R₆ is an alkyl. In another embodiment, R₆ is a methyl group. In another embodiment, R₇* and R₈* form together with the oxygen a 5-6 member ring optionally substituted. In another embodiment, R₇* and R₈* form together a 5-member ring substituted with additional ring in a form of a spiro.

In another embodiment, R₇* and R₈* are as shown below:

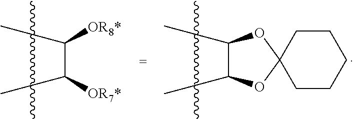

In another embodiment, the compound of Formula I11 is represented by the structure of Compound A11 or isomer thereof

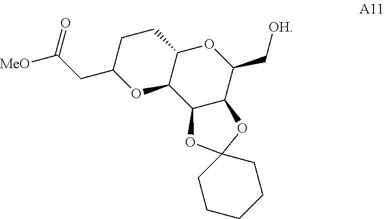

In one embodiment, provided herein is a compound represented by the structure of Formula I16(R)(1) or isomer thereof

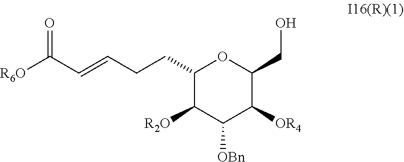

wherein R₂ and R₄ are each independently an acyl group; and R₆ is an alkyl. In another embodiment, R₂, R₃ and R₄ are each independently benzoyl group. In another embodiment, R₂, and R₄ are each independently benzoyl group. In another embodiment, R₃ is benzoyl group. In another embodiment, R₃ is benzyl group. In another embodiment, R₆ is methyl. In another embodiment, the compound of Formula I16(R)(1) is represented by the structure of A16 or isomer thereof:

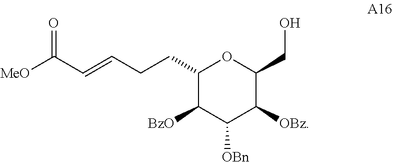

In one embodiment, provided herein is a compound represented by the structure of Formula I17(1) or isomer thereof:

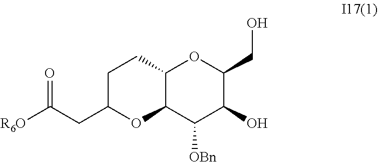

wherein R₆ is an alkyl group. In another embodiment, R₆ is methyl.

In another embodiment, the compound of Formula I17(1) is represented by the structure of A17 or isomer thereof:

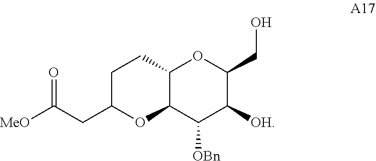

In one embodiment, provided herein is a compound represented by the structure of Formula I18:

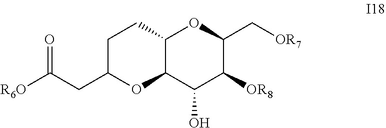

wherein R₇ and R₈ are each independently an alcohol protecting group or R₇ and R₈ form together with the oxygen a 6-7 member ring optionally substituted; and R₆ is an alkyl. In another embodiment, R₆ is methyl. In another embodiment, R₆ is methyl, In another embodiment, R₇ and R₈ form together a 6-member ring as shown below:

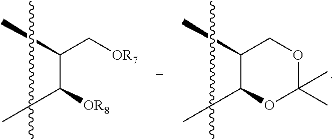

In another embodiment, the compound of Formula I18 is represented by the structure of A18 or isomer thereof:

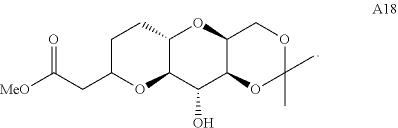

In one embodiment, provided herein is a compound represented by the structure of Formula I19 or isomer thereof:

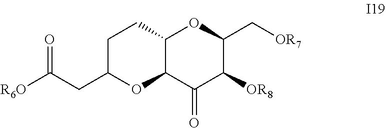

wherein R₇ and R₈ are each independently an alcohol protecting group or R₇ and R₈ form together with the oxygen a 6-7 member ring optionally substituted; and R₆ is an alkyl group. In another embodiment, R₆ is methyl. In another embodiment, R₆ is methyl, In another embodiment, R₇ and R₈ form together a 6-member ring as shown below:

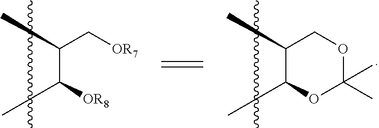

In another embodiment, the compound of Formula I19 is represented by the structure of A19 or isomer thereof:

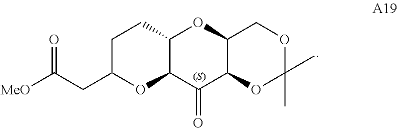

In one embodiment, provided herein is a compound represented by the structure of Formula I20 or isomer thereof:

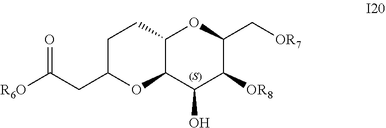

wherein R₇ and R₈ are each independently an alcohol protecting group or R₇ and R₈ form together with the oxygen a 5-6 member ring optionally substituted; and R₆ is an alkyl group. In another embodiment, R₆ is methyl. In another embodiment, R₆ is methyl, In another embodiment, R₇ and R₈ form together a 6-member ring as shown below:

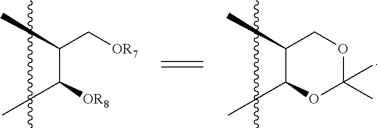

In another embodiment, the compound of Formula I20 is represented by the structure of A20 or isomer thereof:

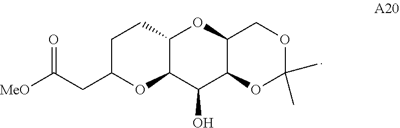

In one embodiment, provided herein is a compound represented by the structure of Compound B26:

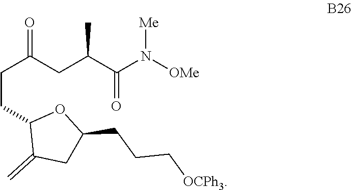

In one embodiment, provided herein is a compound represented by the structure of Formula II27 or isomer thereof:

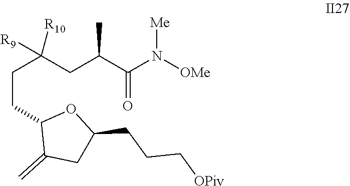

wherein R₉ and R₁₀ are each independently O-alkyl or S-alkyl; or R₉ and R₁₀ form together a 5-6-member acetal ring, optionally substituted. In another embodiment, R₉ and R₁₀ form together a 6-member acetal ring represented by the following structure

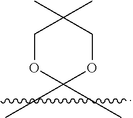

In another embodiment, the compound of Formula II27 or isomer thereof is represented by the structure of B27 or isomer thereof:

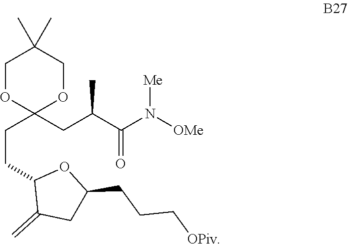

In one embodiment, provided herein is a compound represented by the structure of Formula II28(1) or isomer thereof:

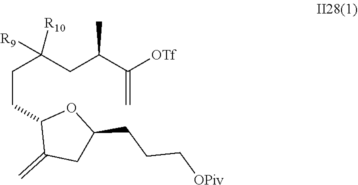

wherein R₉ and R₁₀ are each independently O-alkyl or S-alkyl; or R₉ and R₁₀ form together a 5-6-member acetal ring, optionally substituted; and Piv refers to pivaloyl. In another embodiment, R₉ and R₁₀ form together a 6-member acetal ring represented by the following structure

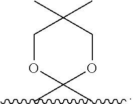

In another embodiment, the compound of Formula II28(1) is represented by the structure of Compound B28(1) or isomer thereof:

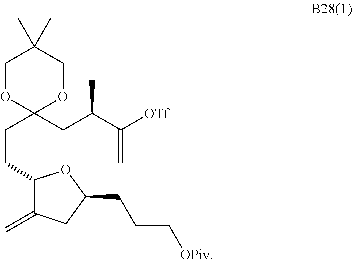

In one embodiment, provided herein is a compound represented by the structure of Compound B31 or isomer thereof:

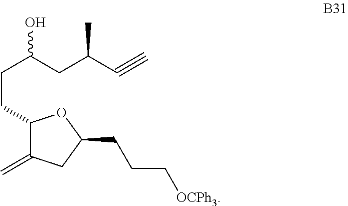

In one embodiment, provided herein is a compound represented by the structure of Compound B32 or isomer thereof:

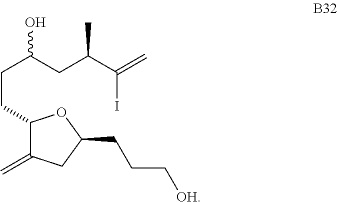

In one embodiment, provided herein is a compound represented by the structure of Formula II28(2) or isomer thereof:

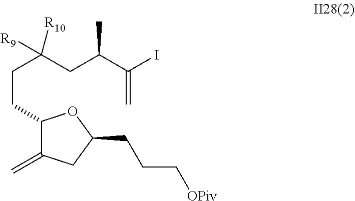

wherein R₉ and R₁₀ are each independently O-alkyl or S-alkyl; or R₉ and R₁₀ form together a 5-6-member acetal ring, optionally substituted; and Piv refers to pivaloyl. In another embodiment, R₉ and R₁₀ form together a 6-member acetal ring represented by the following structure

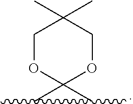

In another embodiment, the compound of Formula II28(2) or isomer thereof is represented by the structure of Compound B28(2) or isomer thereof:

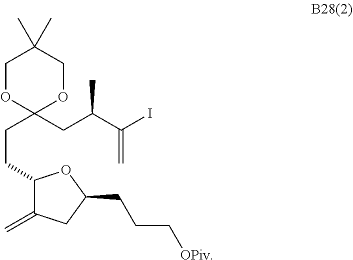

In one embodiment, provided herein is a compound represented by the structure of Formula III4 or isomer thereof:

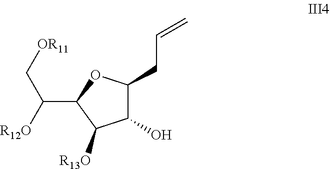

wherein OR₁₁ and OR₁₂ are each independently an ester group; and R₁₃ is —CH₂-aryl or

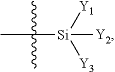

wherein Y₁, Y₂, and Y₃ are each independently an alkyl or an aryl. In another embodiment, the compound of Formula III4 or isomer thereof is represented by the structure of Compound C4 or isomer thereof:

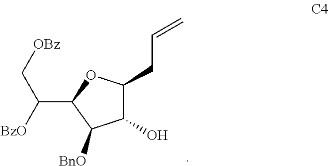

In one embodiment, provided herein is a compound represented by the structure of Formula III5 or isomer thereof:

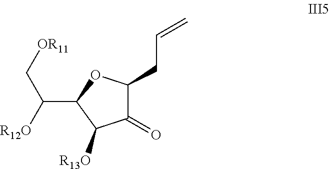

wherein OR₁₁ and OR₁₂ are each independently an ester group; and R₁₃ is —CH₂-aryl or

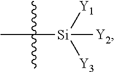

wherein Y₁, Y₂, and Y₃ are each independently an alkyl or an aryl. In another embodiment, R₁₃ is benzyl. In another embodiment, R₁ and R₁₂ are each independently benzoyl group. In another embodiment, the compound of Formula III5 or isomer thereof is represented by the structure of Compound C5 or isomer thereof:

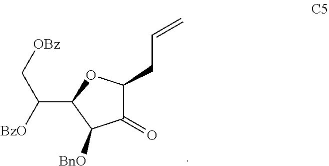

In one embodiment, provided herein is a compound represented by the structure of Formula III6 or isomer thereof:

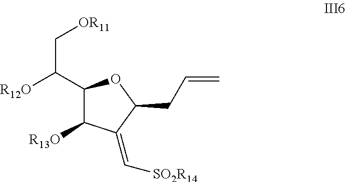

wherein OR₁₁ and OR₁₂ are each independently an ester group; and R₁₃ is —CH₂-aryl or

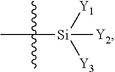

wherein Y₁, Y₂, and Y₃ are each independently an alkyl or an aryl; R₁₄ is an alkyl or aryl. In another embodiment, R₁₄ is phenyl. In another embodiment, R₁₁ and R₁₂ are each independently benzoyl group. In another embodiment, R₁₃ is benzyl. In another embodiment, the compound of Formula III6 or isomer thereof is represented by the structure of C6 or isomer thereof:

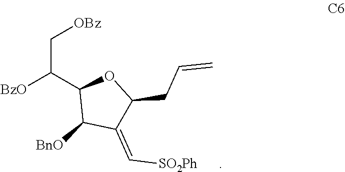

In one embodiment, provided herein is an intermediate of Process 6 (scheme 26) represented by the structure of Formula III7 or isomer thereof:

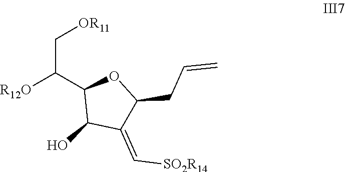

wherein OR₁₁ and OR₁₂ are each independently an ester group; and R₁₄ is an alkyl or aryl. In one embodiment, provided herein is a compound of Formula III7. In another embodiment, R₁₄ is phenyl. In another embodiment, R₁₁ and R₁₂ are each independently benzoyl group. In another embodiment, the compound of Formula III7 is represented by the structure of C7 or isomer thereof:

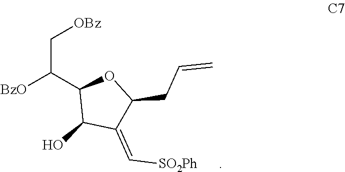

In one embodiment, provided herein is an intermediate of Process 6 (scheme 26) represented by the structure of Formula III8 or isomer thereof:

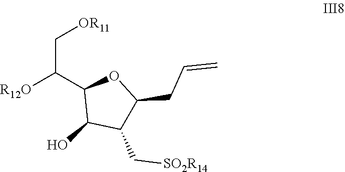

wherein OR₁₁ and OR₁₂ are each independently an ester group; and R₁₄ is an alkyl or aryl. In one embodiment, provided herein is a compound of Formula III8 or isomer thereof. In another embodiment, R₁₄ is phenyl. In another embodiment, R₁₁ and R₁₂ are each independently benzoyl group. In another embodiment, the compound of Formula III8 is represented by the structure of C8 or isomer thereof:

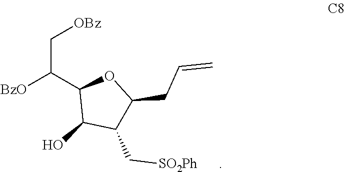

In one embodiment, provided herein is a compound represented by the structure of Formula III9 or isomer thereof:

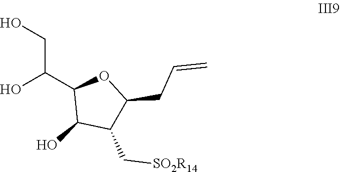

wherein R₁₄ is an alkyl or an aryl. In another embodiment, R₁₄ is phenyl. In another embodiment, the compound of Formula III9 or isomer thereof is represented by the structure of Compound C9 or isomer thereof:

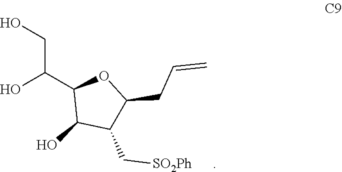

In one embodiment, provided herein is an intermediate of Process 6 (scheme 27) represented by the structure of Formula III0 or isomer thereof:

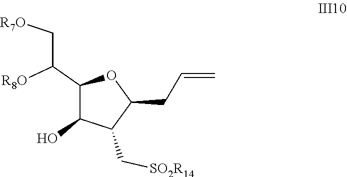

wherein R₇ and R₈ are each independently an alcohol protecting group or R₇ and R₈ form together with the oxygen a 5-6 member ring optionally substituted; and R₁₄ is an alkyl or aryl. In one embodiment, provided herein is a compound represented by the structure of Formula III0 or isomer thereof:

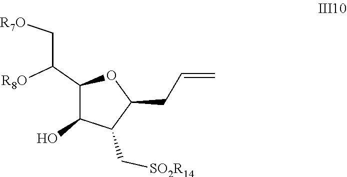

wherein R₇ and R₈ are each independently an alcohol protecting group or R₇ and R₈ form together with the oxygen a 5-6 member ring optionally substituted; and R₁₄ is an alkyl or aryl. In another embodiment, R₇ and R₈ form together with the oxygen a 5-member ring optionally substituted. In another embodiment, R₇ and R₈ form together with the oxygen a 5-member ring as shown:

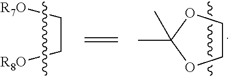

In another embodiment, the compound of Formula III10 is represented by the structure of Compound C10 or isomer thereof:

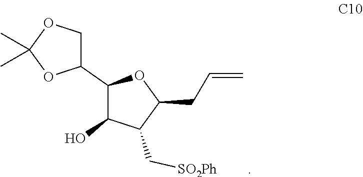

In one embodiment, provided herein is a compound represented by the structure of Formula III11 or isomer thereof:

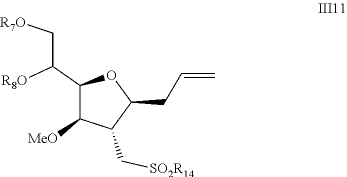

wherein R₇ and R₈ are each independently an alcohol protecting group or R₇ and R₈ form together with the oxygen a 5-6 member ring optionally substituted; and R₁₄ is an alkyl or an aryl. In another embodiment, R₇ and R₈ form together with the oxygen a 5-member ring as shown:

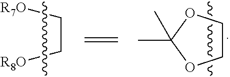

In another embodiment, the compound of Formula III11 or isomer thereof is represented by the structure of Compound C11 or isomer thereof:

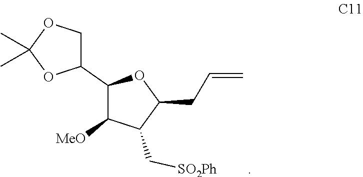

In one embodiment, provided herein is a compound represented by the structure of Formula III12 or isomer thereof:

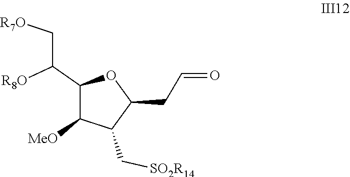

wherein R₇ and R₈ are each independently an alcohol protecting group or R₇ and R₈ form together with the oxygen a 5-6 member ring optionally substituted; and R₁₄ is an alkyl or an aryl. In another embodiment, R₇ and R₈ form together with the oxygen a 5-member ring as shown:

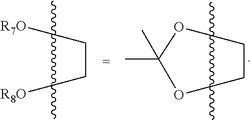

In another embodiment, the compound of Formula III12 or isomer thereof is represented by the structure of Compound C12 or isomer thereof:

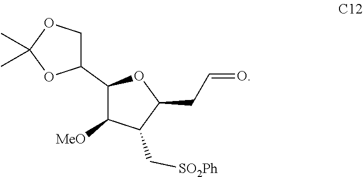

In another embodiment, the compound of Formula III12 or isomer thereof is represented by the structure of Compound C12a or isomer thereof

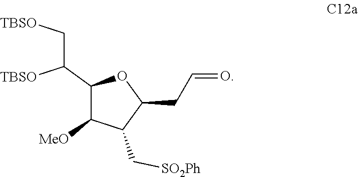

In one embodiment, provided herein is a compound represented by the structure of Formula IV1 or isomer thereof:

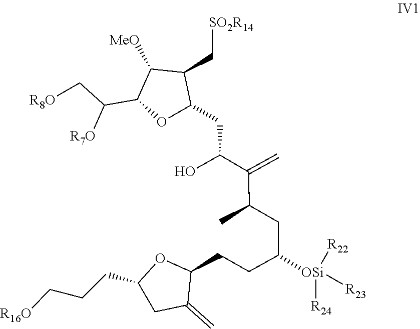

wherein R₇, R₈ and R₁₆ are each independently an alcohol protecting group; or R₇ and R₈ form together with the oxygen a 5-6-member ring optionally substituted; R₁₄, R₂₂, R₂₃ and R₂₄ are each independently an alkyl or aryl group. In another embodiment, R₇ and R₈ form together with the oxygen a 5-member ring as shown:

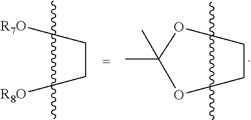

another embodiment, R₁₆ is pivaloyl group. In another embodiment, R₂₂, R₂₃ and R₂₄ are each independently t-butyl group. In another embodiment, R₂₂, R₂₃ and R₂₄ are each independently methyl group. In another embodiment, at least two of R₂₂, R₂₃ and R₂₄ are methyl group. In another embodiment, two of R₂₂, R₂₃ and R₂₄ are methyl group, and one is t-butyl group. In another embodiment, R₁₄ is phenyl. In another embodiment, the compound of Formula IV1 is represented by the structure of D1:

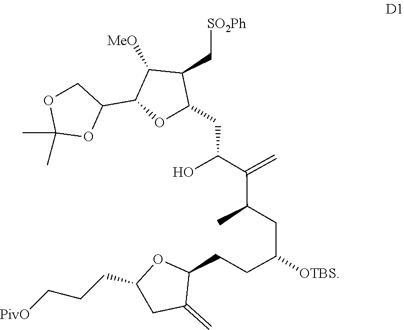

In one embodiment, provided herein is a compound represented by the structure of Formula IV2 or isomer thereof:

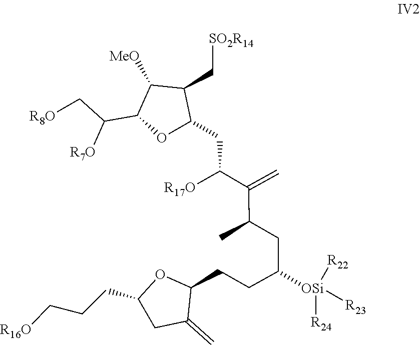

wherein OR₁₇ is an ester; R₇, R₈ and R₁₆ are each independently an alcohol protecting group; or R₇ and R₈ form together with the oxygen a 5-6-member ring optionally substituted; R₁₄, R₂₂, R₂₃ and R₂₄ are each independently an alkyl or aryl group. In another embodiment, R₇ and R₈ form together with the oxygen a 5-member ring as shown:

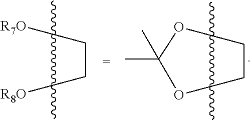

In another embodiment, R₁₆ is pivaloyl group. In another embodiment, R₁₇ is —C(═O)CH₃ group. In another embodiment, R₂₂, R₂₃ and R₂₄ are each independently t-butyl group. In another embodiment, R₂₂, R₂₃ and R₂₄ are each independently methyl group. In another embodiment, at least two of R₂₂, R₂₃ and R₂₄ are methyl group. In another embodiment, two of R₂₂, R₂₃ and R₂₄ are methyl group, and one is t-butyl group. In another embodiment, R₁₄ is phenyl. In another embodiment, the compound is represented by the structure of D2 or isomer thereof:

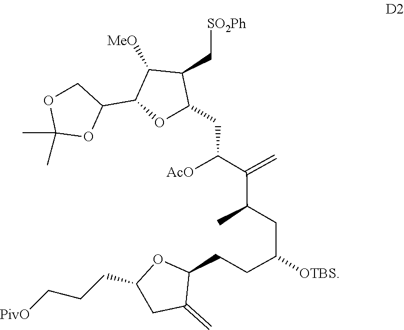

In one embodiment, provided herein is a compound represented by the structure of Formula IV3 or isomer thereof:

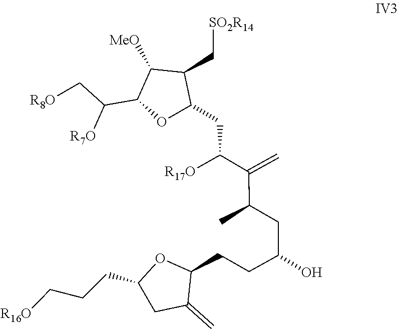

wherein R₇, R₈ and R₁₆ are each independently an alcohol protecting group; or R₇ and R₈ form together with the oxygen a 5-6-member ring; R₁₄ is an alkyl or aryl; and OR₁₇ is an ester. In another embodiment, R₇ and R₈ form together with the oxygen a 5-member ring as shown:

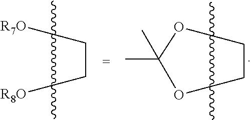

In another embodiment, R₁₆ is pivaloyl group. In another embodiment, R₁₇ is —C(═O)CH₃ group. In another embodiment, R₁₄ is phenyl. In another embodiment, the compound of Formula IV3 or isomer thereof is represented by the structure of D3 or isomer thereof:

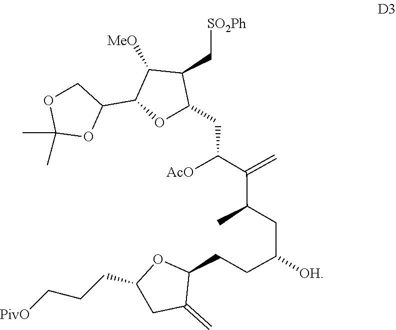

In one embodiment, provided herein is a compound represented by the structure of Formula IV4 or isomer thereof:

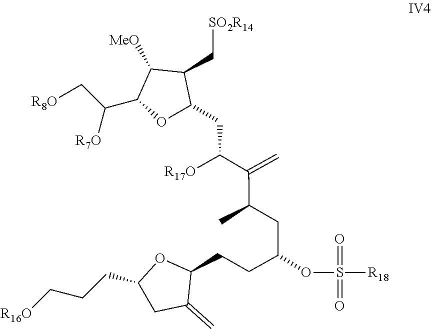

wherein R₇, R₈, R₁₅ and R₁₆ are each independently an alcohol protecting group; or R₇ and R₈ form together with the oxygen a 5-6-member ring; R₁₄ is an alkyl or aryl; and OR₁₇ is an ester; and R₁₈ is an alkyl or an aryl. In another embodiment, R₇ and R₈ form together with the oxygen a 5-member ring as shown:

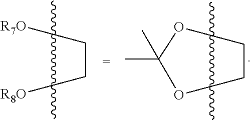

In another embodiment, R₁₆ is pivaloyl group. In another embodiment, R₁₇ is —C(═O)CH₃ group. In another embodiment, R₁₈ is methyl. In another embodiment, R₁₄ is phenyl. In another embodiment, the compound of Formula IV4 or isomer thereof is represented by the structure of D4 or isomer thereof:

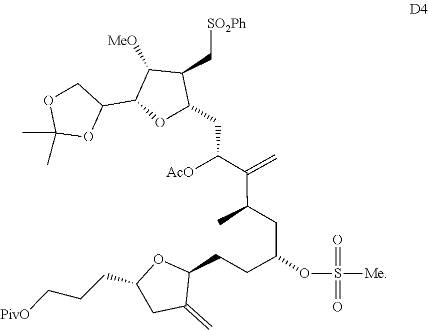

In one embodiment, provided herein is a compound represented by the structure of Formula IV5 or isomer thereof:

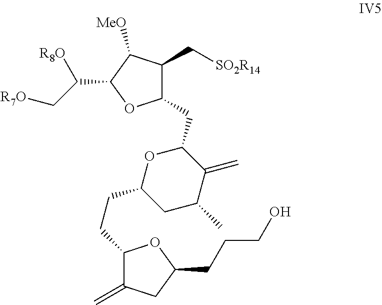

wherein R₇ and R₈ are each independently an alcohol protecting group; or R₇ and R₈ form together with the oxygen a 5-6-member ring; and R₁₄ is an alkyl or aryl. In another embodiment, R₇ and R₈ form together with the oxygen a 5-member ring as shown:

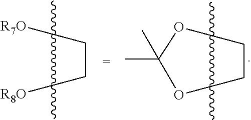

In another embodiment, R₁₄ is phenyl.

In another embodiment, the compound of Formula IV6 is represented by the structure of D5 or isomer thereof:

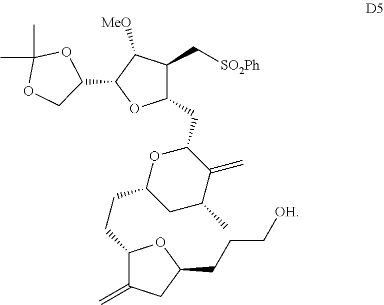

In one embodiment, provided herein is a compound represented by the structure of Formula IV6 or isomer thereof:

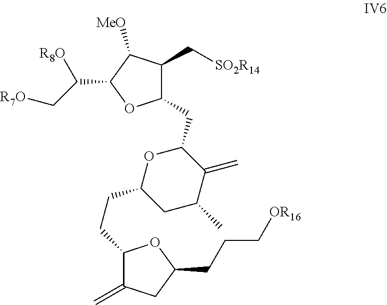

wherein R₇ and R₈ are each independently an alcohol protecting group; or R₇ and R₈ form together with the oxygen a 5-6-member ring; R₁₄ is an alkyl or aryl; and R₁₆ is an alcohol protecting group. In another embodiment, R₇ and R₈ form together with the oxygen a 5-member ring as shown:

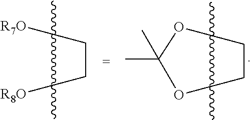

In another embodiment, R₁₉ is H. In another embodiment, R₁₉ is pivaloyl group. In another embodiment, R₁₄ is phenyl. In another embodiment, the compound of Formula IV6 or isomer thereof is represented by the structure of D6 or isomer thereof:

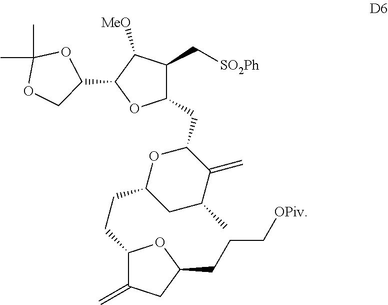

In one embodiment, provided herein is a compound represented by the structure of Formula IV7 or isomer thereof:

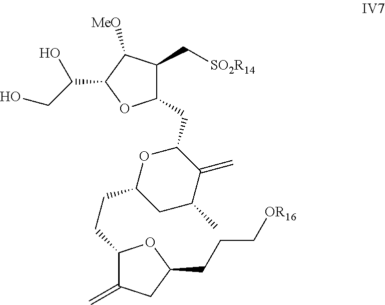

wherein R₁₄ is an alkyl or aryl; and R₁₆ is an alcohol protecting group. In another embodiment, R₁₆ is pivaloyl group. In another embodiment, R₁₄ is phenyl. In another embodiment, the compound of Formula IV7 or isomer thereof is represented by the structure of D7 or isomer thereof:

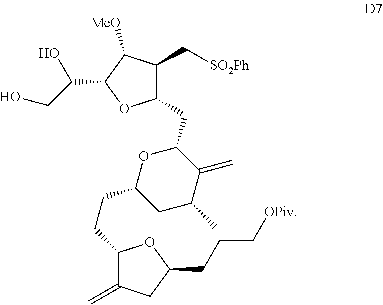

In one embodiment, provided herein is a compound represented by the structure of Formula IV8 or isomer thereof:

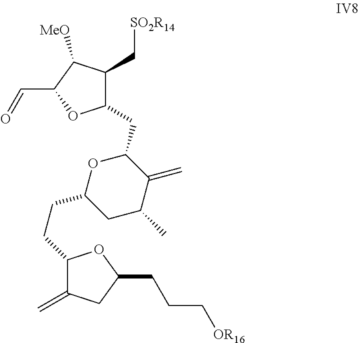

wherein R₁₄ is an alkyl or aryl; and R₁₆ is an alcohol protecting group. In another embodiment, R₁₆ is pivaloyl group. In another embodiment, R₁₄ is phenyl. In another embodiment, the compound of Formula IV8 is represented by the structure of D8 or isomer thereof:

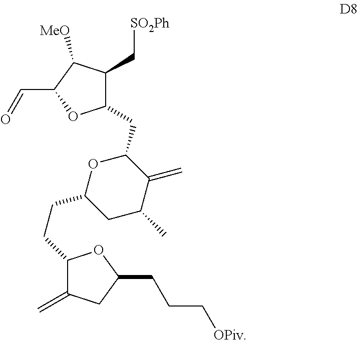

In one embodiment, provided herein is a compound represented by the structure of Formula IV9 or isomer thereof:

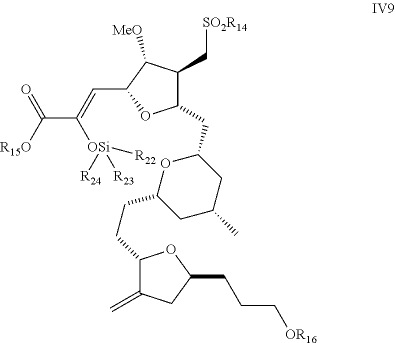

wherein R₁₄, R₁₅, R₂₂, R₂₃ and R₂₄ are each independently an alkyl or an aryl group; and R₁₆ is an alcohol protecting group. In another embodiment, R₁₄ is phenyl. In another embodiment, R₁₅ is methyl. In another embodiment, R₁₆ is pivaloyl group. In another embodiment, R₂₂, R₂₃ and R₂₄ are each independently t-butyl group. In another embodiment, R₂₂, R₂₃ and R₂₄ are each independently methyl group. In another embodiment, at least two of R₂₂, R₂₃ and R₂₄ are methyl group. In another embodiment, two of R₂₂, R₂₃ and R₂₄ are methyl group, and one is t-butyl group. In another embodiment, the compound of Formula IV9 or isomer thereof is represented by the structure of D9 or isomer thereof:

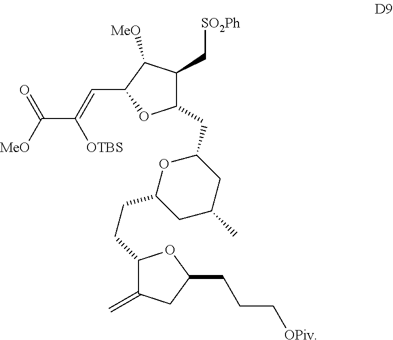

In one embodiment, provided herein is a compound represented by the structure of Formula IV10 or isomer thereof:

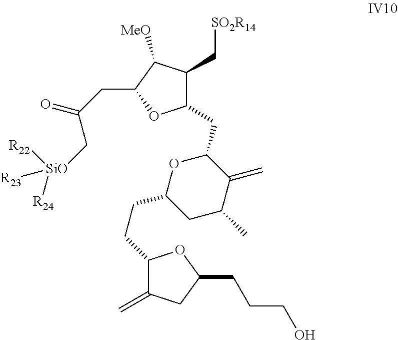

wherein R₁₄, R₂₂, R₂₃ and R₂₄ are each independently an alkyl or an aryl group; and R₁₆ is an alcohol protecting group. In another embodiment, R₁₄ is phenyl. In another embodiment, R₂₂, R₂₃ and R₂₄ are each independently t-butyl group. In another embodiment, R₂₂, R₂₃ and R₂₄ are each independently methyl group. In another embodiment, at least two of R₂₂, R₂₃ and R₂₄ are methyl group. In another embodiment, two of R₂₂, R₂₃ and R₂₄ are methyl group, and one is t-butyl group. In another embodiment, R₁₆ is pivaloyl group. In another embodiment, the compound of Formula IV10 or isomer thereof is represented by the structure of D10 or isomer thereof:

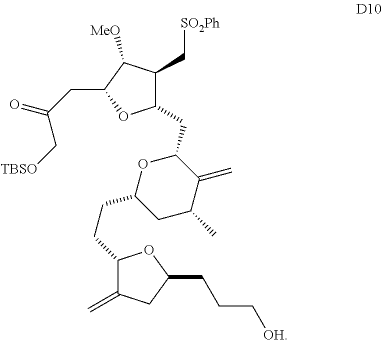

In one embodiment, provided herein is a compound represented by the structure of Formula IV1 or isomer thereof:

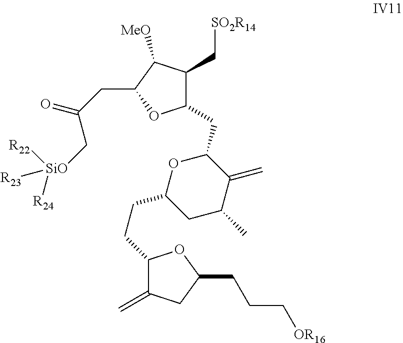

wherein R₁₄, R₂₂, R₂₃ and R₂₄ are each independently an alkyl or an aryl group; and R₁₆ is an alcohol protecting group. In another embodiment, R₁₄ is phenyl. In another embodiment, R₂₂, R₂₃ and R₂₄ are each independently t-butyl group. In another embodiment, R₂₂, R₂₃ and R₂₄ are each independently methyl group. In another embodiment, at least two of R₂₂, R₂₃ and R₂₄ are methyl group. In another embodiment, two of R₂₂, R₂₃ and R₂₄ are methyl group, and one is t-butyl group. another embodiment, R₁₆ is pivaloyl group. In another embodiment, the compound Formula IV11 or isomer thereof is represented by the structure of D11 or isomer thereof:

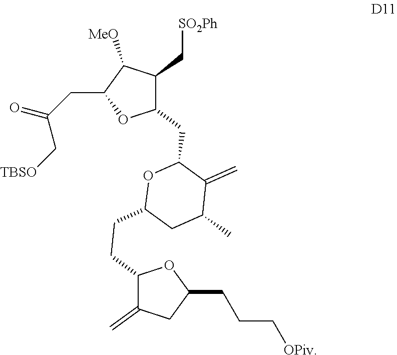

In one embodiment, provided herein is a compound represented by the structure of Formula IV12 or isomer thereof:

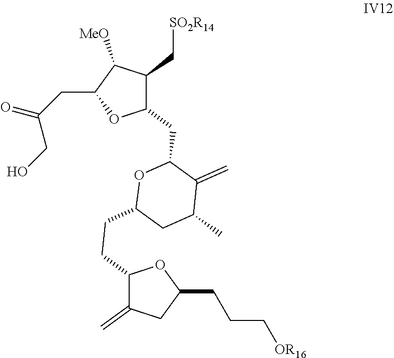

wherein R₁₄ is an alkyl or aryl; and R₁₆ is an alcohol protecting group. In another embodiment, R₁₄ is phenyl. In another embodiment, R₁₆ is pivaloyl group. In another embodiment, the compound of Formula IV12 or isomer thereof is represented by the structure of D12 or isomer thereof:

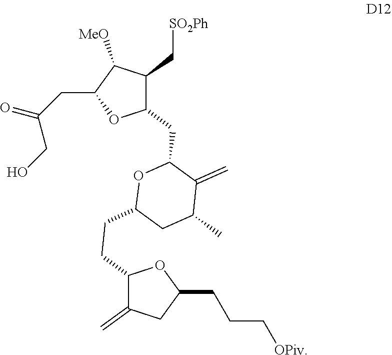

In one embodiment, provided herein is a compound represented by the structure of Formula IV17 or isomer thereof:

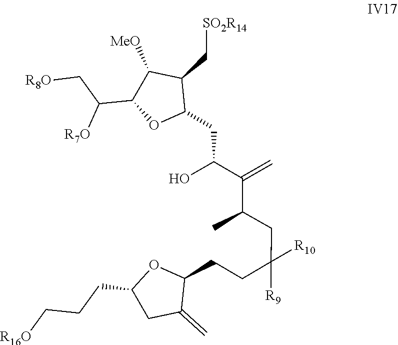

wherein R₇ and R₈ are each independently an alcohol protecting group; or R₇ and R₈ form together with the oxygen a 5-6-member ring optionally substituted; R₉ and R₁₀ are each independently O-alkyl or S-alkyl; or R₉ and R₁₀ form together a 5-6-member acetal ring; R₁₄ is an alkyl or an aryl; and R₁₆ is an alcohol protecting group. In another embodiment, R₁₄ is phenyl. In another embodiment, R₁₆ is pivaloyl group. In another embodiment, R₇ and R₈ are each independently a TBS group. In another embodiment, R₉ and R₁₀ form together a 6-member acetal ring represented by the following structure

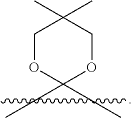

In another embodiment, the compound of Formula IV17 or isomer thereof is represented by the structure of D17 or isomer thereof:

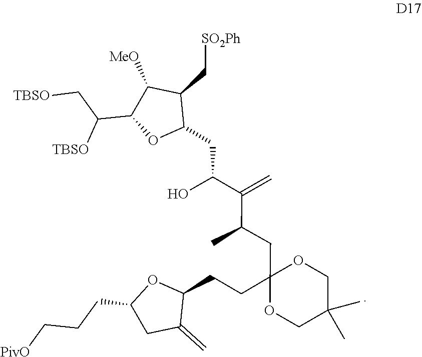

In one embodiment, provided herein is a compound represented by the structure of Formula IV16 or isomer thereof:

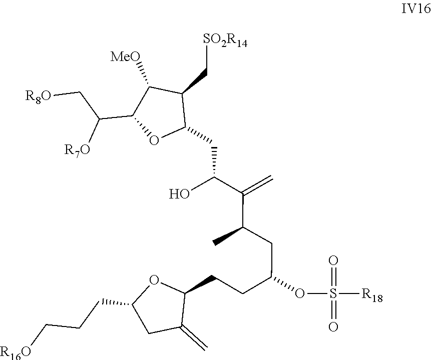

wherein R₇, R₈ and R₁₆ are each independently an alcohol protecting group; or R₇ and R₈ form together with the oxygen a 5-6-member ring; and R₁₄ and R₁₈ are each independently an alkyl or an aryl group. In another embodiment, R₇ and R₈ form together with the oxygen a 5-member ring as shown:

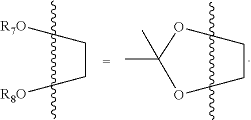

In another embodiment, the compound is represented by the structure of D16 or isomer thereof:

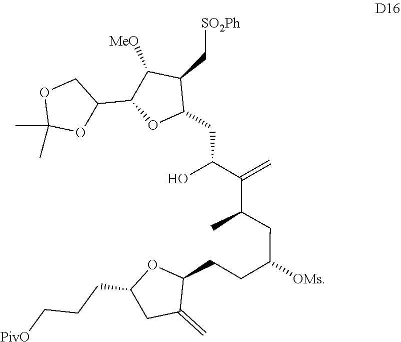

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

The following examples are presented in order to more fully illustrate the preferred embodiments of the invention. They should in no way be construed, however, as limiting the broad scope of the invention.

### EXAMPLES

### Example 1—Preparation of A12 (See Also FIG. 1)

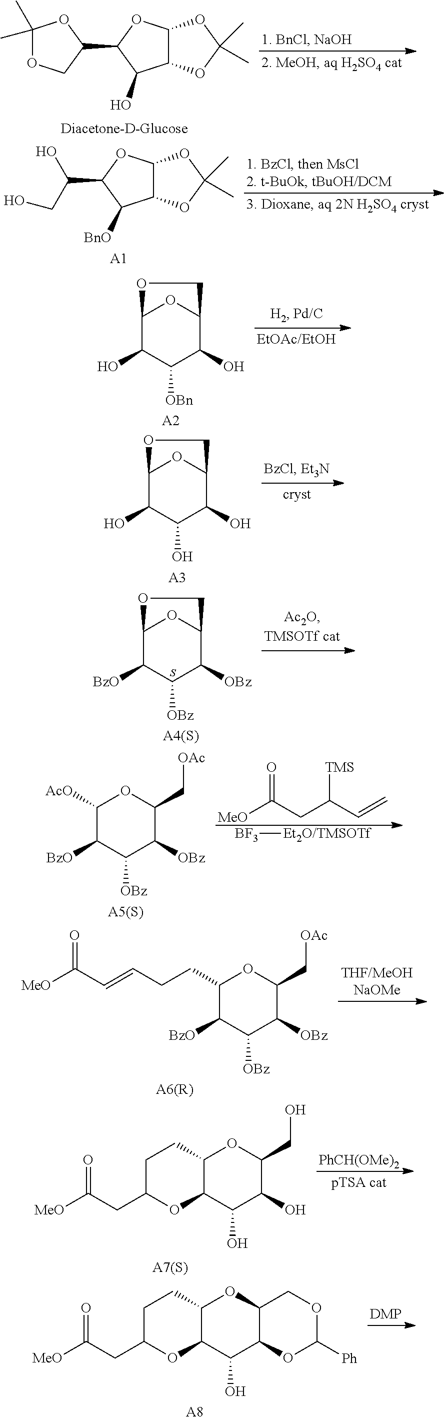

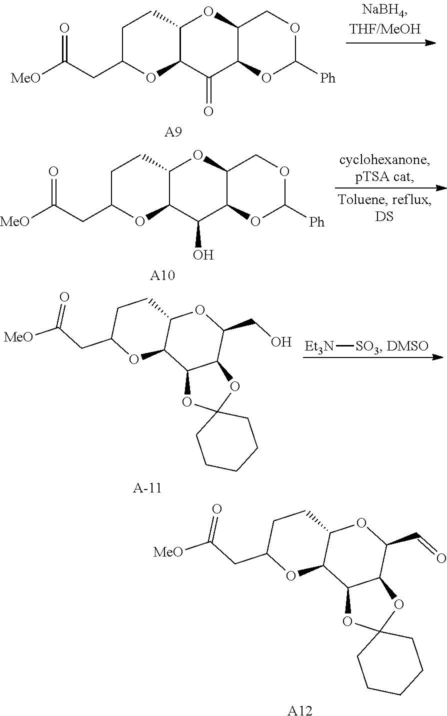

### Preparation of 1,6-anhydro-3-O-benzyl-β-L-idopyranose A2

### Lit., for Example, JACS, 2001, 123, 3153-3154

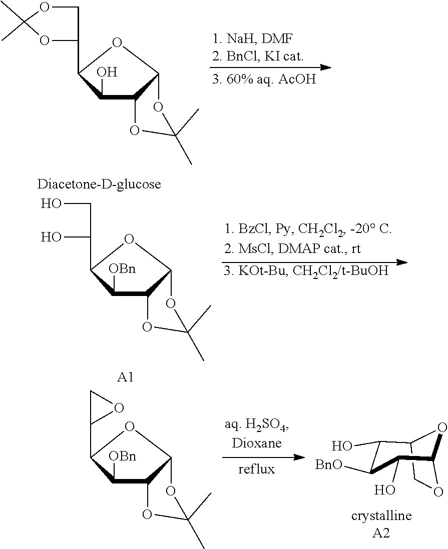

1) A solution of 1,2;5,6-di-O-isopropylidene-α-D-glucofuranose (260 g, 1 mol) in dry DMF (800 ml) was slowly added at 0° C. to a suspension of Sodium hydride (60% mineral oil dispersion, 48 g, 1.2 mol, 1.2 eq) and Sodium iodide (22.5 g, 0.15 mol) in dry DMF (800 ml). The resulted mixture was stirred at 0° C. for 1 h under Argon (hydrogen evolution!) and Benzyl chloride (145.6 g, 132.3 ml, 1.15 mol) was slowly added. The reaction was stirred for 8 h at 25° C. and quenched by addition of saturated aqueous NH₄Cl (50 ml). The most of DMF was evaporated under reduced pressure and the residue was portioned between MTBE (1500 ml) and water. The phases were separated; the organic one was washed with brine, dried over Na₂SO₄, filtered and evaporated under reduced pressure to give 370 g of the residual syrup.

2) The residual syrup (370 g) was dissolved in Acetic acid-water (4:1, 1500 ml) and the resulted mixture was stirred for 12 h at 50° C. The solvents were removed off under reduced pressure and the residue was co-evaporated twice with toluene to dryness.

3) The remained semisolid (330 g) was dissolved in dry CH₂Cl₂ (1500 ml) and dry Pyridine (800 ml, 10 mol) and the resulted mixture was cooled to −25° C. under Argon. A solution of Benzoyl chloride (140.6 g, 116.2 ml, 1.0 mol) in CH₂Cl₂ (200 ml) was slowly added at the rate which kept reaction temperature below −20° C. (˜1 h period) and the reaction was stirred for additional 2 h at −20° C. (TLC monitoring). Then, Methanesulfonyl chloride (229.1 g, 154.8 ml, 2.0 mol) was added followed by addition of DMAP (24.2 g, 0.2 mol). The reaction was gradually heated to 20° C. and stirred for additional 12 h. Then, the reaction mixture was poured into ice-water (2 L), stirred for 1 h and phases were separated. The aqueous one was extracted with CH₂Cl₂ (1500 ml) and the combined extracts were washed twice with 3N aqueous HCl (2×2 L), 9% aqueous NaHCO₃ (1 L) and brine, dried over Na₂SO₄, filtered and concentrated under reduced pressure to give 560 g of yellow sticky residue. The residue was crystallized from MeOH (3 L) afforded after drying 507 g (92.5% yield for 3 steps) of the desired Mesylate as a white solid

4) A solution of Mesylate (507 g, 0.925 mol) in dry CH₂Cl₂ (3500 ml) and t-Butanol (1000 ml) was cooled to 0° C. under Argon and Potassium t-butoxide was added (247 g, 2.2 mol, 2.4 eq) by portions kept the reaction temperature below 5° C. The resulted mixture was stirred for 2 h at 0° C., then for 12 h at 20° C. (TLC monitoring, Heptane/EtOAc 1:1) and concentrated under reduced pressure. The residue was portioned between MTBE (2500 ml) and ice-cold water (1 L), the pH was adjusted to 7 by addition of acetic acid and the mixture was filtered through Celite, The cake was washed with MTBE (2×500 ml), the combined filtrates were separated, the aqueous one was extracted with MTBE (500 ml) and the combined extracts were washed with water (1 L) and brine, dried over Na₂SO₄, filtered and concentrated under reduced pressure to give ˜400 g of the brown oil. The residue was purified on a short Silica gel column (800 g, Heptane/Ethyl acetate 10:1-4:1) to give 260 g of the desired 5,6-anhydro-L-idofuranose as yellow oil with ˜90% purity by HPLC.

5) A solution of 3-O-Benzyl-5,6-anhydro-L-idofuranose (260 g) in 2M aqueous H₂SO₄ (500 ml) and 1,4-Dioxane (500 ml) was stirred under reflux for 12 h. The reaction was cooled to 10° C. and neutralized with ION aqueous NaOH. The most of Dioxane was evaporated under reduced pressure, more water (1 L) was added and the aqueous mixture was extracted with EtOAc (3×800 ml). The combined organics were washed with Water and brine, dried over Na₂SO₄, filtered and concentrated under reduced pressure to give ˜200 g of semisolid residue. The residue was crystallized from EtOH (700 ml) afforded 152 g (60% from Diacetone-D-glucose) of 1,6-anhydro-3-O-benzyl-β-L-idopyranose as off-white solid, mp 157-158° C. (lit. 158-159° C.; JACS, 2001, 123, 3153-3154). The filtrate was concentrated to a one-third of volume and refrigerated for 12 h to give second crop (15 g, 6%) of A2 as beige solid.

### Preparation of 1,6-di-O-acetyl-2,3,4-tri-O-benzoyl-α-L-idopyranose (A4 (S))

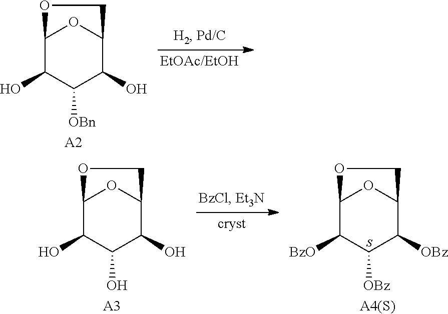

1,6-anhydro-3-O-benzyl-β-L-idopyranose, A2 (30 g, 119 mmol) was dissolved in EtOAc/MeOH (1:1, 250 ml) and hydrogenated over 10% Pd/C at 50° C., 5 atm for 4 h. The mixture was cooled to 20° C., flushed with Nitrogen (three times), filtered through Celite and evaporated under reduced pressure. A solid residue and DMAP (4.4 g, 36 mmol) were dissolved in CH₂Cl₂ (80 ml) and Pyridine (80.8 ml, 1.0 mol) and the resulted mixture was cooled to 0° C. Benzoyl chloride (55.2 g, 45.7 ml, 0.393 mmol) was slowly added and the reaction was stirred for 3 h at 20° C. The most of volatiles were evaporated under reduced pressure and the semisolid residue was portioned between EtOAc (500 ml) and water (500 ml). The phases were separated, the organic one was washed with 2N aqueous HCl, 9% aqueous NaHCO₃ and brine, dried over Na₂SO₄, filtered and concentrated under reduced pressure to give ˜60 g of solids which were crystallized from Isopropanol afforded 53 g (94% yield) of the desired A4(S) as off-white solid.

### Preparation of A6(R)

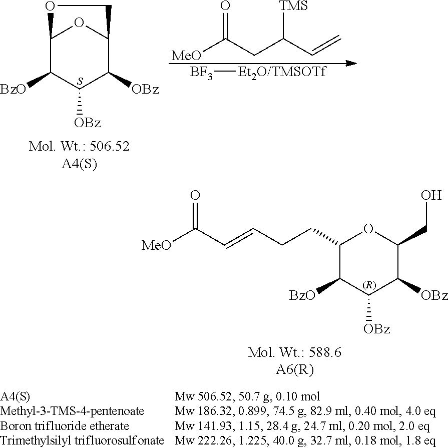

A4 (S) and Methyl-3-TMS-4-pentenoate were dissolved in dry AcN (1 L) and the resulted solution was cooled to 10° C. under Argon. BF₃-Et₂O was slowly added followed by TMSOTf kept the reaction temperature below 20° C. Then, the resulted mixture was warmed to 20° C. and stirred for 40 h (UPC²/TLC control, Heptane/EtOAc 1:1). The reaction was carefully (gas evolution!) poured into vigorously stirred 9% aqueous NaHCO₃ (1.2 L), the resulted mixture was stirred for 30 min and the phases were separated. The organic one was evaporated under reduced pressure, the aqueous phase was extracted with EtOAc (2×300 ml) and the extracts were combined with the residue remained after evaporation. The resulted solution was washed with brine, dried over Na₂SO₄, filtered and evaporated under reduced pressure at 40° C. afforded ˜65 g of crude A6(R) which was used in the next step

### Preparation of A7(S)

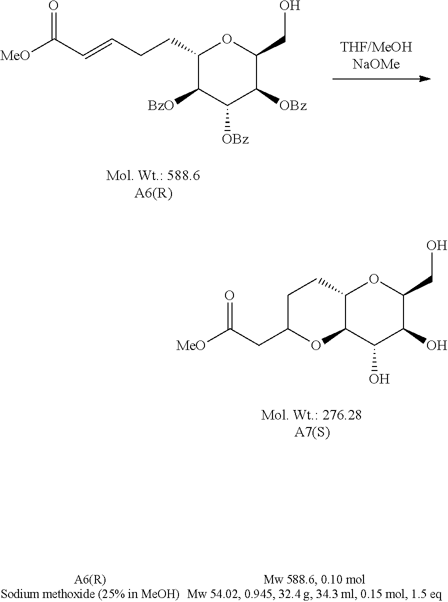

A solution of A6(R) (70 g) in dry THF (500 ml) was cooled to 0° C. under Argon and a half of NaOMe (17.2 ml) was slowly added. The reaction was stirred for 1 h at 0° C. (UPC²/TLC control, EtOAc) and more of NaOMe (17.1 ml) was added. Then, the mixture was stirred for additional 6 h at 0° C. to complete isomerization process (UPC² monitoring), diluted with MeOH (50 ml) and quenched with 20% aqueous NH₄Cl (50 ml). The volatiles were evaporated under reduced pressure and the semi solid residue was stirred with EtOAc (300 ml) and filtered through Celite. The cake was washed with EtOAc (2×100 ml), the combined filtrates were dried over Na₂SO₄, filtered and evaporated under reduced pressure afforded a brown oily residue. This residue (˜50 g) was triturated twice with Heptane (2×200 ml) as follows: The mixture was vigorously stirred at 40° C. for 1 h, cooled to 0° C. and allowed to stand for 2 h. The upper layers were decanted and discarded, the residual solvent was evaporated under reduced pressure and the remained semisolid (30 g) was purified on a short Silica column (100 g) eluted with gradient DCM/MeOH. 20.2 g (73% yield) of A7(S) were obtained after drying

### Preparation of A8

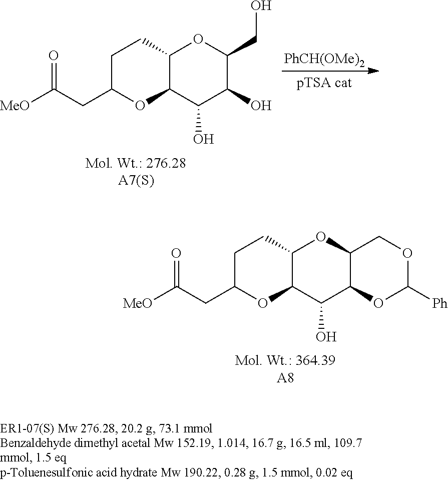

A solution of A7(S), Benzaldehyde dimethyl acetal and p-TSA in dry DMF (100 ml) was stirred for 2 h at 40° C. (UPC²/TLC control, EtOAc), cooled to 20° C. and quenched with 9% aqueous NaHCO₃ (5 ml). The most of DMF was evaporated under reduced pressure and the residue was portioned between EtOAc (250 ml) and Water (50 ml). After 15 min of stirring, the phases were separated, the organic one was washed with brine, dried over Na₂SO₄, filtered and evaporated under reduced pressure afforded 26.6 g (quant) of the crude A8 which was used in the next step

### Preparation of A9

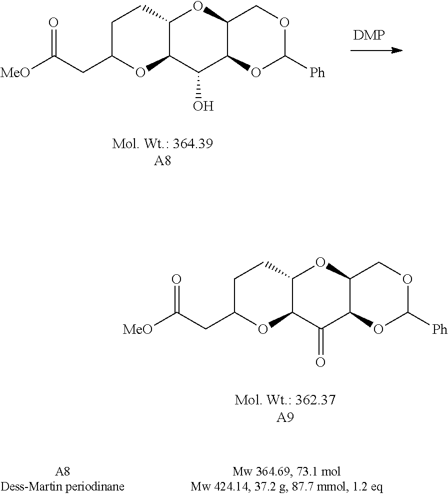

A solution of DMP in dry DCM (250 ml) was cooled to 0° C. under Argon and a solution of A8 (26.6 g) in dry DCM (100 ml) was slowly added kept the temperature inside below 10° C. The reaction was stirred for 1 h at 0° C., then for 2 h at 20° C. (UPC²/TLC control, Heptane/EtOAc 1:2) and poured (gas evolution!) into a mixture of 9% aqueous NaHCO₃ (330 ml, ˜4 eq) and 10% aqueous Na₂SO₃ (220 ml, ˜2 eq). The resulted mixture was stirred for 2 h until complete dissolution of solids, the phases were separated, and the aqueous one was extracted with DCM (150 ml). The combined organics were washed with 9% aqueous NaHCO₃ (100 ml), dried over Na₂SO₄, filtered and evaporated under reduced pressure to give 27.1 g (quant) of the crude A9 which was used in the next step

### Preparation of A10

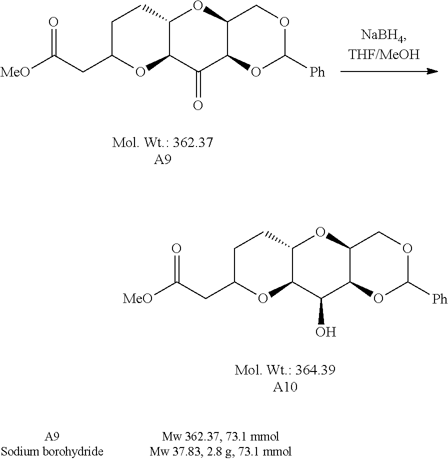

A solution of the crude A9 (27.1 g) in dry THF (240 ml) and absolute MeOH (30 ml) was cooled to 0° C. and NaBH₄ was carefully (gas evolution!) added portion wise and the stirring was continued for 1 h at 0° C. (UPC²/TLC control, Heptane/EtOAc 1:2). A reaction was quenched by addition (gas evolution!) of 9% aqueous NaHCO₃ (250 ml, ˜3 eq) and the most of organic solvents were evaporated under reduced pressure. The aqueous residue was extracted with MTBE (2×150 ml), the combined extracts were washed with brine, dried over Na₂SO₄, filtered and evaporated under reduced pressure afforded 25.7 g (quant) of the crude A10 which was used in the next step

### Preparation of A11

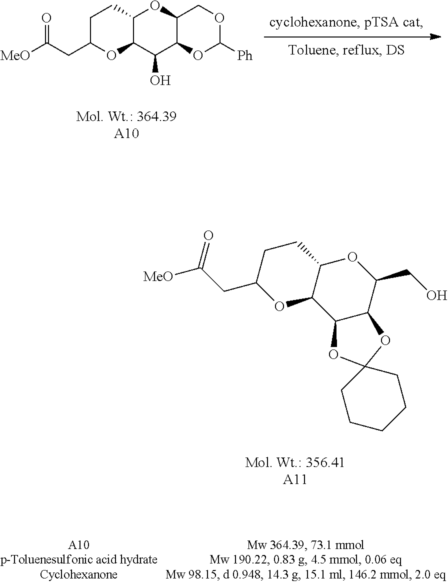

A solution of the crude A10 (25.7 g), Cyclohexanone and p-TSA in dry Toluene (250 ml) was stirred for 12 h at 80° C. (UPC²/TLC control, Heptane/EtOAc 1:2), cooled to 40° C. and MeOH (10 ml) was added. After 1 h of stirring, the reaction was cooled to 25° C. and quenched with 9% aqueous NaHCO₃ (50 ml). The phases were separated, the aqueous one was extracted with Toluene and the combined organics were washed with brine, dried over Na₂SO₄, filtered and evaporated under reduced pressure to give 28.2 g of the brown honey-like mass. The residue was crystallized from the mixture MTBE/Heptane afforded 13.1 g (50% for 4 steps) of the desired A11 as beige powder.

### Preparation of A12

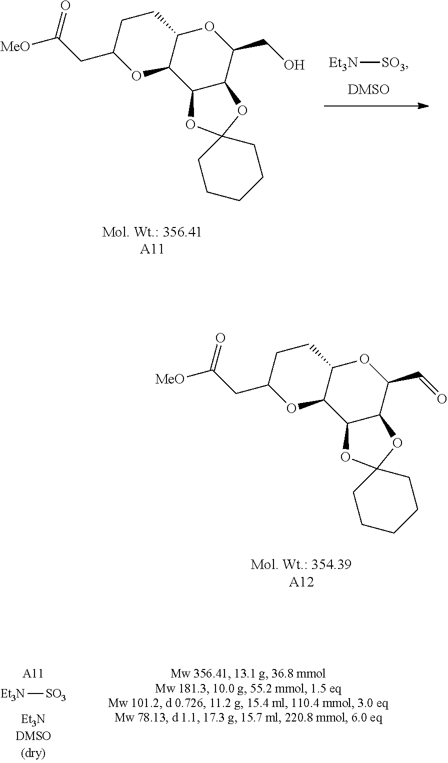

A solution of A11, Et₃N and DMSO in dry DCM (50 ml) was cooled to 0° C. under Argon and a solution of Et₃N—SO₃ in dry DCM (20 ml) doped with Et₃N (0.5 ml) was slowly added kept the reaction temperature below 15° C. The reaction was stirred at 0° C. for 2 h, then 4 h at 25° C. (TLC control; Hept/EtOAc 2:1) and quenched with Water (50 ml). After 15 min of stirring, the mixture was diluted with EtOAc (150 ml), the phases were separated and the aqueous one was extracted with EtOAc (50 ml). The combined organic were washed with 10% aqueous Citric acid (2×50 ml), 9% aqueous NaHCO₃ (50 ml) and brine, dried over Na₂SO₄, filtered through silica gel plug (20 g) and evaporated under reduced pressure at 30° C. afforded 12.8 g (near quant) of A12 as yellow foam, which was stored in the freezer.

### Example 2: Alternative Process for the Preparation of all (See Also FIG. 2)

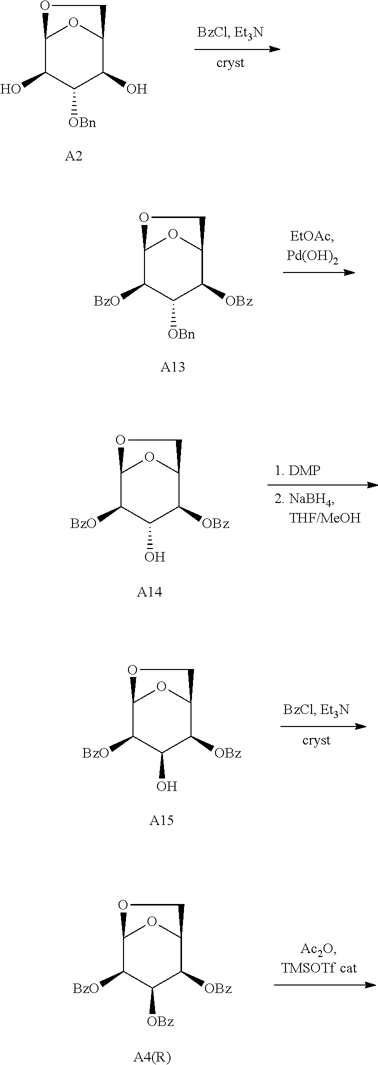

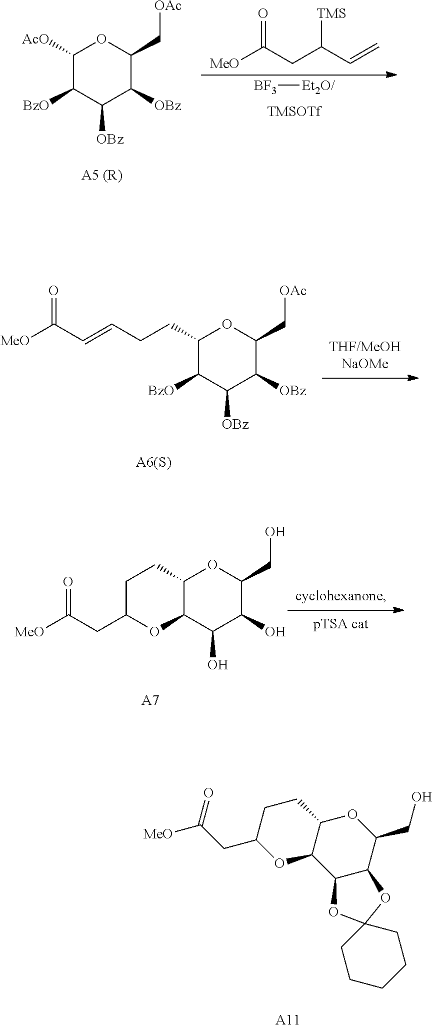

### Preparation of A13

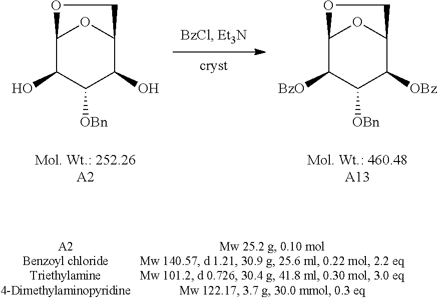

A2, DMAP and Et₃N were dissolved in dry DCM (300 ml) and the resulted mixture was cooled to 0° C. under Argon. Benzoyl chloride was slowly added and the reaction was stirred for 20 h at 20° C. (UPC²/TLC control, Heptane/EtOAc 1:2). The most of volatiles were evaporated under reduced pressure a semisolid residue was portioned between EtOAc (400 ml) and water (200 ml) and the resulted mixture was stirred for 1 h. The phases were separated, the organic one was washed with 2N aqueous HCl (300 ml), 9% aqueous NaHCO₃ (300 ml) and brine, dried over Na₂SO₄, filtered and concentrated under reduced pressure to give ˜50 g of beige solids which were crystallized from Isopropanol (250 ml) afforded 43.8 g (95% yield) of the desired A13 as off-white solid.

### Preparation of A14

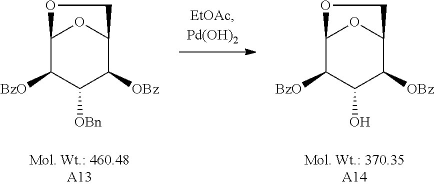

A solution of A13 (43.8 g, 95.1 mmol) in EtOAc (400 ml) was hydrogenated over 20% Pd(OH)₂/C (2.5 g) at 30° C. and 5 atm for 8 h.

The mixture was cooled to 20° C., flushed with Nitrogen (three times), filtered through Celite and evaporated under reduced pressure afforded 36.2 g of crude A14 as off-white foam contaminated with products of 3→4 and 5→4 O-benzoyl migrations (0.3% and 0.4% respectively).

### Preparation of A15

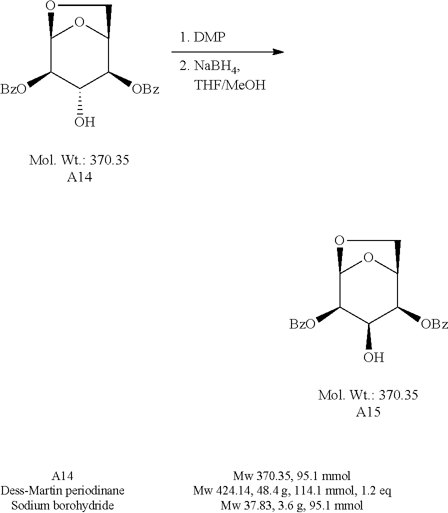

A solution of DMP in dry DCM (400 ml) was cooled to 0° C. under Argon and a solution of A14 (36.2 g) in dry DCM (100 ml) was slowly added kept the temperature inside below 10° C. The reaction was stirred for 1 h at 0° C., then for 2 h at 20° C. (UPC²/TLC control, Heptane/EtOAc 1:1) and poured (gas evolution!) into a mixture of 9% aqueous NaHCO₃ (360 ml, ˜4 eq) and 10% aqueous Na₂SO₃ (240 ml, ˜2 eq). The resulted mixture was stirred for 2 h until complete dissolution of solids, the phases were separated, and the aqueous one was extracted with DCM (100 ml). The combined organics were washed with 9% aqueous NaHCO₃ (100 ml), dried over Na₂SO₄, filtered and evaporated under reduced pressure to give 37.6 g (quant) of the crude A15 as off-white foam which was used in the next step

A solution of the crude A15 (37.6 g) in dry THF (320 ml) and absolute MeOH (40 ml) was cooled to 0° C. and NaBH₄ was carefully (gas evolution!) added portionwise, the stirring was continued for 1 h at 0° C. (UPC²/TLC control, Heptane/EtOAc 1:1) and the reaction was quenched by slow addition (gas evolution!) of 9% aqueous NaHCO₃ (270 ml, ˜3 eq). The most of organic solvents were evaporated under reduced pressure, the aqueous residue was extracted with EtOAc (2×200 ml) and the combined extracts were washed with brine, dried over Na₂SO₄, filtered and evaporated under reduced pressure afforded 35.3 g (quant) of the crude A15 in form of off-white foam.

### Preparation of A4(R)

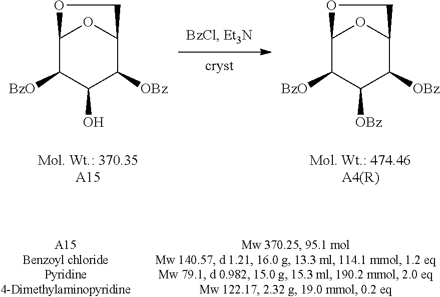

Crude A15 (35.3 g), DMAP and Pyridine were dissolved in dry DCM (300 ml) and the resulted mixture was cooled to 0° C. under Argon. Benzoyl chloride was slowly added and the reaction was stirred for 20 h at 20° C. (UPC²/TLC control, Heptane/EtOAc 1:1). The most of volatiles were evaporated under reduced pressure a semisolid residue was portioned between EtOAc (500 ml) and water (200 ml) and the resulted mixture was stirred for 1 h. The phases were separated, the organic one was washed with 2N aqueous HCl (300 ml), 9% aqueous NaHCO₃ (300 ml) and brine, dried over Na₂SO₄, filtered and concentrated under reduced pressure to give ˜50 g of a yellow solids which were crystallized from Isopropanol (400 ml) afforded 33.8 g (75% for 4 steps) of the desired A4(R) as off-white solid.

### Preparation of A7(R)

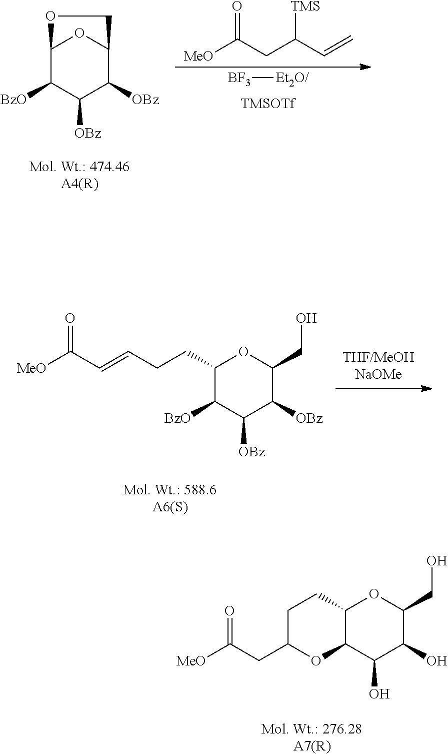

The procedures were performed as described above (Example 1) for preparation of A7(S) from A4(S).

13.8 g (70% for 3 steps) of A7(R) was obtained as yellow foam starting from 33.8 g of A4(R).

### Preparation of A11

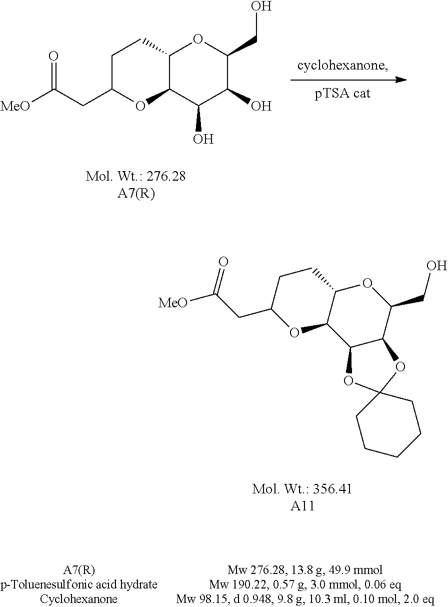

A solution of the crude A7(R), Cyclohexanone and p-TSA in dry Toluene (200 ml) was reflux for 4 h with continuous water separation (UPC²/TLC control, Heptane/EtOAc 1:2), cooled to 20° C. and quenched with 9% aqueous NaHCO₃ (30 ml). The phases were separated, the aqueous one was extracted with Toluene (50 ml) and the combined organics were washed with brine, dried over Na₂SO₄, filtered and evaporated under reduced pressure to give 20.3 g of the brown honey-like mass.

The residue was crystallized from the mixture MTBE/Heptane afforded 15.1 g (85% yield) of the desired A11 as yellowish powder.

### Example 3: Alternative Process for the Preparation of A7(R) (See Also FIG. 3)

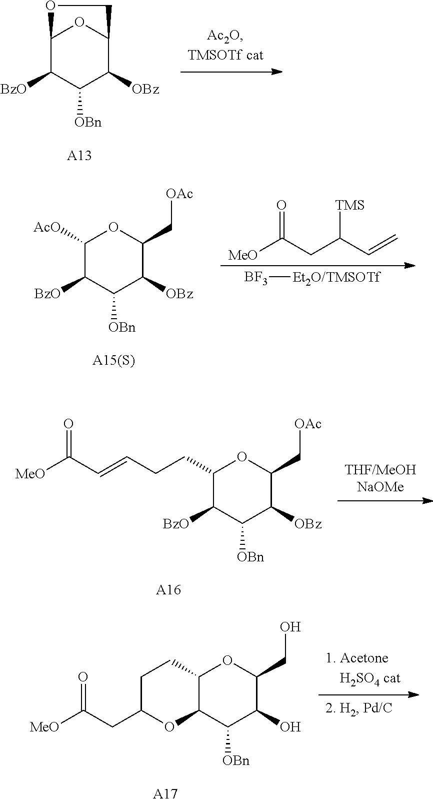

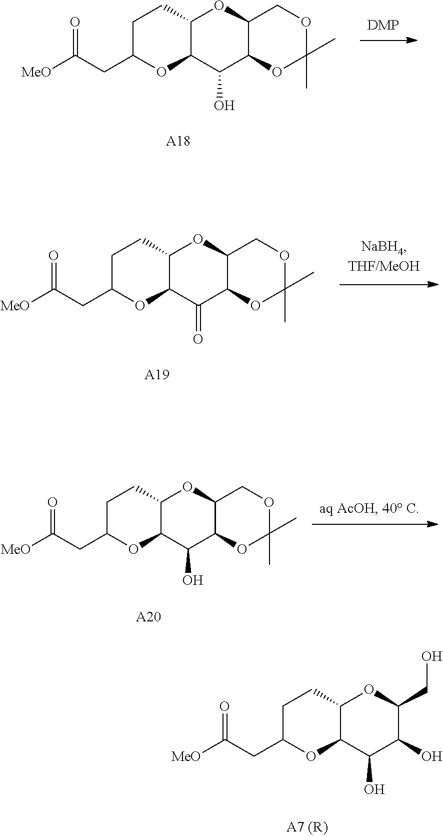

A17 was prepared from A13 in the same manner as described in Example 1 for preparation of A7(S) from A4(S). The well-known standard isopropylidene protection (T. W. Greene and P. G. M. Wuts; Protecting Groups in Organic Synthesis, 3rd edition, John Wiley & Sons, 1999) followed by reduction of benzyl protective group led to formation of A18. The transformation of A18 into A20 was performed as described in Example 2 for preparation of A15 from A14. Deprotection of isopropylidene group under standard conditions formed the desired A7(R) in 35% total yield (7 steps) starting from A13.

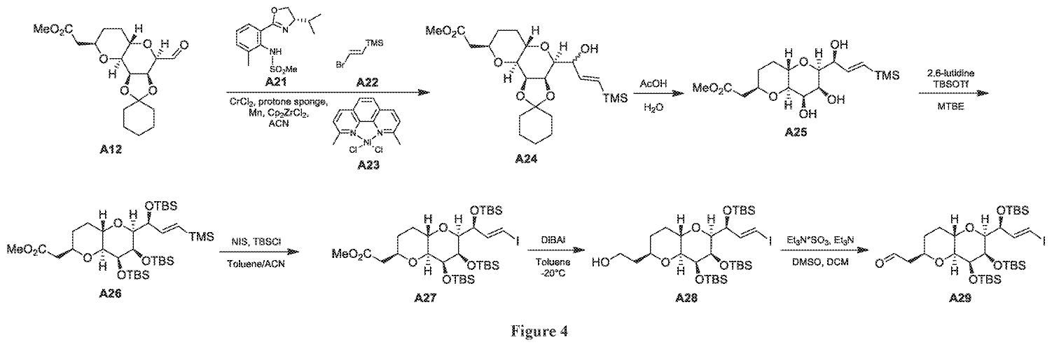

### Example 4: Preparation of A29 (See Also FIG. 4)

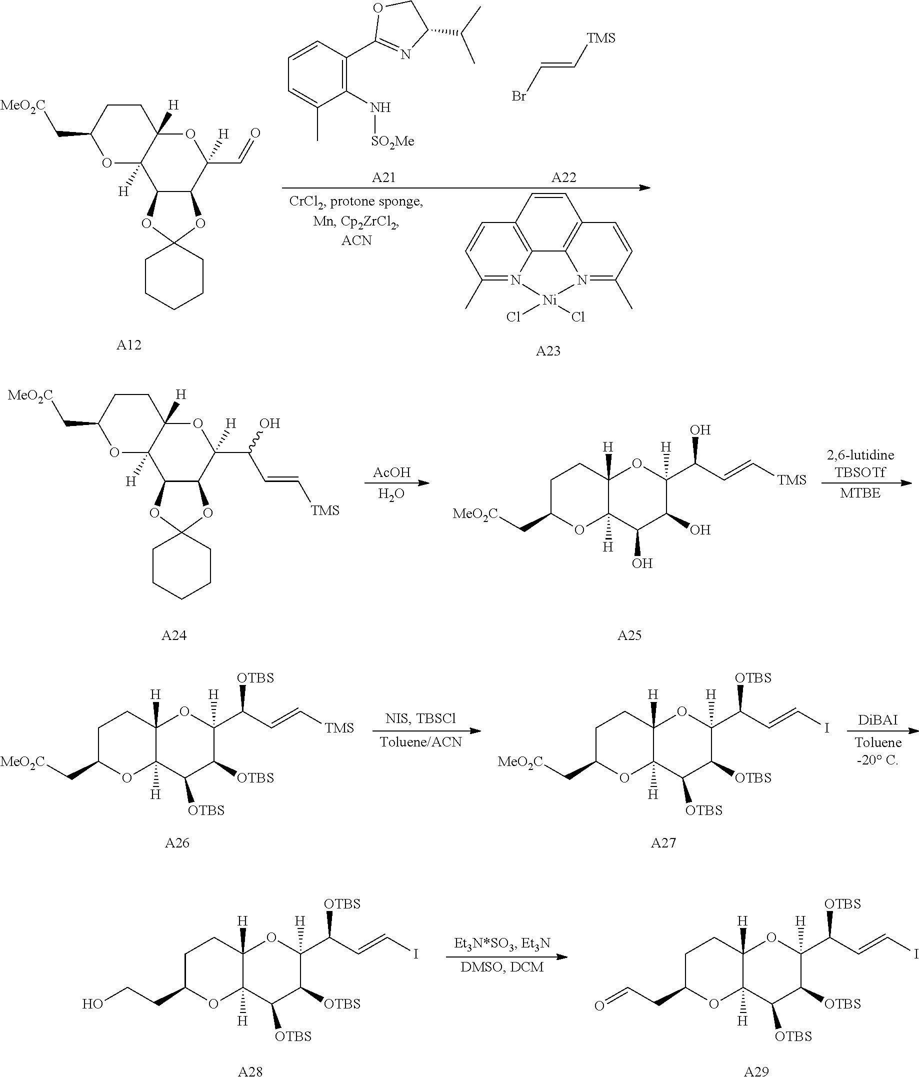

### Preparation of A24

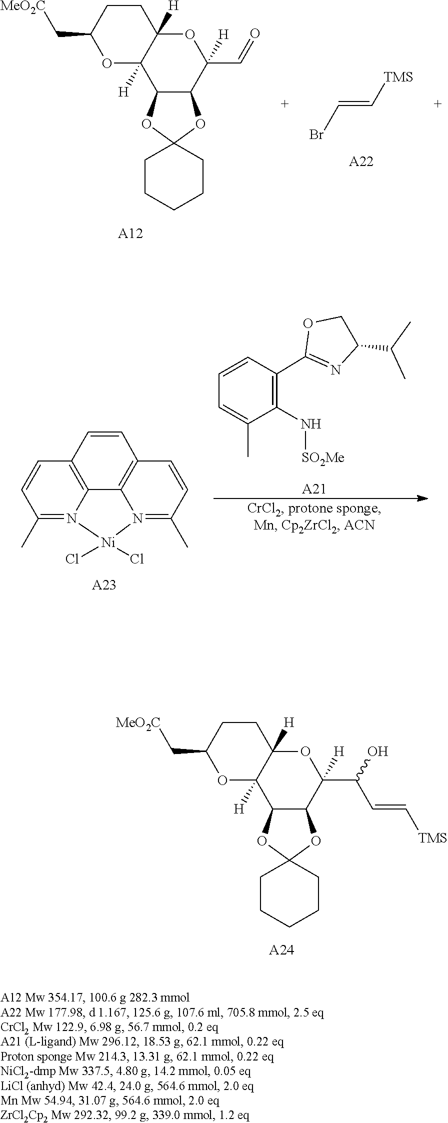

A three-necked flask was dried, equipped with stirrer, septum and two taps and flushed with Argon by three vacuum-Argon cycles. CrCl₂, A21 and proton sponge were loaded into the flask and flushed with Argon. Then anhydrous AcN (300 ml) was introduced through the septum and the resulted green mixture was stirred for 1 h at 25° C. until complete dissolution.

In the separated three-necked flask, prepared as described above, A12, A22, LiCl, Mn and NiCl₂-dmp were mixed under Argon and then anhydrous AcN (1000 ml) was introduced through the septum. The resulted suspension was stirred for 10 min, cooled to 0° C. and the prepared Cr-ligand solution was added by syringe. The reaction was stirred for 10 min and then ZrCl₂Cp₂ was loaded in one portion under positive Argon pressure. The mixture was warmed to 20-25° C. and stirred for 4 h (TLC/UPC² control, Hept/EtOAc 1:2; complete). The most of AcN was evaporated under reduced pressure, and the residue was portioned between MTBE (1200 ml) and 1N aq HCl (1000 ml). The mixture was stirred for 20 min and the phases were separated. The aqueous one was extracted with MTBE (2×400 ml) and the combined organics were washed with sat aqueous NaHCO₃ (300 ml) and brine, dried over Na₂SO₄ and evaporated under reduced pressure at 40° C. afforded ˜160 g of the crude A24 which was used in the next step

### Preparation of A25

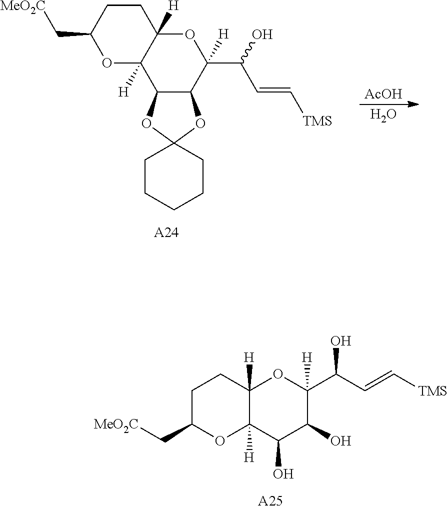

Crude A24 (˜160 g) was dissolved in the mixture of Acetic acid (650 ml) and Water (650 ml) at 95-100° C. and the reaction was stirred for 15 h (UPC²—limit not more than 2% of 24—complete. TLC control; Hept/EtOAc 1:2). Most of the volatiles were evaporated under reduced pressure at 60° C. and the residue was dissolved in EtOAc (1000 ml) and treated with 10% aqueous NaHCO₃ (1000 ml; careful—gas evolution. If after addition pH is still less than 7.5, more NaHCO₃ will be added). The mixture was stirred for 1 h and passed through Celite pad. The phases were separated, the aqueous one was extracted with EtOAc (2×300 ml) and the combined organics were washed with 10% aqueous NaHCO₃ (200 ml) and brine, dried over Na₂SO₄ and evaporated under reduced pressure afforded ˜110 g of the crude A25. This crude was dissolved in Toluene (400 ml), loaded onto short Silica gel column (450 g) and eluted with Hept/EtOAc (6:1, 2 L), Hept/EtOAc (2:1, 3 L), Hept/EtOAc (1:2, 3 L) and EtOAc (2 L). All factions (each fraction was 500 ml) contained the ER1-15 were combined and evaporated under reduced pressure. The solid residue (65 g) was triturated with MTBE (200 ml) at 55° C. and Heptane (400 ml) pre-heated to 60° C. was slowly added with stirring. The resulted suspension was cooled to 25° C. during 1 h period, stirred for 1 h at 25° C., cooled to 0° C. and stirred for additional 4 h. The precipitate was filtered, washed with cold Hept/MTBE 2:1 (2×100 ml) and Heptane (2×100 ml) and dried under reduced pressure to give the desired A25 (46 g, 43.3% yield) as beige solid contaminated with ˜1% of isomer. All filtrates were combined, evaporated under reduced pressure and the residue was passed through short Silica gel column afforded additional 11.4 g (10.7% yield) of A25 as brownish solid with two isomers—8 and 6% respectively.

Total yield—54% starting from ER1-13

### Preparation of A26

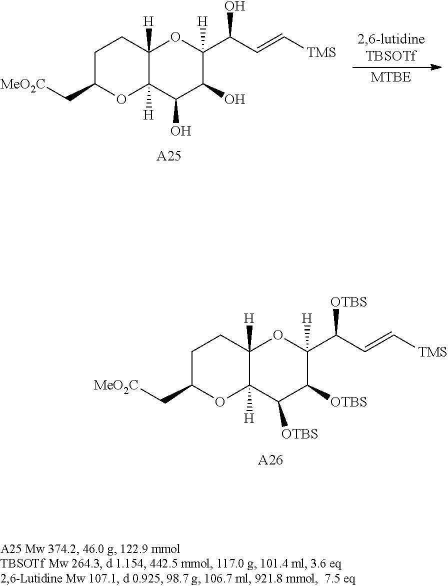

A25 and 2,6-Lutidine were dissolved in dry MTBE (500 ml) and the mixture was cooled to 0° C. under Argon. TBSOTf was slowly added kept the reaction temperature below 10° C., the resulted mixture was stirred for 1 h at 0° C. and then 12 h at 25° C. (UPC² control, if sum of self-products is more than 5%, more TBSOTf has to be added; TLC control, Hept/EtOAc 2:1 and 1:2). The reaction was quenched by addition of Water (250 ml), the resulted mixture was stirred for 30 min and then the phases were separated. The aqueous one was extracted with MTBE (300 ml) and the combined organics were washed with 10% aqueous Citric acid (300 ml), 10% aqueous NaHCO₃ (100 ml) and brine, dried over Na₂SO₄, filtered and evaporated under reduced pressure afforded ˜100 g of the crude A26. This crude was dissolved in MeOH (400 ml) at 40° C., the resulted solution was cooled to −15° C. with stirring and the resulted suspension was stirred for additional 6 h at −15° C. The precipitate was filtered, washed with cold (−15° C.) MeOH (80 ml) and dried under reduced pressure afforded 60 g of the desired A26 as white solid. All filtrates were combined and evaporated. The residue (˜40 g) was crystallized from MeOH (120 ml) as described above to give additional A26 (15 g) as white powder.
Total—75 g (85% yield)

### Preparation of A27

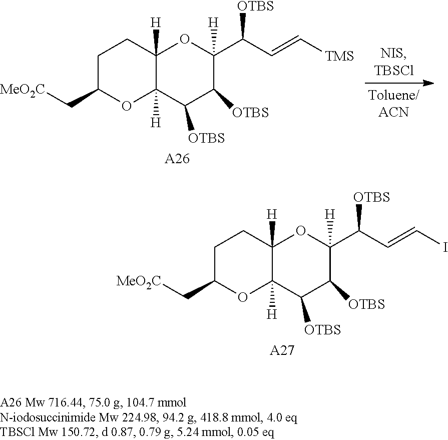

A26 was dissolved in the mixture of anhyd AcN (600 ml) and dry Toluene (200 ml) under Argon and NIS with TBSCl were added. The resulted mixture was stirred for 48 h at 25° C. (UPC² control; TLC-Hept/EtOAc 4:1). The most of AcN was evaporated under reduced pressure at 40° C. and the residue was taken off with EtOAc (800 ml). The resulted mixture was treated with the mixture 10% aqueous Na₂S₂O₃/10% aqueous NaHCO₃ (1:1 v/v; 1000 ml), stirred for 20 min (discoloration occurred) and phases were separated. The organic one was washed with the mixture 10% aqueous Na₂S₂O₃/10% aqueous NaHCO₃ (1:1 v/v; 300 ml) and brine, dried over Na₂SO₄, filtered and evaporated under reduced pressure at 40° C. afforded ˜81 g (quant) of the crude A27 as yellowish semi-solid mass.

### Preparation of A28

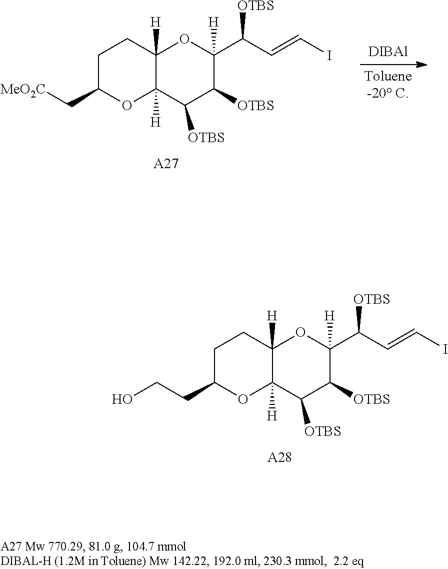

A solution of crude A27 in dry Toluene (700 ml) was cooled to −30° C. under Argon and DIBAL-H was slowly added kept the reaction temperature below −20° C. The reaction was stirred for 1 h at −20° C. (UPC² control; TLC-Hept/EtOAc 2:1) and quenched by slow addition of 10% aqueous Citric acid (500 ml). The resulted mixture was stirred at 20° C. until almost complete dissolution of solids, the phases were separated and the aqueous one was extracted with MTBE (2×300 ml). The combined organics were washed with 10% aqueous NaHCO₃ (300 ml) and brine, dried over Na₂SO₄, filtered and evaporated under reduced pressure afforded ˜78 g (quant) of the crude A28 as foam which transformed to sticky mass during evaporation.

Purification on a short Silica gel column (3 g/1 g of crude) may be performed.

### Preparation of A29

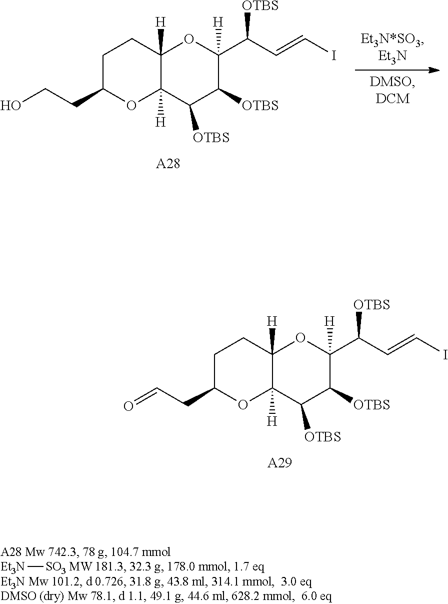

A28 crude, Et₃N and DMSO were mixed into dry DCM (300 ml) and the resulted solution was cooled to 0° C. under Argon. A solution of Et₃N—SO₃ in dry DCM (60 ml) was slowly added kept the reaction temperature below 10° C. and the resulted mixture was stirred for 1 h at 0° C. and then 4 h at 20-25° C. (UPC² control; TLC-Hept/EtOAc 2:1). The reaction was diluted with MTBE (500 ml) and quenched by slow addition of Water (500 ml). The resulted mixture was stirred for 15 min at 20° C., the phases were separated and the organic one was washed with 10% aqueous Citric acid (500 ml), 10% aqueous NaHCO₃ (300 ml) and brine, dried over Na₂SO₄, filtered and evaporated under reduced pressure afforded ˜82 g of the crude A29 as sticky semi-solid mass. The crude was dissolved in Heptane, the insoluble inorganics were filtered of and washed with Heptane (2×70 ml). The combined filtrates were loaded on a short Silica gel column (250 g) and eluted with Hept/EtOAc 20:1 (3 L) and then Hept/EtOAc 10:1 (1.5 L). All fractions contained the desired product were combined and evaporated to give 62 g (80% yield for 3 steps) of A29 as sticky yellowish glass-like mass.

A29 was stored at −20° C. under Argon.

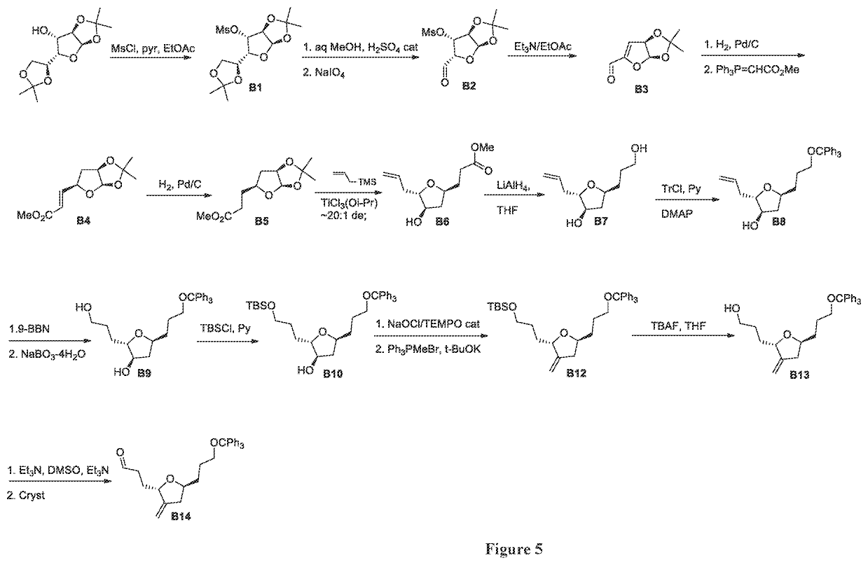

### Example 5: Preparation of B14 (See Also FIG. 5)

B14 was prepared according the well-known literature scheme. The crystallization of B14 is provided at this invention

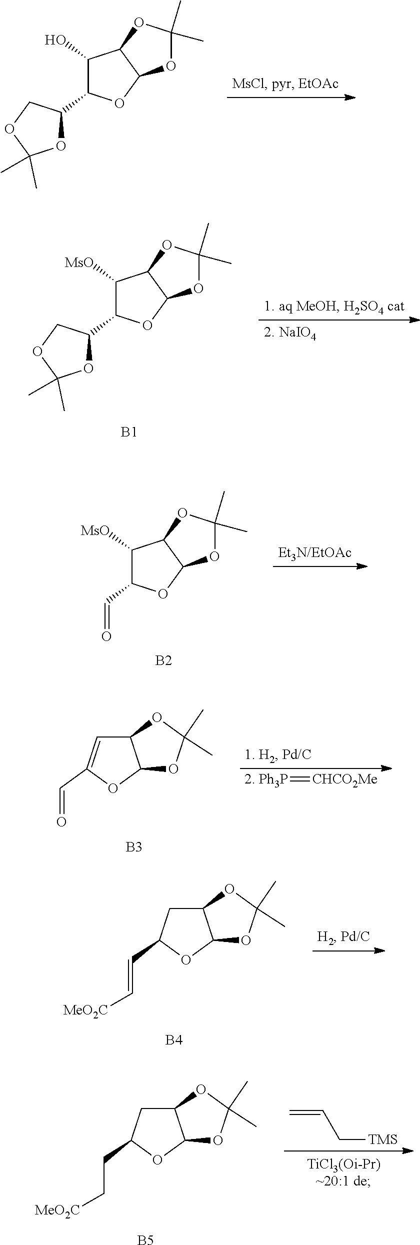

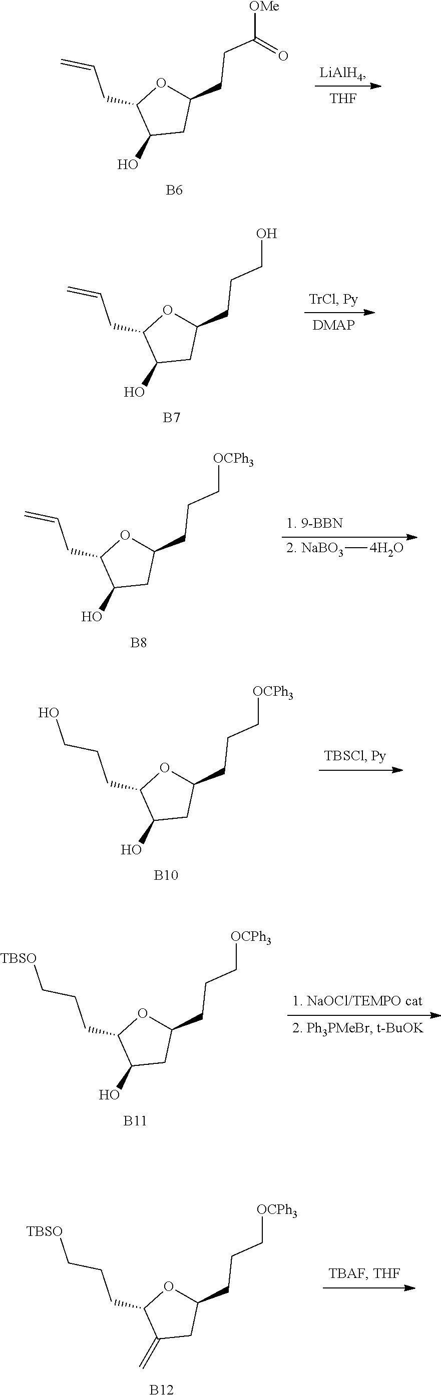

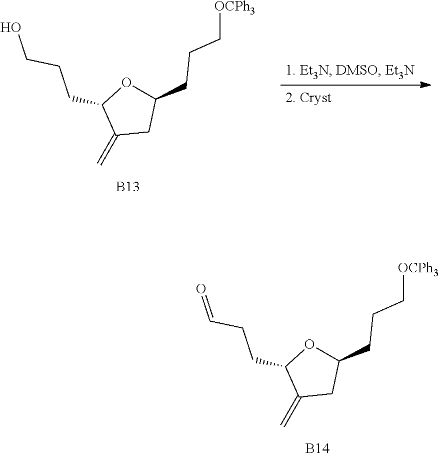

### Preparation of B1

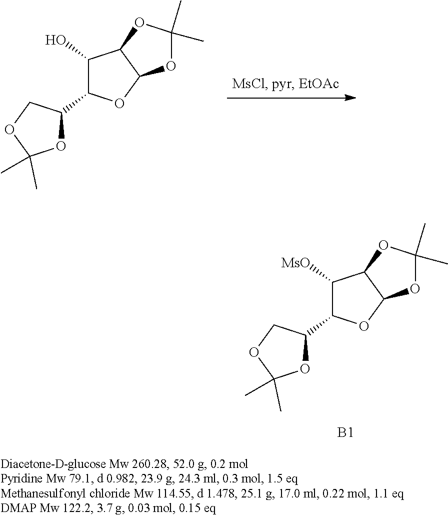

A solution of Diacetone-D-glucose (0.2 mol, 52.0 g), Pyridine (1.5 eq, 0.3 mol, 24.3 ml) and DMAP (0.15 eq, 0.03 mol, 3.7 g) in dry EtOAc (300 ml) was cooled to 0° C. under Argon and Methanesulfonyl chloride (1.1 eq, 0.22 mol, 17.0 ml) was added dropwise kept the reaction temperature below 15° C. The reaction was heated slowly to 25° C., stirred for 1 h (TLC control, Heptane/EtOAc 1:2), and cooled again to 0° C. After that, the cold suspension was filtered, the cake was washed with cold EtOAc (50 ml) and the combined filtrates were washed with 10% aqueous citric acid (2×100 ml), 10% aqueous NaHCO₃ (100 ml) and brine, dried over Na₂SO₄, filtered and evaporated under reduced pressure afforded 68 g (near quant) of the solid crude. The crude was crystallized from MeOH (250 ml) afforded 60 g (89% yield) of pure B1 as white solid

### Preparation of B2

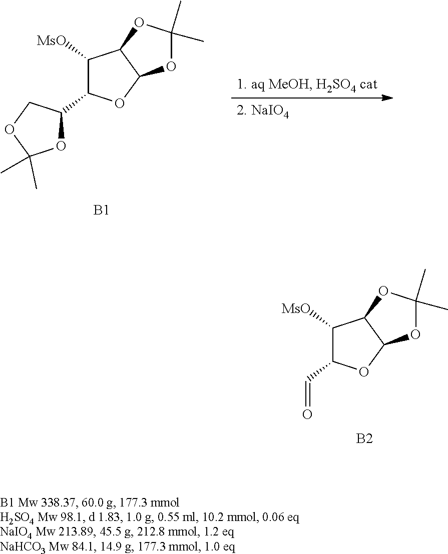

B1 (60.0 g) was suspended in MeOH (300 ml) and DCM (50 ml) and the mixture was treated with 1% aqueous H₂SO₄ (100 ml). The reaction was stirred at 30° C. for 48-72 h (TLC control; Hep/EtOAc 1:2) and quenched by addition of solid NaHCO₃ (14.9 g). Slow gas evolution was observed.

After 1 h of progressive stirring the mixture was cooled to 0° C. and treated by portionwise addition of NaIO₄ (45.5 g; 5 portions during 1 h period) and the resulted suspension was stirred for 3 h at 0° C. (TLC control; Hep/EtOAc 1:2). The mixture was filtered, the solids were washed with MeOH (2×100 ml) and the combined filtrates were evaporated under reduced pressure. The residue was portioned between EtOAc (500 ml) and Water (100 ml). The phases were separated, the aqueous one was extracted with EtOAc (2×100 ml) and the combined organics were washed with brine, dried over Na₂SO₄ and filtered to give ˜ 700 ml solution of the crude B2.

### Preparation of B3

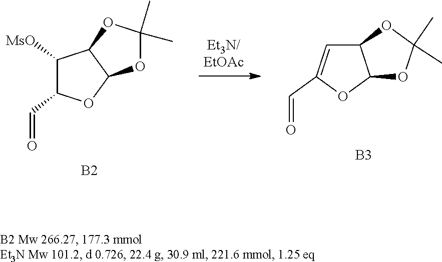

Et₃N (1.25 eq, 30.9 ml) was added into the solution of B2 prepared above and the reaction was stirred for 4 h at 80° C. (TLC control; Hep/EtOAc 1:2). After that, the mixture was cooled to 25° C. and washed with Water (100 ml), 10% aqueous Citric acid (80 ml), 10% aqueous NaHCO₃ (80 ml) and brine, dried over Na₂SO₄, filtered and diluted with MeOH (100 ml) and the resulted solution was used as such for the next step.

### Preparation of B4

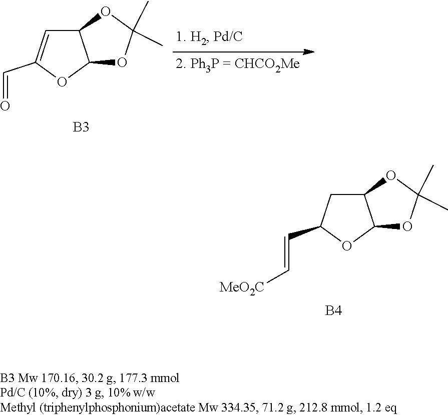

This solution of B3 (˜800 ml) was hydrogenated (˜1 h) on 10% Pd/C (3 g) at 80 psi (TLC control), flushed with nitrogen and catalyst was filtered off and washed with DCM (2×50 ml). The combined filtrates were cooled to 0° C. and ylide Ph₃P═CHCO₂Me (72 g) was added by portions into the stirred mixture. The reaction was heated to 25° C., stirred for 6 h (TLC control, Heptane/EtOAc 1:1) and quenched by addition of 10% aqueous Citric acid (40 ml). All volatiles were evaporated under reduced pressure and the solid residue was triturated with MTBE (400 ml) at 40° C., cooled to 0° C. and stirred for 4 h. The solids were filtered off, washed with cold MTBE (2×100 ml) and the combined filtrates was concentrated to one-third volume (˜200 ml), and allowed to stand at −18° C. for 12 h. Then, the solids were filtered off again, washed with cold MTBE (2×50 ml) and the combined filtrates were passed through silica gel plug (80 g). Plug was washed with additional Heptane/MTBE 1:1 (200 ml) and all solvents were evaporated under reduced pressure to give ˜45 g of the crude B4 as yellow syrup.

The filtration through Silica gel plug is optional. The crude could be taken to hydrogenation without purification.

### Preparation of B5

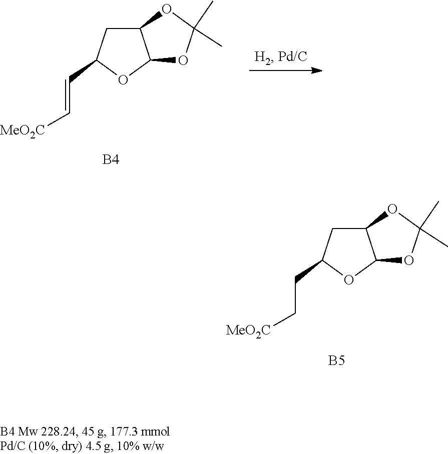

Crude B4 was dissolved in MeOH (300 ml) and hydrogenated on 10% Pd/C (4.5 g) at 80 psi for 2 h (GC/TLC control, Hept/EtOAc 1:1). The reaction mixture was flushed with Nitrogen and filtered through plug of Celite. The cake was rinsed with MeOH (2×100 ml). The combined filtrate was evaporated under reduced pressure to yield ˜45 g of the crude B5 as pale yellow viscous oil. This crude was purified on a Silica gel column (200 g) eluted with Hept/EtOAc 10:1 to 7:1 and all fractions contained the product were combined and evaporated resulted in 35 g (75% starting from Diacetone-D-glucose) of the desired ester as yellowish honey-like mass

### Preparation of B6

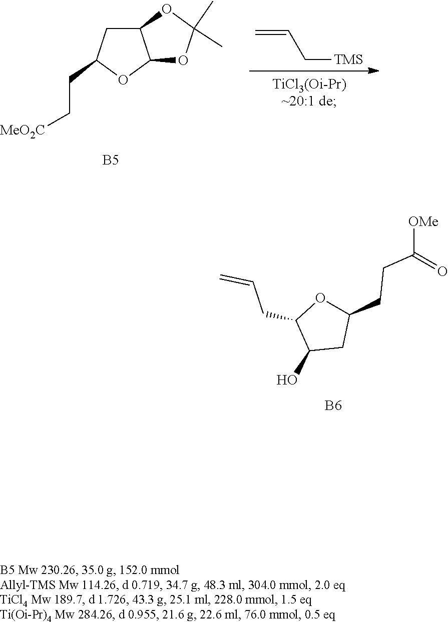

A solution of Titanium tetrachloride in dry DCM (200 ml) was cooled to 0° C. under Argon and a solution of Ti(Oi-Pr)₄ in dry DCM (50 ml) was slowly (exothermic reaction) added kept the reaction temperature below 10° C. The resulted yellow solution was stirred for 15 min, cooled to −20° C. and a mixture of B5 and Allyl-TMS in dry DCM (250 ml) was slowly added kept the temperature inside below −15° C. The reaction was stirred for 24 h at −20° C. (GC/TLC control; Hept/EtOAc 1:1), quenched with 1N aqueous HCl (300 ml) and after 30 min of stirring the phases were separated. The organic one was washed with 1N aqueous HCl (200 ml), 10% aqueous NaHCO₃ (100 ml) and brine, dried over Na₂SO₄, filtered and evaporated under reduced pressure. The residue (38 g) was dissolved in THF (100 ml) and passed through Silica gel pad (30 g). The solids were washed with THF (80 ml) and the combined filtrates (˜200 ml) were used in the next step

### Preparation of B7

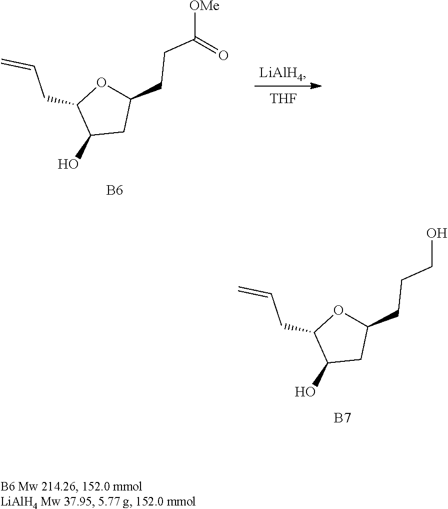

A suspension of LiAlH₄ in dry THF (150 ml) was cooled to 0° C. under Argon and a solution of B6 was slowly added kept the reaction temperature below 15° C. The reaction was stirred for 2 h at 0° C. (TLC control, Hept/EtOAc 1:2) and quenched by slow addition (exothermic, gas evolution!) of Water (6 ml), 15% aqueous NaOH (6 ml) and then more Water (17 ml). The resulted slurry was stirred for 2 h at 25° C. and filtered through Celite.

The cake was washed with THF (2×80 ml) and the combined organics were evaporated under reduced pressure. Then, the residue was co-evaporated with Toluene (2×80 ml) afforded 27 g of the yellowish crude B7.

### Preparation of B8

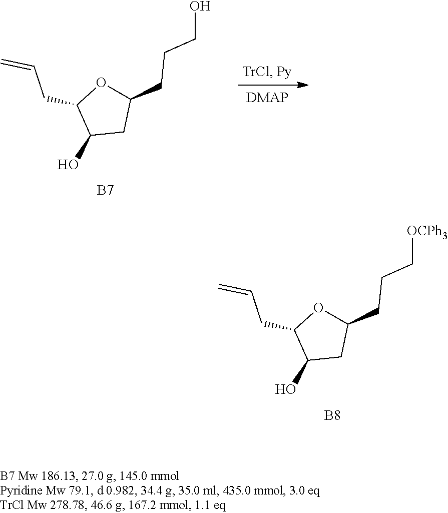

A solution of crude B7 and Pyridine in dry DCM (300 ml) was cooled to 0° C. under Argon. Trityl chloride was added in one portion and the reaction was left to warm to ambient temperature and stirred for 12 h (UPC²/TLC control Hept/EtOAc 1:1). The excess of Trityl chloride was quenched by addition of MeOH (3 ml), the mixture was stirred for additional 1 h and washed with 10% aqueous Citric acid (2×150 ml), 10% aqueous NaHCO₃ (100 ml) and brine, dried over Na₂SO₄, filtered and evaporated under reduced pressure afforded ˜70 g of oily residue. The residue was chromatographed on a Silica gel (400 g) and eluted with gradually with Hept/EtOAc from 20:1 to 5:1. All fractions contained the desired ER2-10 were combined and evaporated afforded 49.0 g (75% for 3 steps) of B8 as yellow mass.

### Preparation of B9

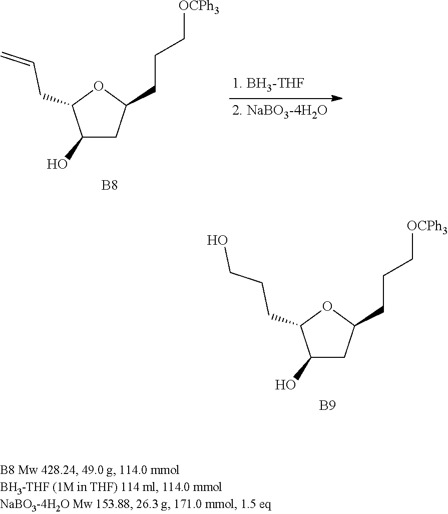

A solution of B8 in dry THF (250 ml) was cooled to 0° C. under Argon. BH₃-THF was slowly added kept the reaction temperature below 15° C. The reaction was stirred for 2 h at 0° C., then 12 h at 15-20° C. (TLC control, Hept/EtOAc 1:1) and quenched by slow addition (exothermic, gas evolution!) of 3% aqueous NaHCO₃ (100 ml). NaBO₃-4H₂O was added by portions (exothermic) and the reaction stirred for 12 h at 25° C. (TLC control, Hept/EtOAc 1:1). The solids were filtered off, washed with EtOAc (2×70 ml) and the combined filtrates were evaporated under reduced pressure. The aqueous residue was extracted with EtOAc (2×250 ml), the combined organics were washed with 10% aqueous NaHCO₃ (100 ml) and brine, dried over Na₂SO₄, filtered and evaporated under reduced pressure afforded ˜52 g of the crude B9.

### Preparation of B10

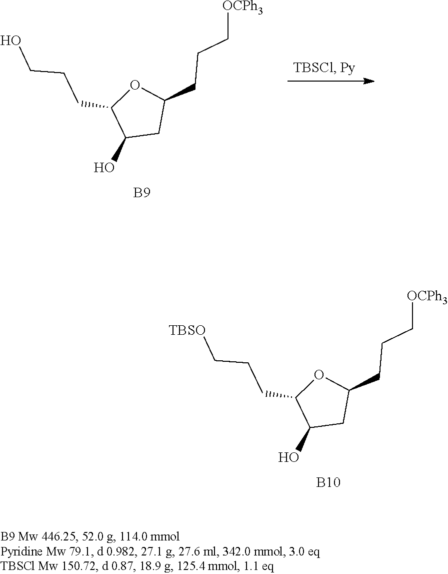

A solution of crude B9 and Pyridine in dry DCM (350 ml) was cooled to 0° C. under Argon. A solution of TBSCl in dry DCM (50 ml) was slowly added and the reaction was left to warm to ambient temperature and stirred for 12 h (UPC²/TLC control; Hept/EtOAc 2:1). The reaction was quenched with Water (50 ml), stirred for additional 1 h and washed with 10% aqueous Citric acid (2×150 ml), 10% aqueous NaHCO₃ (100 ml) and brine, dried over Na₂SO₄, filtered and evaporated under reduced pressure afforded ˜65 g of crude B10 which was used in the next step without purification.

### Preparation of B11

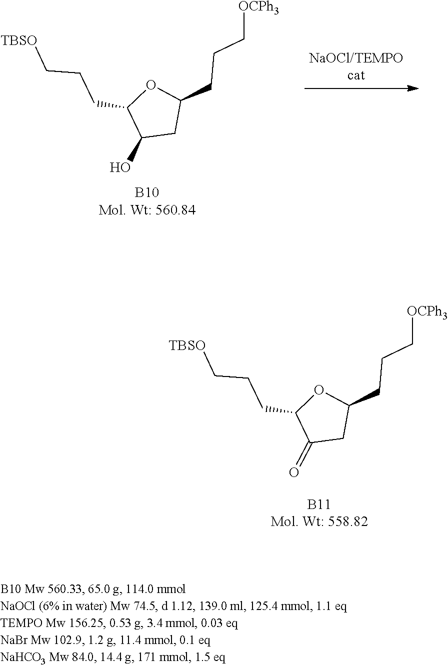

A solution of NaHCO₃ and NaBr in Water (150 ml) was mixed with solution of B10 and TEMPO in DCM (300 ml) and the mixture was cooled to 0° C. A solution of NaOCl was treated with 10% aqueous NaHCO₃ (50 ml) and slowly added into the mixture kept the temperature inside below 5° C. The reaction was stirred for 1 h at 0° C. (TLC control; Hept/EtOAc 2:1) quenched with IPA (10 ml), stirred for additional 20 min and phases were separated. The aqueous one was washed with DCM (200 ml) and the combined organics were dried over Na₂SO₄, filtered and evaporated under reduced pressure afforded ˜65 g of the crude B11 which was used in the next step without purification.

### Preparation of B12

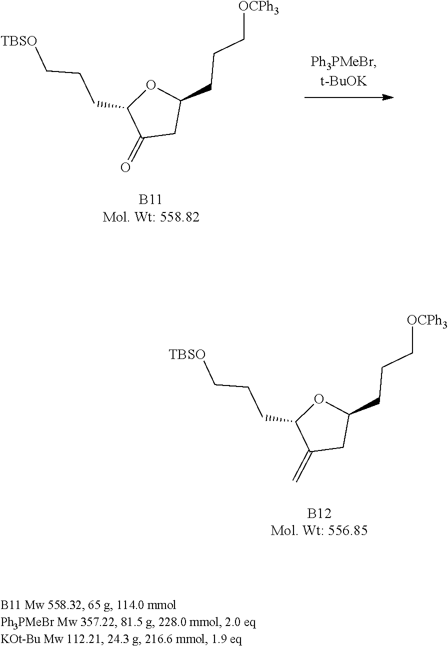

A mixture of crude B11 and Ph₃PMeBr in dry THE (800 ml) was cooled to 0° C. under Argon and t-BuOK was added by portions during 1 h period kept the reaction temperature below 10° C. The reaction was stirred at 0° C. for 2 h, then 4 h at 25° C. (TLC control; Hept/EtOAc 4:1) and quenched with 20% aqueous NH₄Cl (50 ml). After 15 min of stirring, the most of volatiles were evaporated under reduced pressure, and the semi-solid residue was triturated with Heptane/MTBE mixture (2:1; 500 ml) at 40° C., cooled to 0° C. and stirred for 4 h. The solids were filtered off, washed with cold Heptane/MTBE mixture (2:1; 2×100 ml) and the combined filtrates evaporated under reduced pressure to give ˜70 g of crude B12 contaminated with 3-5% of Ph₃PO.

The crude could be purified on a short Silica gel column (250 g) eluted with Heptane to Hept/EtOAc 20:1.

### Preparation of B13

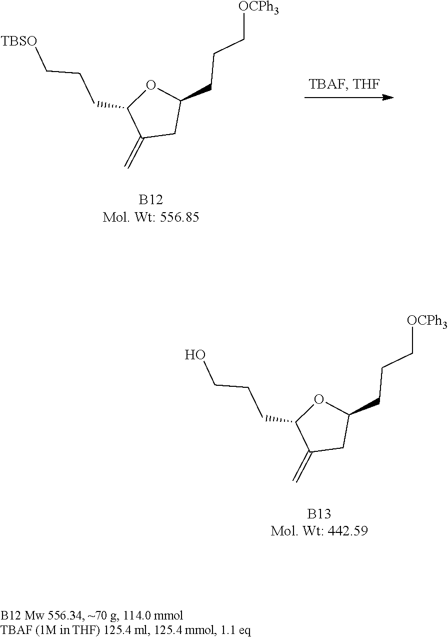

A solution of crude B12 in dry THE (400 ml) was cooled to 10° C. and TBAF was slowly added. The reaction was stirred for 2 h at 25° C. (TLC control; Hept/EtOAc 2:1) and quenched with 10% aqueous NH₄Cl (200 ml). After 15 min of stirring, the most of volatiles were evaporated under reduced pressure, and the aqueous residue was extracted with MTBE (2×300 ml). The combined organic were washed with 5% aqueous NH₄Cl (2×100 ml), 10% aqueous NaHCO₃ (100 ml) and brine, dried over Na₂SO₄, filtered and evaporated under reduced pressure afforded ˜60 g of crude ER2-15 which was purified on a Silica gel column (300 g), eluted with gradually with Hept/EtOAc from 20:1 to 2:1. All fractions contained the desired product were combined and evaporated afforded 35.3 g (70% from ER2-07) of B13 as yellowish sticky mass.

### Preparation of B14

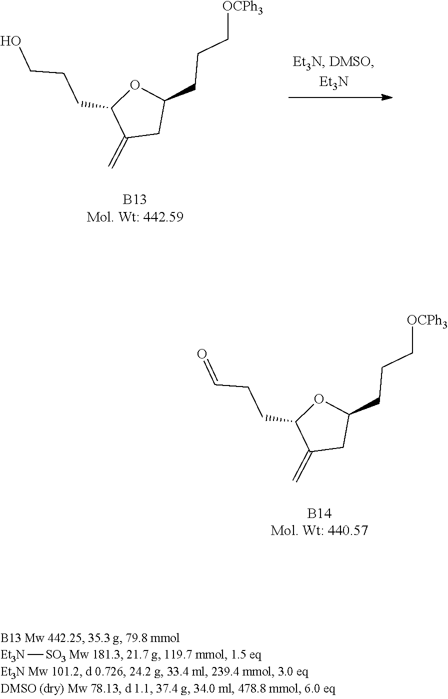

A solution of B13, Et₃N and DMSO in dry DCM (100 ml) was cooled to 0° C. under Argon and a solution of Et₃N—SO₃ in dry DCM (30 ml) doped with Et₃N (1 ml) was slowly added kept the reaction temperature below 15° C.

The reaction was stirred at 0° C. for 2 h, then 4 h at 25° C. (TLC control; Hept/EtOAc 2:1) and quenched with Water (200 ml). After 15 min of stirring, the mixture was diluted with MTBE (300 ml), the phases were separated and the aqueous one was extracted with MTBE (100 ml). The combined organic were washed with 10% aqueous Citric acid (2×150 ml), 10% aqueous NaHCO₃ (100 ml) and brine, dried over Na₂SO₄, filtered and evaporated under reduced pressure afforded ˜35 g of solid mass. The residue was dissolved in Heptane/MTBE mixture (2:1; 100 ml) and passed through silica gel plug (60 g). Plug was washed with additional Heptane/MTBE 2:1 (200 ml) and all solvents were evaporated under reduced pressure.

The residue (36 g) was crystallized from Heptane/MTBE (10:1 v/v; 140 ml) afforded 30 g (85% yield) of the desired B14 as off-white solid with 97.3% de.

Second crystallization afforded 26 g of B14 with more than 99% de

The filtration through Silica gel plug is optional. The crude could be taken to crystallization without purification afforded 75% yield.

After crystallization all filtrates were evaporated, the residue was passed through Silica gel column and crystallized to give additional 12% of B14.

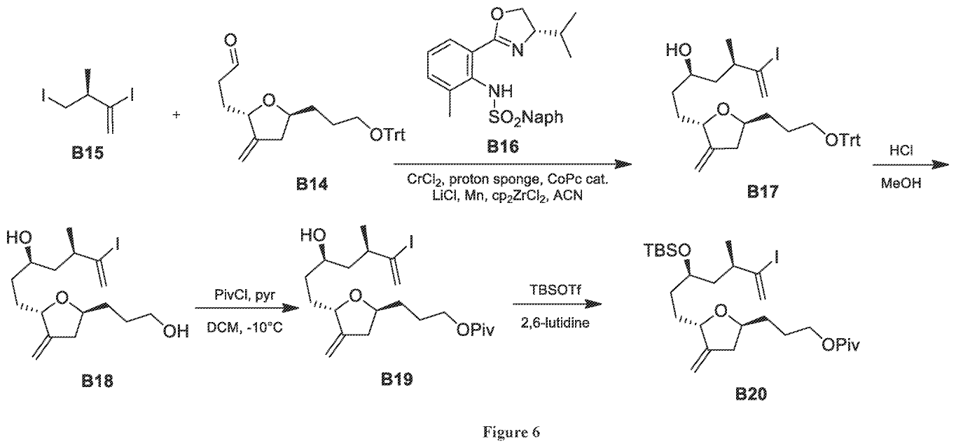

### Example 6: Preparation of B20 from B14 (See Also FIG. 6)

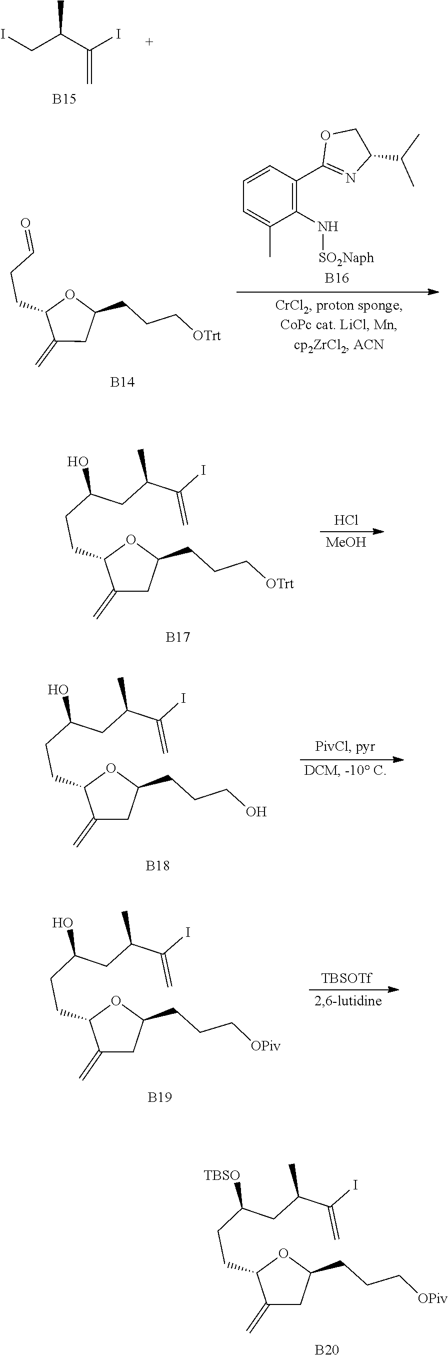

### Preparation of B17

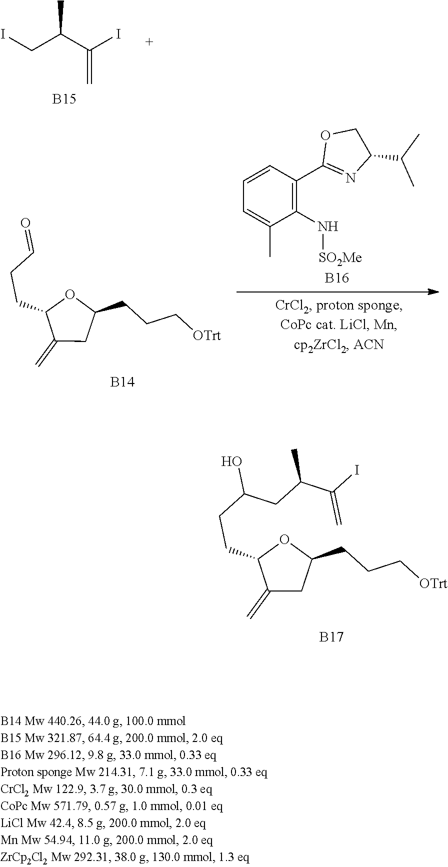

A three-necked flask was dried, equipped with stirrer, septum and two taps and flushed with Argon by three vacuum-Argon cycles. CrCl₂ (very sensitive to moisture), B16 and Proton sponge were loaded into the flask and flushed with Argon. Then anhydrous AcN (250 ml) was introduced through the septum and the resulted green mixture was stirred for 1 h at 25° C. until complete dissolution. In the separated three-necked flask, flushed as described above, B14, B15 LiCl, Mn and CoPc were mixed under Argon, flushed twice and then anhydrous AcN (700 ml) was introduced through the septum. The resulted suspension was stirred for 10 min, and the prepared Cr-ligand solution was quickly added under positive pressure of Argon. The mixture was stirred for 10 min and then ZrCl₂Cp₂ was loaded in one portion under positive Argon pressure. The reaction was stirred for 48-72 h at 25° C. (TLC/UPC² control, Hept/EtOAc 2:1; if necessary, more CoPc (0.01 eq) may be added). The reaction was quenched with Fluorisil (80 g), stirred for 15 min, diluted with MTBE (1.5 L) and stirred for additional 2 h. The suspension was filtered through Celite, the cake was washed with EtOAc (2×300 ml), and the combined filtrates were concentrated under reduced pressure. The residue was taken off with Toluene (700 ml; if need DCM (100 ml) may be added), Celite (70 g) was added in one portion, the resulted mixture was stirred for 30 min and filtered. The cake was washed with Toluene (2×150 ml) and the combined filtrates were evaporated under reduced pressure to give ˜100 g of the sticky residue which was hydrolyzed without any purification.

### Preparation of B18

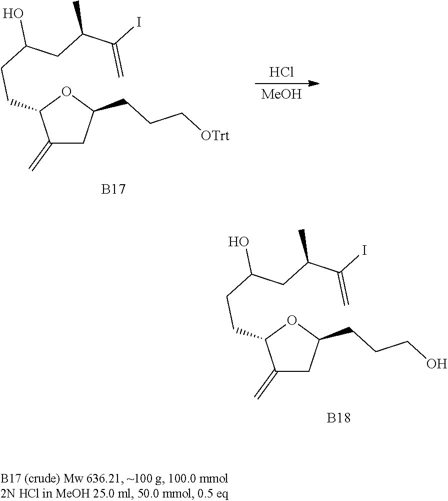

The crude B17 (˜100 g) was dissolved in anhydrous MeOH (500 ml; if necessary, dry DCM (100 ml) may be added), a mixture was cooled to 0° C. under Argon and 2N HCl in MeOH was added in one portion. The reaction was stirred at 0-4° C. until completeness (˜4 h)-UPC²/TLC monitoring; Hept/EtOAc 2:1 or 1:1) and quenched by addition (slow gas evolution) of solid NaHCO₃ (9 g). After 1 h of stirring at 25° C., the mixture was cooled back to 0° C. (DCM has to be evaporated at this stage if previously added), stirred for 30 min and the formed precipitate was filtered off and washed with cold MeOH (2×100 ml). The combined filtrates were evaporated under reduced pressure and the residue (˜50 g) was purified on a Silica gel (250 g) eluted with gradient Hept/EtOAc 6:1 to EtOAc. All fractions contained the desired diol were combined and concentrated under reduced pressure afforded 19.7 g (50% from B14) of B18 as greenish oil with 7:1 ratio of diastereomers.

### Preparation of B19

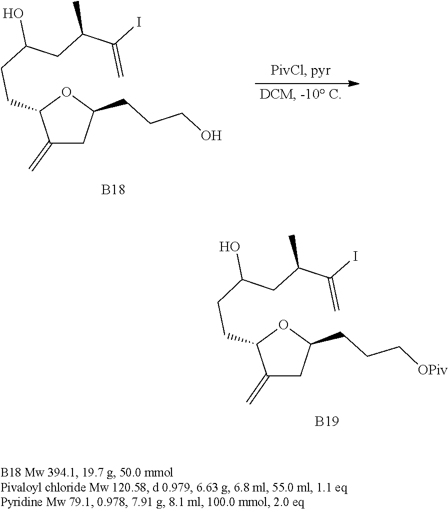

A solution of B18 and Pyridine in dry DCM (200 ml) was cooled to 0° C. under Argon and Pivaloyl chloride was slowly added kept the reaction temperature below 5° C. The reaction was stirred for 4 h at 0° C. (UPC²/TLC control; Heptane/EtOAc 1:1) and quenched with Water (70 ml). The mixture was stirred for 30 min, and all volatiles were evaporated under reduced pressure at 40° C. The aqueous residue was extracted with MTBE (2×150 ml), the combined extracts were washed with 10% aqueous Citric acid (100 ml), 9% aqueous NaHCO₃ (50 ml) and brine, dried over Na₂SO₄, filtered and evaporated under reduced pressure afforded 24.0 (quant) of the crude B19 (90-95% chemical purity) which was used without purification.

The crude may be purified on a short Silica gel column (150 g) eluted with gradient Heptane to Hept/EtOAc 3:1 or on a preparative HPLC with separation of diastereomers.

### Preparation of B20

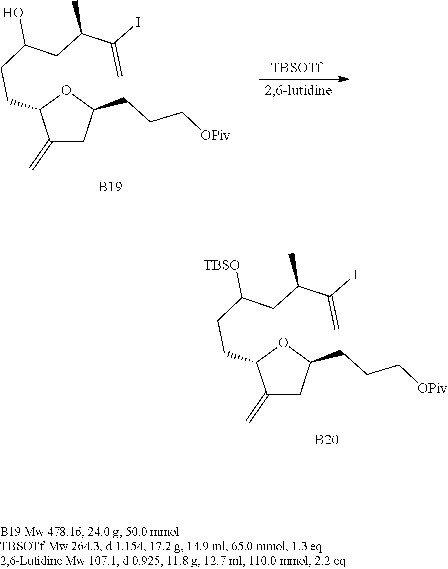

B19 and 2,6-Lutidine were dissolved in dry MTBE (200 ml) and a solution was cooled to 0° C. under Argon. TBSOTf was slowly added kept the reaction temperature below 10° C., the resulted mixture was stirred for 2 h at 0° C. (UPC² control, if B19 is still present stir for 1 h at 25° C.; TLC control, Hept/EtOAc 2:1). The reaction was quenched by addition of Water (100 ml), the resulted mixture was stirred for 30 min and then the phases were separated. The aqueous one was extracted with MTBE (50 ml) and the combined organics were washed with 10% aqueous Citric acid (100 ml), 10% aqueous NaHCO₃ (50 ml) and brine, dried over Na₂SO₄, filtered and evaporated under reduced pressure. The residue (˜30 g) was co-evaporated with Heptane (150 ml) afforded 29.5 g (near quant) of B20 as yellow oil which was used in the next step.

### Second Approach—Preparation of B29(1)

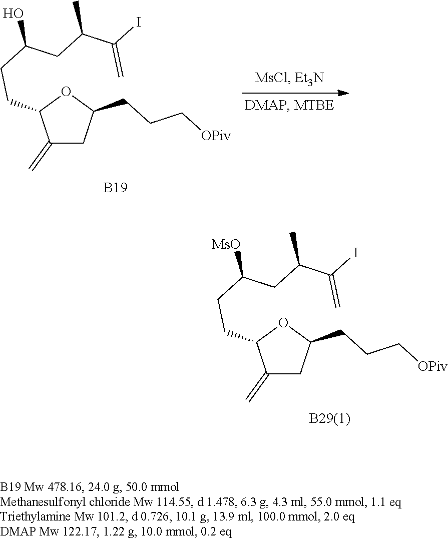

A solution of B19, Et₃N and DMAP in dry MTBE (250 ml) was cooled to 0° C. under Argon and MsCl was slowly added kept the temperature inside below 10° C. The reaction was stirred for 1 h at 0° C. (UPC²/TLC control; DCM) and quenched with Water (70 ml). After 20 min of stirring the phases were separated, the aqueous one was extracted with MTBE (80 ml) and the combined organics were washed with 10% aqueous Citric acid (70 ml) and 10% aqueous NaHCO₃ (70 ml), dried over Na₂SO₄, filtered and evaporated under reduced pressure afforded 24.9 g (near quant) of the crude BII29(1) as yellowish oil which was used without additional purification (stored at −20° C.)

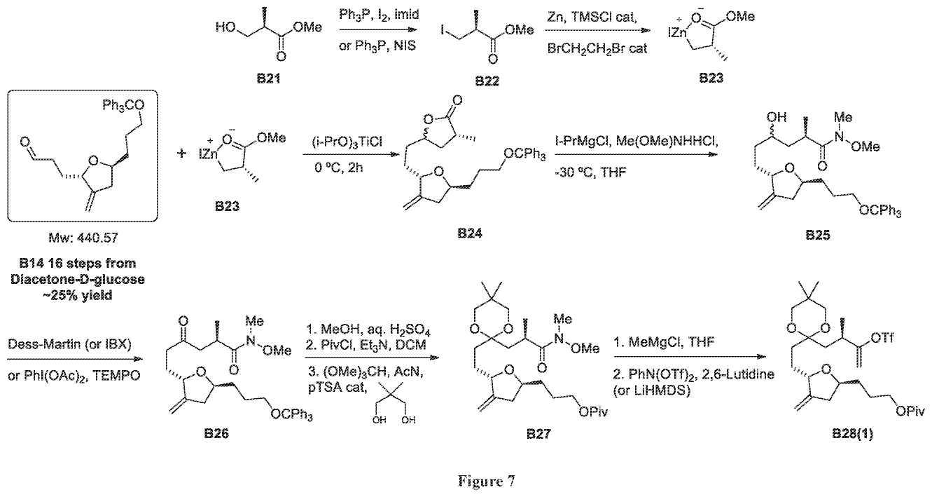

### Example 7: Preparation of B Fragment Via Zn-Mediated Aldol-Type Condensation (B28(1))—See Also FIG. 7

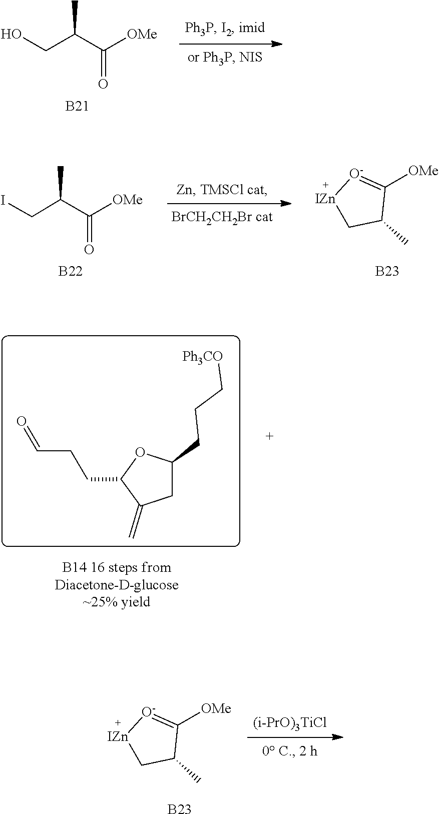

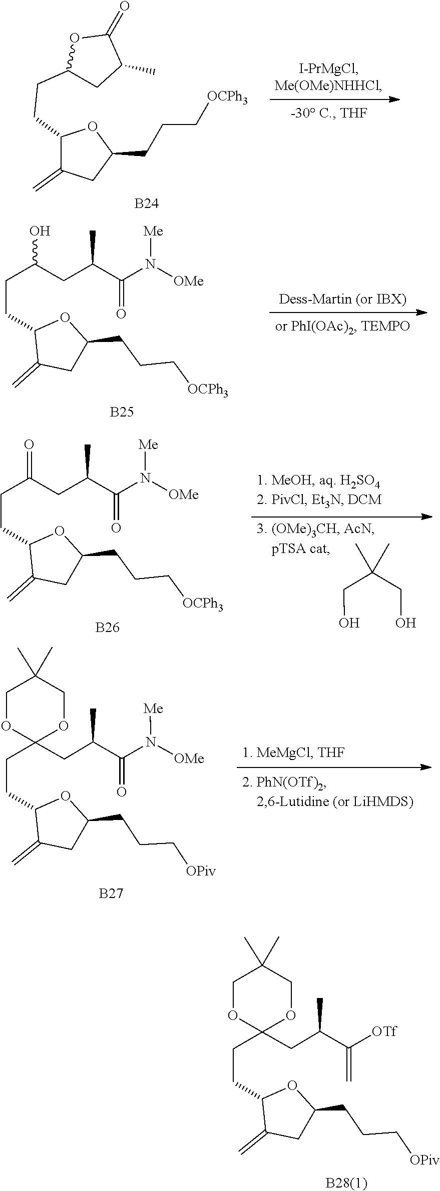

### Step 1 Preparation of B23—Zn-Adduct

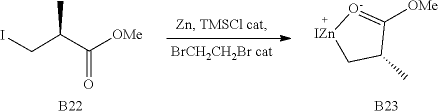

The dry flask equipped with reflux condenser, heating-cooling system, thermometer and magnetic stirrer was charged under Argon with Zn dust (Mw 65.38, 6.54 g, 100 mmol), Note 1, and anhyd THF (50 ml). This dispersion was stirred for 30 min, and 1,2-dibromoethane (0.5 ml) was poured in one portion. The mixture was refluxed for 10 min, cooled to 25° C. and TMSCl (0.5 ml) was dropwised into the flask. After 10 min of vigorous stirring, a solution of iodine (0.64 g, 2.5 mmol) in dry THF (5 ml) was added in one portion and the resulted mixture was stirred until complete disappearance of the resultant brown color (2-5 min). Then, a solution of (S)-methyl 3-iodo-2-methylpropanoate (Mw 228.1, 11.4 g, 50 mmol) in dry THF was poured into the flask and the resulted mixture was vigorously stirred for 18 h at 50° C., cooled to 25° C. and allowed to stand, Note 2, under Argon until formation of clear supernatant which was used for reaction.

Note 1 Zn may be pre-activated as follows: vigorous stirring for 10 min with 3N HCl, quick filtration, washing with water, acetone and MTBE, then drying under reduced pressure and storing under Argon.

Note 2 A suspension may be filtered under Argon.

Step 2 Preparation of B24—Aldol-Type Reaction. Lactone Formation

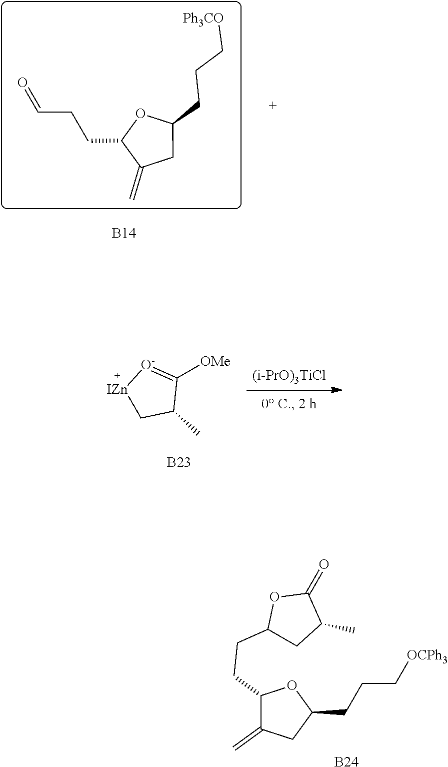

A freshly prepared solution of (i-OPr)₃TiCl (7.8 g, 30 mmol) in dry DCM (40 ml) was cooled to −30° C. under Argon and the clear solution of Zn-reagent prepared above (30 mmol) was slowly added kept the reaction temperature below −20° C. After 15 min of stirring, a solution of B14 (8.8 g, 20 mmol) in dry DCM (30 ml) was added slowly, the reaction was heated to 0° C. and stirred for 2-8 h (UPC²/TLC monitoring). The reaction was quenched by addition of cold 5% aqueous ammonia (50 ml) and the volatiles were evaporated under reduced pressure, Note 1. The residue was diluted with in MTBE (100 ml), filtered through Celite and cake was washed with MTBE (2×25 ml). The filtrates were combined, the phases were separated and the organic one was quickly washed with cold 0.5M aq HCl, 10% aq NaHCO₃ and brine, dried over Na₂SO₄, filtered and evaporated to give 13.0 g of crude lactone B24 as yellow oil which was used in the next step, Note 2.

Note 1 A filtration may be performed before evaporation.

Note 2 The crude may be purified on a Silica gel column.

### Step 3 Formation of Amide

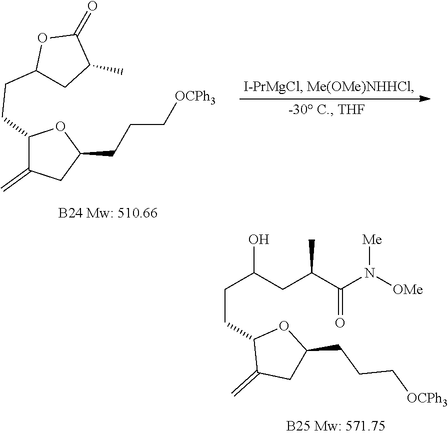

A mixture of N,O-dimethyl hydroxylamine hydrochloride (2.9 g, 29.8 mmol) and the crude Lactone B24 (13.0 g, 20 mmol) in dry THF (100 ml) was cooled to −25° C. under Argon and I-PrMgCl (2M in THF, 30 ml, 60 mmol) was slowly added kept the temperature inside below −15° C. The reaction was stirred at −20° C. for 1 h and quenched with 20% aqueous NH₄Cl (30 ml). The most of THE was evaporated under reduced pressure, the residue was extracted with EtOAc (2×60 ml) and the combined organics were washed with 10% aq NaHCO₃ and brine, dried over Na₂SO₄, filtered and evaporated under reduced pressure. The residue (13.6 g) was purified on a short Silica gel column (70 g) afforded 9.4 g (82% yield) of the desired product as yellowish oil.

### Step 4 Oxidation

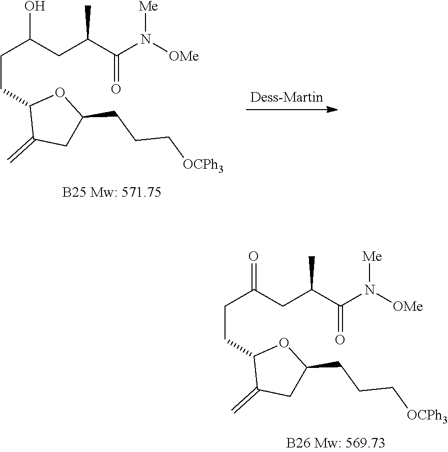

A solution of B25 (9.4 g, 16.5 mmol) in dry DCM (100 ml) was cooled to 0° C. under Argon and Dess-Martin periodinane (9.8 g, 23.1 mmol, 1.4 eq), Note 2 was added in three portions. The reaction was stirred for 1 h at 0° C., then warmed to 20° C. and stirred for 6 h until complete disappearance of the starting alcohol (UPC²/TLC control). A mixture was poured (gas evolution) into a solution of 9% aqueous NaHCO₃ (60 ml, ˜4 eq) and 10% aqueous Na₂SO₃ (40 ml, ˜2 eq). The resulted mixture was stirred for 2 h until complete dissolution of solids, the phases were separated, and the aqueous one was extracted with DCM (50 ml). The combined organics were washed again with 9% aqueous NaHCO₃ (30 ml), dried over Na₂SO₄, filtered and evaporated under reduced pressure to give 10.1 g (quant) of the crude ketone as yellow oil.

Note 2Instead of DMP, IBX or Diacetoxyiodobenzene TEMPO may be used for oxidation.

### Step 5 Protection of Ketone

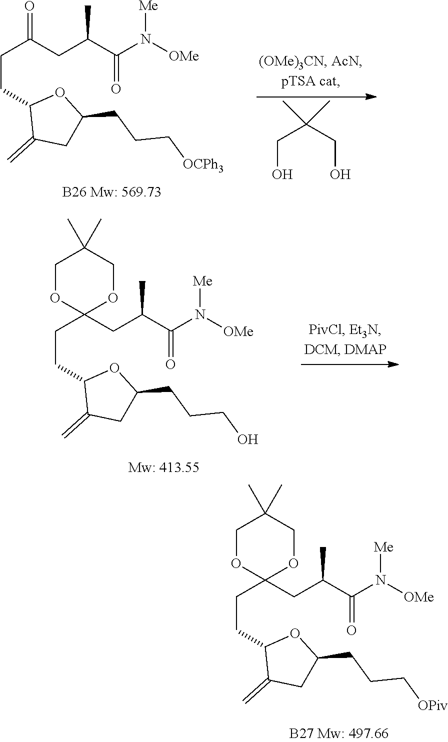

A solution of crude ketoamide B26 (6.6 g, 11.6 mmol), Neopentyl glycol (2.4 g, 23.1 mmol, 2.0 eg), p-TSA-H₂O (67.9 mg, 0.34 mmol, 0.03 eg) and Trimethyl ortoformate (1.9 ml, 17.34 mmol, 1.5 eq) in anhyd AcN (90 ml) was stirred at 20-25° C. for 12 h under Argon (UPC² or TLC Hept/EtOAc 1:1). Then, the reaction was quenched with 10% aq NaHCO₃ (50 ml) and all organic volatiles were evaporated under reduced pressure. The aqueous residue was extracted with MTBE (2×100 ml), the combined organics were washed with 9% aqueous NaHCO₃ (30 ml) and brine, dried over Na₂SO₄, filtered and evaporated under reduced pressure. The residue (7.3 g) was purified on a short Silica gel column (50 g), when the unipolar impurities were eluted till Hept/EtOAc 6:1 and the desired compound was eluted gradually from Hept/EtOAc 6:1 to Hept/EtOAc 1:1. After evaporation 4.5 g (93.8% yield) of the desired alcohol was obtained as yellowish oil.

Alcohol (4.5 g, 10.9 mmol), Et₃N (3.0 ml, 21.8 mmol, 2.0 eq) and DMAP (0.27 g, 2.2 mmol, 0.2 eq) were dissolved in dry DCM (50 ml) under Argon and Pivaloyl chloride (1.60 ml, 13.1 mmol, 1.2 eq) was added dropwise. The reaction was stirred for 6 h at 25° C. (UPC² or TLC Hept/EtOAc 1:1) and quenched by addition of Water (20 ml). The resulted two-phase mixture was stirred for 30 min, the phases were separated and he aqueous one was extracted with DCM (20 ml). The combined organics were washed with 10% aqueous Citric acid (30 ml), 9% aqueous NaHCO₃ (30 ml) and brine, dried over Na₂SO₄, filtered and evaporated under reduced pressure to get 5.5 g (quant) of the crude as an yellow liquid which was used in the next step without purification.

Step 6 Preparation of Ketone from B27

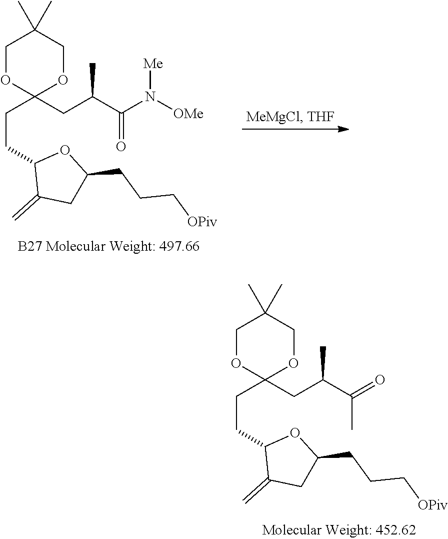

A solution of Amide B27 (6.0 g, 12.1 mmol) in dry THF (70 ml) was cooled to ˜30° C. under Argon and MeMgCl (3M in THF, 8.1 ml, 24.2 mmol) was slowly added kept the reaction temperature below −20° C. After 30 min of stirring at −30° C. (UPC²/TLC control Hept/EtOAc 2:1), the reaction was quenched with 20% aq NH₄Cl (30 ml), the most of volatiles were evaporated under reduced pressure and the aqueous residue was extracted with MTBE (2×50 ml). The combined organics were washed with brine, dried over Na₂SO₄, and passed through Silica gel plug (10 g). After evaporation of solvent, 5.5 g (quant) of the desired ketone was obtained as yellow oil. Note 1.

Note 1 The crude may be purified on a Silica gel column.

### Step 7 Preparation of B28

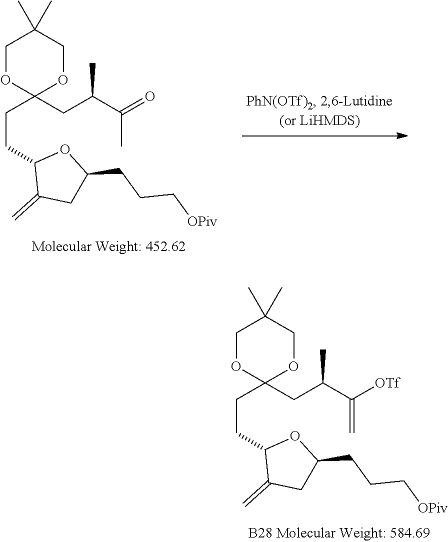

To a stirred solution of ketone (5.5 g, 12.1 mmol) in dry THF (100 ml) at −40° C. under Argon LiHMDS (1M in toluene, 18.2 ml, 18.2 mmol, 1.5 eq) was slowly added kept the reaction temperature below −30° C. The resulted mixture was stirred for 30 min at −40° C. and then a solution of PhN(OTf)₂ (6.1 g, 18.2 mmol) in dry THE (30 ml) was slowly added. The reaction was warmed to 0° C. during 1 h, stirred for additional 1 h and then quenched with 9% aqueous NaHCO₃ (100 ml). The most of solvents were evaporated under reduced pressure, the aqueous residue was extracted with MTBE (2×60 ml) and the combined organics were washed with water (2×50 ml) and brine, dried over Na₂SO₄, filtered and evaporated under reduced pressure. The crude (12.9 g) was purified on a Silica gel column (90 g), doped with Et₃N afforded after evaporation 6.5 g (92% yield) of the desired triflate as pale yellow oil.

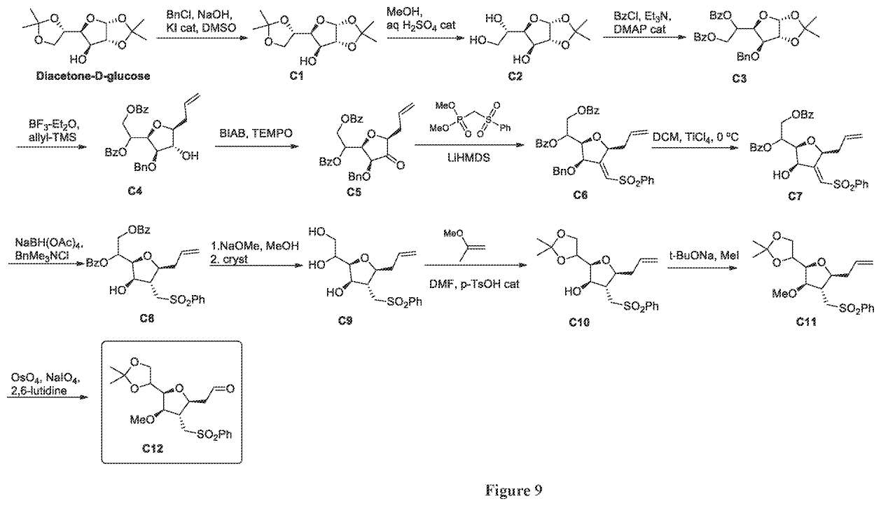

### Example 8: Preparation of C12 (See Also FIG. 9)

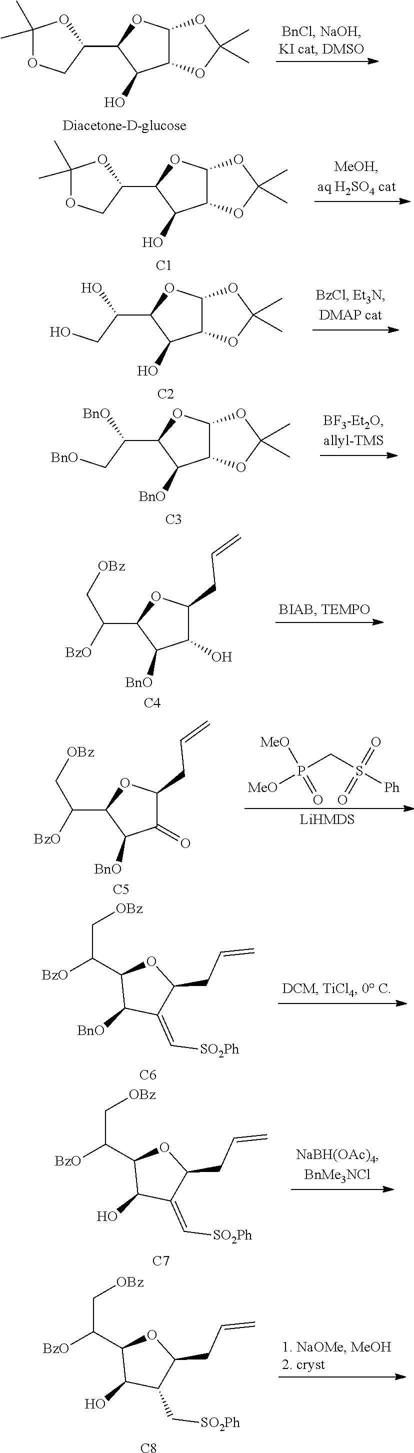

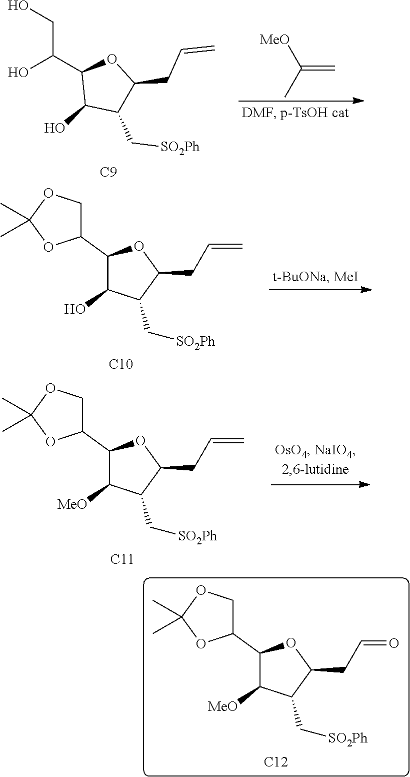

### Preparation of C1

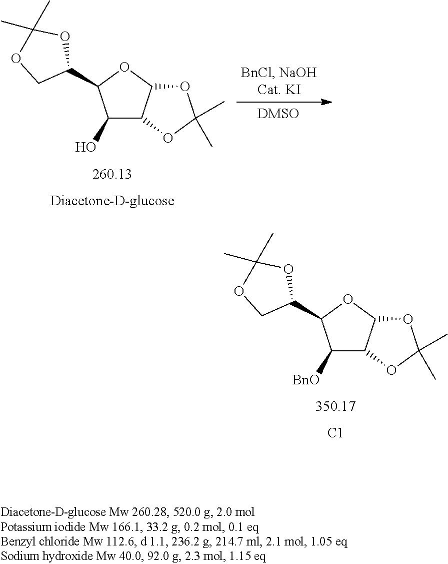

NaOH (92.0 g, 2.3 mol) was added in one portion under Nitrogen into the stirred solution of Diacetone-D-glucose (520.0 g, 2.0 mol) and KI (33 g, 0.2 mol) in dry DMSO (1650 g) and the resulted mixture was stirred for 15 min at 25° C. till almost complete dissolution of solids. Then, Benzyl chloride (236.21 g, 2.10 mol) was slowly added kept the reaction temperature in the range 25-33° C. and the reaction was stirred at 35° C. for 14 h (GC/TLC control; EtOAc/Hep 2:1. At this point GC showed presence of 97.9% of the desired product)

Then, the reaction was diluted with MTBE (2959 g) and quenched by addition of Water (5000 g). The resulted two-phase mixture was stirred for 30 min, the phases were separated and the aqueous one was extracted with MTBE (3×1100 g). The combined organics were washed with water (1000 g) and brine (500 g), dried over Sodium Sulfate (160 g), filtered and evaporated under reduced pressure to get 689.6 g (98.4% yield) of Cl.

### Preparation of C2

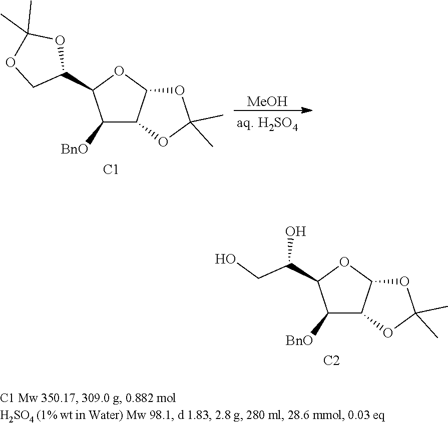

C1 (309 g, 0.882 mol) was dissolved in MeOH (620 g), the resulted solution was cooled to 10° C. and treated with 1% aqueous H₂SO₄ (280 g). The reaction was stirred at 30° C. for 36 h (UPC²/TLC control; EtOAc/Hep 1:1) and quenched by addition of solid NaHCO₃ (20 g). Slow gas evolution was observed. After 1 h of the progressive stirring the most of organic volatiles were evaporated under reduced pressure and the aqueous residue was extracted with EtOAc (3×700 g). The combined organics were washed with brine (200 g) dried over Sodium Sulfate and filtered. This solution was used into the next step.

### Preparation of C3

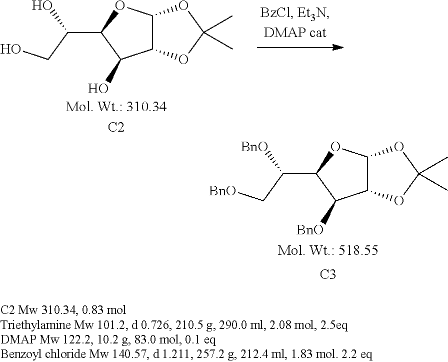

Triethylamine (290 ml, 2.08 mol, 2.5 eq) and DMAP (10.2 g, 0.083 mol) were added into a solution of C2 from the previous step (contained 0.833 mol of C2 by GC) and the resulted mixture was cooled to 0° C. Benzoyl chloride (212.4 ml, 1.83 mol, 2.2 eq) was slowly added dropwise kept the temperature inside below 15° C., The reaction was allowed to warm to 25° C. and stirred for 14 h (UPC²/TLC control Heptane/EtOAc 1:1). The most of volatiles were evaporated under reduced pressure and the residue was portioned between MTBE (1000 ml) and water (1000 ml). The phases were separated, the aqueous one was extracted with MTBE (2×500 ml) and the combined organics were washed with 10% aqueous Citric acid (2×200 ml), 10% aqueous NaHCO₃ (200 ml) and brine (2×200 ml), dried over Na₂SO₄, filtered and evaporated under reduced pressure afforded 431 g (near quant) of the desired product as yellowish honey-like mass with ˜95% purity by UPC².

### Preparation of C4

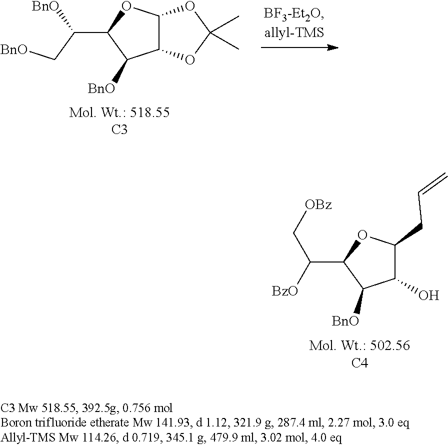

Before reaction C3 was co-evaporated with Toluene (1000 ml) under reduced pressure to remove traces of Water

A solution of C3 (392.5 g, 0.756 mol) and Allyl-TMS (480 ml, 3.02 mol) in dry DCM (2000 ml) was cooled to 0° C. under Argon. BF₃-Et₂O (287.4 ml, 2.27 mol) was slowly added kept the reaction temperature below 15° C. and then the reaction was stirred at 25° C. for 12 h (UPC²/TLC control, Heptane/EtOAc 1:1; R_{f}C3 0.6, R_{f}C4 0.5). The reaction mixture was slowly (Gas evolution) poured into a cold 7% aqueous NaHCO₃ (2.5 L), the stirring was continued for 1 h and the layers were separated. The aqueous one was extracted with DCM (2×250 ml) and the combined organic extracts were washed with 7% aqueous NaHCO₃ (300 ml), brine (200 ml), dried over Na₂SO₄, filtered and evaporated under reduced pressure. The residue (˜420 g) was purified on a short Silica gel column (1 kg) afforded after evaporation and drying 360 g (95% yield) of C4 as honey-like yellow mass with 94.1% purity by UPC²(C3 1.0%, C4(R) 4.0%).

### Preparation of C5

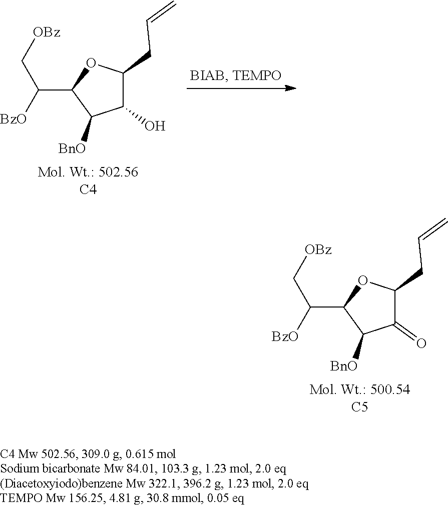

TEMPO (4.8 g, 30.8 mmol, 0.05 eq) was added in one portion into a suspension of C4 (309.0 g, 0.615 mol), NaHCO₃ (103.3 g, 1.23 mol, 2.0 eq) and (Diacetoxyiodo)benzene (396.2 g, 1.23 mol, 2.0 eq) in dry DCM (2000 ml) stirred under Argon at 25° C. The stirring was continued for 12 h (UPC²/TLC monitoring EtOAc/Heptane 1:1), the solvent was evaporated under reduced pressure and the residue was taken up with MTBE (800 ml). The resulted suspension was stirred for 20 min, filtered and the cake was washed with MTBE (3×200 ml). The combined filtrates were treated with the mixture of 7% aqueous NaHCO₃ (1000 ml) and 10% aqueous Na₂SO₃ (500 ml) and the resulted mixture was vigorously stirred for 30 min. The layers were separated, the aqueous one was extracted with MTBE (500 ml) and the combined organics were washed with water (200 ml) and brine (200 ml), dried over Na₂SO₄, filtered and evaporated under reduced pressure. The residue (˜500 g) was triturated three times with Heptane (500 ml each portion) as follows: the resulted two phase mixture was vigorously stirred for 30 min at 20° C., stirring was stopped and the mixture was cooled to −10° C. and the upper layer was decanted off. The residue was dried under reduced pressure until constant weight afforded 315 g (quant) of C5 as yellowish sticky mass with 92.6% purity by UPC² and contaminated with traces of iodobenzene.

### Preparation of C6

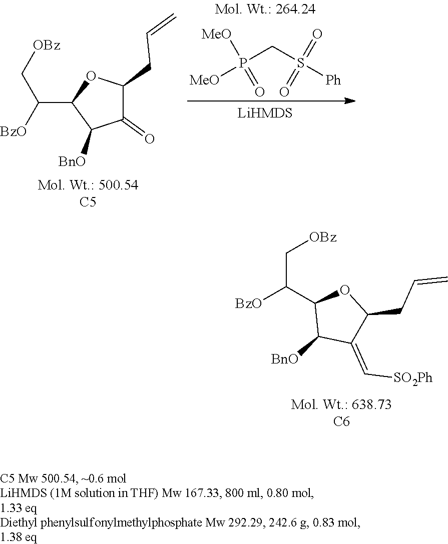

A solution of Diethyl phenylsulfonylmethylphosphate (242.8 g, 0.83 mol, 1.38 eq) in dry Toluene (8000 ml) was cooled to 0° C. under Argon and LiFMDS (800 ml, 0.80 mol, 1.33 eq) was slowly added kept the temperature inside below 20° C. The resulted mixture was stirred for 30 min at 25° C., during which time the clear solution turned into a yellow gel. Then, a solution of C5 (315 g, ˜0.60 mol, based on the calculation by purity) in dry Toluene (500 ml) was slowly added and the resulted mixture was stirred for 12 h at 25° C. (UPC²/TLC control Heptane/EtOAc 1:1). The reaction was quenched with 10% aqueous Citric acid (2000 ml), stirred for 10 min and the phases were separated. The organic one was washed with 10% aqueous Citric acid (2000 ml) and the combined aqueous layers were back-extracted with Toluene (2×500 ml). Then, the combined organics were washed with Water (1000 ml) and brine (1000 ml), dried over Na₂SO₄, filtered and evaporated under reduced pressure afforded 440 g of the crude C6 (Z/E ratio 10:1). The crude was purified on a Silica gel (1.2 kg), eluted with the gradient mixture Heptane/EtOAc 10:1, 5:1, 2:1 and 1:1. All fractions contained the desired product were combined and evaporated under reduced pressure afforded 366 g (93.3% yield for 2 steps) of C6 as yellow sticky oil with 89.1% purity by UPC² contaminated with 9.0% of trans isomer (Z/E ratio ˜10:1).

### Preparation of C7

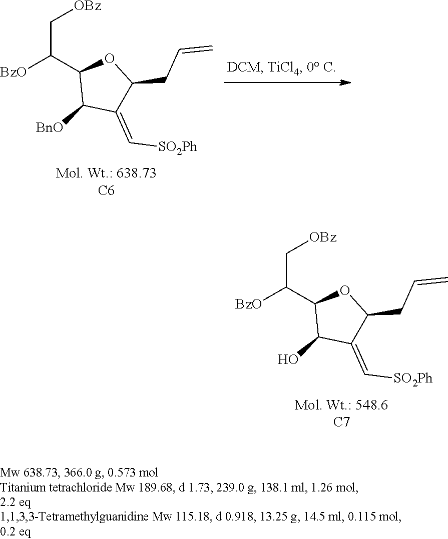

A solution of TiCl₄ (138.1 ml, 1.26 mol, 2.2 eq) in dry DCM (1600 ml) was cooled to −5° C. under Argon and Tetramethylguanidine (14.5 ml, 0.115 mol, 0.2 eq) was slowly added kept the reaction temperature below 5° C. The dark-brown mixture was stirred at 0° C. for 30 min and a solution of C6 (366.0 g, 0.573 mol) in dry DCM (800 ml) was slowly added at the rate which kept the inside temperature below 5° C. Then, the reaction was stirred for 12 h at 0° C. (UPC²/TLC monitoring, Heptane/EtOAc 1:1) and slowly poured into vigorously stirred mixture of 25% aqueous ammonia solution (500 ml) with ice (500 g). The resulted suspension was stirred for 30 min and filtered through Celite. The cake was washed with DCM (2×400 ml) and the layers were separated. The organic one was washed with water (500 ml) and brine, dried over Na₂SO₄, filtered and evaporated under reduced pressure afforded 342 g (314.4 g by theory) of the crude C7 as brownish oil, which was used in next step without any purification.

### Preparation of C8

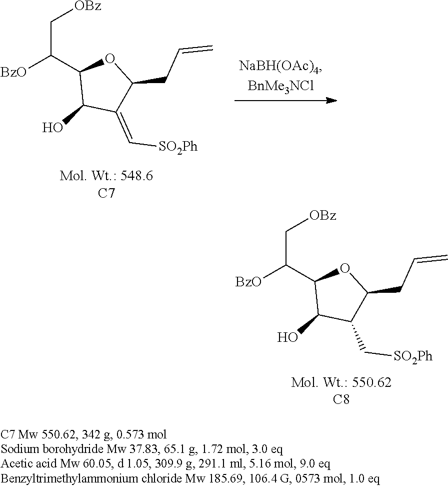

A dispersion of NaBH₄ (65.1 g, 1.72 mol, 3.0 eq) in dry THF (2000 ml) was cooled to 0° C. under Argon and Acetic acid (295.1 ml, 5.16 mol, 9.0 eq) was slowly (Gas evolution) added kept the temperature inside below 20° C. Then, the resulted suspension was stirred for 30 min at 25° C. until gas evolution almost stopped. BnMe₃NCl (106.4 g, 0.573 mol) was poured into reaction vessel in three equal portions, the mixture was heated to 60° C. and stirred for additional 30 min. Then, a solution of the crude C7 (342 g) in dry THF (500 ml) was dropwised inside and the reaction was stirred at 60° C. for 12 h (UPC²/TLC control, Heptane/EtOAc 1:1), cooled to 10° C. and quenched by slow (Gas evolution) addition of water (1000 ml). The most of THF was evaporated under reduced pressure, MTBE (1000 ml) was added and the resulted mixture was stirred for 30 min. The layers were separated, the aqueous layer was extracted with MTBE (2×500 ml) and the combined organics were slowly (Gas evolution!) poured into saturated aqueous NaHCO₃ (5000 ml) for neutralization of the remained Acetic acid. After 30 min of stirring, the phases were separated, the organic one was washed with water (500 ml) and brine (500 ml), dried over Na₂SO₄, filtered and evaporated under reduced pressure afforded 340 g (315.5 g by theory) of the crude C8 as brownish oil. This product was used in next step without additional treatment.

### Preparation of C9

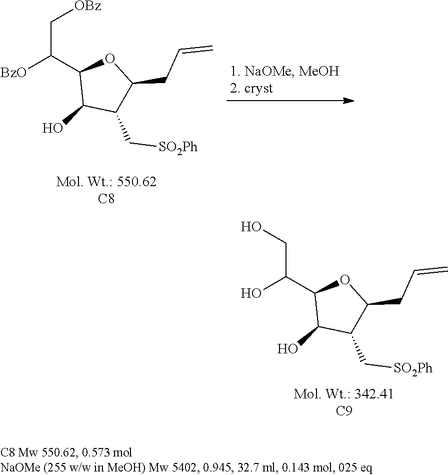

C8 (340 g from previous step, 0.573 mol) was dissolved in dry MeOH (1000 ml) under Argon and the resulted solution was treated with NaOMe (32.7 ml, 0.143 mol, 0.25 eq). The reaction was stirred for 14 h at 25° C. (UPC²/TLC control, Heptane/EtOAc 1:1 and DCM/MeOH 9:1), quenched by addition of 10% aqueous Citric acid (600 ml) and the most of MeOH was evaporated under reduced pressure. The aqueous residue was extracted at 40° C. with Heptane (3×500 ml) and the combined extracts were discarded. The aqueous phase was saturated with Sodium chloride (˜160 g) and extracted with DCM (3×600 ml). The combined organics were dried over Na₂SO₄, filtered and evaporated under reduced pressure afforded 230 g of a semisolid residue, which was passed through short Silica gel column (500 g) eluted with DCM and EtOAc. After evaporation of eluent, a solid residue (˜180 g) was crystallized from EtOAc (300 ml) to give 117 g (60% yield for 4 step) of C9 as off-white crystals.

### Preparation of C10

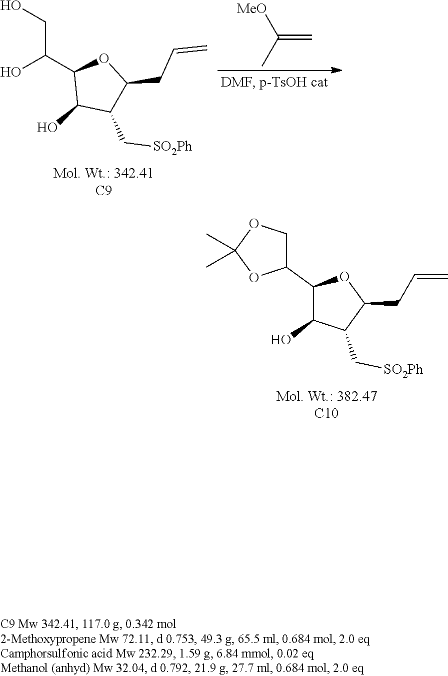

A solution of C9 (117.0 g, 0.342 mol) and 2-Methoxypropene (65.5 ml, 0.684 mol, 2.0 eq) in dry DMF (600 ml) was cooled to 0° C. under Argon and Camphorsulfonic acid was added in one portion (0.8 g, 3.42 mmol, 0.01 eq). The resulted solution was stirred for 2 h at 0° C. (UPC²/TLC control, EtOAc) and anhydrous MeOH (27.7 ml, 0.684 mol, 2.0 eq) was poured into the mixture. The reaction was stirred for additional 1 h at 0° C. and quenched by addition of 7% aqueous NaHCO₃ (2000 ml). After 10 min of stirring, the aqueous mixture was extracted with MTBE (3×300 ml) and the combined organic extracts were washed with water (150 ml) and brine (100 ml), dried over Na₂SO₄, filtered and evaporated under reduced pressure afforded 132 g of the crude ER3-32 as yellowish solid. This crude was dissolved in MTBE (500 ml) at 50° C. and Heptane (100 ml) was slowly added into the vigorously stirred solution. The precipitation was started immediately, the resulted suspension was stirred for additional 30 min and more Heptane (400 ml) was slowly added. The stirring was continued for 12 h at 25° C., the mixture was cooled to 0° C. and filtered. The cake was washed with cold Heptane/MTBE mixture (1:1, 2×100 ml) and Heptane (100 ml) and dried at 40° C. under reduced pressure afforded 111.2 g (85% yield) of C10 as white solid with 99% purity by UPC² and free from isomers.

### Preparation of C11

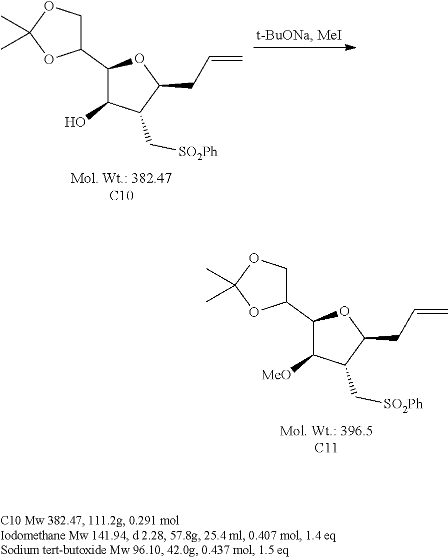

A solution of t-BuONa (42.0 g, 0.437 mol, 1.5 eq) in dry THE (400 ml) and dry DMF (50 ml) was stirred for 10 min under Argon and cooled to 0° C. A solution of C10 (111.2 g, 0.291 mol) in dry THF (200 ml) was slowly added kept the reaction temperature below 10° C. and stirring was continued for 30 min at 0° C. Methyl iodide (25.4 ml, 0.407 mol, 1.4 eq) was slowly introduced into reaction and the resulted mixture was heated for 25° C. and stirred for 10 h (UPC²/TLC control, Heptane/EtOAc 1:2). The reaction was quenched with 10% aqueous NH₄Cl (200 ml) and the most of volatiles were evaporated under reduced pressure. The residue was extracted with MTBE (3×300 ml), the combined organics were washed with water (200 ml) and brine, dried over Na₂SO₄, filtered and evaporated to give 115.0 g (near quant) of the crude C11 as yellow oil with 95% purity, Note 1.

Note 1 Optional: The Crude was purified on a silica gel column (350 g) to give 106 g of C11 with 98% purity.

### Preparation of C12

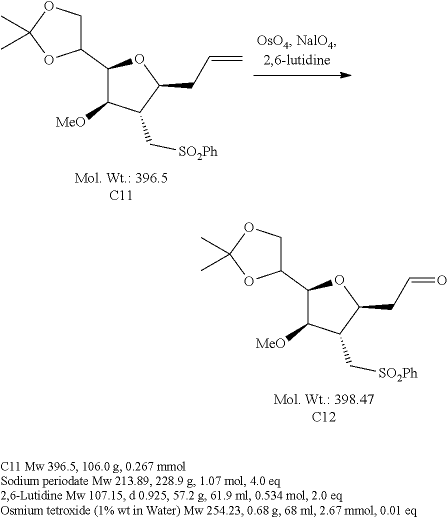

A suspension of C11 (106.0 g, 0.267 mol), 2,6-Lutidine (61.9 ml, 0.534, 2.0 eq) and NaIO₄ (228.9 g, 1.07 mol, 4.0 eq) in Dioxane (1200 ml) and Water (400 ml) was cooled to 0° C. and 1% aqueous solution of OsO₄ (68 ml, 2.67 mmol, 0.01 eq) was added dropwise. The suspension was slowly warmed to 25° C., allowed to stir for 6 h (UPC²/TLC monitoring, Heptane/EtOAc 1:2) and filtered through Celite (100 g). The cake was washed with EtOAc (3×300 ml) under UPC²/TLC control), the combined filtrates were mixed with 20% aqueous Na₂SO₃ (810 ml, ˜5.0 eq) and NaHCO₃ (44.9 g, 0.534 mol, 2.0 eq) and stirred for 30 min. The most of organics were evaporated under reduced pressure at 40° C., and NaCl (50 g) and EtOAc (800 ml) were added into aqueous residue. After 20 min of the vigorous stirring the layers were separated and the aqueous one was extracted with EtOAc (3×300 ml). The combined organic one was washed with brine (150 ml), dried over Na₂SO₄, filtered and evaporated under reduced pressure to get 105 g of the crude product with 93% purity. The crude was purified from the polar traces (mostly inorganics) by passing through a short Silica gel column (200 g) eluted with MTBE. After evaporation and drying under reduced pressure 91.3 g (86% yield) of the desired aldehyde C12 was obtained as yellowish oil with 95% purity by UPC².

### UPC² Sample Preparation (for IPC):

2 ml of RM was stirred with 20% aqueous Na₂SO₃ (1 ml) and EtOAc (4 ml), filtrated and organic phase was separated and evaporated under reduced pressure.

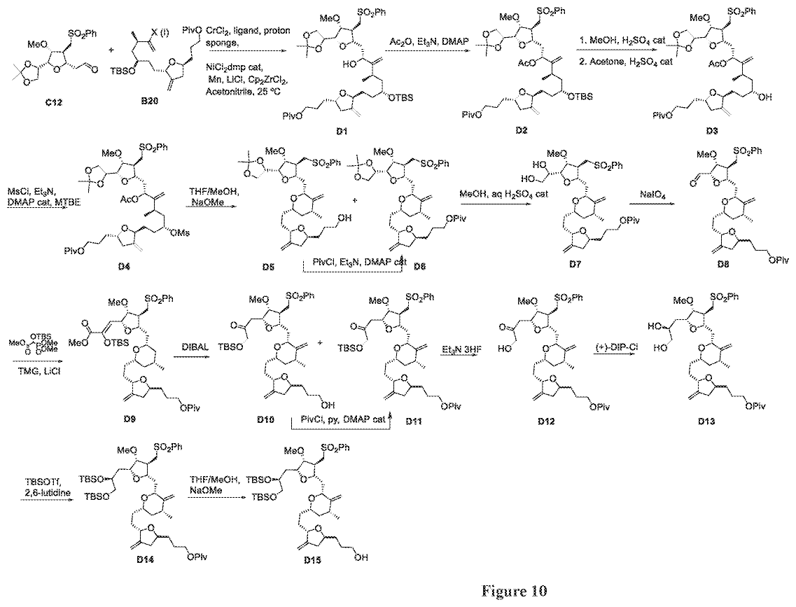

### Example 9: Preparation of D15 from C12 and B20 (See Also FIG. 10)

### Preparation of D1

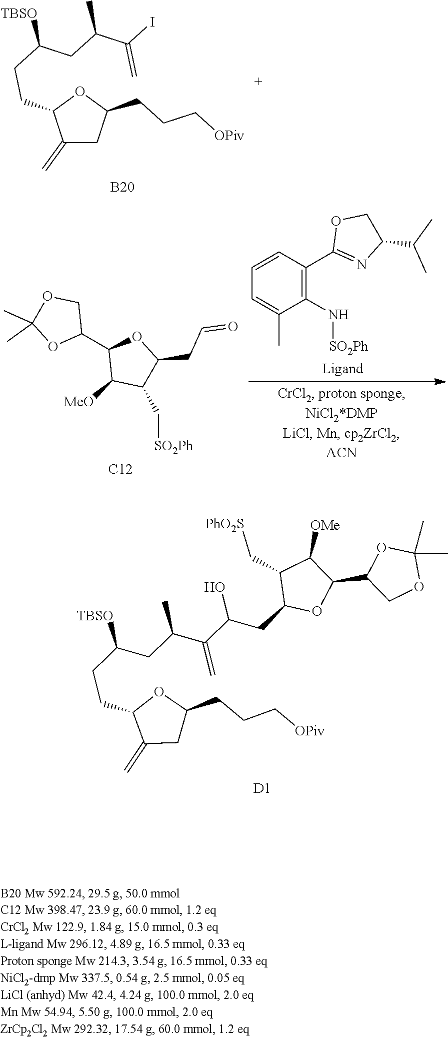

A three-necked flask was vacuum-dried and flushed with Argon by three vacuum-Argon cycles. CrCl₂, L-ligand and proton sponge were loaded into the flask and flushed with Argon. Then anhydrous oxygen-free AcN (150 ml) was introduced into the reaction vessel and the resulted green mixture was stirred for 1 h at 25° C. until complete dissolution.

In the separated three-necked flask, prepared as described above, B20, C12, Note 1, LiCl, Mn, NiCl₂-dmp and ZrCp₂Cl₂ were mixed under Argon and then anhydrous oxygen-free AcN (450 ml) was added in one portion under positive Argon pressure. The resulted suspension was flushed with Argon and stirred for 30 min. The prepared Cr-ligand solution was slowly loaded into the reaction flask by cannula with vigorous stirring. The heterogeneous reaction was stirred (sticky gum mass may beformed after 24 h of stirring) at 25° C. for 48 h (UPC²/TLC control, Heptane/EtOAc 1:1). The reaction was quenched by addition a mixture Fluorisil/Silica gel (1:1, 60 g), stirred for 15 min, diluted with MTBE (1.0 L) and stirred for additional 2 h. The suspension was filtered through Celite, the cake was washed with a mixture MTBE/EtOAc 1:1 (2×100 ml), and the combined filtrates were concentrated under reduced pressure. The residue (˜50 g) was purified on a short Silica gel column (300 g) eluted with gradient Heptane/EtOAc 15:1 to Hept/EtOAc 2:1. All fraction contained the desired product were combined and evaporated under reduced pressure to get 38.7 g (85% purity by UPC²) of D1 as yellow sticky mass.

Note 1Before reaction, a mixture of B20 and C12 was co-evaporated with anhydrous AcN (150 ml) to remove the traces of moisture.

Note 2 A sticky gum mass may be formed after 24 h of stirring.

### Preparation of D2

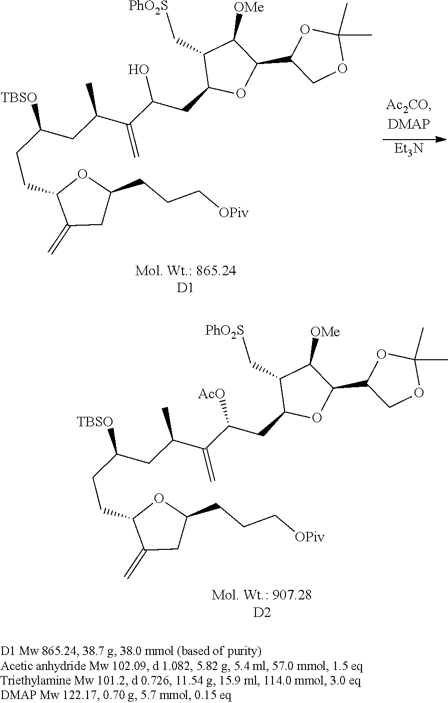

A solution of D1, Et₃N and DMAP in dry DCM (200 ml) was cooled to 0° C. under Argon and Ac₂O, Note 1, was slowly added. The reaction was stirred for 2 h at 10° C. (TLC control, Hept/EtOAc 1:1), quenched with Water (80 ml) and stirring was continued for 30 min at 20° C. The phases were separated, the aqueous one was extracted with DCM (100 ml) and the combined organics were washed with 10% aqueous Citric acid (100 ml) and 10% aqueous NaHCO₃ (100 ml), dried over Na₂SO₄, filtered and evaporated under reduced pressure afforded ˜34.5 g (near quant) of the crude D2 as yellowish oil which was used without additional purification.

Note 1Benzoyl chloride (1.1 Eq) may be used instead of acetic anhydride to give the more stable benzoate.

### Preparation of D3

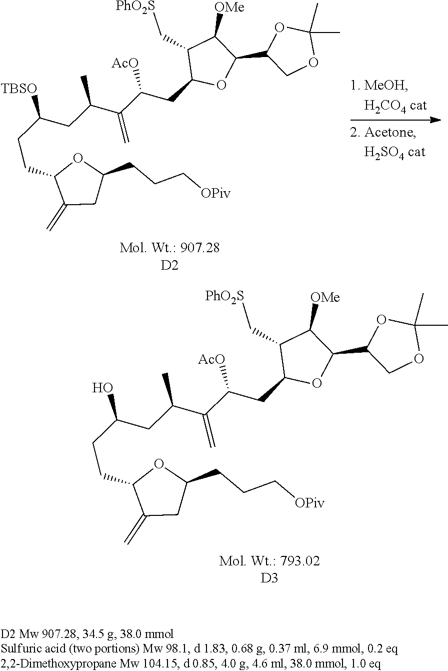

A solution of D2 in absolute MeOH (200 ml) was cooled to 0° C. under Argon and H₂SO₄ (˜0.1 eq, 0.19 ml) was added in one portion. The reaction was stirred for 1 h at 0° C., then for 6 h at 25° C. until complete deprotection of Silyl group (UPC²/TLC control; Heptane/EtOAc 1:2) The product of the partial deprotection of isopropylidene group (triol, ˜20-30%) was also detected. The reaction was quenched with saturated aqueous NaHCO₃ (50 ml) and the most of MeOH was evaporated under reduced pressure. The aqueous residue was extracted with EtOAc (2×200 ml), the combined organics were washed with water (50 ml) and brine, dried over Na₂SO₄, filtered and evaporated under reduced pressure afforded 33.7 g of the yellow oil. A residue and 2,2-Dimethoxypronane were dissolved in dry Acetone (200 ml)under Argon, cooled to 0° C. and treated with H₂SO₄ (˜01 eq, 0.18 ml). The reaction was warmed to 25° C. and stirred for additional 3 h until completeness. Then, the saturated aqueous NaHCO₃ (50 ml) was added and the most of organic volatiles were evaporated under reduced pressure. The aqueous residue was extracted with EtOAc (2×200 ml), the combined organics were washed with brine, dried over Na₂SO₄, filtered and evaporated under reduced pressure at 40° C. until constant weight to give 30.2 g (near quant) of D3 as yellow oil.

### Preparation of D4

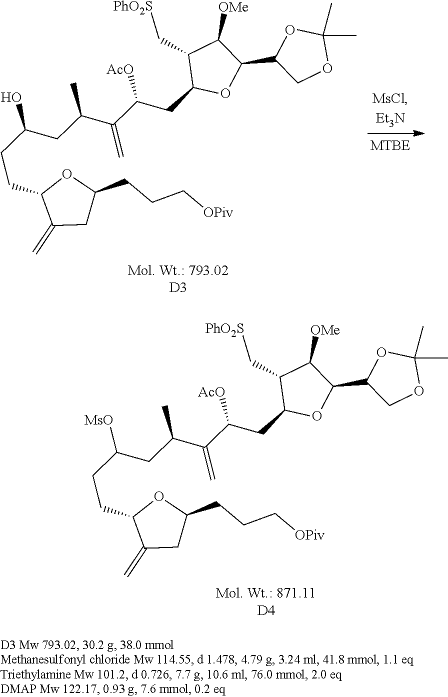

A solution of D3, Et₃N and DMAP in dry MTBE (250 ml) was cooled to 0° C. under Argon and MsCl was slowly added kept the temperature inside below 10° C. The reaction was stirred for 1 h at 0° C. (UPC²/TLC control; DCM/EtOAc 5:2) and quenched with Water (70 ml). After 20 min of stirring the phases were separated, the aqueous one was extracted with MTBE (80 ml) and the combined organics were washed with 10% aqueous Citric acid (70 ml) and 10% aqueous NaHCO₃ (70 ml), dried over Na₂SO₄, filtered and evaporated under reduced pressure afforded 33.0 g (near quant) of the crude D4 as yellow oil which was used without additional purification.

### Preparation of D5 and D6

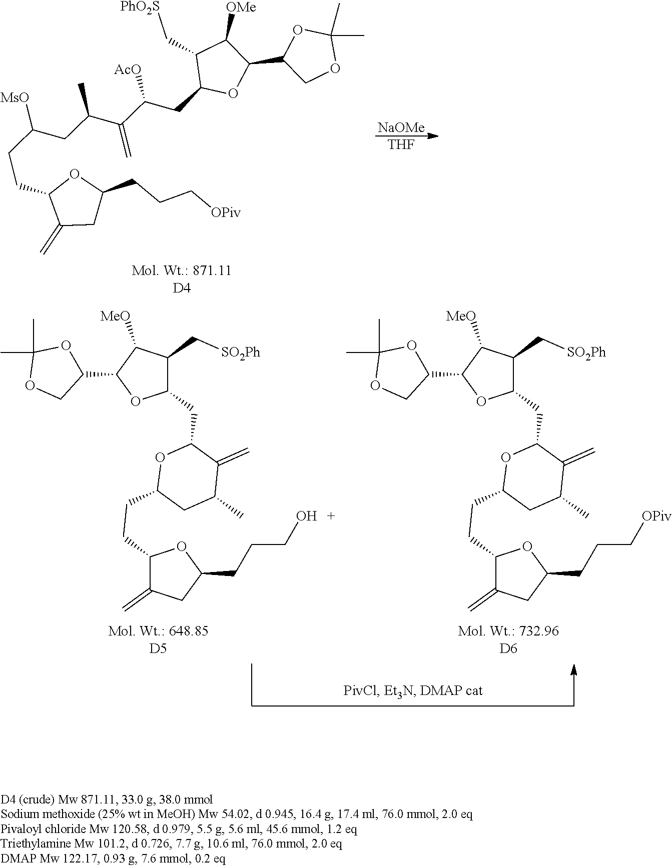

A solution of crude D4 in dry THE (600 ml) was cooled to 0° C. under Argon and a solution of NaOMe in Methanol was slowly added kept the temperature inside below 10° C. Then, the reaction was warmed to 25° C., stirred for 6 h (UPC²/TLC control; Hept/EtOAc 1:2), Note 1, and quenched with 20% aqueous NH₄Cl (100 ml). The most of THF was evaporated under reduced pressure, the aqueous residue was extracted with MTBE (2×200 ml) and the combined extracts were washed with Water (100 ml) and brine, dried over Na₂SO₄, filtered and evaporated under reduced pressure.

The yellow oily residue (27.3 g, with D5/D6 ratio 9:1), Et₃N and DMAP were dissolved in dry DCM (150 ml), the resulted solution was cooled to 0° C. under Argon and Pivaloyl chloride was slowly added. The reaction was stirred for 1 h at 25° C. (UPC²/TLC control; Hept/EtOAc 1:2) and quenched by addition of MeOH (2 ml). All volatiles were evaporated under reduced pressure and the residue was portioned between MTBE (300 ml) and Water (100 ml). The phases were separated, the aqueous one was extracted with MTBE (50 ml) and the combined organics were washed with 10% aqueous Citric acid (70 ml) and 9% aqueous NaHCO₃ (70 ml), dried over Na₂SO₄, filtered and evaporated under reduced pressure afforded 28 g of the crude D6 as yellowish oil.

Note 2. The crude was purified.

Note 3, on a Silica gel column (250 g) eluted with:
Hept/EtOAc 8:1 to 6:1—Upper spots
Hept/EtOAc 6:1 to 4:1—Main product (19.5 g);
Hept/EtOAc 4:1 to 2:1—Isomers mostly (2.3 g).
19.5 g (70% for 5 steps) of D6 contaminated with 4.9% of isomer was obtained as colorless oil.

Note 1 If intermediates are still presence, more NaOMe (6.1 ml, 0.7 eq) should be added and stirring has to be continued for additional 4 h with UPC²/TLC monitoring.

Note 2 Crude may be used without additional purification.

Note 3 Crude may be purified on a preparative HPLC (Luna CN, direct phase, Heptane/MTBE) afforded ˜70% of the isomeric pure D6.

### Preparation of D8

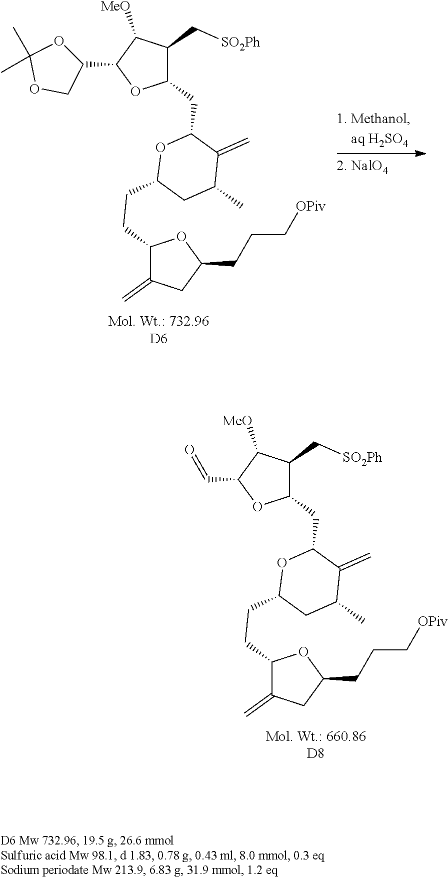

A solution of D6 in MeOH (100 ml) was treated with a solution of H₂SO₄ in Water (30 ml) and the resulted mixture was stirred at 25° C. for 6 h (UPC²/TLC control; Hept/EtOAc 1:2) and quenched by addition of solid NaHCO₃ (5 g). Slow gas evolution was observed. After 1 h of the progressive stirring the mixture was diluted with MeOH (100 ml) and water (60 ml) and the resulted mixture was cooled to 0° C. NaIO₄ (6.83 g) was added by portions during 1 h kept the reaction temperature below 5° C. The resulting suspension was stirred for 12 h at 0° C., filtered through Celite545 and the cake was rinsed with MeOH (2×50 ml). Then, the most of organic volatiles were evaporated at 40° C. under reduced pressure, the cake was washed with EtOAc (3×100 ml) under TLC control (DCM/EtOAc 5:2). The combined filtrates were mixed with aqueous residue, stirred for 30 min and separated. The organic phase was washed with brine, dried over Na₂SO₄, filtered and evaporated under reduced pressure. The residue was co-evaporated with Toluene (50 ml) afforded 17.4 g of the crude D8 as yellowish oil.

### Preparation of D9

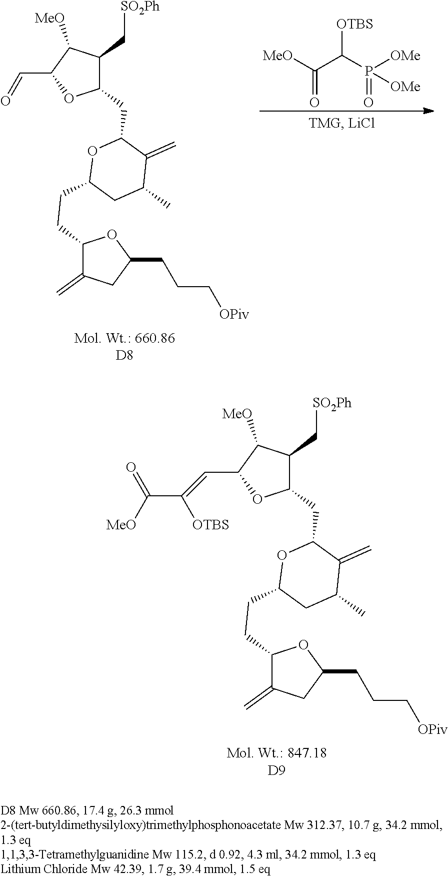

D8 and Phosphonate were dissolved in Toluene (100 ml) and evaporated at 40° C. under reduced pressure to remove the traces of water. The residue was dissolved in dry THF (150 ml) under Argon, a solution was cooled down to 0° C. and LiCl was added in one portion. A suspension was warmed to 25° C., stirred for 30 min, and then cooled back to −5° C. Tetramethylguanidine (288 g, 2.5 mol) was slowly (Precipitate formed) added kept the reaction temperature below 5° C. and the reaction was stirred at 0° C. for 2 h (UPC²/TLC control EtOAc/Heptane 2:1, Note 1) and quenched by addition of 20% aqueous NH₄Cl (30 ml). The most of THE was evaporated under reduced pressure and the aqueous residue was mixed with MTBE (150 ml). The layers were separated and the aqueous one was extracted with MTBE (2×30 ml). The combined organics were washed with water (50 ml) and brine, dried over Na₂SO₄, filtered and evaporated under reduced pressure. The residue was dissolved in Toluene (100 ml) and passed through a short Silica gel column (100 g). The column was sequentially eluted with n-Heptane/EtOAc 10:1 and then 3:1. All fractions contained the desired product were combined and evaporated under reduced pressure to give 20.1 g (90% yield) of D9 as yellowish oil.

Note 1 Sample preparation: 0.2 ml of the reaction mixture was added into the mixture of 0.5 ml 10% aqueous Citric Acid and 0.5 ml MTBE and shake for 1 min.

If D8 still present—add Tetramethylguanidine (0.1 eq).

### Preparation of D11

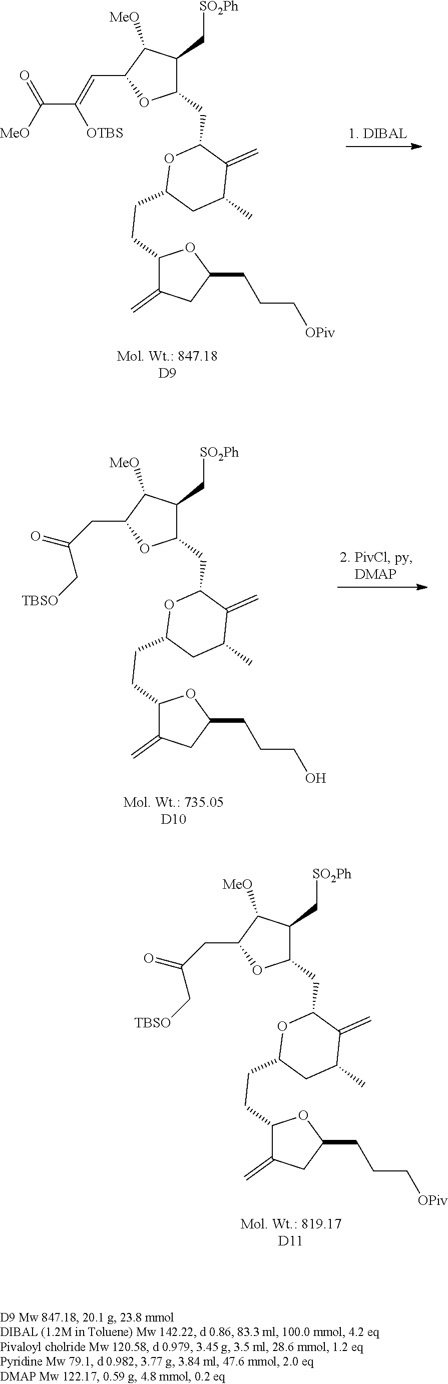

A solution of D9 in dry Toluene (120 ml) was cooled to −25° C. and Diisobutylaluminium hydride was slowly added kept the temperature inside below −20° C. The mixture was stirred for 30 min at −25° C. (UPC²/TLC control, Hept/EtOAc 1:2) and quenched by slow addition of 10% aqueous citric acid (230 ml, ˜5 eq) when the temperature inside kept below 10° C. The resulted mixture was stirred for 8 h at 10° C. until almost complete dissolution of solids and the layers were separated. The aqueous one was extracted with MTBE (3×100 ml) and the combined organics was sequentially washed with 9% aqueous NaHCO₃ (100 ml) and brine, dried over Na₂SO₄, filtered and evaporated under reduced pressure afforded ˜18 g of the crude D10.

A crude was dissolved in dry DCM (150 ml), Pyridine and DMAP were poured into reaction vessel and the resulted solution was cooled to 0° C. under Argon. Pivaloyl chloride was slowly added, a reaction was stirred for 2 h at 0° C. (UPC²/TLC control, Hept/EtOAc 1:2) and quenched by addition of Water (50 ml). The mixture was warmed to 25° C., stirred for 30 min, and the phases were separated. The aqueous one was extracted with DCM (100 ml), and the combined organics were sequentially washed with 10% aqueous citric acid (100 ml), 9% aqueous NaHCO₃ (100 ml) and brine, dried over Na₂SO₄, filtered and evaporated under reduced pressure afforded 20.2 g of crude D11 which was used in the next step.

### Preparation of D12

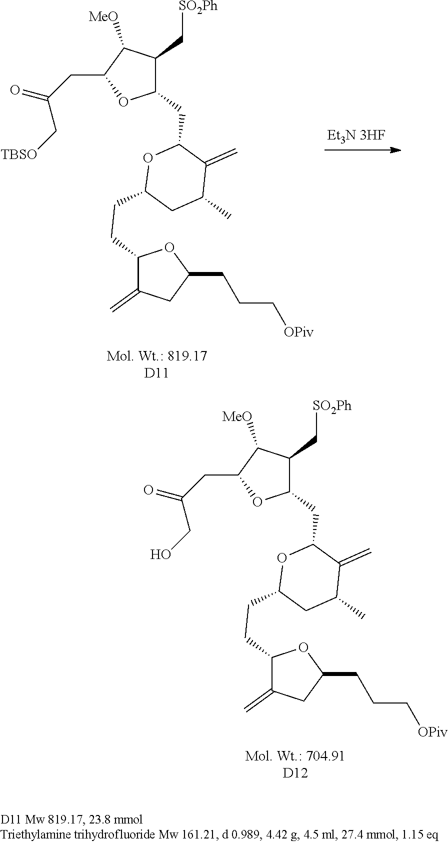

A solution of crude D11 (20.2 g) from earlier step in dry THF (200 ml) was cooled down to 0° C. under Argon and Et₃N-3HF was slowly added during 1 h period kept the reaction temperature below 5° C. The mixture was stirred for 18 h at 0° C. (UPC²/TLC control Heptane/EtOAc 1:2) and quenched by addition 9% aqueous NaHCO₃ (110 ml, ˜5 eq). The most of THF were evaporated under reduced pressure at 30° C. and the aqueous residue was extracted with MTBE (2×150 ml). The combined organics were washed with brine, dried over Na₂SO₄, filtered and evaporated under reduced pressure for 12 h at 30° C. to give 16.7 g (near quant) of crude D12 which was used without any purification.

### Preparation of D13

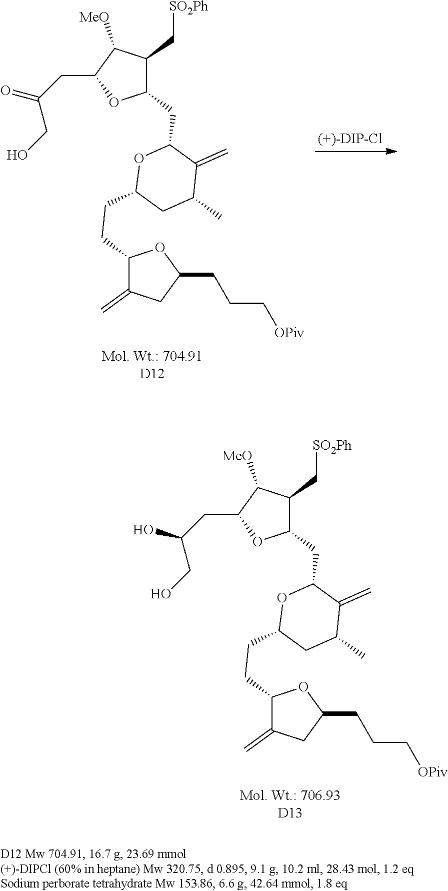

A solution of crude D12 (16.7 g from previous step) in dry THF (150 ml) was cooled to −5° C. under Argon and (+)-DIPCl (60% solution in heptane) was slowly added kept the reaction temperature below 2° C. Then, the reaction was stirred for 24 h at 0° C. (UPC²/TLC control Heptane/EtOAc 1:2), warmed to 20° C. and quenched by slow (Gas evolution) addition of 5% aqueous Sodium bicarbonate (120 ml, ˜3.0 eq). After 30 min of stirring, the mixture was cooled to 0° C. and NaBO₃×4H₂O was loaded in several portions (slight exothermic reaction). The resulted mixture was stirred for 1 h at 0° C., slowly warmed to 25° C. and stirred for 14 h. The most of volatiles were evaporated under reduced pressure. The aqueous suspension was mixed with EtOAc (100 ml), stirred for 15 min and filtered. The cake was washed with EtOAc (2×50 ml), the filtrates were combined and the phases were separated. The aqueous one was extracted with EtOAc (2×50 ml), the combined organics were washed with Water (50 ml) and brine, dried over Na₂SO₄, filtered and evaporated under reduced pressure. The oily residue (˜25 g) was triturated with Heptane (60 ml) for 1 h at 25° C. and then allowed to stand at −18° C. for 12 h. The upper layer was decanted, the residue was dissolved in Toluene (60 ml) and purified on a Silica gel column (120 g) sequentially eluted with gradient n-Heptane/EtOAc from 10:1 to 1:3. All fractions contained the desired product were combined and evaporated under reduced pressure to give 13.9 g (83% yield for 4 steps) of D13 as yellowish oil.

### Preparation of D15

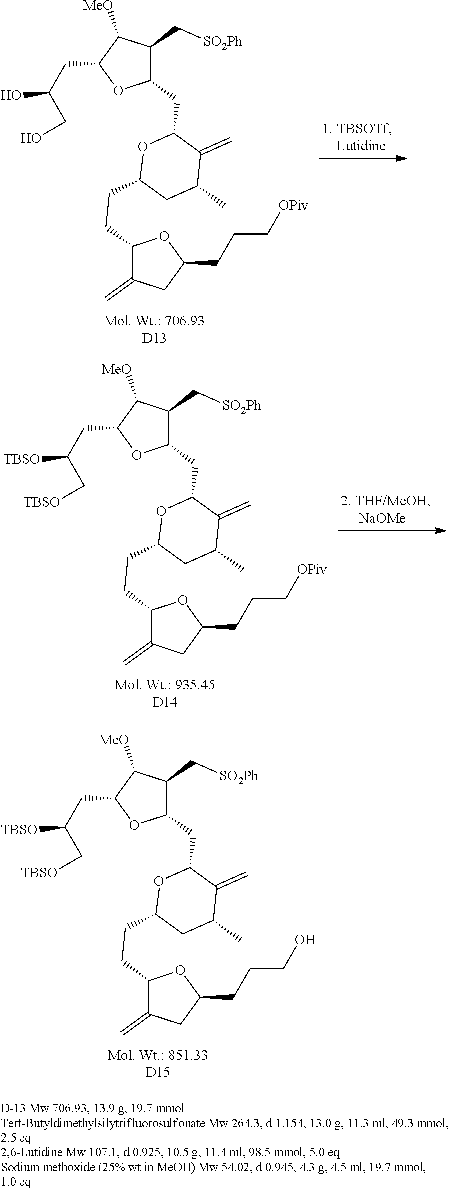

A solution of D13 and 2,6-Lutidine in dry MTBE (200 ml) was cooled to 0° C. under Argon and TBSOTf was slowly added kept the reaction temperature below 15° C. The reaction was stirred for 3 h at 20° C. (UPC²/TLC control; Hept/EtOAc 1:1), quenched with Water (100 ml) and stirred for additional 1 h. The phases were separated, the aqueous one was extracted with MTBE (100 ml) and the combined organics were washed with 10% aqueous Citric acid (200 ml), 9% aqueous NaHCO₃ (100 ml) and brine, dried over Na₂SO₄, filtered and evaporated under reduced pressure afforded 18.5 g (near quant) of the crude D14 which was used without purification A solution of crude D14 in dry THE (150 ml) was cooled to 0° C. under Argon and methanolic solution of NaOMe was slowly added. A reaction was warmed to 25° C., stirred for 4 h (UPC²/TLC control Heptane/EtOAc 1:2) and quenched with 20% aqueous NH₄Cl (10 ml). The most of volatiles were evaporated under reduced pressure, the residue was diluted with water (50 ml) and extracted with MTBE (2×150 ml). The combined organics were washed with 9% aqueous NaHCO₃ (50 ml) and brine, dried over Na₂SO₄, filtered through Silica gel pad (20 g), which was eluted with MTBE (100 ml) and then the combined filtrates were evaporated under reduced pressure afforded 16.9 g (quant) of the D15 as yellowish oil.

### Preparation of D6 (See Also FIG. 11)[Ref for the Procedure 36-39]

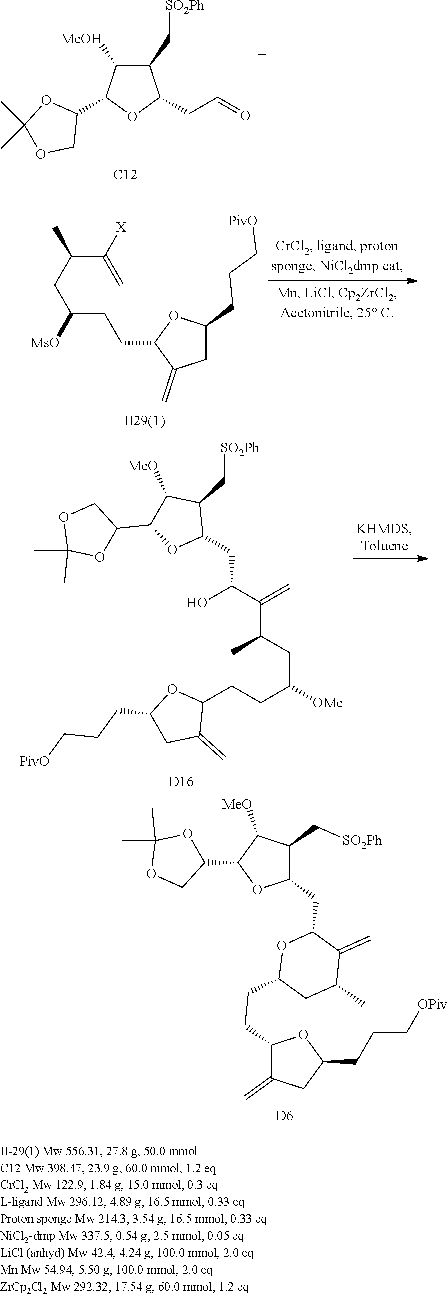

A three-necked flask was vacuum-dried and flushed with Argon by three vacuum-Argon cycles. CrCl₂, L-ligand and proton sponge were loaded into the flask and flushed with Argon. Then anhydrous oxygen-free AcN (150 ml) was introduced into the reaction vessel and the resulted green mixture was stirred for 1 h at 25° C. until complete dissolution.

In the separated three-necked flask, prepared as described above, B20, C12, Note 1, LiCl, Mn, NiCl₂-dmp and ZrCp₂Cl₂ were mixed under Argon and then anhydrous oxygen-free AcN (450 ml) was added in one portion under positive Argon pressure. The resulted suspension was flushed with Argon and stirred for 30 min. The prepared Cr-ligand solution was slowly loaded into the reaction flask by cannula with vigorous stirring. The heterogeneous reaction was stirred (sticky gum mass may beformed after 24 h of stirring) at 25° C. for 48 h (UPC²/TLC control, Heptane/EtOAc 1:1). The reaction was quenched by addition a mixture Fluorisil/Silica gel (1:1, 60 g), stirred for 15 min, diluted with MTBE (1.0 L) and stirred for additional 2 h. The suspension was filtered through Celite, the cake was washed with a mixture MTBE/EtOAc 1:1 (2×100 ml), and the combined filtrates were concentrated under reduced pressure.

The residue (˜50 g) was dissolved in dry THE (1 L) under Argon and a solution was cooled down to −25° C. 0.7 M KHDMS in Toluene (totally ˜2 eq according to HPLC determined conversion) was slowly added kept the reaction temperature below −15° C. Upon complete reaction, the mixture was transferred to 10% aq NH₄Cl (300 ml) at 0° C., stirred for 15 min and the most of THF was evaporated under reduced pressure. The aqueous residue was extracted with MTBE (2×200 ml), the combined organics were washed with brine, dried over Na₂SO₄, filtered and evaporated. The crude (46 g) was purified on a short Silica gel column (400 g) eluted with gradient Heptane/EtOAc 15:1 to Hept/EtOAc 4:1. All fraction contained the desired product were combined and evaporated under reduced pressure to get 25.7 g (70% from C-12(ER3-12)) of D6 as yellow sticky mass.

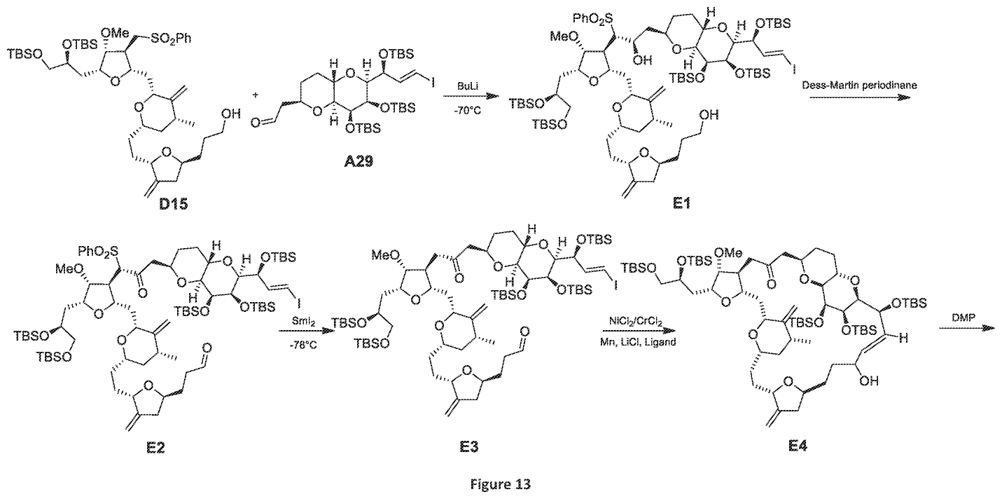

### Preparation of D7 with B28 (See Also FIG. 12) [Ref for the Procedure 36-39]

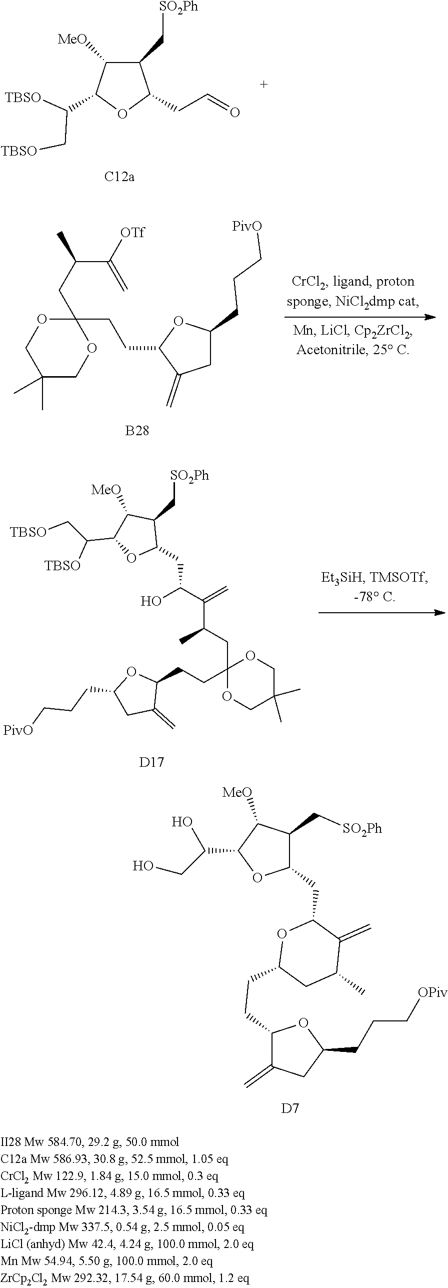

A three-necked flask was vacuum-dried and flushed with Argon by three vacuum-Argon cycles. CrCl₂, L-ligand and proton sponge were loaded into the flask and flushed with Argon. Then anhydrous oxygen-free THF (150 ml) was introduced into the reaction vessel and the resulted green mixture was stirred for 1 h at 25° C. until complete dissolution.

In the separated three-necked flask, prepared as described above, C12a, B28, LiCl, Mn, NiCl₂-dmp and ZrCp₂Cl₂ were mixed under Argon and then anhydrous oxygen-freeTHF (450 ml) was added in one portion under positive Argon pressure. The resulted suspension was flushed with Argon and stirred for 30 min. The prepared Cr-ligand solution was slowly loaded into the reaction flask by cannula with vigorous stirring. The heterogeneous reaction was stirred at 25° C. for 48 h (UPC²/TLC control, Heptane/EtOAc 2:1). The reaction was quenched by addition of Fluorisil (70 g), stirred for 15 min, diluted with MTBE (1.0 L) and stirred for additional 2 h. The suspension was filtered through Celite, the cake was washed with a mixture MTBE/EtOAc 1:1 (2×100 ml), and the combined filtrates were concentrated under reduced pressure. The residue (˜60 g) was dissolved in dry CH₂Cl₂ (1 L) and Et₃SiH (10 eq) under argon and the resulted mixture was cooled to −78° C. TMSOTf (5 eq) was slowly added at the rate that kept the temperature inside below −60° C. and the reaction was stirred for 1 h at −70° C. (UPC²/TLC control, Heptane/EtOAc 2:1 and Heptane/EtOAc 1:2) and quenched by slow addition of Et₃N (70 ml). The mixture was warmed to 0° C. and poured into 9% aq NAHCO₃ (1 L). After 30 min of stirring, the phases were separated, the organic one was washed with brine, dried over Na₂SO₄, filtered and evaporated under reduced pressure. The crude was purified on a short Silica gel column (400 g) eluted with gradient Heptane/EtOAc 7:1 to EtOAc. All fraction contained the desired product were combined and evaporated under reduced pressure to get 26.0 g (75% from ER2-13) of D7 as yellow sticky mass.

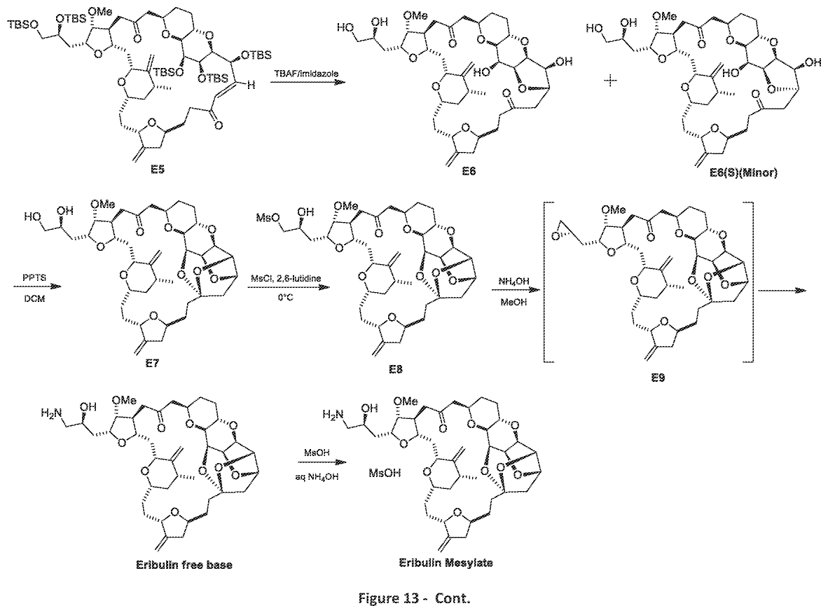

### Example 10: Preparation of Eribulin from D15 and A29 (See Also FIG. 13)

### Preparation of E1

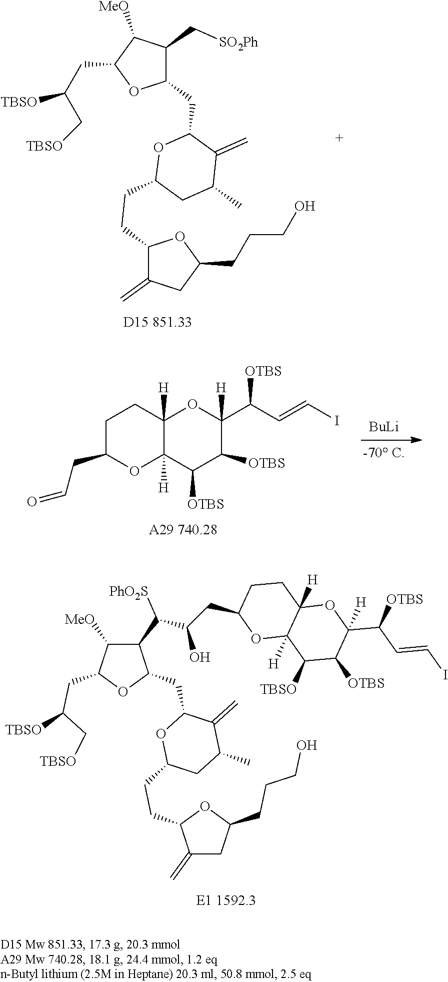

A solution of D15 in dry THF (170 ml) was cooled to −15° C. under Argon and BuLi was slowly added kept the temperature inside below 3° C. (Firstly, n-BuLi was added until occurring the stable yellow color of the solution. From this point, 1.0 eq more of n-BuLi was added. Totally was 2.5 eq). The mixture was stirred for 30 min at 0° C., cooled to ˜78° C. and a solution of A29 in dry THE (70 ml) was slowly introduced into reactor kept the temperature inside below −60° C. The reaction was stirred for 2 h in the range −60-70° C., quenched by slow (exothermic) addition of 20% aqueous NH₄Cl (50 ml), warmed to 20° C., stirred for additional 15 min and the most of organic volatiles were evaporated under reduced pressure. The aqueous residue was extracted with MTBE (2×200 ml), the combined extracts were washed with brine, dried over Na₂SO₄, filtered and evaporated under reduced pressure to give ˜35 g of the foam residue. The residue was dissolved in Heptane (200 ml) and passed through Silica gel column (250 g) eluted with gradient Heptane/EtOAc from 10:1 to 1:1 (8:1—unreacted A29; 4:1 to 2:1—main product; 1:1—unreacted D15). 24 g (74% yield) of the desired product E1 was obtained.

### Preparation of E2

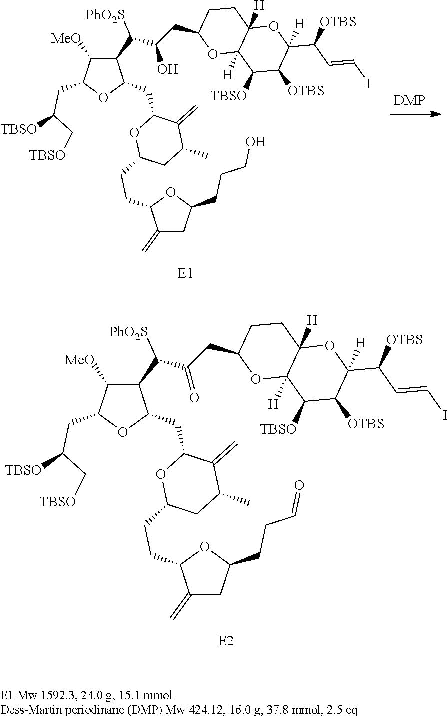

A solution of E1 in dry DCM (250 ml) was cooled to 0° C. under Argon and DMP was added in two portions. The reaction was stirred at 20° C. for 2 h (TLC control, Hept/EtOAc 2:1; intermediate still present) and more DMP (7.6 g, 0.5 eq) was added. After additional 1 h of stirring (TLC monitoring), the reaction was carefully (gas evolution!) quenched by slow addition of 10% aqueous NaHCO₃ (150 ml) and 10% aqueous Na₂SO₃ (150 ml) and the resulted mixture was stirred for 1 h until complete dissolution of solids. The phases were separated, the aqueous one was extracted with DCM (100 ml) and the combined organics were washed with mixture 10% aqueous NaHCO₃/10% aqueous Na₂SO₃ 1:1 (200 ml) and Water (200 ml), dried over Na₂SO₄, filtered and evaporated under reduced pressure to give ˜26 g of the foam residue. The residue was dissolved in Heptane (100 ml) and passed through Silica gel column (100 g) eluted with gradient Heptane to Heptane/EtOAc 4:1. 22.0 g (92% yield) of the desired E2 was obtained.

### Preparation of E3

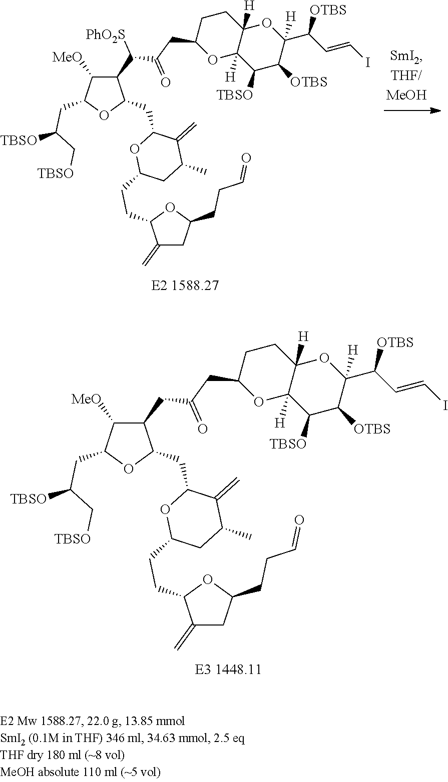

A solution of SmI₂ was cooled to −78° C. under Argon and a solution of E2 in THF/MeOH was slowly added kept the reaction temperature below −60° C. (˜1 h period). At this point, the stable green color may be formed. If not, more SmI₂ (up to additional 2.5 eq) should be added until obtaining the desired coloration. The reaction was stirred for 30 min at −70° C. (TLC control, Hept/EtOAc 2:1) and quenched by slow addition of the aqueous solution K₂CO₃/Na₂ tartrate/Water 1:1:10 (350 ml) under vigorous stirring at the rate which kept the temperature inside below −50° C. Then, 250 ml of MTBE was added in one portion, the reaction was allowed to warm until 20° C. and poured into the mixture MTBE (650 ml) with K₂CO₃/Na₂ tartrate/Water 1:1:10 (650 ml). The resulted mixture was stirred for 15 min, diluted with Heptane (250 ml) and the phases were separated. The aqueous one was extracted with Heptane (2×250 ml) and the combined organics were washed with water (500 ml) and brine, dried over Na₂SO₄, filtered and evaporated under reduced pressure to give ˜20 g of the foam residue. The residue was dissolved in Heptane (100 ml) and passed through Silica gel column (200 g) eluted with gradient Heptane to Heptane/EtOAc 5:1. 17.1 g (85% yield) of the desired product was obtained as foam.

### Preparation of E4

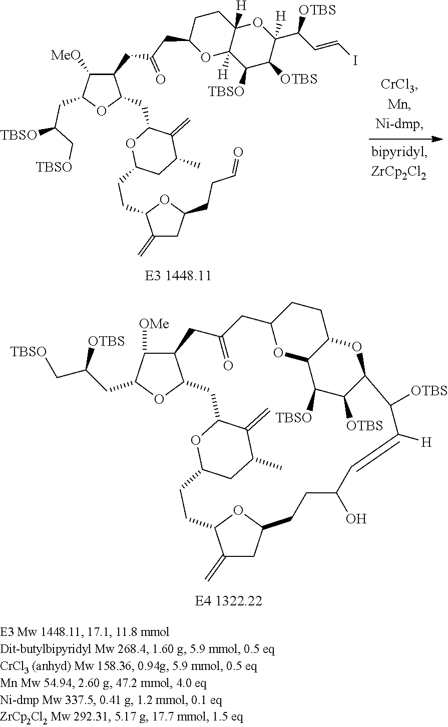

Bipyridyl, CrCl₃, Mn and ZrCp₂Cl₂ were weighed under Argon and loaded into vacuum-dried Argon-flushed flask. Anhydrous THF (750 ml) was added under positive pressure of Argon, the resulted mixture was degassed by two vacuum-Argon cycles and stirred for 2 h. Ni-dmp was added in one portion and stirring was continued for 20 min. A solution of E3 in anhydrous THE (250 ml) was loaded by syringe under Argon, the reaction was stirred at 20° C. for 4 h (UPC²/TLC control; Hept/EtOAc 4:1 with two runs) and quenched with Fluorisil (20 g). The most of THF was evaporated under reduced pressure, the residue was taken up with MTBE (300 ml), stirred for 2 h and filtered through Celite. The cake was washed with MTBE (2×100 ml) and the combined filtrates were concentrated to a half under reduced pressure. The resulted solution was washed with cold 0.5M aqueous HCl (200 ml), 10% aqueous NaHCO₃ (100 ml), water (100 ml) and brine, dried over Na₂SO₄, filtered and evaporated under reduced pressure to give 11.7 g (75% yield) of the crude E4.

### Preparation of E5

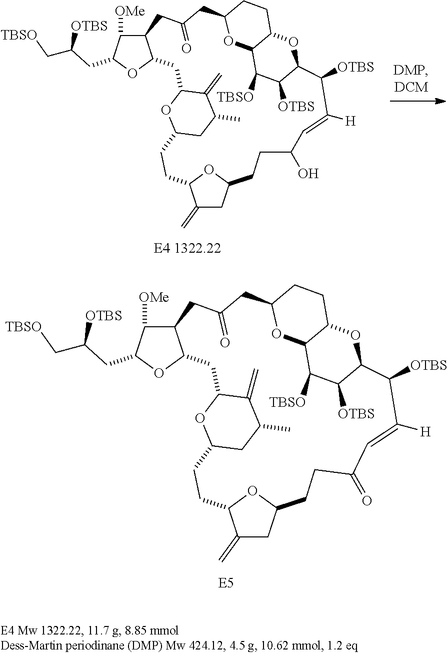

A solution of E4 in dry DCM (150 ml) was cooled to 0° C. under Argon and DMP was added in one portion. The reaction was stirred for 1 h at 20° C. (TLC control, Hept/EtOAc 2:1) and more DMP (1.5 g, 0.4 eq) was added. After additional 1 h of stirring (TLC monitoring), the reaction was carefully (gas evolution!) quenched by slow addition of 10% aqueous NaHCO₃ (50 ml) and 10% aqueous Na₂SO₃ (50 ml) and the resulted mixture was stirred for 1 h until complete dissolution of solids. DCM was evaporated under reduced pressure and the aqueous residue was extracted with MTBE (2×100 ml) and the combined organics were washed with mixture 10% aqueous NaHCO₃/10% aqueous Na₂SO₃ 1:1 (60 ml), Water (50 ml) and brine, dried over Na₂SO₄, filtered and evaporated under reduced pressure to give ˜13 g of the foam residue. The residue was dissolved in Heptane (100 ml) and passed through Silica gel column (100 g) eluted with gradient Heptane to Heptane/EtOAc 8:1. 10.6 g (90% yield) of the desired E5 was obtained as white powder.

7.5 g of this E5 were separated on a preparative UPC² afforded 4.5 g of the chemical and isomeric pure E5.

### Preparation of E6

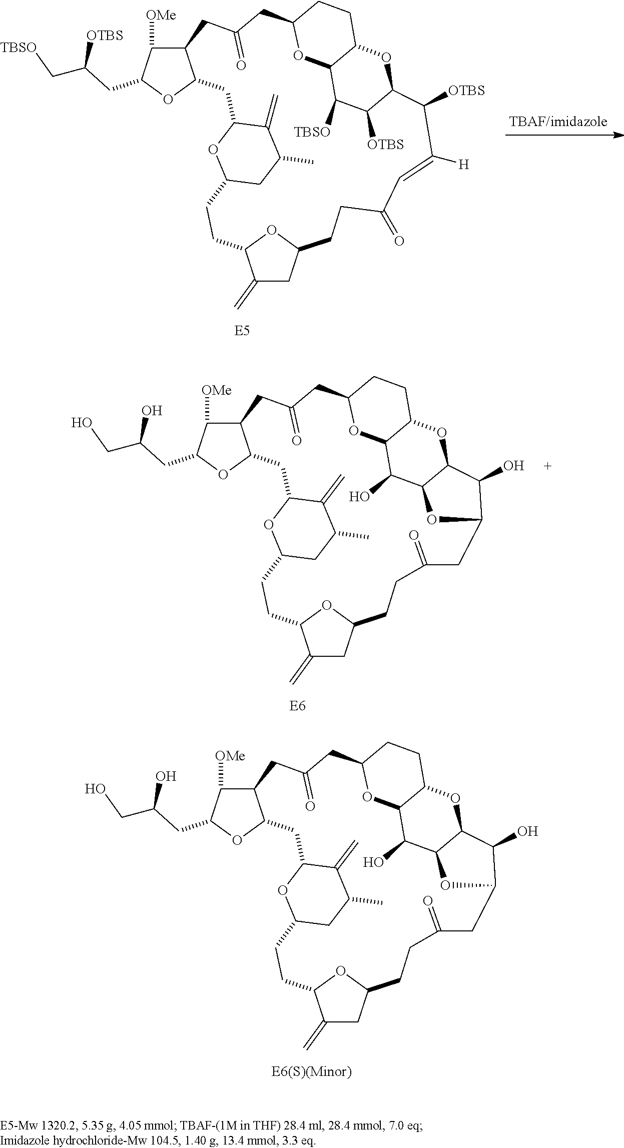

A solution of Imidazole hydrochloride in TBAF (as 1M in THF) was added into a solution of E5 in dry THF (180 ml) under Argon and the reaction was stirred at 25° C. with TLC/UPC² monitoring. After 40 h (˜2.5% of trisilyl and ˜0.5% of monosilyl was still present) the reaction was quenched with Water (85 ml) and diluted with Toluene (85 ml). After 15 min of stirring, the phases were separated, the aqueous was extracted with the mixture Toluene/THF 1:1 (120 ml) and the combined organics were evaporated under reduced pressure at 35-40° C. (less than 5% of pentaol remained) and the residue was co-evaporated under reduced pressure with Acetonitrile (60 ml) and then with DCM (60 ml) afforded ˜4.0 g of the crude E6 as yellowish foam.

### Preparation of E7

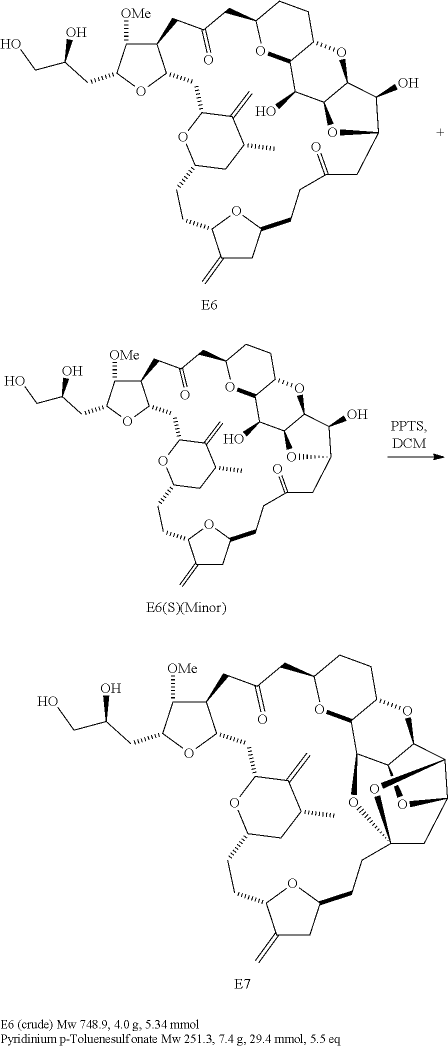

A solution of the crude E6 in dry DCM (70 ml) was cooled to 0° C. under Argon and a solution of PPTS in dry DCM (20 ml) was added in one portion. The reaction was stirred for 6 h at 20° C. (UPC²/TLC control; EtOAc) and loaded onto Silica gel column (100 g) pre-equilibrated with MTBE. The reaction flask was rinsed with DCM (2×15 ml) and rinses were also loaded onto a column. The column was sequentially eluted with MTBE (600 ml), MTBE/AcN 20:1 (600 ml), MTBE/AcN 1:1 (600 ml) and AcN (500 ml). The desired product began come out at MTBE/AcN 20:1. All fractions, contained the desired E7 were combined and evaporated under reduced pressure at 30-35° C. afforded 3.0 g of E7 as white foam. The traces of E7 and minor isomer of E6 were also combined and evaporated to give additional 0.5 g as yellowish foam.

### Preparation of Eribulin (Free Base)

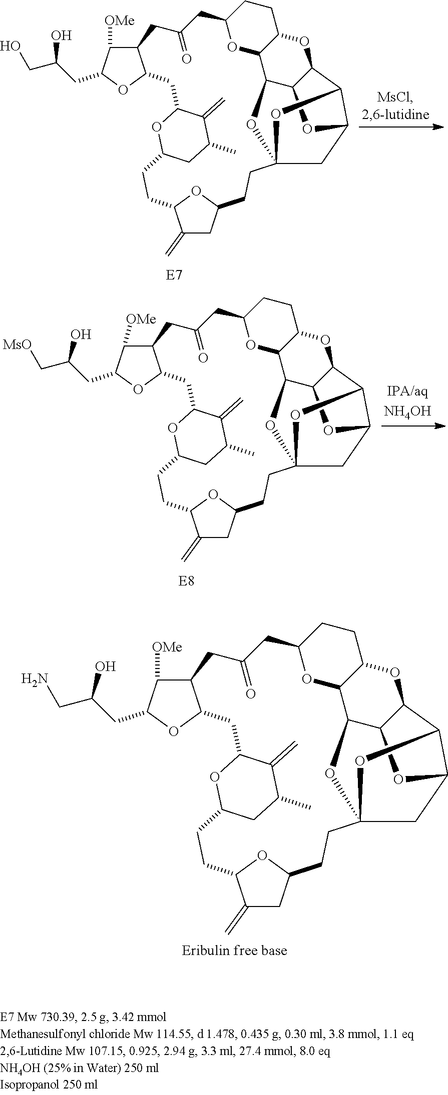

A solution of E7 and 2,6-lutidine in dry DCM (80 ml) was cooled to −5° C. under Argon and a solution of MsCl in dry DCM (30 ml) was slowly added kept the reaction temperature below 5° C. Then the reaction was stirred at 0-5° C. with UPC²/TLC (EtOAc) monitoring. After 145 h (stop-point: less than 3% of E7 or more than 5% of dimesylate) IPA (250 ml) and NH₄OH (250 ml) were added and the stirring was continued at 20-25° C. with UPC²/TLC (EtOAc) control. After 48 h of stirring, the reaction was concentrated under reduced pressure at 30° C. and the residue was portioned between DCM (150 ml) and NaHCO₃/Na₂CO₃/Water solution (9/9/182; 50 ml). The phases were separated, the aqueous on was extracted with DCM (70 ml) and the combined organics were dried over Na₂SO₄, filtered and concentrated under reduced pressure at 30° C. until ˜30 ml volume. The resulted solution was loaded onto Silica gel column (100 g) and eluted with AcN (500 ml), AcN/Water/0.2M aq NH₄OAc (540/45/15 ml), AcN/Water/0.2M aq NH₄OAc (515/70/15 ml) and AcN/Water/0.2M aq NH₄OAc (500/85/15 ml). All fractions, contained the pure product were combined and concentrated under reduced pressure at 40° C. The residue was portioned between DCM (120 ml) and NaHCO₃/Na₂CO₃/Water solution (9/9/182; 50 ml) and phases were separated. The aqueous on was extracted with DCM (50 ml) and the combined organics were dried over Na₂SO₄, filtered and concentrated under reduced pressure at 35° C. afforded 2.1 g (86% yield from E5) of Eribulin (free base) as white solid.

### Preparation of Eribulin Mesylate

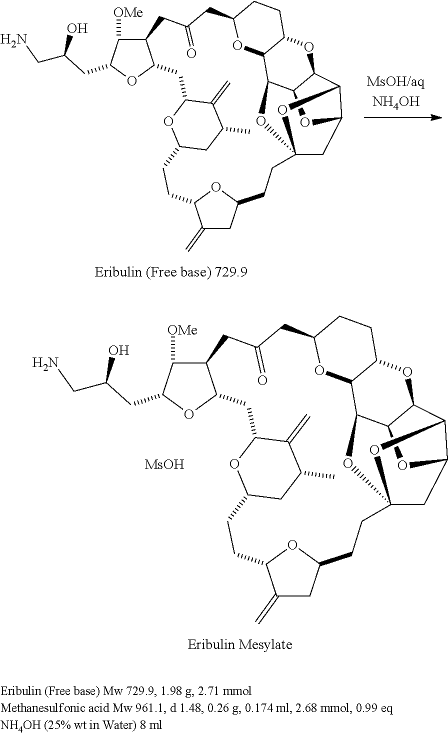

A solution of Eribuline (free base) in Acetonitrile (25 ml) was treated with a solution of Methanesulfonic acid and NH₄OH (8 ml) in Water (28 ml). The resulted mixture was stirred for 10 min, concentrated under reduced pressure at 30° C. and the residue was co-evaporated with Acetonitrile (10 ml) under reduced pressure at 30° C. Co-evaporation was repeated twice to remove the traces of Water. The residue was then dissolved in the mixture DCM/Pentane (3:1 v/v, 60 ml) and filtered through filter with pour size 4 (˜0.45 micron). The solids were washed with the mixture DCM/Pentane (3:1 v/v, 2×10 ml) and the combined filtrates were evaporated under reduced pressure at 30° C.

The residue was re-dissolved in the mixture DCM/Pentane (1:1 v/v, 60 ml) and the resulted solution (if solids still present, filter again) was slowly poured into vigorously stirred dry Pentane (250 ml). The resulted suspension was stirred under Argon for 4 h at 20° C. and filtered under Argon. The solids were washed with dry Pentane (2×30 ml) and dried under reduced pressure at 25° C. afforded 1.9 g (85% yield) of Eribulin Mesylate as white powder.

All filtrates were combined and evaporated to give additional 0.25 g of the white foam.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound represented by the structure of Formula IV12 or isomer thereof: 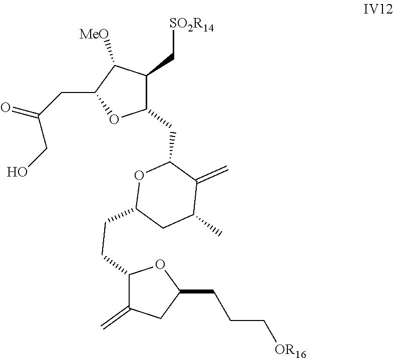
wherein R₁₄ is an alkyl or an aryl; and R₁₆ is an alcohol protecting group.

2. The compound claim 1, wherein the compound of Formula IV12 is represented by the structure of D12 or isomer thereof: 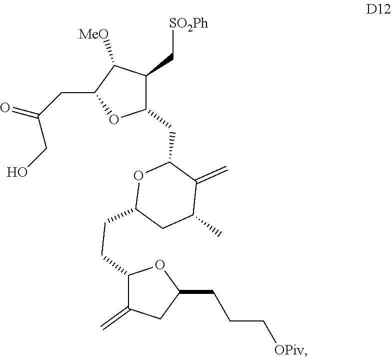
wherein Piv is a pivaloyl group

3. A process for the preparation of a compound of claim 1
wherein the process (Process 7) comprises the following steps:
a) preparing a compound of Formula IV8 or isomer thereof from a compound of Formula IV7 or isomer thereof, according to scheme 29: 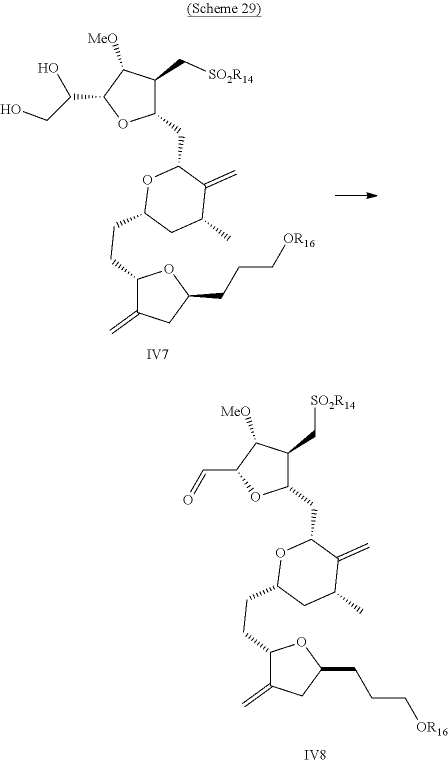
wherein R₁₄ is an alkyl or aryl and R₁₆ is an alcohol protecting group;
b) preparing a compound of Formula IV9 or isomer thereof from a compound of Formula IV8 or isomer thereof, according to scheme 30: 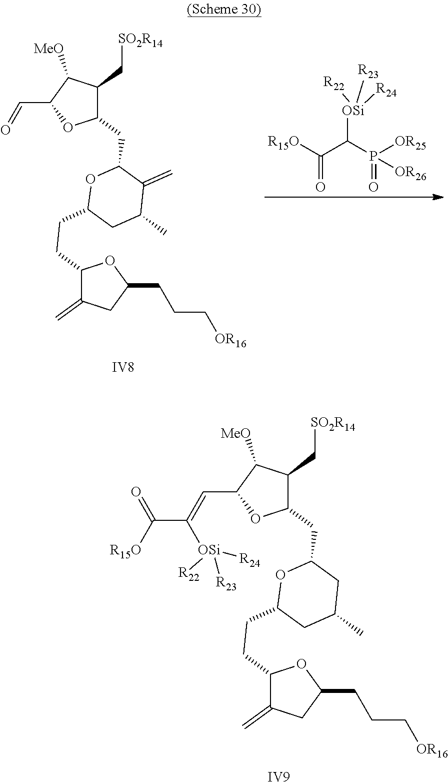
wherein R₁₄ is an alkyl or aryl; R₁₆ is an alcohol protecting group;
and R₁₅, R₂₂, R₂₃, R₂₄, R₂₅ and R₂₆ are each independently an alkyl or an aryl group;
c) preparing a compound of Formula IV1 or isomer thereof from a compound of Formula IV9 or isomer thereof, according to scheme 31: 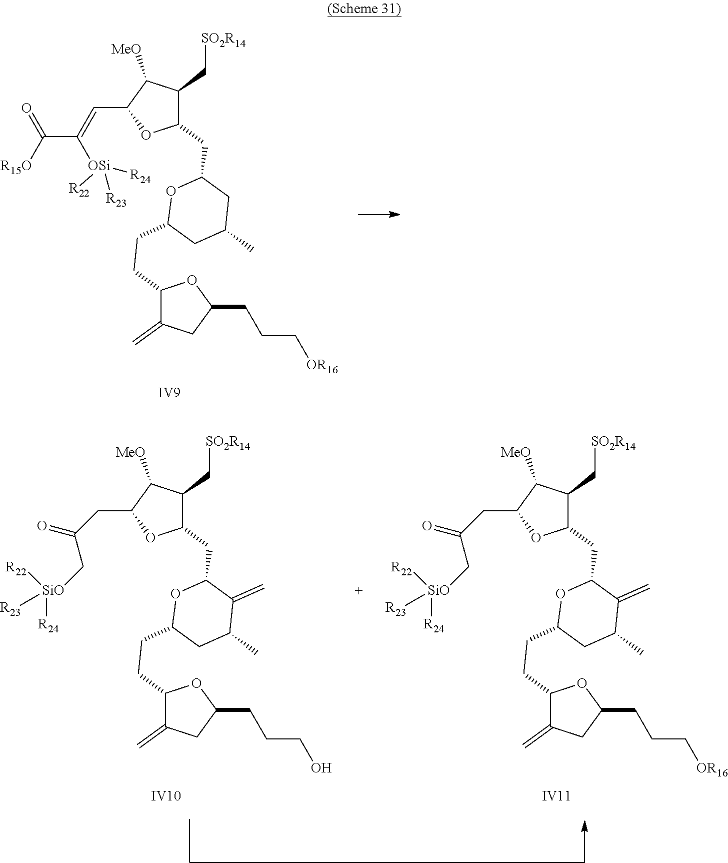
wherein R₁₄ is an alkyl or aryl; R₁₆ is an alcohol protecting group; and R₁₅, R₂₂, R₂₃, and R₂₄ are each independently an alkyl or an aryl;
wherein,
wherein R₁₄ is an alkyl or aryl; R₁₆ is an alcohol protecting group;
R₁₅, R₂₂, R₂₃, and R₂₄, are each independently an alkyl or an aryl group; and
wherein,
the compound of Formula IV10 or isomer thereof is reacted with an alcohol protecting group moiety to obtain a compound of Formula IV1 or isomer thereof; and
d) preparing a compound of Formula IV12 or isomer thereof from a compound of Formula IV11 or isomer thereof, according to scheme 32: 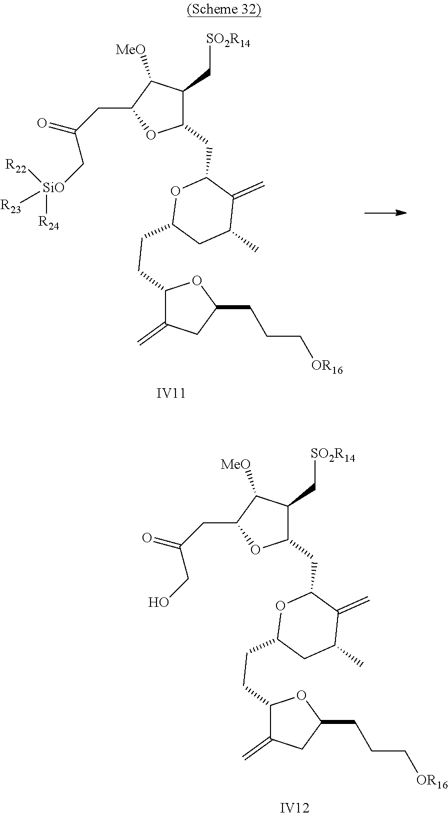
wherein R₁₄ is an alkyl or aryl; R₁₆ is an alcohol protecting group; and R₁₅, R₂₂, R₂₃, and R₂₄, are each independently an alkyl or an aryl group.

4. A process for the preparation of Eribulin, wherein the process comprises the following steps:
(i) preparing a compound of formula IV12 or isomer thereof: 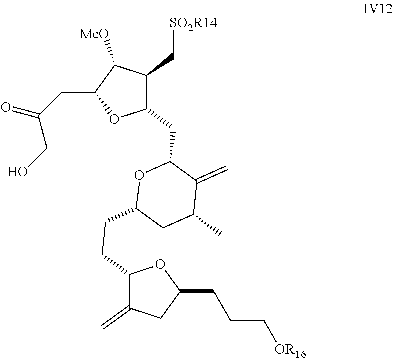
wherein R₁₄ is an alkyl or an aryl; and R₁₆ is an alcohol protecting group;
from a compound of formula IV7 or isomer thereof: 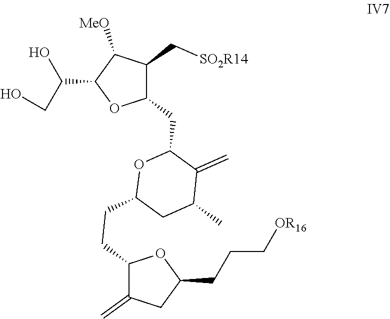
wherein R₁₄ is an alkyl or an aryl; and R₁₆ is an alcohol protecting group by elongating the chain of the diol; and
(ii) preparing Eribulin from a compound of formula IV12 or isomer thereof, wherein the process comprises reducing the ketone group of IV12 or isomer thereof to form diol, protecting the diol group and coupling reaction to obtain Eribulin.

5. The process of claim 3, wherein the reaction of step (a) as described in scheme 29 comprises NaIO₄, H₅IO₆, KO₄, LiIO₄, HIO₄ or combination of NMM and RuCl₃.

6. The process of claim 3, wherein the compound of Formula IV7 or isomer thereof is D7 or isomer thereof; the compound of Formula IV8 or isomer thereof is D8 or isomer thereof; the compound of Formula IV9 or isomer thereof is D9 or isomer thereof; the compound of Formula IV10 or isomer thereof is D10 or isomer thereof; the compound of Formula IV11 or isomer thereof is D11 or isomer thereof; the compound of Formula IV12 or isomer thereof is D12 or isomer thereof

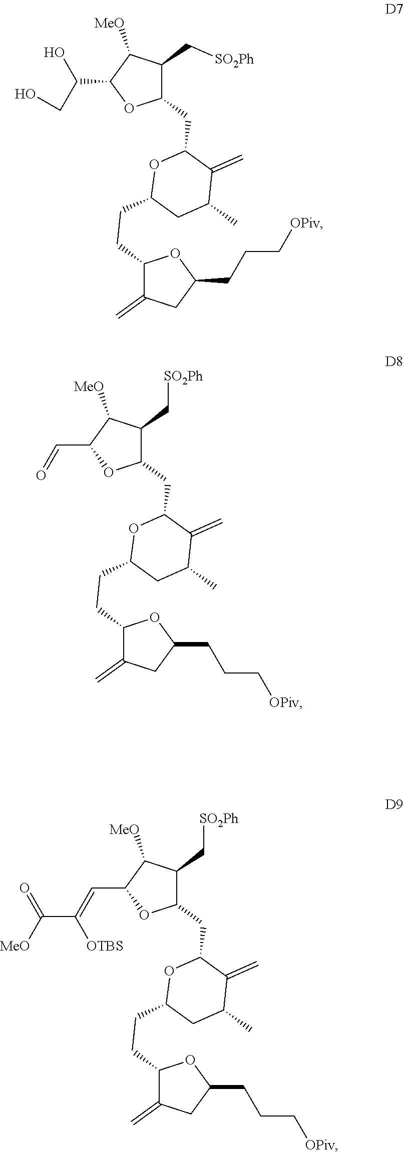

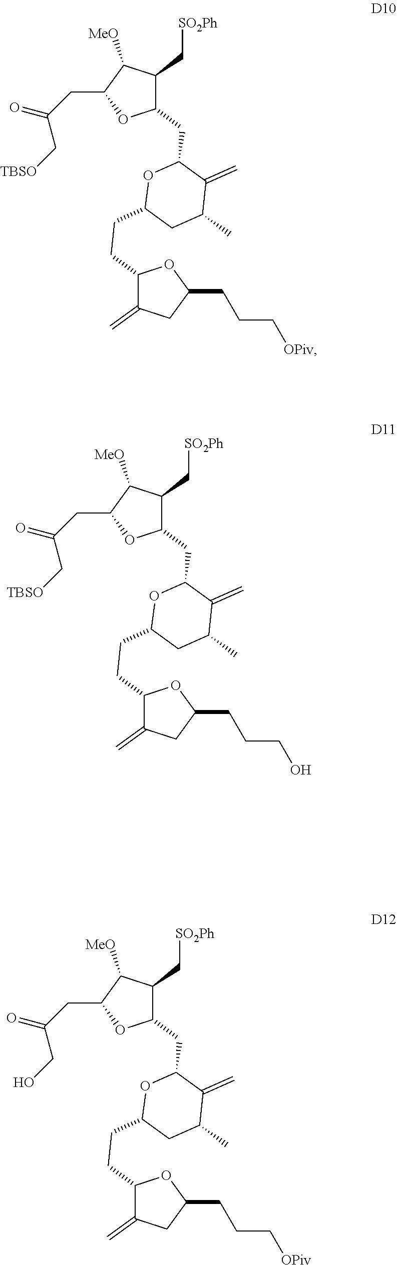

7. A compound represented by the structure of Formula IV7 or isomer thereof: 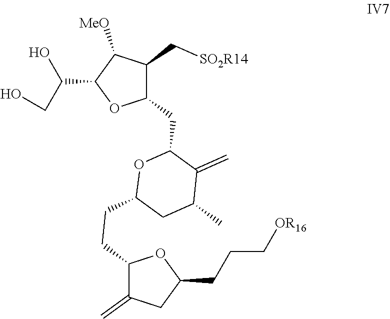
wherein R₁₄ is an alkyl or an aryl; and R₁₆ is an alcohol protecting group.

8. The compound of claim 7, wherein the compound of Formula IV7 or isomer thereof is represented by the structure of D7 or isomer thereof:

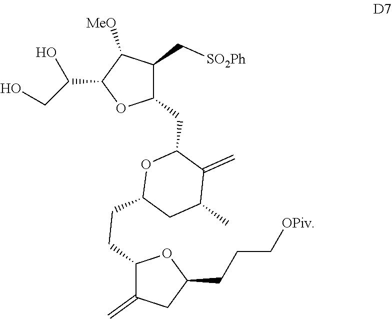

9. The process of claim 3, wherein the compound of Formula IV7 or isomer thereof: 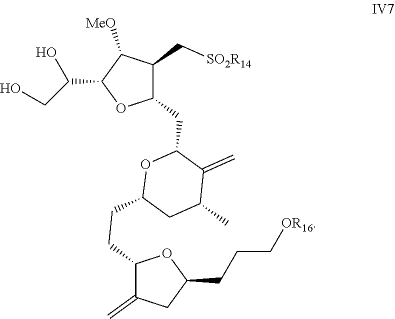
wherein R₁₄ is alkyl or aryl; and R₁₆ is an alcohol protecting group;
is prepared from a compound of Formula III12 or isomer thereof 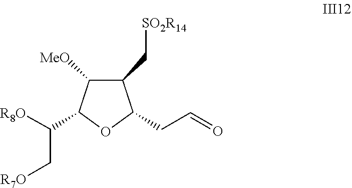
wherein R₇, and R₈ are each independently an alcohol protecting group; or R₇ and R₈ form together with the oxygen a 5-6-member ring optionally substituted; R₁₄, is an alkyl or aryl group.

10. The process of claim 9, wherein the process (Process 8) for the preparation of a compound of Formula IV7 or isomer thereof is prepared from a compound of Formula III12, or isomer thereof 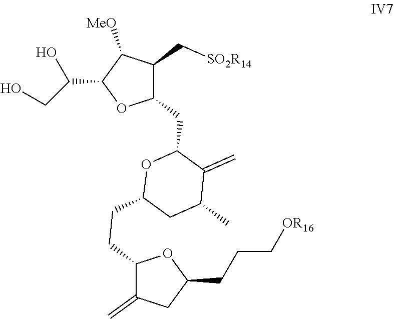
wherein R₁₄ is alkyl or aryl; and R₁₆ is an alcohol protecting group;
comprises the following steps:
a) preparing a compound of Formula IV1 or isomer thereof by reacting a compound of Formula III12 or isomer thereof with a compound of Formula II20 or isomer thereof, according to scheme 33; 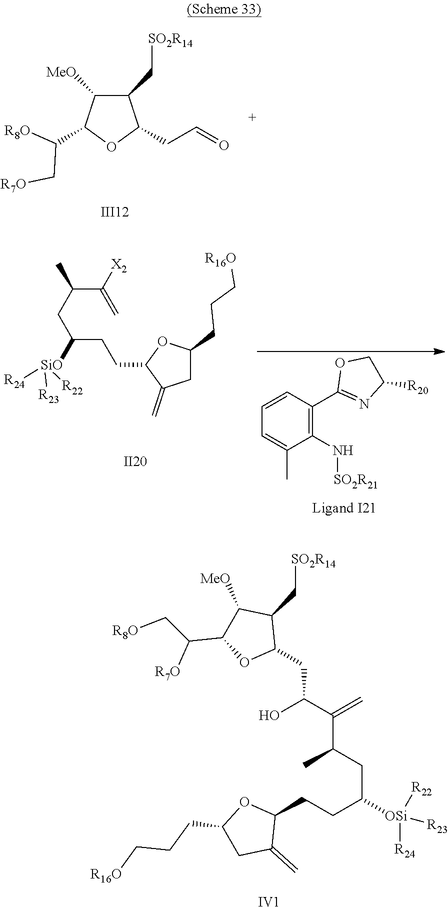
wherein R₇, R₈ and R₁₆ are each independently an alcohol protecting group; or R₇ and R₈ form together with the oxygen a 5-6-member ring optionally substituted; R₁₄, R₂₀, R₂₁ R₂₂, R₂₃ and R₂₄ are each independently an alkyl or aryl group; and X₂ is a halogen;
b) preparing a compound of Formula IV2 or isomer thereof from a compound of Formula IV1 or isomer thereof according to scheme 34: 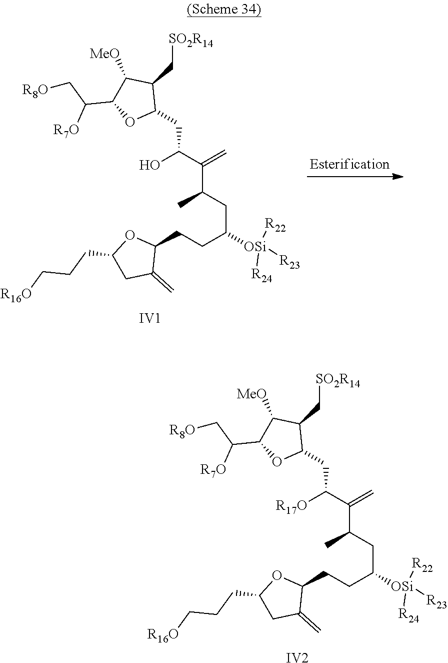
wherein OR₁₇ is an ester; R₇, R₈ and R₁₆ are each independently an alcohol protecting group; or R₇ and R₈ form together with the oxygen a 5-6-member ring optionally substituted; and R₁₄, R₂₂, R₂₃ and R₂₄ are each independently an alkyl or aryl group;
c) preparing a compound of Formula IV3 or isomer thereof from a compound of Formula IV2 or isomer thereof according to scheme 35; 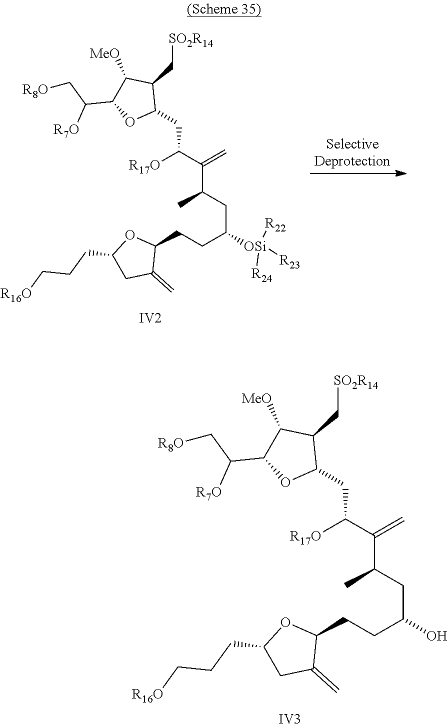
wherein OR₁₇ is an ester; R₇, R₈ and R₁₆ are each independently an alcohol protecting group; or R₇ and R₈ form together with the oxygen a 5-6-member ring optionally substituted; and R₁₄, R₂₂, R₂₃ and R₂₄ are each independently an alkyl or aryl group;
d) preparing a compound of Formula IV4 or isomer thereof from a compound of Formula IV3 or isomer thereof according to scheme 36: 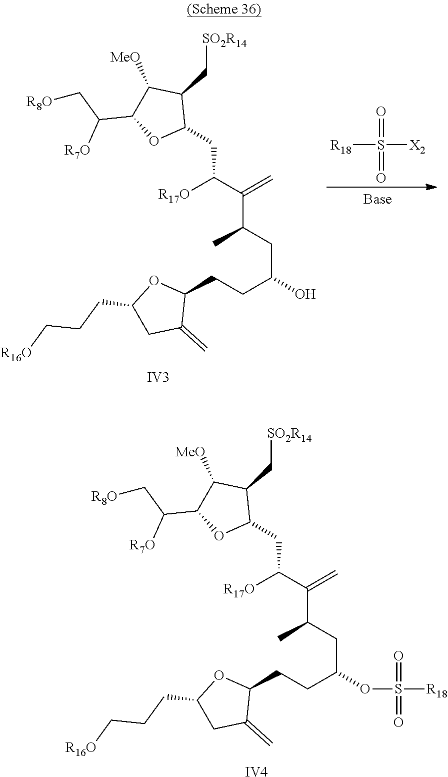
wherein X₂ is a halogen; R₁₈ and R₁₄ are each independently an alkyl or an aryl; OR₁₇ is an ester; R₇, R₈ and R₁₆ are each independently an alcohol protecting group; or R₇ and R₈ form together with the oxygen a 5-6-member ring optionally substituted; R₁₄, is an alkyl or aryl group;
e) preparing a compound of Formula IV6 or isomer thereof from a compound of Formula IV4 or isomer thereof according to scheme 37: 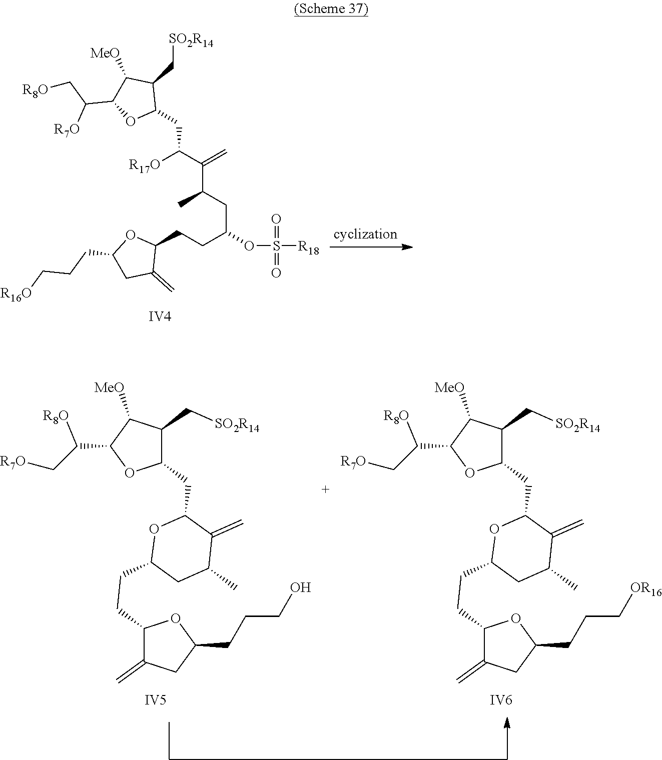
wherein R₁₈ is an alkyl or an aryl; OR₁₇ is an ester; R₇, R₈ and R₁₆ are each independently an alcohol protecting group; or R₇ and R₈ form together with the oxygen a 5-6-member ring optionally substituted; and
wherein, the compound of Formula IV5 or isomer thereof is reacted with an alcohol protecting group moiety to obtain a compound of Formula IV6 or isomer thereof; and
f) preparing a compound of Formula IV7 or isomer thereof from a compound of Formula IV6 or isomer thereof according to scheme 38: 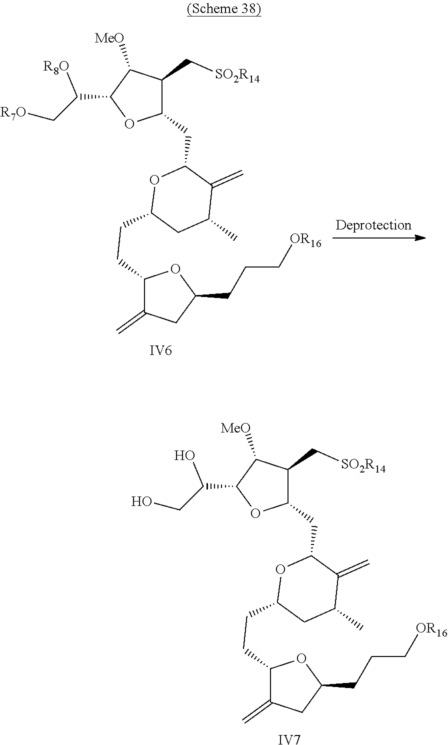
wherein R₁₄ are each independently an alkyl or an aryl; OR₁₇ is an ester; R₇, R₈ and R₁₆ are each independently an alcohol protecting group; or R₇ and R₈ form together with the oxygen a 5-6-member ring optionally substituted; R₁₄, is an alkyl or aryl group.

11. The process of claim 10, wherein the esterification reaction of step (b) as described in scheme 34 comprises (i) acyl anhydride or acyl halide and (ii) a base.

12. The process of claim 10, wherein the selective deprotection reaction of step (c) as described in scheme 35 comprises acidic conditions.

13. The process of claim 12, wherein the acidic condition comprises a catalytic amount of acid or a fluoride anion source.

14. The process of claim 10, wherein the cyclization reaction step (e) as described in scheme 37 comprises a strong base.

15. The process of claim 10, wherein the compound of Formula IV1 or isomer thereof is D1 or isomer thereof; the compound of Formula IV2 or isomer thereof is D2 or isomer thereof; the compound of Formula IV3 or isomer thereof is D3 or isomer thereof; the compound of Formula IV4 or isomer thereof is D4 or isomer thereof; the compound of Formula IV5 or isomer thereof is D5 or isomer thereof; the compound of Formula IV6 or isomer thereof is D6 or isomer thereof; the compound of Formula IV7 or isomer thereof is D7 or isomer thereof:

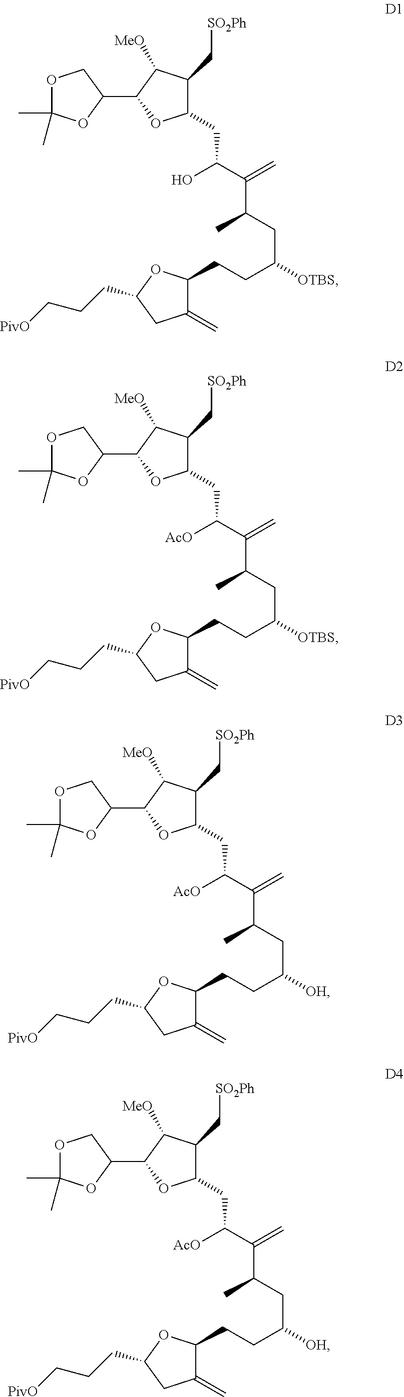

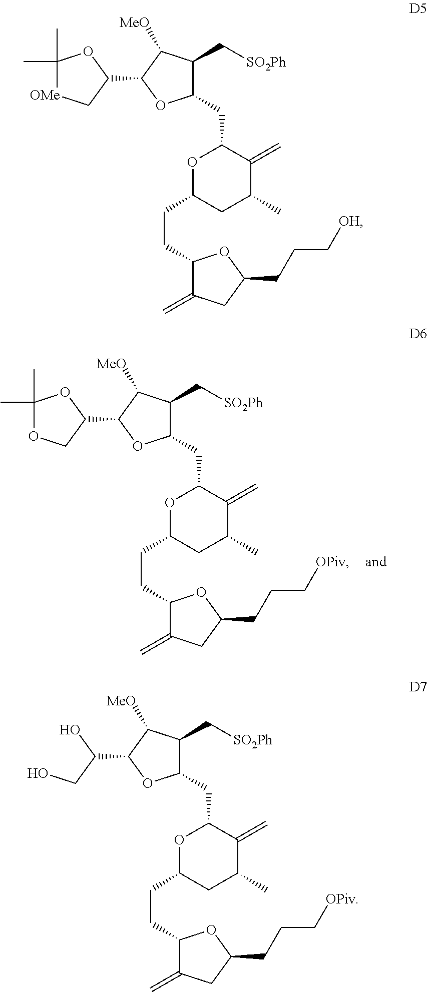

16. The process of claim 3, wherein the process (Process 9) a compound of Formula IV7 or isomer thereof is prepared from a compound of Formula III12 or isomer thereof, 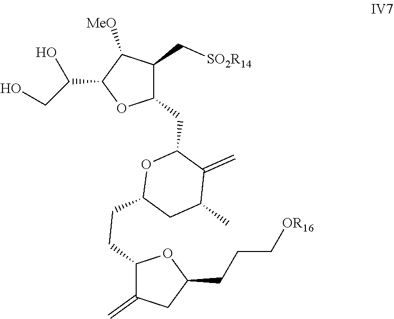
wherein R₁₄ is alkyl or aryl; and R₁₆ is an alcohol protecting group;
comprises the following steps:
a) preparing a compound of Formula IV17 or isomer thereof by reacting a compound of Formula III12 or isomer thereof with a compound of Formula II28 or isomer thereof, according to scheme 39: 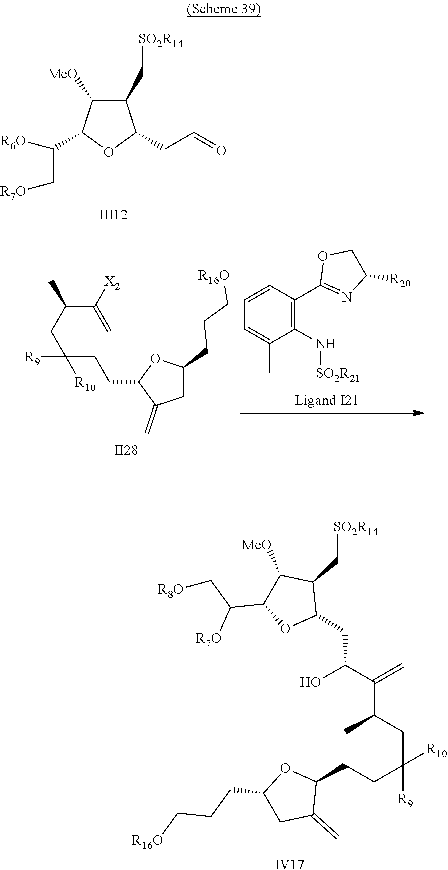
wherein R₇ and R₈ are each independently an alcohol protecting group; or R₇ and R₈ form together with the oxygen a 5-6-member ring optionally substituted; R₉ and R₁₀ are each independently O-alkyl or S-alkyl; or R₉ and R₁₀ form together a 5-6-member acetal ring optionally substituted; X₂ is a halogen; R₁₆ is an alcohol protecting group; and R₁₄, R₂₀ and R₂₁ are each individually an alkyl or an aryl group;
b) preparing a compound of Formula IV7 or isomer thereof from a compound of Formula IV17 or isomer thereof, according to scheme 40: 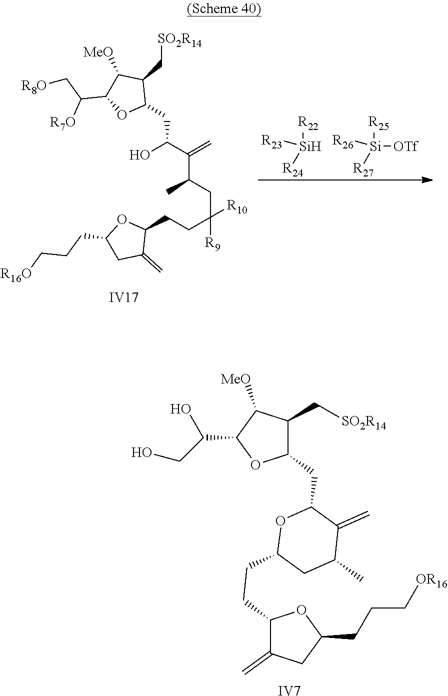
wherein R₇, R₈ and R₁₆ are each independently an alcohol protecting group; or R₇ and R₈ form together with the oxygen a 5-6-member ring optionally substituted; R₉ and R₁₀ are each independently O-alkyl or S-alkyl; or R₉ and R₁₀ form together a 5-6-member acetal ring optionally substituted; and R₁₄, R₂₂, R₂₃, R₂₄, R₂₅, R₂₆ and R₂₇ are each individually an alkyl or an aryl group.

17. The process of claim 16, wherein the compound of Formula IV17 or isomer thereof is 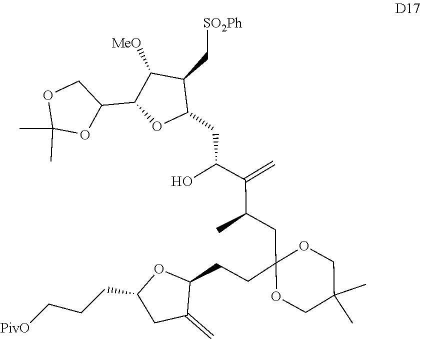
or isomer thereof.

18. The process of claim 3, wherein the process (Process 10) of a compound of Formula IV7 or isomer thereof is prepared from a compound of Formula III12 or isomer thereof, 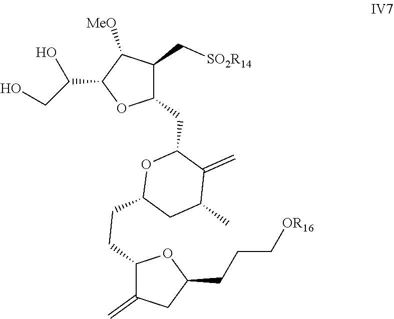
wherein R₁₄ is alkyl or aryl; and R₁₆ is an alcohol protecting group;
comprises the following steps:
a) preparing a compound of Formula IV16 or isomer thereof by reacting a compound of Formula III12 or isomer thereof with a compound of Formula II29 or isomer thereof, according to scheme 41: 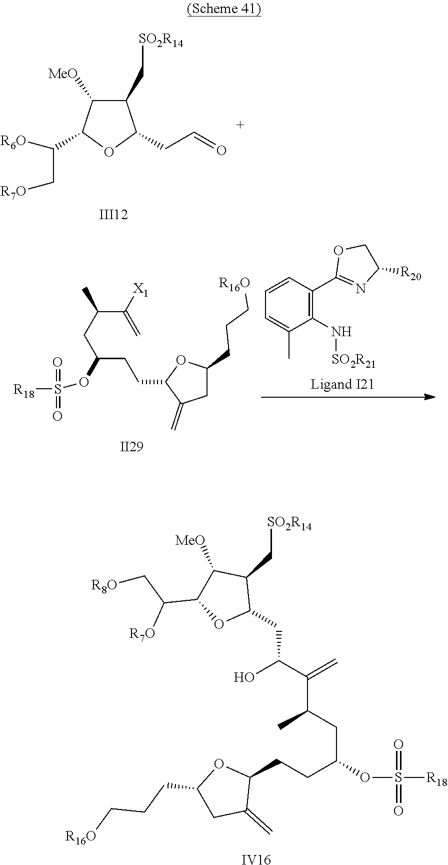
wherein R₇ and R₈ are each independently an alcohol protecting group; or R₇ and R₈ form together with the oxygen a 5-6-member ring optionally substituted; X₁ is a leaving group; R₁₄ and R₁₈ are each independently an alkyl or an aryl; R₁₆ is an alcohol protecting group; and R₂₀ and R₂₁ are each independently an alkyl or an aryl group;
b) preparing a compound of Formula IV6 or isomer thereof from a compound of Formula IV16 or isomer thereof, according to scheme 42: 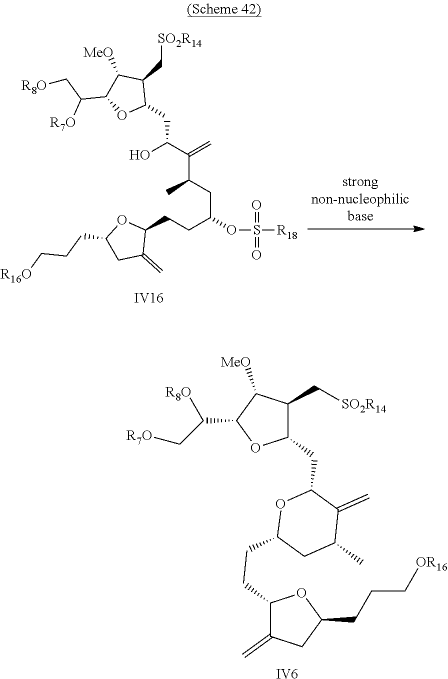
wherein R₇ and R₈ are each independently an alcohol protecting group; or R₇ and R₈ form together with the oxygen a 5-6-member ring optionally substituted; X₁ is a leaving group; R₁₄, R₁₈, R₂₀, and R₂₁ are each independently an alkyl or an aryl; R₁₆ is an alcohol protecting group; and
c) preparing a compound of Formula IV7 or isomer thereof from a compound of Formula IV6 or isomer thereof, according to scheme 43: 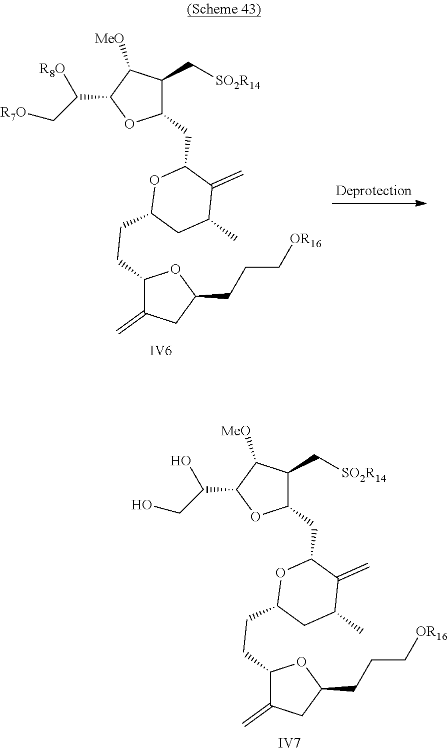
wherein R₇, R₈ and R₁₆ are each independently an alcohol protecting group; or R₇ and R₈ form together with the oxygen a 5-6-member ring optionally substituted; R₁₄ is an alkyl or an aryl

19. The process of claim 18, wherein the compound of Formula IV16 or isomer thereof is

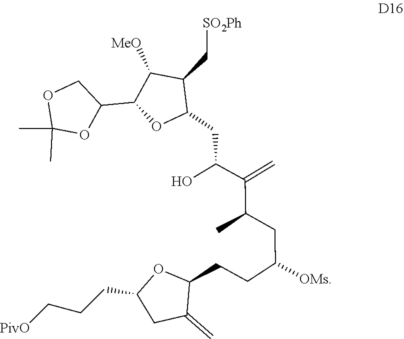

20. The process of claim 9,
wherein the process for the preparation of a compound of Formula III12 or isomer thereof: 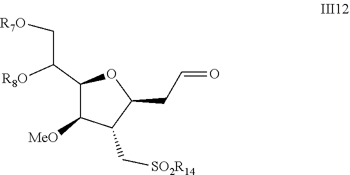
wherein R₇ and R₈ are each independently an alcohol protecting group or R₇ and R₈ form together with the oxygen a 5-6 member ring optionally substituted; and R₁₄ is an alkyl or an aryl;
wherein the process (Process 6) comprises the following steps:
a) preparing a compound of Formula III5 or isomer thereof from a compound of Formula III4 or isomer thereof according to scheme 24: 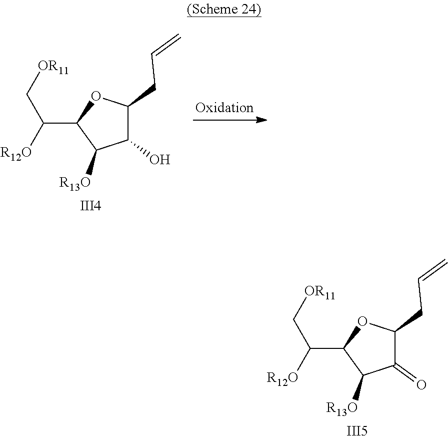
wherein OR₁₁ and OR₁₂ are each independently an ester group; R₁₃ is —CH₂-aryl or 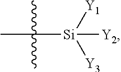
wherein Y₁, Y₂, and Y₃ are each independently an alkyl or an aryl;
b) preparing a compound of Formula III6 or isomer thereof from a compound of Formula III5 or isomer thereof according to scheme 25: 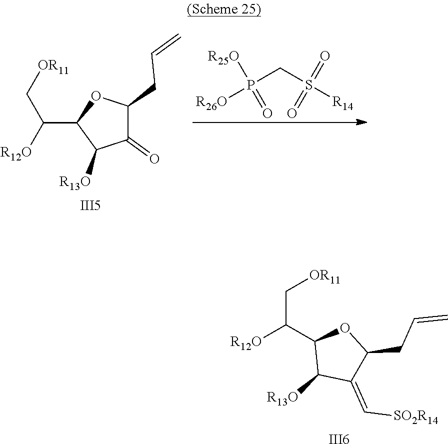
wherein R₁₄, R₂₅, and R₂₆ are each independently an alkyl or an aryl group;
OR₁₁ and OR₁₂ are each independently an ester group; R₁₃ is —CH₂-aryl or 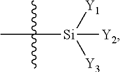
wherein Y₁, Y₂, and Y₃ are each independently an alkyl or an aryl;
c) preparing a compound of Formula III9 or isomer thereof from a compound of Formula III6 or isomer thereof according to scheme 26: 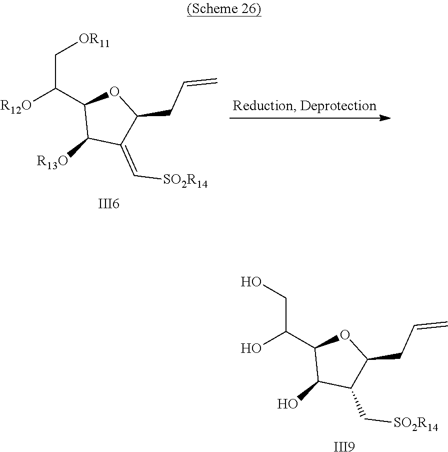
wherein OR₁₁ and OR₁₂ are each independently an ester group; R₁₃ is —CH₂-aryl or 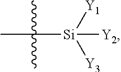
wherein Y₁, Y₂, and Y₃ are each independently an alkyl or an aryl; R₁₄ is an alkyl or an aryl group;
wherein OR₁₁, OR₁₂ and OR₁₃ are deprotected and the non-terminal double bond is reduced in any sequence;
d) preparing a compound of Formula III11 or isomer thereof from a compound of Formula III9 or isomer thereof according to scheme 27: 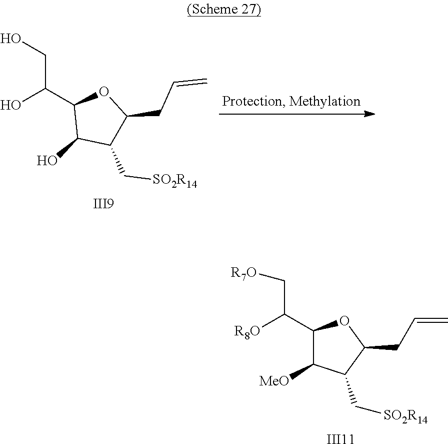
wherein R₇ and R₈ are each independently an alcohol protecting group or R₇ and R₈ form together with the oxygen a 5-6 member ring optionally substituted; and R₁₄ is an alkyl or an aryl; and
e) preparing a compound of Formula III12 or isomer thereof from a compound of Formula III11 or isomer thereof according to scheme 28: 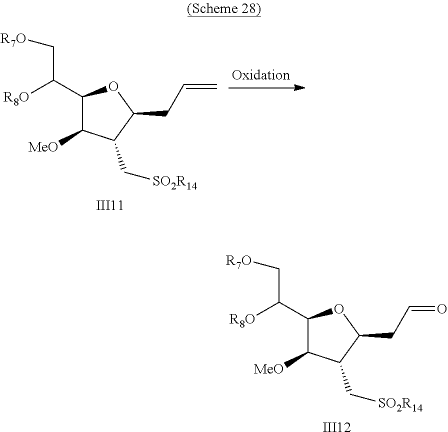
wherein R₇ and R₈ are each independently an alcohol protecting group or R₇ and R₈ form together with the oxygen a 5-6 member ring optionally substituted; and R₁₄ is an alkyl or an aryl.

21. The process of claim 18, wherein the oxidation reaction of step (a) as described in scheme 24 comprises DMSO-based oxidation, IBX, DMP, Swern oxidation, radical oxidation, PDC, PCC, or BAIB/TEMPO.

22. The process of claim 18, wherein the condensation and reduction reaction of step (b) as described in scheme 25 comprises reaction with a non nucleophilic base.

23. The process of claim 18, wherein the oxidation step of step (e) scheme 28 comprises OsO₄ and NaIO₄, or, a combination of O₃ with triphenylphosphine or dimethylsulfide or poisoned Pd.

24. The process of claim 18, wherein the compound of Formula III5 or isomer thereof is C5 or isomer thereof; the compound of Formula III6 or isomer thereof is C6 or isomer thereof; the compound of Formula III9 or isomer thereof is C9 or isomer thereof; the compound of Formula III11 or isomer thereof is C11 or isomer thereof; the compound of Formula III12 or isomer thereof is C12 or isomer thereof or C12a or isomer thereof

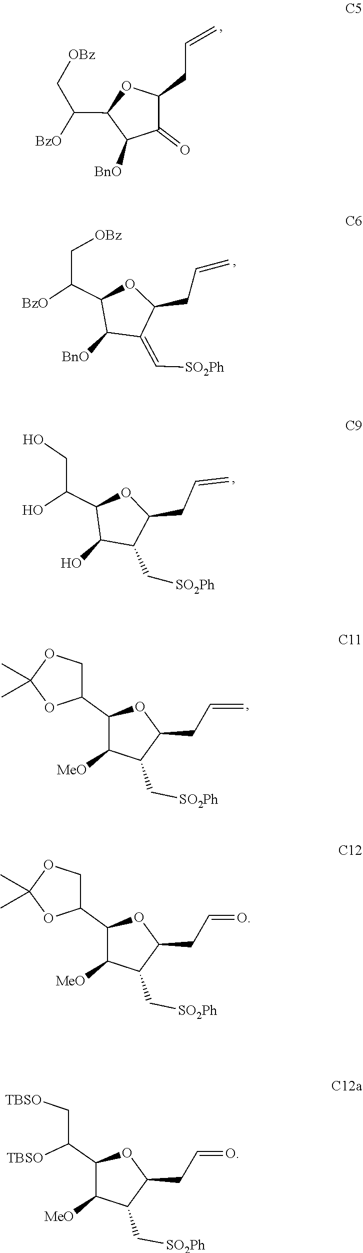

25. A compound represented by the structure of Formula III12 or isomer thereof: 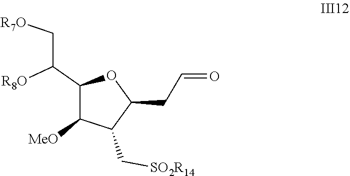
wherein R₇ and R₈ are each independently an alcohol protecting group or R₇ and R₈ form together with the oxygen a 5-6 member ring optionally substituted; and R₁₄ is an alkyl or an aryl.

26. The compound of claim 25, wherein the compound of Formula III12 or isomer thereof is represented by the structure of C12 or isomer thereof or C12a or isomer thereof: [chemical structure]
